(19) United States
(12) Patent Application Publication
(10) Pub. No.: US 2009/0325936 A1
(43) Pub. Date: Dec. 31, 2009

(54) IMIDAZOPYRIDINE ANALOGS AS CB2 RECEPTOR MODULATORS, USEFUL IN THE TREATMENT OF PAIN, RESPIRATORY AND NON-RESPIRATORY DISEASES

(76) Inventors: Mark T. Bilodeau, Lansdale, PA (US); Christopher S. Burgey, Philadelphia, PA (US); Zhengwu James Deng, Eagleville, PA (US); Nathan R. Kett, Perkiomenville, PA (US); Jeffrey Melamed, North Wales, PA (US); Peter M. Munson, Harleysville, PA (US); Kausik K. Nanda, Morristown, PA (US); Wayne Thompson, Lansdale, PA (US); B. Wesley Trotter, Glenside, PA (US); Zhicai Wu, Montvale, NJ (US); John C. Hartnett, Philadelphia, PA (US)

Correspondence Address: MERCK AND CO., INC, P O BOX 2000, RAHWAY, NJ, 07065-0907, US

(21) Appl. No.: 12/519,789

(22) PCT Filed: Dec. 14, 2007
(86) PCT No.: PCT/US07/25641

§ 371 Date: Jun. 18, 2009

Related U.S. Application Data
(60) Provisional application No. 60/876,105, filed on Dec. 20, 2006.

(51) Int. Cl. A61K 31/437; C07D 471/04; A61K 31/506; A61K 31/497; A61K 31/5377; A61K 31/551; A61K 31/541; A61P 19/00; A61P 25/00

(52) U.S. Cl. 514/218; 546/121; 544/328; 544/405; 544/127; 540/575; 544/58.2; 514/300; 514/256; 514/255.05; 514/233.2; 514/228.2

(57) ABSTRACT

The present invention relates to compounds represented by Formula (I) and Formula (II): (I) (II) or pharmaceutically acceptable salts thereof. The present invention also provides pharmaceutical compositions comprising the instant compounds. This invention further provides methods to treat and prevent pain, respiratory and non-respiratory diseases.

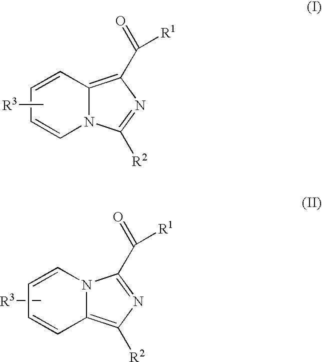

### FIELD OF THE INVENTION

This invention relates to compounds useful as cannibinoid receptor modulators. More particularly, this invention relates to compounds that are CB2 modulators. Even more particularly, this invention relates to compounds that are selective CB2 agonists. The compounds of the invention are useful in the treatment pain and an array of respiratory and non-respiratory diseases, as further discussed infra.

### BACKGROUND OF THE INVENTION

Cannabinoids are psychoactive natural products present in Cannabis sativa L. and have been used as therapeutic agents for thousands of years. They have been shown to have myriad effects in humans, notably in the central nervous system and the cardiovascular system. The therapeutic utility of cannabis is significantly limited due to adverse central effects. The effects of cannabinoids have been shown to occur through their action on two G-protein coupled receptors. A first receptor, CB1, is primarily a centrally-expressed receptor with more limited expression in a variety of peripheral sites, and is believed to be primarily responsible for the central effects of cannabinoids. A second receptor, CB2, is preferentially expressed in the periphery, primarily in cells of the immune system, although it has been identified in central locations to a lesser extent. CB2, expressed in immune cells such as T cells, B cells, macrophages and mast cells, has been shown to have a specific role in mediating immune and inflammatory responses. Given the role of the CB2 receptor in immunomodulation, it is an attractive target for chronic inflammatory pain. CB2 modulators also may have a role in the treatment of osteoporosis, atheroschlerosis, immune disorders, arthritis and other pathological conditions, as discussed infra.

The effects of cannabinoids are due to interaction with specific high affinity receptors, coupled to G proteins, present at the central level (Devane et al., Molecular Pharmacology (1988), 34, 605-613) and the peripheral level (Nye et al., J. Pharmacol. and Exp. Ther. (1985), 234, 784-791; Kaminski et al., Molecular Pharmacol. (1992), 42, 736-742; Munro et al., Nature (1993), 365, 61-65).

The central effects of cannabinoids relate to a first type of cannabinoid receptor (CB1) which is present mainly in the brain but also in the periphery. Munro et al. [Nature, (1993) 365, 61-65] have cloned a second type of cannabinoid receptor, CB2, which is present in the periphery and more particularly on cells of immune origin. The presence of CB2 cannabinoid receptors on lymphoid cells may explain the immunomodulation mentioned above exerted by agonists for cannabinoid receptors.

The psychotropic effects of cannabinois as well as their influence on immune function has been described. [HOLLISTER L. E., J. Psychoact. Drugs, 24 (1992), 159-164]. Most of the in vitro studies have shown immunosuppressant effects for cannabinoids: the inhibition of the proliferative responses in T lymphocytes and B lymphocytes induced by mitogens [Luo, Y. D. et al., Int. J. Immunopharmacol., (1992) 14, 49-56, Schwartz, H. et al., J. Neuroimmunol., (1994) 55, 107-115], the inhibition of the activity of cytotoxic T cells [Klein et al., J. Toxicol. Environ. Health, (1991) 32, 465-477], the inhibition of the microbiocidal activity of macrophages and of the synthesis of TNF-α. [Arata, S. et al., Life Sci., (1991) 49, 473-479; Fisher-Stenger et al., J. Pharm. Exp. Ther., (1993) 267, 1558-1565], the inhibition of the cytolytic activity and of the production of TNF-α. of large granular lymphocytes [Kusher et al., Cell. Immun., (1994) 154, 99-108]. In some studies, amplification effects were observed: increase in the bioactivity of interleukin-1 by mice resident macrophages or differentiated macrophage cell lines, due to increased levels of TNF-α. [Zhu et al., J. Pharm. Exp. Ther., (1994) 270, 1334-1339; Shivers, S. C. et al., Life Sci., (1994) 54, 1281-1289].

Certain Imadazo[1,5-a]pyridine analogs have been disclosed as useful for the inhibition of fibroblast growth factor. See WO2006097625, published Sep. 21, 2006.

### SUMMARY OF THE INVENTION

The present invention relates to compounds represented by Formula (I) and Formula (II):

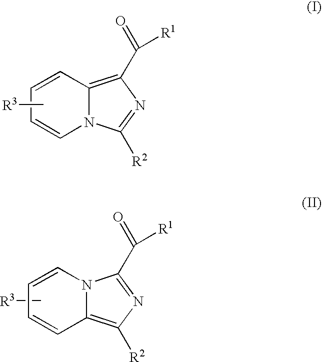

or pharmaceutically acceptable salts thereof. The present invention also provides pharmaceutical compositions comprising the instant compounds. This invention further provides methods to treat and prevent pain, respiratory and non-respiratory diseases.

### DETAILED DESCRIPTION OF THE INVENTION

In one embodiment the present invention relates to compounds represented by Formula (I) and Formula (II):

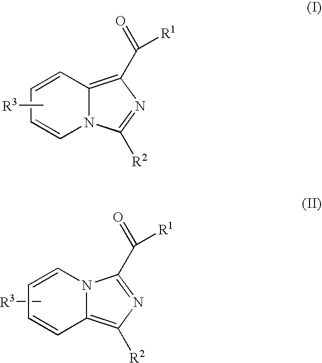

and pharmaceutically acceptable salts thereof, wherein

R¹ is selected from the group consisting of:
(1) —NH—R⁴,
(2) —O—R⁴,
(3) —CH2-C(O)—R⁴, and
(4) —R⁴;

R² is selected from the group consisting of:
(1) H,
(2) halo,
(3) —CN,
(4) —CF₃,
(5) —C1-6alkyl,
(6) —C(O)—NH-C₁₋₃alkyl-CF₃,
(7) —C(O)—NH-C₁₋₃alkyl-heteroaryl, wherein the heteroaryl is optionally mono, di or tri-substituted with substituents independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl, is optionally mono or di-substituted with substituents independently selected from halo, —CH₃, —CF₃, —CN and —OC₁₋₆alkyl,
(8) —C(O)-heteroaryl, wherein the heteroaryl is optionally mono, di or tri-substituted with substituents independently selected from —CN, —CH₃, —CF₃, —CHF₂, —O₁₋₆alkyl, —O—CF₃, hydroxy, —CH₂—OH, halo, —S(O)₂—CH₃, —C(O)—O—C₁₋₆alkyl-C(O)—NHC₁₋₆alkyl, —C(O)—N(C₁₋₆alkyl)₂, —C(O)—O—C(CH₃)₃, —C₃₋₆cycloalkyl, —NH₂, —NH₂—C(O)—CF₃, —NH₂—C(O)—N(CH₃)₂, —NC(O)—NH₂, —NH—S(O)₂—CH₃ and —C(O)-heteroaryl optionally mono or di-substituted with substituents independently selected from halo, —CH₃, —CF₃, —CN and —OC₁₋₆alkyl,
(9) —C(O)-heterocycle, wherein the heterocycle is optionally mono, di- or tri-substituted with substituents selected from —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and oxo, wherein the heteroaryl portion of —C(O)-heteroaryl, is optionally mono or di-substituted with substituents independently selected from halo, —CH₃, —CF₃, —CN and —OC₁₋₆alkyl,
(10) —NR⁵R⁶,
(11) —C₁₋₄alkyl-NR⁵R⁶,
(12) —C₁₋₄alkyl-heterocycle, wherein the alkyl is optionally substituted with hydroxyl and wherein the heterocycle is optionally mono, di- or tri-substituted with substituents independently selected from —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, oxo and —C(O)-heteroaryl, wherein the heteroaryl portion of —C(O)-heteroaryl, is optionally mono or di-substituted with substituents independently selected from halo, —CH₃, —CF₃, —CN and —OC₁₋₆alkyl,
(13) —C₁₋₄alkyl-heteroaryl, optionally mono, di- or tri-substituted with substituents independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl, is optionally mono or di-substituted with substituents independently selected from halo, —CH₃, —CF₃, —CN and —OC₁₋₆alkyl,
(14) heterocycle, optionally mono, di- or tri-substituted with substituents independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, and oxo, wherein the heteroaryl portion of —C(O)-heteroaryl, is optionally mono or di-substituted with substituents independently selected from halo, —CH₃, —CF₃, —CN and —OC₁₋₆alkyl,
(15) heteroaryl, optionally mono, di- or tri-substituted with substituents independently selected from —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl, is optionally mono or di-substituted with substituents independently selected from halo, —CH₃, —CF₃, —CN and —OC₁₋₆alkyl,
(16) aryl, optionally mono, di- or tri-substituted with substituents independently selected from the group consisting of wherein the aryl is optionally mono or di-substituted with substituents selected from —CH₃, —O—C₁₋₆alkyl, hydroxy, —CH₂—OH, halo, —S(O)₂—CH₃, C(O)—O—C₁₋₆alkyl, —C(O)—NHC₁₋₆alkyl, —C(O)—N(C₁₋₆alkyl)₂, —(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, wherein the heteroaryl portion of —C(O)-heteroaryl, is optionally mono or di-substituted with substituents independently selected from halo, —CH₃, —CF₃, —CN and —OC₁₋₆alkyl,
(17) O-aryl, optionally mono, di- or tri-substituted with substituents independently selected from —CH₃, —OC₁₋₆alkyl, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, wherein the heteroaryl portion of —C(O)-heteroaryl, is optionally mono or di-substituted with substituents independently selected from halo, —CH₃, —CF₃, —CN and —OC₁₋₆alkyl,
(18) —O-heteroaryl, optionally mono, di- or tri-substituted with substituents independently selected from —CN, —CH₃, —CF₃, —CHF₂, —O—C₁₋₆alkyl, —O—CF₃, hydroxy, —CH₂—OH, halo, —S(O)₂—CH₃, —C(O)—O—C₁₋₆alkyl, —C(O)—N(CH₃)2, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl, is optionally mono or di-substituted with substituents independently selected from halo, —CH₃, —CF₃, —CN and —OC₁₋₆alkyl,
(19) —NH-heteroaryl, wherein the heteroaryl is optionally mono, di- or tri-substituted with substituents independently selected from —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl, is optionally mono or di-substituted with substituents independently selected from halo, —CH₃, —CF₃, —CN and —OC₁₋₆alkyl, and
(20) —C₃₋₆cycloalkyl, optionally mono, di- or tri-substituted with substituents selected —CH₃, —O—C₁₋₆alkyl, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, oxo, C(O)—O—C(CH₃)₃, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN;

R³ is selected from the group consisting of
(1) H,
(2) halo,
(3) —C₁₋₄alkyl, optionally substituted with hydroxyl,
(4) —CF₃, and
(5) —OC₁₋₆alkyl;
(6) —CN,
(7) —CHF₂,
(8) —O—CF₃,
(9) hydroxy,
(10) —S(O)₂—CH₃,
(11) —C(O)—O—C₁₋₆alkyl,
(12) —C(O)—NHC₁₋₆alkyl,
(13) —C(O)—N(C₁₋₆alkyl)₂,
(14) —C(O)—O—C(CH₃)₃,
(15) —C(O)-heteroaryl,
(16) —C₃₋₆cycloalkyl,
(17) —NH₂,
(18) —NH₂—C(O)—CF₃,
(19) —NH₂—C(O)—N(CH₃)₂,
(20) —NC(O)—NH₂,
(21) —NH—S(O)₂—CH₃,
(22) heteroaryl, optionally mono, di- or tri-substituted with substituents independently selected from —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl, is optionally mono or di-substituted with substituents independently selected from halo, —CH₃, —CF₃, —CN and —OC₁₋₆alkyl, and
(23) aryl, optionally mono, di- or tri-substituted with substituents selected from the group consisting of wherein the aryl is optionally mono or di-substituted with substituents selected from —CH₃, —O—C₁₋₆alkyl, hydroxy, —CH₂—OH, halo, —S(O)₂—CH₃, C(O)—O—C₁₋₆alkyl, —C(O)—NHC₁₋₆alkyl, —C(O)—N(C₁₋₆alkyl)₂, —(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, wherein the heteroaryl portion of —C(O)-heteroaryl, is optionally mono or di-substituted with substituents independently selected from halo, —CH₃, —CF₃, —CN and —OC₁₋₆alkyl;

R⁴ is selected from the group consisting of:
(1) adamantane, optionally mono and di-substituted with substituents independently selected from —CH₃ and hydroxyl,
(2) aryl, optionally mono, di- or tri-substituted with substituents independently selected from —CH₃, —OC₁₋₆alkyl, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, wherein the heteroaryl portion of —C(O)-heteroaryl is optionally mono, di- or tri-substituted with substituents selected from the group consisting of halo, —CH₃, —CF₃, —CN, —OC₁₋₆alkyl,
(3) heteroaryl, optionally mono, di- or tri-substituted with a substituents independently selected from —CN, —CH₃, —CF₃, —CHF₂, —OC₁₋₆alkyl, —O—CF3, hydroxy, —CH₂—OH, halo, —S(O)₂—CH₃, —C(O)—O—CH₃, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl is optionally mono, di- or tri-substituted with substituents selected from the group consisting of halo, —CH₃, —CF₃, —CN, —OC₁₋₆alkyl,
(4) heterocycle, optionally mono, di-, tri- or tetra substituted with a substituent selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl,—C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, oxo and aryl, and wherein aryl and the heteroaryl portion of —C(O)-heteroaryl is optionally mono, di- or tri-substituted with substituents selected from the group consisting of halo, —CH₃, —CF₃, —CN, —OC₁₋₆alkyl,
(5) C₃₋₆cycloalkyl, optionally mono, di-, tri- or tetra substituted with substituents independently selected from the group consisting of hydroxyl, halo, phenyl,
(6) C₅₋₁₀carbocycle, wherein the carbocycle is optionally mono, di- or tri-substituted with substituents independently selected from halo, hydroxyl, —OC₁₋₆alkyl, CF₃,
(7) C₁₋₆alkyl, optionally mono or di-substituted with substituents independently selected from the group consisting of CF₃, hydroxyl, —CN, —CHF2,
(8) C₁₋₆alkyl-C₃₋₆cycloalkyl, wherein the cycloalkyl is optionally mono or di-substituted with substituents independently selected from hydroxyl, phenyl, —CH₂OH, and —C₃₋₆cycloalkyl,
(9) —C₁₋₆alkyl-heteroaryl, wherein the heteroaryl is optionally mono, di- or tri-substituted with substitutents independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and the C₁₋₆alkyl is optionally mono or di-substituted with a substitutent selected from the group consisting of —CF₃, —CHF₂, hydroxyl and —OC₁₋₆alkyl, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(10) —C₁₋₆alkyl-aryl, wherein the aryl is optionally mono, di- or tri-substituted with substitutents independently selected from —CH₃, —OC₁₋₆alkyl, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and the C₁₋₆alkyl is optionally mono or di-substituted with a substitutent selected from the group consisting of —CF₃, —CHF₂, hydroxyl and —OC₁₋₆alkyl, and wherein heteroaryl portion of —C(O)-heteroaryl is optionally mono, di- or tri-substituted with substituents selected from the group consisting of halo, —CH₃, —CF₃, —CN, —OC₁₋₆alkyl, and
(11) —C₁₋₆alkyl-heterocycle, wherein the heterocycle is optionally mono, di- or tri-substituted with substitutents independently selected from the —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, oxo and aryl, and the C₁₋₆alkyl is optionally mono or di-substituted with a substitutent selected from the group consisting of —CF₃, —CHF₂, hydroxyl and —OC₁₋₆alkyl and wherein aryl and the heteroaryl portion of —C(O)-heteroaryl is optionally mono, di- or tri-substituted with substituents independently selected from the group consisting of halo, —CH₃, —CF₃, —CN, —OC₁₋₆alkyl;

R⁵ is selected from the group consisting of hydrogen and methyl;

R⁶ is selected from the group consisting of:
(1) C₁₋₄alkyl, optionally mono or di-substituted, with substituents independently selected from the group consisting of C₃₋₆cycloalkyl, —CF₃, heteroaryl, —C₁₋₃alkyl-CF₃, CH₃, tetrahydrofuran, and
(2) —C₁₋₃alkyl-C₃₋₆cycloalkyl, wherein the cycloalkyl is optionally mono or di-substituted with substituents independently selected from the group consisting of halo, CF₃, CH₃, C₁₋₃alkyl, —OC₁₋₆alkyl, or

R5 and R6 are joined together so that along with the nitrogen to which they are attached, there is formed a heterocycle, wherein said heterocycle is optionally mono, di or tri-substituted with substituents independently selected from the group consisting of —OC₁₋₆alkyl, —NH—C(O)—O—C(CH₃)₃, hydroxy, —CH₃, —CF₃, —CH₂—OH, halo, —S(O)₂—CH₃, C(O)—O—C₁₋₆alkyl, —C(O)—N(CH₃)2, oxo, —C(O)—O—C(CH₃)₃, —C(O)-heteroaryl, —C₃₋₆cycloalkyl, —NH₂, —NH—C(O)—CF3, —C(O)—NHC₁₋₆alkyl, —C(O)—N(C₁₋₆alkyl)₂, —NC(O)—NH₂, —NH—S(O)₂—CH₃, —O—CF₃, —S—CH₃, and wherein the heteroaryl portion of —C(O)-heteroaryl is optionally mono- di- or tri-substituted with substituents selected from the group consisting of halo, —CH₃, —CF₃, —CN and —O—C₁₋₆alkyl;
provided that when the compound is of Formula (II), and R¹ is optionally substituted aryl, then R² is other than hydrogen, halo, cyano, optionally substituted aryl, optionally substituted heteroaryl or NR⁵R⁶ wherein both of R⁵ and R⁶ are hydrogen, or unsubstituted alkyl.

Within this embodiment there is a genus wherein

R¹ is selected from the group consisting of:
(1) —NH—R⁴,
(2) —O—R⁴, and
(3) —R⁴.

Within this genus there is a sub-genus wherein

R¹ is selected from the group consisting of:
(1) —NH—R⁴, and
(2) —R⁴.

Within this embodiment there is a genus wherein

R² is selected from the group consisting of:
(1) H,
(2) halo,
(3) —CN,
(4) —CF3,
(5) —C1-6alkyl,
(6) —C(O)-heterocycle, wherein the heterocycle is optionally mono, di- or tri-substituted with substituents independently selected from —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and oxo, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(7) —NR⁵R⁶,
(8) —C₁₋₄alkyl-NR⁵R⁶,
(9) —C₁₋₄alkyl-heterocycle, wherein the alkyl is optionally substituted with hydroxyl and wherein the heterocycle is optionally mono, di- or tri-substituted with substituents independently selected from —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, and oxo, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(10) —C₁₋₄alkyl-heteroaryl, optionally mono, di- or tri-substituted with substituents independently selected —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(11) heterocycle, optionally mono, di- or tri-substituted with substituents independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, and oxo, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(12) heteroaryl, optionally mono, di- or tri-substituted with substituents independently selected from —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and wherein the heteroaryl portion of —C(O)-heteroaryl is optionally mono, di- or tri-substituted with substituents selected from the group consisting of halo, —CH₃, —CF₃, —CN, —OC₁₋₆alkyl
(13) aryl, optionally mono, di- or tri-substituted with substituents independently selected from the group consisting of wherein the aryl is optionally mono or di-substituted with substituents selected from —CH₃, —O—C₁₋₆alkyl, hydroxy, —CH₂—OH, halo, —S(O)₂—CH₃, C(O)—O—C₁₋₆alkyl, —C(O)—NHC₁₋₆alkyl, —C(O)—N(C₁₋₆alkyl)₂, —(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and wherein the heteroaryl portion of —C(O)-heteroaryl is optionally mono, di- or tri-substituted with substituents selected from the group consisting of halo, —CH₃, —CF₃, —CN, —OC₁₋₆alkyl, and
(14) —C₃₋₆cycloalkyl, optionally mono or di-substituted with substituents independently selected from —CH₃, —O—C₁₋₆alkyl, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, oxo, C(O)—O—C(CH₃)₃, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN.

Within this genus there is a sub-genus wherein

R² is selected from the group consisting of:
(1) H,
(2) —CF3,
(3) —C(O)-heterocycle, wherein the heterocycle is optionally mono, di- or tri-substituted with substituents independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and oxo, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(4) —NR⁵R⁶,
(5) —C₁₋₄alkyl-NR⁵R⁶,
(6) —C₁₋₄alkyl-heterocycle, wherein the alkyl is optionally substituted with hydroxyl and wherein the heterocycle is optionally mono, di- or tri-substituted with substituents independently selected from —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, and oxo, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(7) heterocycle, optionally mono, di- or tri-substituted with substituents independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, and oxo, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(8) heteroaryl, optionally mono, di- or tri-substituted with substituents independently selected from —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and wherein the heteroaryl portion of —C(O)-heteroaryl is optionally mono, di- or tri-substituted with substituents selected from the group consisting of halo, —CH₃, —CF₃, —CN, —OC₁₋₆alkyl, and
(9) aryl, optionally mono, di- or tri-substituted with substituents independently selected from the group consisting of —CH₃, —O—C₁₋₆alkyl, hydroxy, —CH₂—OH, halo, —S(O)₂—CH₃, C(O)—O—C₁₋₆alkyl, —C(O)—NHC₁₋₆alkyl, —C(O)—N(C₁₋₆alkyl)₂, —(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and wherein the heteroaryl portion of —C(O)-heteroaryl is optionally mono, di- or tri-substituted with substituents selected from the group consisting of halo, —CH₃, —CF₃, —CN, —OC₁₋₆alkyl.

Within this sub-genus there is a class wherein

R² is selected from the group consisting of:
(1) —CF3,
(2) —C₁₋₄alkyl-heterocycle, wherein the alkyl is optionally substituted with hydroxyl and wherein the heterocycle is optionally mono, di- or tri-substituted with substituents independently selected from —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, and oxo, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(3) heterocycle, optionally mono, di- or tri-substituted with substituents independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, and oxo, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(4) heteroaryl, optionally mono, di- or tri-substituted with substituents independently selected from —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and wherein the heteroaryl portion of —C(O)-heteroaryl is optionally mono, di- or tri-substituted with substituents selected from the group consisting of halo, —CH₃, —CF₃, —CN, —OC₁₋₆alkyl, and
(5) aryl, optionally mono, di- or tri-substituted with substituents independently selected from the group consisting of wherein the aryl is optionally mono or di-substituted with substituents selected from —CH₃, —O—C₁₋₆alkyl, hydroxy, —CH₂—OH, halo, —S(O)₂—CH₃, C(O)—O—C₁₋₆alkyl, —C(O)—NHC₁₋₆alkyl, —C(O)—N(C₁₋₆alkyl)₂, —(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and wherein the heteroaryl portion of —C(O)-heteroaryl is optionally mono, di- or tri-substituted with substituents selected from the group consisting of halo, —CH₃, —CF₃, —CN, —OC₁₋₆alkyl.

Within this embodiment there is a genus wherein

R³ is selected from the group consisting of
(1) H,
(2) halo,
(3) —C₁₋₄alkyl, optionally substituted with hydroxyl,
(4) —CF₃,
(5) —OC₁₋₆alkyl, and
(6) —CN.

Within this genus there is a sub-genus wherein

R³ is selected from the group consisting of
(1) H, and
(2) halo.

Within this embodiment there is a genus wherein

R⁴ is selected from the group consisting of:
(1) adamantane, optionally mono and di-substituted with substituents independently selected from —CH₃ and hydroxyl,
(2) aryl, optionally mono, di- or tri-substituted with substituents independently selected from —CH₃, —OC₁₋₆alkyl, hydroxy, —CH₃—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and wherein the heteroaryl portion of —C(O)-heteroaryl is optionally mono, di- or tri-substituted with substituents independently selected from the group consisting of halo, —CH₃, —CF₃, —CN, —OC₁₋₆alkyl,
(3) heteroaryl, optionally mono or di-substituted with a substituents independently selected from —CN, —CH₃, —CF₃, —CHF₂, —OC₁₋₆alkyl, —O—CF3, hydroxy, —CH₃—OH, halo, —S(O)₂—CH₃, —C(O)—O—CH₃, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(4) heterocycle, optionally mono, di-, tri- or tetra substituted with substituents independently selected from —CN, —CH₃, —CF₃, —CHF₂, —OC₁₋₆alkyl, —O—CF3, hydroxy, —CH₃—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, oxo and aryl, and wherein aryl and the heteroaryl portion of —C(O)-heteroaryl is optionally mono, di- or tri-substituted with substituents selected from the group consisting of halo, —CH₃, —CF₃, —CN, —OC₁₋₆alkyl,
(5) C₁₋₆alkyl-C₃₋₆cycloalkyl, wherein the cycloalkyl is optionally mono or di-substituted with substituents selected from hydroxyl, phenyl, —CH₂OH, —C₃₋₆cycloalkyl,
(6) —C₁₋₆alkyl-heteroaryl, wherein the heteroaryl is optionally mono, di- or tri-substituted with substitutents independently selected from —CN, —CH₃, —CF₃, —CHF₂, —OC₁₋₆alkyl, —O—CF3, hydroxy, —CH₃—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and the C₁₋₆alkyl is optionally mono or di-substituted with substitutents independently selected from the group consisting of —CF₃, —CHF₂, hydroxyl and —OC₁₋₆alkyl, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(7) —C₁₋₆alkyl-aryl, wherein the aryl is optionally mono, di- or tri-substituted with substitutents independently selected from —CH₃, —OC₁₋₆alkyl, hydroxy, —CH₃—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and the C₁₋₆alkyl is optionally mono or di-substituted with a substitutent selected from the group consisting of —CF₃, —CHF₂, hydroxyl and —OC₁₋₆alkyl, and wherein the heteroaryl portion of —C(O)-heteroaryl is optionally mono, di- or tri-substituted with substituents selected from the group consisting of halo, —CH₃, —CF₃, —CN, —OC₁₋₆alkyl, and
(8) —C₁₋₆alkyl-heterocycle, wherein the heterocycle is optionally mono, di- or tri-substituted with substitutents independently selected from the —CN, —CH₃, —CF₃, —CHF₂, —OC₁₋₆alkyl, —O—CF3, hydroxy, —CH₃—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, oxo and aryl, and the C₁₋₆alkyl is optionally mono or di-substituted with a substitutent selected from the group consisting of —CF₃, —CHF₂, hydroxyl and —OC₁₋₆alkyl, and wherein aryl and the heteroaryl portion of —C(O)-heteroaryl is optionally mono, di- or tri-substituted with substituents selected from the group consisting of halo, —CH₃, —CF₃, —CN, —OC₁₋₆alkyl.

Within this genus there is a sub-genus wherein

R⁴ is selected from the group consisting of:
(1) aryl, optionally mono, di- or tri-substituted with substituents independently selected from —CH₃, —OC₁₋₆alkyl, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and wherein the heteroaryl portion of —C(O)heteroaryl is optionally mono or di-substituted with substituents independently selected from the group consisting of halo, —CH₃, —CF₃, —CN, —OC₁₋₆alkyl,
(2) heterocycle, optionally mono, di-, tri- or tetra substituted with substituents independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, oxo and aryl, and wherein aryl and the heteroaryl portion of —C(O)-heteroaryl is optionally mono, di- or tri-substituted with substituents selected from the group consisting of halo, —CH₃, —CF₃, —CN, —OC₁₋₆alkyl,
(3) —C₁₋₆alkyl-heteroaryl, wherein the heteroaryl is optionally mono, di- or tri-substituted with substitutents independently selected from —CN, —CH₃, —CF₃, —CHF₂, —OC₁₋₆alkyl, —O—CF3, hydroxy, —CH₃—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and the C₁₋₆alkyl is optionally mono or di-substituted with substitutents independently selected from the group consisting of —CF₃, —CHF₂, hydroxyl and —OC₁₋₆alkyl, and wherein heteroaryl portion of —C(O)-heteroaryl is optionally mono, di- or tri-substituted with substituents selected from the group consisting of halo, —CH₃, —CF₃, —CN, —OC₁₋₆alkyl,
(4) —C₁₋₆alkyl-aryl, wherein the aryl is optionally mono, di- or tri-substituted with substitutents independently selected from —CH₃, —OC₁₋₆alkyl, hydroxy, —CH₃—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and the C₁₋₆alkyl is optionally mono or di-substituted with substitutents independently selected from the group consisting of —CF₃, —CHF₂, hydroxyl and —OC₁₋₆alkyl and wherein the heteroaryl portion of —C(O)-heteroaryl is optionally mono, di- or tri-substituted with substituents selected from the group consisting of halo, —CH₃, —CF₃, —CN, —OC₁₋₆alkyl.

Within this embodiment there is a genus wherein

R5 and R6 are joined together so that along with the nitrogen to which they are attached, there is formed a heterocycle, wherein said heterocycle is optionally mono, di or tri-substituted with substituents independently selected from the group consisting of —OC₁₋₆alkyl, —NH—C(O)—O—C(CH₃)₃, hydroxy, —CH₃, —CF₃, —CH₂—OH, halo, —S(O)₂—CH₃, C(O)—O—C₁₋₆alkyl, —C(O)—N(CH₃)2, oxo, —C(O)—O—C(CH₃)₃, —C(O)-heteroaryl, —C₃₋₆cycloalkyl, —NH₂, —NH—C(O)—CF₃, —C(O)—NHC₁₋₆alkyl, —C(O)—N(C₁₋₆alkyl)₂, —NC(O)—NH₂, —NH—S(O)₂—CH₃, —O—CF₃, —S—CH₃, and wherein the heteroaryl portion of —C(O)-heteroaryl is optionally mono- or di-substituted with substituents independently selected from the group consisting of halo, —CH₃, —CF₃, —CN and —O—C₁₋₆alkyl.

Within this embodiment there is a genus wherein

R¹ is selected from the group consisting of:
(1) —NH—R⁴, and
(2) —R⁴;

R² is selected from the group consisting of:
(1) —CF3,
(2) —C₁₋₄alkyl-heterocycle, wherein the alkyl is optionally substituted with hydroxyl and wherein the heterocycle is optionally mono, di- or tri-substituted with substituents independently selected from —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, and oxo, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(3) heterocycle, optionally mono, di- or tri-substituted with substituents independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, and oxo, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(4) heteroaryl, optionally mono, di- or tri-substituted with substituents independently selected from —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and wherein the heteroaryl portion of —C(O)-heteroaryl is optionally mono, di- or tri-substituted with substituents selected from the group consisting of halo, —CH₃, —CF₃, —CN, —OC₁₋₆alkyl, and
(5) aryl, optionally mono, di- or tri-substituted with substituents independently selected from the group consisting of —CH₃, —O—C₁₋₆alkyl, hydroxy, —CH₂—OH, halo, —S(O)₂—CH₃, C(O)—O—C₁₋₆alkyl, —C(O)—NHC₁₋₆alkyl, —C(O)—N(C₁₋₆alkyl)₂, —(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and wherein the heteroaryl portion of —C(O)-heteroaryl is optionally mono, di- or tri-substituted with substituents selected from the group consisting of halo, —CH₃, —CF₃, —CN, —OC₁₋₆alkyl;

R³ is selected from the group consisting of
(1) H, and
(2) halo; and

R⁴ is selected from the group consisting of:
(1) aryl, optionally mono, di- or tri-substituted with substituents independently selected from —CH₃, —OC₁₋₆alkyl, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and wherein the heteroaryl portion of —C(O)-heteroaryl is optionally mono, di- or tri-substituted with substituents independently selected from the group consisting of halo, —CH₃, —CF₃, —CN, —OC₁₋₆alkyl,
(2) heterocycle, optionally mono, di-, tri- or tetra substituted with substituents independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, oxo and aryl, and wherein aryl and the heteroaryl portion of —C(O)-heteroaryl is optionally mono, di- or tri-substituted with substituents selected from the group consisting of halo, —CH₃, —CF₃, —CN, —OC₁₋₆alkyl,
(3) —C₁₋₆alkyl-heteroaryl, wherein the heteroaryl is optionally mono, di- or tri-substituted with substitutents independently selected from —CN, —CH₃, —CF₃, —CHF₂, —OC₁₋₆alkyl, —O—CF3, hydroxy, —CH₃—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and the C₁₋₆alkyl is optionally mono or di-substituted with substitutents independently selected from the group consisting of —CF₃, —CHF₂, hydroxyl and —OC₁₋₆alkyl, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(4) —C₁₋₆alkyl-aryl, wherein the aryl is optionally mono, di- or tri-substituted with substitutents independently selected from —CH₃, —OC₁₋₆alkyl, hydroxy, —CH₃—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and the C₁₋₆alkyl is optionally mono or di-substituted with substitutents independently selected from the group consisting of —CF₃, —CHF₂, hydroxyl and —OC₁₋₆alkyl, and wherein the heteroaryl portion of —C(O)-heteroaryl is optionally mono, di- or tri-substituted with substituents selected from the group consisting of halo, —CH₃, —CF₃, —CN, —OC₁₋₆alkyl.

Within this embodiment there is a genus wherein

R¹ is selected from the group consisting of:
(1) —NH—R⁴, and
(2) —R⁴;

R² is selected from the group consisting of:
(1) —NR⁵R⁶,
(2) —C₁₋₄alkyl-NR⁵R⁶,
(3) heteroaryl, optionally mono, di- or tri-substituted with substituents independently selected from —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and wherein the heteroaryl portion of —C(O)-heteroaryl is optionally mono, di- or tri-substituted with substituents selected from the group consisting of halo, —CH₃, —CF₃, —CN, —OC₁₋₆alkyl and
(4) aryl, optionally mono, di- or tri-substituted with substituents independently selected from the group consisting of wherein the aryl is optionally mono or di-substituted with substituents selected from —CH₃, —O—C₁₋₆alkyl, hydroxy, —CH₂—OH, halo, —S(O)₂—CH₃, C(O)—O—C₁₋₆alkyl, —C(O)—NHC₁₋₆alkyl, —C(O)—N(C₁₋₆alkyl)₂, —(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and wherein the heteroaryl portion of —C(O)-heteroaryl is optionally mono, di- or tri-substituted with substituents selected from the group consisting of halo, —CH₃, —CF₃, —CN, —OC₁₋₆alkyl;

R³ is selected from the group consisting of
(1) H, and
(2) halo; and

R⁴ is selected from the group consisting of:
(1) aryl, optionally mono, di- or tri-substituted with substituents independently selected from —CH₃, —OC₁₋₆alkyl, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and wherein the heteroaryl portion of —C(o)-heteroaryl is optionally mono, di- or tri-substituted with substituents independently selected from the group consisting of halo, —CH₃, —CF₃, —CN, —OC₁₋₆alkyl,
(2) heterocycle, optionally mono, di-, tri- or tetra substituted with a substituent independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, oxo and aryl, and wherein aryl and the heteroaryl portion of —C(O)-heteroaryl is optionally mono, di- or tri-substituted with substituents selected from the group consisting of halo, —CH₃, —CF₃, —CN, —OC₁₋₆alkyl,
(3) —C₁₋₆alkyl-heteroaryl, wherein the heteroaryl is optionally mono, di- or tri-substituted with substitutents independently selected from —CN, —CH₃, —CF₃, —CHF₂, —OC₁₋₆alkyl, —O—CF3, hydroxy, —CH₃—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and the C₁₋₆alkyl is optionally mono or di-substituted with substitutents independently selected from the group consisting of —CF₃, —CHF₂, hydroxyl and —OC₁₋₆alkyl, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(4) —C₁₋₆alkyl-aryl, wherein the aryl is optionally mono, di- or tri-substituted with substitutents independently selected from —CH₃, —OC₁₋₆alkyl, hydroxy, —CH₃—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and the C₁₋₆alkyl is optionally mono or di-substituted with substitutents independently selected from the group consisting of —CF₃, —CHF₂, hydroxyl and —OC₁₋₆alkyl, and wherein the heteroaryl portion of —C(O)-heteroaryl is optionally mono, di- or tri-substituted with substituents selected from the group consisting of halo, —CH₃, —CF₃, —CN, —OC₁₋₆alkyl; and

R5 and R6 are joined together so that along with the nitrogen to which they are attached, there is formed a heterocycle, wherein said heterocycle is optionally mono, di or tri-substituted with substituents independently selected from the group consisting of —OC₁₋₆alkyl, —NH—C(O)—O—C(CH₃)₃, hydroxy, —CH₃, —CF₃, —CH₂—OH, halo, —S(O)₂—CH₃, C(O)—O—C₁₋₆alkyl, —C(O)—N(CH₃)2, oxo, —C(O)—O—C(CH₃)₃, —C(O)-heteroaryl, —C₃₋₆cycloalkyl, —NH₂, —NH—C(O)—CF₃, —C(O)—NHC₁₋₆alkyl, —C(O)—N(C₁₋₆alkyl)₂, —NC(O)—NH₂, —NH—S(O)₂—CH₃, —O—CF₃, —S—CH₃, and wherein the heteroaryl portion of —C(O)-heteroaryl is optionally mono- di- or tri-substituted with substituents independently selected from the group consisting of halo, —CH₃, —CF₃, —CN and —O—C₁₋₆alkyl.

As used herein, “alkyl” as well as other groups having the prefix “alk” such as, for example, alkoxy, alkanoyl, alkenyl, alkynyl and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains containing at least one unsaturated C—C bond.

As used here a “cycloalkyl”, is a saturated monocyclic hydrocarbon ring.

As used here a “carbocycle”, is a mono cyclic or bi-cyclic carbocyclic non-aromatic ring having at least one double bond.

The term “aryl”, unless specifically stated otherwise, refers to single and multi-cyclic aromatic ring systems in which the ring members are all carbon, for example, phenyl or naphthyl.

The term “heteroaryl”, unless specifically stated otherwise, refers to single and multi-cyclic aromatic ring systems in which at least one of the ring members is other than carbon. Heteroaryl includes, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridine, pyridazine, pyrimidine, pyrazine, triazine, including 1,2,4-triazine and 1,3,5-triazine, tetrazine, including 1,2,4,5-tetrazine, and the like.

The term “heterocycle”, unless specifically stated otherwise, refers to single and multi-cyclic non-aromatic ring systems in which at least one of the ring members is other than carbon. Heterocycle includes pyrrolidine, piperidine, piperazine, morpholine, azetidine, tetrahydropyran, tetrahydrofuran, dioxane, and the like.

The term “amine” unless specifically stated otherwise includes primary, secondary and tertiary amines.

The term “halogen” includes fluorine, chlorine, bromine and iodine atoms.

The term “oxide” of heteroaryl groups is used in the ordinary well-known chemical sense and include, for example, N-oxides of nitrogen heteroatoms.

Compounds described herein contain one or more double bonds and may thus give rise to cis/trans isomers as well as other conformational isomers. The present invention includes all such possible isomers as well as mixtures of such isomers.

Compounds described herein can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above compounds of the invention may be shown without a definitive stereochemistry at certain positions. The present invention includes all stereoisomers of the compounds of the invention and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The term “aryl”, unless specifically stated otherwise, refers to single and multi-cyclic aromatic ring systems in which the ring members are all carbon, for example, phenyl or naphthyl.

The term “heteroaryl”, unless specifically stated otherwise, refers to single and multi-cyclic aromatic ring systems in which at least one of the ring members is other than carbon. Heteroaryl includes, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridine, pyridazine, pyrimidine, pyrazine, triazine, including 1,2,4-triazine and 1,3,5-triazine, tetrazine, including 1,2,4,5-tetrazine, and the like.

The term “heterocycle”, unless specifically stated otherwise, refers to single and multi-cyclic non-aromatic ring systems in which at least one of the ring members is other than carbon. Heterocycle includes pyrrolidine, piperidine, piperazine, morpholine, azetidine, tetrahydropyran, tetrahydrofuran, dioxane, and the like.

The term “optionally substituted” is intended to include both substituted and unsubstituted. Thus, for example, optionally substituted aryl can represent a pentafluorophenyl or a phenyl ring. Further, the substitution can be made at any of the groups. For example, substituted aryl(C₁₋₆)alkyl includes substitution on the aryl group as well as substitution on the alkyl group.

The term “polycyclic ring” means more than 3 fused rings and includes carbon as ring atoms. The polycyclic ring can be saturated or unsaturated. The polycyclic ring can be unsubstituted, singly substituted or, if possible, multiply substituted, with substituent groups in any possible position. The individual rings may or may not be of the same type. Examples of polycyclic rings include adamantane, bicyclooctane, norbornane and bicyclononanes.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

“Pharmaceutically acceptable non-toxic acids”, including inorganic and organic acids, salts prepared from, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric, and tartaric acids.

The pharmaceutical compositions of the present invention comprise a compound represented of the invention (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds of the invention, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles and the like. Further, the compositions can be in a form suitable for use in transdermal devices. These formulations may be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, may also be prepared in powder or liquid concentrate form.

A formulation intended for the oral administration to humans may conveniently contain from about 0.5 mg to about 5 g of active agent, compounded with an appropriate and convenient amount of carrier material which may vary from about 5 to about 95 percent of the total composition. Unit dosage forms can generally contain between from about 1 mg to about 1000 mg of the active ingredient.

The conditions recited herein can be treated or prevented by the administration of from about 0.01 mg to about 140 mg of the instant compounds per kilogram of body weight per day.

It is understood, however, that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy. For example, inflammatory pain may be effectively treated by the administration of from about 0.01 mg to about 75 mg of the present compound per kilogram of body weight per day, or alternatively about 0.5 mg to about 3.5 g per patient per day. Neuropathic pain may be effectively treated by the administration of from about 0.01 mg to about 125 mg of the present compound per kilogram of body weight per day, or alternatively about 0.5 mg to about 5.5 g per patient per day.

It is understood that compounds of this invention can be administered at prophylactically effective dosage levels to prevent the above-recited conditions, as well as to prevent other conditions mediated through CB2 receptor.

The Compounds of the invention may be used with other therapeutic agents such as those described below. Such other therapeutic agent(s) may be administered prior to, simultaneously with, or following the administration of the cannabinoid receptor modulators in accordance with the invention.

Compounds of the invention may be used in combination with other drugs that are used in the treatment/prevention/suppression or amelioration of the diseases or conditions for which compounds of the invention are useful. Such other drugs may be administered, by a route and in an amount commonly used therefor, contemporaneously or sequentially with a compound of the invention. When a compound of the invention is used contemporaneously with one or more other drugs, a pharmaceutical composition containing such other drugs in addition to the compound of the invention is preferred. Accordingly, the pharmaceutical compositions of the present invention include those that also contain one or more other active ingredients, in addition to a compound of the invention. Examples of active ingredients that may be combined with a compound of the invention, either administered separately or in the same pharmaceutical compositions, include, but are not limited to: (1) non-steroidal anti-inflammatory agents, such as ibuprofen and naproxen; (2) COX-2 inhibitors, such as Celebrex and Arcoxia; (3) bradykinin B1 receptor antagonists; (4) sodium channel blockers and antagonists; (5) nitric oxide synthase (NOS) inhibitors; (6) glycine site antagonists; (7) potassium channel openers; (8) AMPA/kainate receptor antagonists; (9) calcium channel antagonists; (10) GABA-A receptor modulators (e.g., a GABA-A receptor agonist); (11) matrix metalloprotease (MMP) inhibitors; (12) thrombolytic agents; (13) opioids such as morphine; (14) neutrophil inhibitory factor (NIF); (15) L-Dopa; (16) carbidopa; (17) levodopa/carbidopa; (18) dopamine agonists such as bromocriptine, pergolide, pramipexole, ropinirole; (19) anticholinergics; (20) amantadine; (21) carbidopa; (22) catechol O-methyltransferase (“COMT”) inhibitors such as entacapone and tolcapone; (23) Monoamine oxidase B (“MAO-B”) inhibitors; (24) opiate agonists or antagonists; (25) 5HT receptor agonists or antagonists; (26) NMDA receptor agonists or antagonists; (27) NK1 antagonists; (28) selective serotonin reuptake inhibitors (“SSRI”) and/or selective serotonin and norepinephrine reuptake inhibitors (“SSNRI”); (29) tricyclic antidepressant drugs, (30) norepinephrine modulators; (31) lithium; (32) valproate; and (33) neurontin (gabapentin).

Additional examples of active ingredients that may be combined with a compound of the invention, either administered separately or in the same pharmaceutical compositions, include, but are not limited to: (34) cyclosporins (e.g., cyclosporin A); (35) CTLA4-Ig, antibodies such as anti-ICAM-3, anti-IL-2 receptor (Anti-Tac), anti-CD45RB, anti-CD2, anti-CD3 (OKT-3), anti-CD4, anti-CD80, anti-CD86, and monoclonal antibody OKT3; (36) agents blocking the interaction between CD40 and gp39, such as antibodies specific for CD40 and/or gp39 (i.e., CD154); (37) fusion proteins constructed from CD40 and gp39 (CD40Ig and CD8gp39), (38) inhibitors, such as nuclear translocation inhibitors of NF-kappa B function, such as deoxyspergualin (DSG); (38) steroids such as prednisone or dexamethasone; (39) gold compounds; (40) antiproliferative agents such as methotrexate, FK506 (tacrolimus, Prograf), mycophenolate mofetil; (41) cytotoxic drugs such as azathiprine and cyclophosphamide; (42) TNF-α. inhibitors such as tenidap; (43) anti-TNF antibodies or soluble TNF receptor such as etanercept (Enbrel); (44) rapamycin (sirolimus or Rapamune); (45) leflunomide (Arava); (46) anticytokines such as antiIL-4 or IL-4 receptor fusion proteins and PDE 4 inhibitors such as Ariflo, and (47) the PTK inhibitors disclosed in the following U.S. patent applications, incorporated herein by reference in their entirety: U.S. Ser. No. 09/097,338, filed Jun. 15, 1998; U.S. Ser. No. 09/094,797, filed Jun. 15, 1998; U.S. Ser. No. 09/173,413, filed Oct. 15, 1998; and U.S. Ser. No. 09/262,525, filed Mar. 4, 1999. See also the following documents and references cited therein and incorporated herein by reference: Hollenbaugh, D., Et Al, “Cleavable CD40Ig Fusion Proteins and the Binding to Sgp39”, J. Immunol. Methods (Netherlands), 188(1), pp. 1-7 (Dec. 15, 1995); Hollenbaugh, D., et al, “The Human T Cell Antigen Gp39, A Member of the TNF Gene Family, Is a Ligand for the CD40 Receptor: Expression of a Soluble Form of Gp39 with B Cell Co-Stimulatory Activity”, EMBO J (England), 11(12), pp. 4313-4321 (December 1992); and Moreland, L. W. et al., “Treatment of Rheumatoid Arthritis with a Recombinant Human Tumor Necrosis Factor Receptor (P75)-Fc Fusion Protein,” New England J. of Medicine, 337(3), pp. 141-147 (1997).

Thus, compounds of the invention may be useful as analgesics. For example they may be useful in the treatment of chronic inflammatory pain (e.g. pain associated with rheumatoid arthritis, osteoarthritis, rheumatoid spondylitis, gouty arthritis and juvenile arthritis) including the property of disease modification and joint structure preservation; musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea.

Compounds of the invention may be particularly useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them.

Neuropathic pain syndromes include: diabetic neuropathy; sciatica; non-specific lower back pain; multiple sclerosis pain; fibromyalgia; HIV-related neuropathy; post-herpetic neuralgia; trigerninal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are incredibly heterogeneous and are often described as spontaneous shooting and laminating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Compounds of the invention may also be useful in the treatment of inflammation, for example in allergies, asthma, autoimmune diseases such as transplant rejection (e.g., kidney, heart, lung, liver, pancreas, skin; host versus graft reaction (HVGR), graft versus host reaction (GVHR) etc.), rheumatoid arthritis, and amyotrophic lateral sclerosis, T-cell mediated autoimmune diseases such as multiple sclerosis, psoraiasis and Sjogren's syndrome, Type II inflammatory diseases such as vascular inflammation (including vasculitis, arteritis, atherosclerosis and coronary artery disease), diseases of the central nervous system such as stroke, pulmonary diseases such as bronchitis obliteraus and primary pulmonary hypertension, and solid, delayed Type IV hypersensitivity reactions, and hematologic malignancies such as leukemia and lymphomas.

Compounds of the invention may also be useful in the treatment of neurodegenerative diseases and neurodegeneration such as dementia, particularly degenerative dementia (including senile dementia, Alzheimer's disease, Pick's disease, Huntingdon's chorea, Parkinson's disease and Creutzfeldt-Jakob disease, motor neuron disease); vascular dementia (including multi-infarct dementia); as well as dementia associated with intracranial space occupying lesions; trauma; infections and related conditions (including HIV infection); dementia in Parkinson's disease; metabolism; toxins; anoxia and vitamin deficiency; and mild cognitive impairment associated with aging, particularly Age Associated Memory Impairment. The compounds may also be useful for the treatment of amyotrophic lateral sclerosis (ALS) and neuroinflammation.

Compounds of the invention may also be useful in the treatment of psychiatric disease for example schizophrenia, depression (which term is used herein to include bipolar depression, unipolar depression, single or recurrent major depressive episodes with or without psychotic features, catatonic features, melancholic features, atypical features or postpartum onset, seasonal affective disorder, dysthymic disorders with early or late onset and with or without atypical features, neurotic depression and social phobia, depression accompanying dementia for example of the Alzheimer's type, schizoaffective disorder or the depressed type, and depressive disorders resulting from general medical conditions.

Compounds of the invention may also be useful in the treatment of cancer, including but not limited to adenomas, meningiomas, glioblastomas and melanoma.

The preferred uses of CB2 agonists are for the treatment of pain and inflammatory conditions. Pain is selected from inflammatory pain, viseral pain, cancer pain, neuropathic pain, lower back pain, muscular skeletal, post operative pain, acute pain, migraine and inflammatory pain associated with rheumatoid arthritis or osteoarthritis. Indications associated with inflammation include allergies, asthma, multiple sclerosis, vasculitis, arteritis, atherosclerosis and coronary artery disease.

Compounds of the invention are effective for treating and preventing pain, respiratory and non-respiratory diseases.

Respiratory diseases for which the compounds of the invention are useful include but are not limited to chronic pulmonary obstructive disorder, emphysema, asthma, and bronchitis. Compounds of the invention are also useful in the treatment and prevention of indications disclosed in European Patent Documents Nos. EP 0570920 and EP 0444451; International Publications Nos. WO 97/29079, WO 99/02499, WO 98/41519, and WO 9412466; U.S. Pat. Nos. 4,371,720, 5,081,122, 5,292,736, and 5,013,387; and French Patent No. FR 2735774.

The compounds of the invention stimulate inhibitory pathways in cells, particularly in leukocytes, lung epithelial cells, or both, and are thus useful in treating respiratory diseases. “Leukocyte activation” is defined herein as any or all of cell proliferation, cytokine production, adhesion protein expression, and production of inflammatory mediators. “Epithelial cell activation” is defined herein as the production of any or all of mucins, cytokines, chemokines, and adhesion protein expression.

The Compounds of the invention are expected to block the activation of lung epithelial cells by moieties such as allergic agents, inflammatory cytokines or smoke, thereby limiting release of mucin, cytokines, and chemokines. Another preferred embodiment of the present invention comprises use of novel cannabinoid receptor modulator compounds to treat respiratory disease wherein the compounds selectively inhibit lung epithelial cell activation.

Thus, Compounds of the invention, in treating leukocyte activation-associated disorders are useful in treating a range of disorders such as: transplant (such as organ transplant, acute transplant, xenotransplant or heterograft or homograft (such as is employed in burn treatment)) rejection; protection from ischemic or reperfusion injury such as ischemic or reperfusion injury incurred during organ transplantation, myocardial infarction, stroke or other causes; transplantation tolerance induction; arthritis (such as rheumatoid arthritis, psoriatic arthritis or osteoarthritis); multiple sclerosis; respiratory and pulmonary diseases including but not limited to chronic obstructive pulmonary disease (COPD), emphysema, bronchitis, and acute respiratory distress syndrome (ARDS); inflammatory bowel disease, including ulcerative colitis and Crohn's disease; lupus (systemic lupus erythematosis); graft vs. host disease; T-cell mediated hypersensitivity diseases, including contact hypersensitivity, delayed-type hypersensitivity, and gluten-sensitive enteropathy (Celiac disease); psoriasis; contact dermatitis (including that due to poison ivy); Hashimoto's thyroiditis; Sjogren's syndrome; Autoimmune Hyperthyroidism, such as Graves' Disease; Addison's disease (autoimmune disease of the adrenal glands); Autoimmune polyglandular disease (also known as autoimmune polyglandular syndrome); autoimmune alopecia; pernicious anemia; vitiligo; autoimmune hypopituatarism; Guillain-Barre syndrome; other autoimmune diseases; glomerulonephritis; serum sickness; uticaria; allergic diseases such as respiratory allergies (asthma, hayfever, allergic rhinitis) or skin allergies; scleracierma; mycosis fungoides; acute inflammatory and respiratory responses (such as acute respiratory distress syndrome and ishchemia/reperfusion injury); dermatomyositis; alopecia areata; chronic actinic dermatitis; eczema; Behcet's disease; Pustulosis palmoplanteris; Pyoderma gangrenum; Sezary's syndrome; atopic dermatitis; systemic schlerosis; and morphea. The term “leukocyte activation-associated” or “leukocyte-activation mediated” disease as used herein includes each of the above referenced diseases or disorders. In a particular embodiment, the compounds of the present invention are useful for treating the aforementioned exemplary disorders irrespective of their etiology. The combined activity of the present compounds towards monocytes, macrophages, T-cells, etc. may be useful in treating any of the above-mentioned disorders.

Exemplary non-respiratory cannabinoid receptor-mediated diseases include transplant rejection, rheumatoid arthritis, multiple sclerosis, inflammatory bowel disease, lupus, graft v. host disease, T-cell mediated hypersensitivity disease, psoriasis, Hashimoto's thyroiditis, Guillain-Barre syndrome, cancer, contact dermatitis, allergic rhinitis, and ischemic or reperfusion injury.

Compounds of the invention also inhibit the Fc gamma dependent production of TNF-α in human monocytes/macrophages. The ability to inhibit Fc gamma receptor dependent monocyte and macrophage responses results in additional anti-inflammatory activity for the present compounds. This activity is especially of value, for example, in treating inflammatory diseases such as arthritis or inflammatory bowel disease. In particular, the present compounds are useful for treating autoimmune glomerulonephritis and other instances of glomerulonephritis induced by deposition of immune complexes in the kidney that trigger Fc gamma receptor responses leading to kidney damage.

Cannabinoid receptors may be expressed on gut epithelial cells and hence regulate cytokine and mucin production and may be of clinical use in treating inflammatory diseases related to the gut. Cannabinoid receptors are also expressed on lymphocytes, a subset of leukocytes. Thus, cannabinoid receptor modulators will inhibit B and T-cell activation, proliferation and differentiation. Thus, such compounds will be useful in treating autoimmune diseases that involve either antibody or cell mediated responses such as multiple sclerosis and lupus.

In addition, cannabinoid receptors regulate the Fc epsilon receptor and chemokine induced degranulation of mast cells and basophils. These play important roles in asthma, allergic rhinitis, and other allergic disease. Fc epsilon receptors are stimulated by IgE-antigen complexes. Compounds of the present invention inhibit the Fc epsilon induced degranulation responses, including the basophil cell line, RBL. The ability to inhibit Fc epsilon receptor dependent mast cell and basophil responses results in additional anti-inflammatory and anti-allergic activity for the present compounds. In particular, the present compounds are useful for treating asthma, allergic rhinitis, and other instances of allergic disease.

The utility of the compounds of the invention can be demonstrated by the following assays.

### Cyclic AMP Assay

Chinese Hamster Ovary cells (CHO) expressing human CB1 or human CB2 (3.3×10⁵ cells/ml) were preincubated for 15 min at room temperature with tested agonist and 3-isobutyl-1-methylxanthine (IBMX; 200 μM) in phosphate buffered saline containing 1 mg/ml BSA (assay buffer) followed by 30 min incubation with forskolin in a total volume of 10 μl. The optimal forskolin concentration for each cell line was established in a separate experiment and adjusted to stimulate 70% of maximal cAMP response. cAMP content was measured using an HTRF assay (CisBio) according to the manufacturer's two step protocol.

In this assay, compounds of the invention have an IP ranging from 1 nM to >17000 nM. The Examples below have an IP ranging from 1 nM to >17000 nM.

| | CB2 cAMP | CB2 Emax | CB1 cAMP |
|---|---|---|---|
| Structure | (nM) | (%) | (nM) |
| 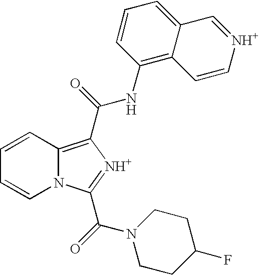 | 23.45 | 79.51 | 2152 |
| 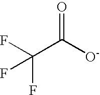 | | | |
| 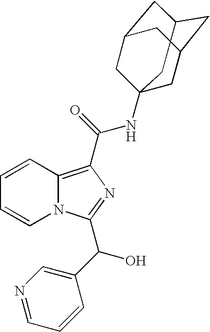 | 46.19 | 80.83 | >17000 |
| 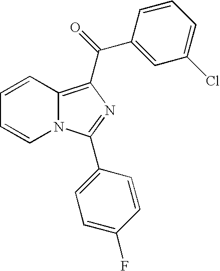 | 176.1 | 63.67 | >17000 |
| 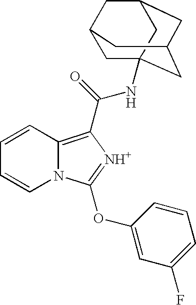 | 1.924 | 91.54 | 140 |
| 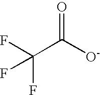 | | | |
| 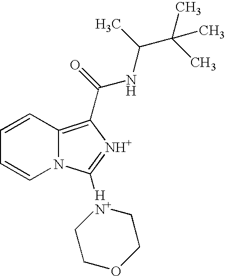 | 5.195 | 99.5 | >17000 |
| 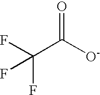 | | | |
| 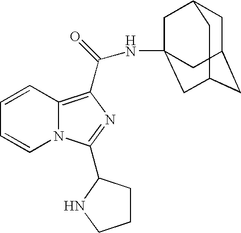 | 5.934 | 87.14 | 844.8 |
| 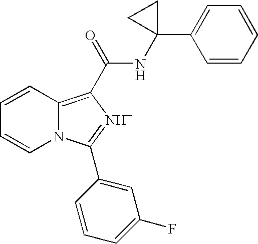 | 61.95 | 69.85 | 544.5 |
| 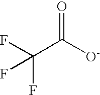 | | | |
| 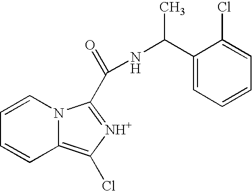 | 18.44 | 83.8 | >17000 |
| 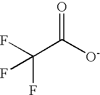 | | | |
| 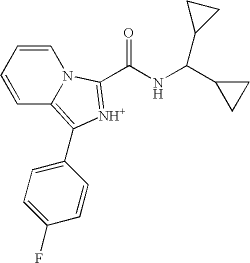 | 2.009 | 96.43 | 405 |
| 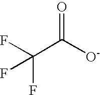 | | | |
| 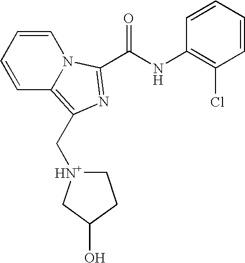 | 260.7 | 69.93 | >17000 |
| 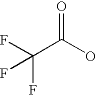 | | | |
| 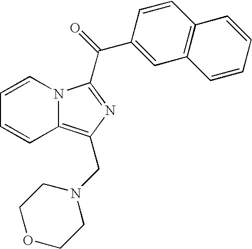 | 2.276 | 76.69 | 878.5 |
| 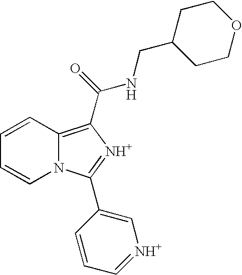 | 6428 | 74.74 | >17000 |
| 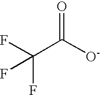 | | | |

### Evaluation of Compounds in the Rat CFA Inflammatory Pain Model and Rat Iodoacetate Model of Osteoarthritis

### Rat Complete Freunds Adjuvant (CFA) Model of Inflammatory Pain

This model is used to determine the efficacy of test compounds against acute inflammatory pain produced by intradermal injection of Complete Freunds adjuvant (CFA) into a hind paw. Male Sprague Dawley rats (150-200 g; Taconic) are tested for baseline mechanical hind paw withdrawal thresholds by wrapping the rat in a towel and placing the hind paw (either left or right) in a modified Randal-Sellito paw pinch apparatus (Stoelting, Wood Dale, Ill.). A plastic plinth is placed on the plantar aspect of the hind paw and an increasing force (measured in grams) is applied to the hind paw. The test is terminated when the rat vocalizes or pulls its hind paw away from the plinth. The rat's hind paw withdrawal threshold (gm.) is recorded at that point. The mechanical stimulus is applied to each hind paw 3 times at each testing time point, and average mechanical hind paw withdrawal thresholds are determined for both the left and right hind paw. A maximal hind paw withdrawal threshold of 450 gm. is used to avoid tissue damage. Following determination of pre-CFA nociceptive thresholds, rats receive an intradermal injection of CFA (100 ul, 1 mg/ml) into the plantar aspect of the left hind paw and are subsequently returned to their cages in the animal holding room where they are maintained on soft bedding. In this model of acute inflammation, the inflammation develops over a 24 hour period, at which time edema and redness of the affected hind paw is observed (Stein et al. Pharmacol Biochem Behav 31:455, 1988). 24 hours following CFA injection, rats are tested for decreased mechanical paw withdrawal thresholds (mechanical hypersensitivity). Effects of the test compound on CFA-induced mechanical hypersensitivity are determined by dosing the test compound, vehicle and naproxen (20 mg/kg, p.o.; positive control) in different groups of rats and testing mechanical hind paw withdrawal thresholds at various times post-dosing depending on the pharmacokinetic properties of the test compound (n=8-10/group). Efficacy in the CFA model is evaluated by determining the % reversal of mechanical hypersensitivity using the formula:

$$\% reversal = \frac{( post \text{-} drug threshold - post \text{-} CFA threshold )}{( pre \text{-} CFA threshold - post \text{-} CFA threshold )} \times 100$$

At the conclusion of the experiment, all rats are immediately euthanized by CO₂.

In this assay, compounds of the invention have a reversal ranging from 0-57%. The Examples below have a reversal ranging from 0-57%.

### Rat Iodoacetate Model of Osteoarthritis Pain

This model is used to evaluate the efficacy of test compounds against chronic osteoarthritic pain produced by intraarticular injection of iodoacetate into a knee joint. Male Sprague Dawley rats (200-300 g; Taconic) are placed in individual plastic chambers on an elevated mesh galvanized steel platform and allowed to acclimate for approximately 60 min. Rats are then tested for baseline mechanical paw withdrawal thresholds by applying a series of calibrated von Frey filaments (0.25-15 g) to the left hind paw and determining the median withdrawal threshold using the Dixon “up-down” method (Chaplan et al., J Neurosci Meth 53:55, 1994). Pre-iodoacetate mechanical hind paw withdrawal thresholds are determined, and rats having a threshold <15 g are excluded from the study. Additionally, hind paw weight bearing is measured using an incapacitance instrument. Rats are tested for hind paw weight bearing by placing the animal in a Plexiglas box (approximately 4″ width, 4″ height, 5″ length) such that the posterior half of the animal is loosely restrained. This box is placed on an incapacitance analgesia meter (Stoelting Co.) such that the rats hind paws are positioned on two mechano-transducers that measure weight bearing (g) on each paw. Rats remain in this box for a period of ˜60 sec. during which average weight bearing on each hind paw is measured and displayed via LCD readout. Following determination of baseline pain related behaviors, rats are briefly anesthetized using isoflurane (1-5% to effect, inhalation) and receive an intraarticular injection of monosodium iodoacetate (2 mg/25 ul) into the left hind limb knee joint. Rats are continuously monitored until full recovery from the anesthetic (<5 min) and are subsequently returned to their cages where they are maintained on soft bedding. Intraarticular injection of iodoacetate has been found to produce degeneration of joint cartilage which is maximum at day 21, although the rats do not exhibit changes in body weight or locomotor activity and are found to be in otherwise good health (Fernihough et al. Pain 112:83, 2004). In-house results have demonstrated that mechanical hypersensitivity (von Frey filaments) and decreased weight bearing (incapacitance instrument) persists for >8 weeks following iodoacetate injection. 6 weeks following iodoacetate injection, rats are tested for these pain-related behaviors. Effects of test compound on iodoacetate-induced mechanical hypersensitivity and decreased weight bearing are determined by dosing the test compound, vehicle and naproxen (20 mg/kg, p.o.; positive control) in different groups of rats and testing mechanical hind paw withdrawal thresholds and weight bearing at various times post-dosing depending on the pharmacokinetic properties of the test compound (n=8-10/group). Efficacy in the iodoacetate model is evaluated by determining the % reversal of mechanical hypersensitivity and weight bearing using the formula:

$$\% reversal = \frac{( \begin{matrix}post \text{-} drug threshold - \\ post \text{-} iodoacetate threshold\end{matrix} )}{( \begin{matrix}pre \text{-} iodoacetate threshold - \\ post \text{-} iodoacetate threshold\end{matrix} )} \times 100$$

At the conclusion of the experiment, all rats are immediately euthanized by CO₂.

### Methods of Synthesis

Several methods for preparing the compounds of this invention are illustrated in the following Examples. Starting materials and the requisite intermediates are in some cases commercially available, or can be prepared according to literature procedures or as illustrated herein. All ¹H NMR spectra were obtained on instrumentation at a field strength of 400 or 500 MHz.

The abbreviations used hereinunder are as follows unless specified otherwise:
4-MeBnOH 4-Methylbenzyl alcohol
CDI 1,1′-Carbonyldiimidazole
TEA Triethylamine
TBSCl t-Butyldimethylsilyl chloride
DMF Dimethylformamide
(+)-BINAP (+)-2,2′-Bis(diphenylphosphino)-1,1′-binaphthyl
NaOtBu Sodium t-butoxide
DIPEA Diisopropylethylamine
EtOAc Ethyl acetate
TBSOTf t-Butyldimethylsilyl triflate
TBS t-butyldimethylsilyl
THF Tetrahydrofuran
DMAP 4-Dimethylaminopyridine
RT Room temperature
h Hours
min Minutes
DCM Dichloromethane
MeCN Acetonitrile
iPrOH 2-Propanol
n-BuOH 1-Butanol
EDC 1-(3-Dimethylaminopropyl)-3-ethylcarbodiimide hydrochloride
HOAt 1-Hydroxy-7-azabenzotriazole

### Intermediates and Examples

The following examples are provided so that the invention might be more fully understood. These examples are illustrative only and should not be construed as limiting the invention in any way.

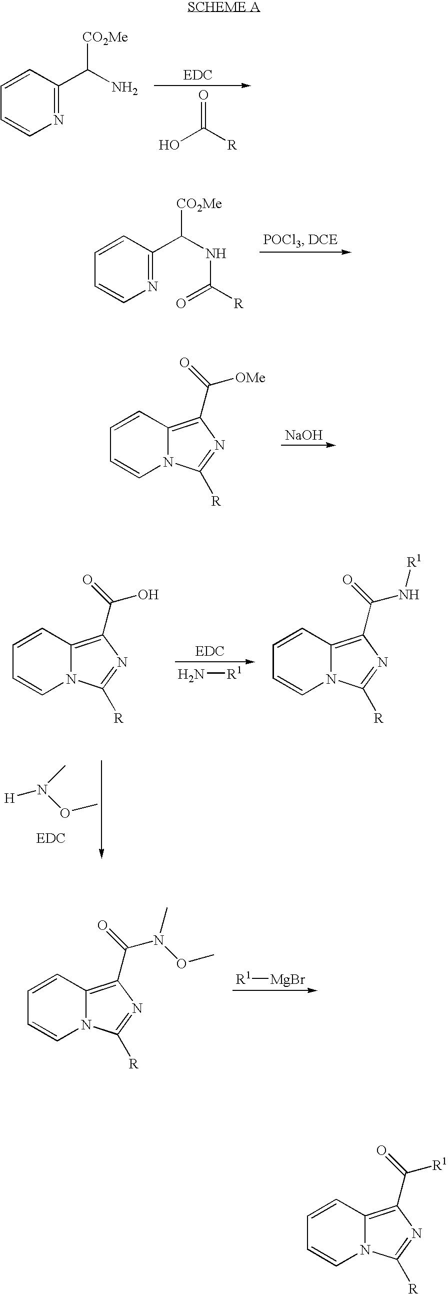

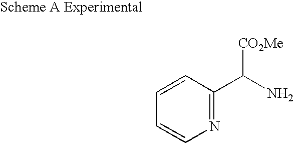

### Methyl amino(pyridin-2-yl)acetate

To a solution of methyl 2-pyridylacetate (25.4 g, 168 mmol) in glacial acetic acid (41 mL) at 0° C. was added portion-wise a solution of sodium nitrite (11.6 g, 168 mmol) in water (20 mL). The reaction mixture was stirred at ambient temperature for 1 h. Water (82 mL) was added and the solution was stirred for an additional 1 h. The mixture was extracted with DCM (3×). The combined organic extracts were dried over MgSO₄, filtered, and concentrated.

To a solution of this intermediate in methanol (275 mL) was added palladium (10% on carbon; 2.75 g) followed by concentrated HCl (20 drops). The mixture was stirred under a balloon of hydrogen for 48 h. The reaction mixture was filtered through Celite and washed with MeOH. The filtrate was concentrated under reduced pressure. Ether was added and the solid formed was filtered off. The filtrate was concentrated and DCM was added followed by HCl (4.0 M in dioxane; 100 mL). The mixture was concentrated to give the HCl salt of the title compound (32.1 g, 80%). ¹H NMR (400 MHz, DMSO-d₆) δ 8.50-8.48 (m, 1H), 7.82-7.78 (m, 1H), 7.52-7.50 (m, 1H), 7.33-7.30 (m, 1H), 4.80 (s, 1H), 3.59 (s, 3H).

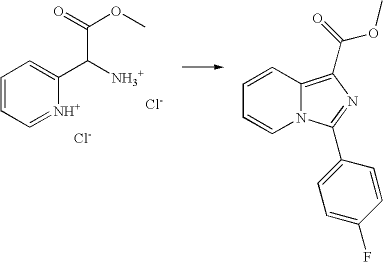

Methyl 3-(4-fluorophenyl)imidazo[1,5-a]pyridine-1-carboxylate. To a mixture of methylamino(pyridin-2-yl)acetate (bis-HCl salt, 3.5 g, 14.64 mmol) in dichloromethane (73.2 ml) and saturated aqueous sodium bicarbonate (73.2 ml) was added 4-fluorobenzoyl chloride (1.902 ml, 16.10 mmol). The reaction was stirred at 25° C. for 75 min, then partitioned between dichloromethane and saturated aqueous sodium bicarbonate. The aqueous solution was extracted twice more with dichloromethane, and the combined organic solutions were dried (Na₂SO₄) and concentrated. This material was dissolved in 1,2-dichloroethane (73.2 ml), and phosphorus oxychloride (13.65 ml, 146 mmol) was added. The reaction was heated at 100-110° C. for 40 h, adding additional phosphorus oxychloride after 4 h (13.65 ml) and 28 h (10 ml). The reaction was remove from heat and concentrated, then partitioned between dichloromethane and saturated aqueous sodium bicarbonate. The aqueous solution was extracted once more with dichloromethane, and the combined organic solutions were dried (Na₂SO₄) and concentrated. The residue was purified by column chromatography on silica gel to give 3.18 g of the title compound as a yellow-brown solid.

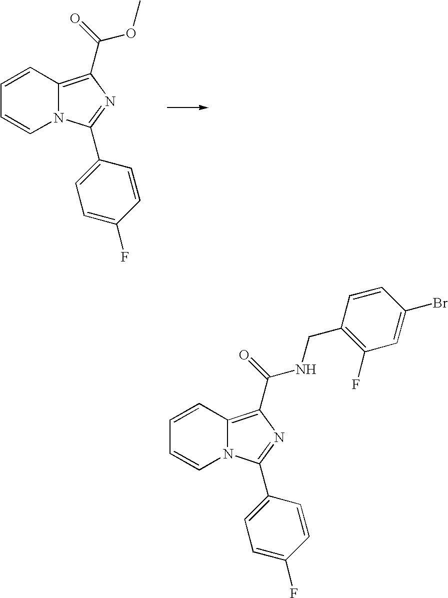

N-(4-bromo-2-fluorobenzyl)-3-(4-fluorophenyl)imidazo[1,5-a]pyridine-1-carboxamide. Trimethylaluminum (0.814 ml, 1.628 mmol) was added to a suspension of methyl 3-(4-fluorophenyl)imidazo[1,5-a]pyridine-1-carboxylate (200 mg, 0.740 mmol) and 4-bromo-2-fluorobenzylamine hydrochloride (196 mg, 0.814 mmol) in toluene (8 ml). The reaction was heated at 90° C. for 3.5 h, then cooled to room temperature. Saturated aqueous Rochelle's salt and EtOAc were added, and the mixture was stirred for 30 min, then partitioned between ethyl acetate and saturated aqueous Rochelle's salt. The organic solutions were dried (Na₂SO₄) and concentrated. The residue was purified by column chromatography on silica gel to give 324 mg of the titled compound as a yellow foam.

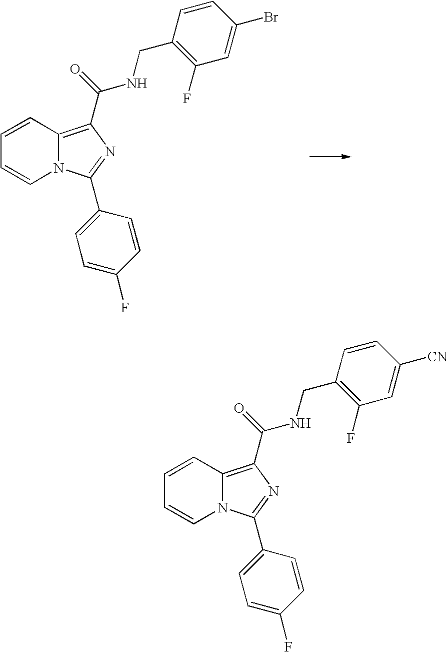

N-(4-cyano-2-fluorobenzyl)-3-(4-fluorophenyl)imidazo[1,5-a]pyridine-1-carboxamide. N-(4-bromo-2-fluorobenzyl)-3-(4-fluorophenyl)imidazo[1,5-a]pyridine-1-carboxamide (70 mg, 0.158 mmol), N,N-dimethylacetamide (1 ml), zinc cyanide (37.2 mg, 0.317 mmol), zinc (1.242 mg, 0.019 mmol), Pd₂dba₃ (5.80 mg, 6.33 μmol), and dppf (0.100 μl, 0.013 mmol) were combined in a screw-cap vial and purged with argon. The vial was sealed and heated at 120° C. for 3 h. The reaction was removed from heat and filtered, washing with DMSO. The solution was purified by preparative HPLC (reverse phase C-18), eluting with Acetonitrile/Water+0.1% TFA, to give 48 mg of the titled compound as a tan solid. ¹H NMR (500 MHz, CDCl₃) δ 8.36 (dt, 1H, J=9, 1 Hz), 8.20 (d, 1H, J=7 Hz), 7.75 (m, 2H), 7.67 (br t, 1H, J=6 Hz), 7.61 (t, 1H), J=8 Hz), 7.42 (dd, 1H, J=8, 1 Hz), 7.35 (dd, 1H, J=9, 1 Hz), 7.26 (m, 2H), 7.08 (m, 1H), 6.77 (m, 1H), 4.78 (d, 1H, J=7 Hz). See Table for HRMS data.

TABLE A

| Example No. | Structure | Name | MS (M + 1)* |
|---|---|---|---|
| 1 | 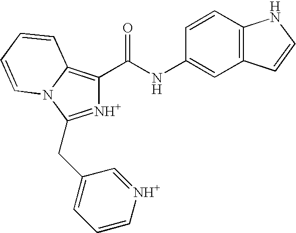 | N-1H-indol-5-yl-3-(pyridin-3- ylmethyl)imidazo[1,5-a]pyridine-1- carboxamide | 368.1521 |
| | 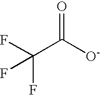 | | |
| 2 | 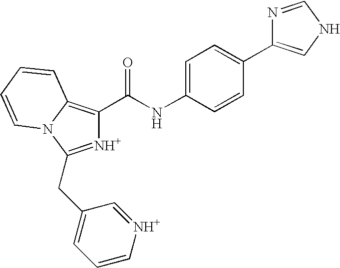 | N-[4-(1H-imidazol-4-yl)phenyl]-3- pyridin-3-ylmethyl)imidazo[1,5- a]pyridine-1-carboxamide | 395.1629 |
| | 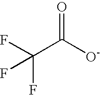 | | |
| 3 | 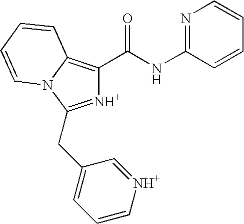 | N-pyridin-2-yl-3-(pyridin-3- ylmethyl)imidazo[1,5-a]pyridine-1- carboxamide | 330.135 |
| | 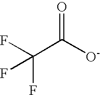 | | |
| 4 | 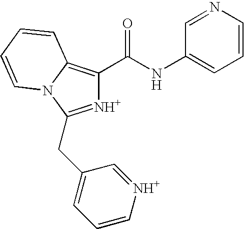 | N-pyridin-3-yl-3-(pyridin-3- ylmethyl)imidazo[1,5-a]pyridine-1- carboxamide | 330.1351 |
| | 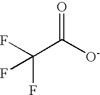 | | |
| 5 | 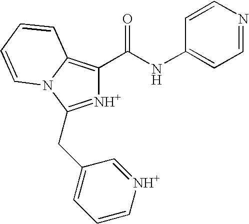 | N-pyridin-4-yl-3-(pyridin-3- ylmethyl)imidazo[1,5-a]pyridine-1- carboxamide | 330.1348 |
| | 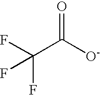 | | |
| 6 | 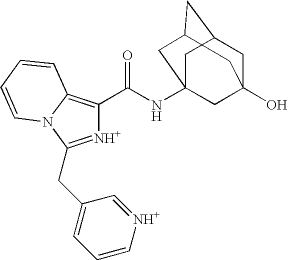 | N-(3-hydroxyadamantan-1-yl)-3- (pyridin-3-ylmethyl)imidazo[1,5- a]pyridine-1-carboxamide | 403.2147 |
| | 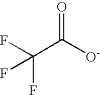 | | |
| 7 | 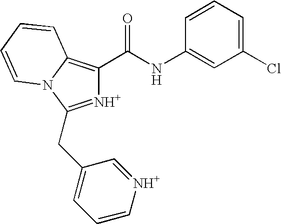 | N-(3-chloropheny1)-3-(pyridin-3- ylmethyl)imidazo[1,5-a]pyridine-1- carboxamide | 363.1019 |
| | 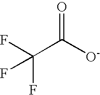 | | |
| 8 | 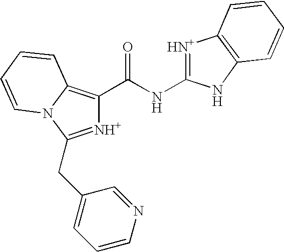 | N-1H-benzimidazol-2-yl-3-(pyridin-3- ylmethyl)imidazo[1,5-a]pyridine-1- carboxamide | 369.1463 |
| | 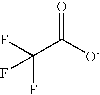 | | |
| 9 | 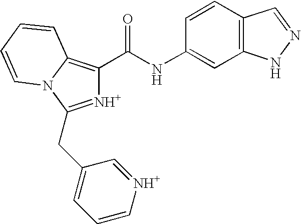 | N-1H-indazol-6-yl-3-(pyridin-3- ylmethyl)imidazo[1,5-a]pyridine-1- carboxamide | 369.1465 |
| | 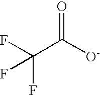 | | |
| 10 | 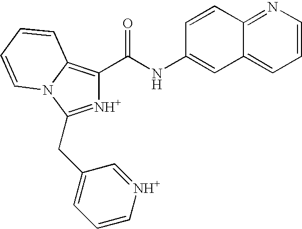 | 3-(pyridin-3-ylmethyl)-N-quinolin-6- ylimidazo[1,5-a]pyridine-1- carboxamide | 380.1509 |
| | 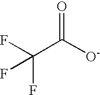 | | |
| 11 | 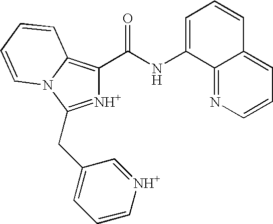 | 3-(pyridin-3-ylmethyl)-N-quinolin-8- ylimidazo[1,5-a]pyridine-1- carboxamide | 380.1512 |
| | 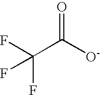 | | |
| 12 | 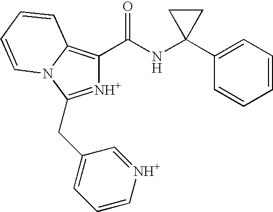 | N-(1-phenylcyclopropyl)-3-(pyridin-3- ylmethyl)imidazo[1,5-a]pyridine-1- carboxamide | 369.1717 |
| | 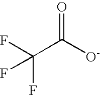 | | |
| 13 | 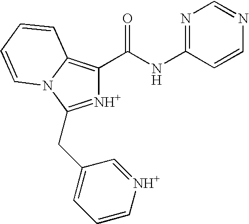 | 3-(pyridin-3-ylmethyl)-N-pyrimidin-4- ylimidazo[1,5-a]pyridine-1- carboxamide | 331.1302 |
| | 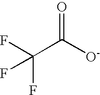 | | |
| 14 | 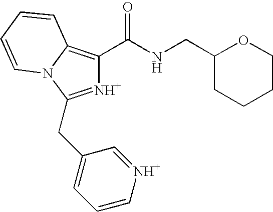 | 3-(pyridin-3-ylmethyl)-N-(tetrahydro- 2H-pyran-2-ylmethyl)imidazo[1,5- a]pyridine-1-carboxamide | 351.1824 |
| | 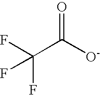 | | |
| 15 | 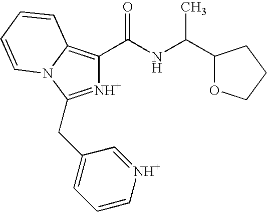 | 3-pyridin-3-ylmethyl)-N-[1- (tetrahydrofuran-2-yl)ethyl]imidazo[1,5- a]pyridine-1-carboxamide | 351.1822 |
| | 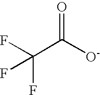 | | |
| 16 | 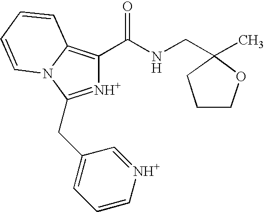 | N-[(2-methyltetrahydrofuran-2- yl)methyl]-3-(pyridin-3- ylmethyl)imidazo[1,5-a]pyridine-1- carboxamide | 351.182 |
| | 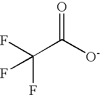 | | |
| 17 | 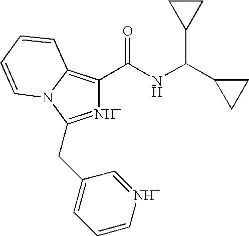 | N-(dicyclopropylmethyl)-3-(pyridin-3- ylmethyl)imidazo[1,5-a]pyridine-1- carboxamide | 347.1872 |
| | 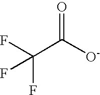 | | |
| 18 | 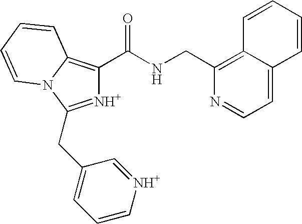 | N-(isoquinolin-1-ylmethyl)-3-(pyridin- 3-ylmethyl)imidazo[1,5-a]pyridine-1- carboxamide | 394.1669 |
| | 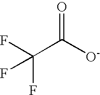 | | |
| 19 | 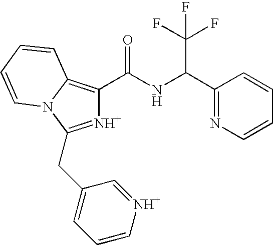 | 3-(pyridin-3-ylmethyl)-N-(2,2,2- trifluoro-1-pyridin-2- ylethyl)imidazo[1,5-a]pyridine-1- carboxamide | 412.1388 |
| | 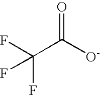 | | |
| 20 | 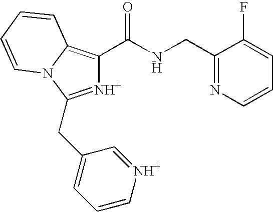 | N-[(3-fluoropyridin-2-yl)methyl]-3- (pyridin-3-ylmethyl)imidazo[1,5- a]pyridine-1-carboxamide | 362.1419 |
| | 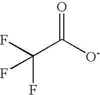 | | |
| 21 | 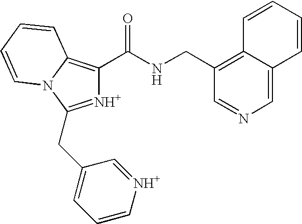 | N-(isoquinolin-4-ylmethyl)-3-(pyridin- 3-ylmethyl)imidazo[1,5-a]pyridine-1- carboxamide | 394.1672 |
| | 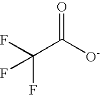 | | |
| 22 | 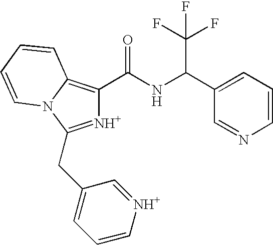 | 3-(pyridin-3-ylmethyl)-N-(2,2,2- trifluoro-1-pyridin-3- ylethyl)imidazo[1,5-a]pyridine-1- carboxamide | 412.1387 |
| | 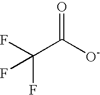 | | |
| 23 | 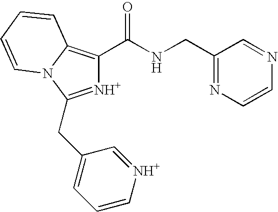 | N-(pyrazin-2-ylmethyl)-3-(pyridin-3- ylmethyl)imidazo[1,5-a]pyridine-1- carboxamide | 367.1294 |
| | 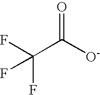 | | |
| 24 | 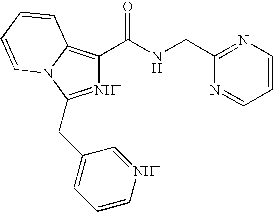 | 3-(pyridin-3-ylmethyl)-N-(pyrimidin-2- ylmethyl)imidazo[1,5-a]pyridine-1- carboxamide | 367.1294 |
| | 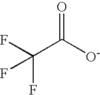 | | |
| 25 | 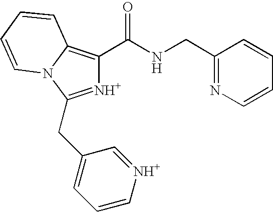 | N-(pyridin-2-ylmethyl)-3-(pyridin-3- ylmethyl)imidazo[1,5-a]pyridine-1- carboxamide | 344.1512 |
| | 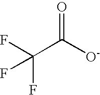 | | |
| 26 | 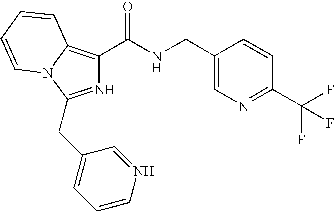 | 3-(pyridin-3-ylmethyl)-N-{[6- (trifluoromethyl)pyridin-3- yl]methyl}imidazo[1,5-a]pyridine-1- carboxamide | 412.1392 |
| | 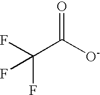 | | |
| 27 | 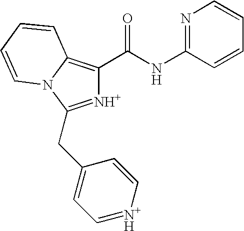 | N-pyridin-2-yl-3-(pyridin-4- ylmethyl)imidazo[1,5-a]pyridine-1- carboxamide | 330.136 |
| | 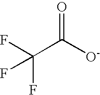 | | |
| 28 | 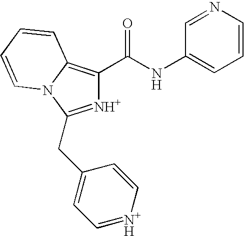 | N-pyridin-3-yl-3-(pyridin-4- ylmethyl)imidazo[1,5-a]pyridine-1- carboxamide | 330.1368 |
| | 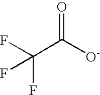 | | |
| 29 | 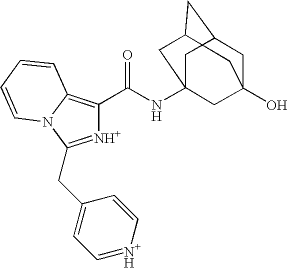 | N-(3-hydroxyadamantan-1-yl)-3- (pyridin-4-ylmethyl)imidazo[1,5- a]pyridine-1-carboxamide | 403.2147 |
| | 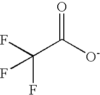 | | |
| 30 | 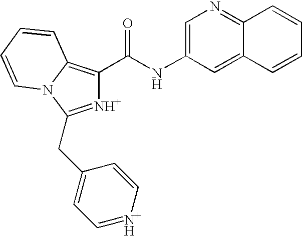 | 3-(pyridin-4-ylmethyl)-N-quinolin-3- ylimidazo[1,5-a]pyridine-1- carboxamide | 380.153 |
| | 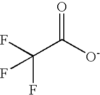 | | |
| 31 | 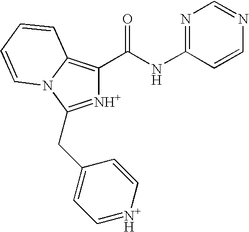 | 3-(pyridin-4-ylmethyl)-N-pyrimidin-4- ylimidazo[1,5-a]pyridine-1- carboxamide | 331.1316 |
| | 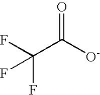 | | |
| 32 | 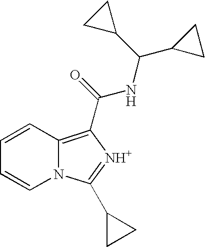 | 3-cyclopropyl-N- (dicyclopropylmethyl)imidazo[1,5- a]pyridine-1-carboxamide | 591.3407 [2M + H]1+ |
| | 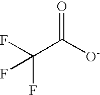 | | |
| 33 | 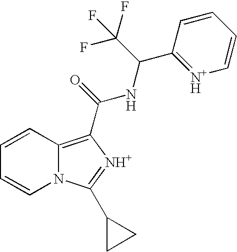 | 3-cyclopropyl-N-(2,2,2-trifluoro-1- pyridin-2-ylethyl)imidazo[1,5- a]pyridine-1-carboxamide | 361.1275 |
| | 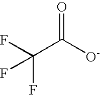 | | |
| 34 | 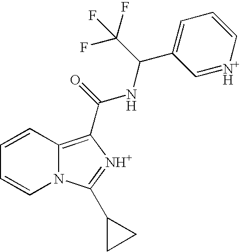 | 3-cyclopropyl-N-(2,2,2-trifluoro-1- pyridin-3-ylethyl)imidazo[1,5- a]pyridine-1-carboxamide | 361.1276 |
| | 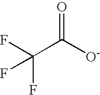 | | |
| 35 | 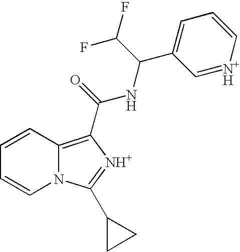 | 3-cyclopropyl-N-(2,2-difluoro-1- pyridin-3-ylethyl)imidazo[1,5- a]pyridine-1-carboxamide | 343.137 |
| | 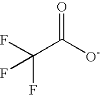 | | |
| 36 | 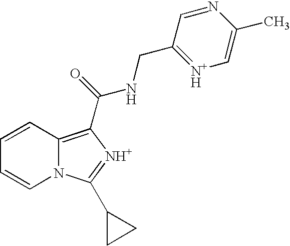 | 3-cyclopropyl-N-[(5-methylpyrazin-2- yl)methyl]imidazo[1,5-a]pyridine-1- carboxamide | |
| | 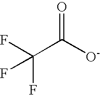 | | |
| 37 | 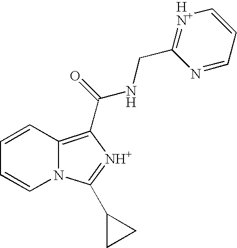 | 3-cyclopropyl-N-(pyrimidin-2- ylmethyl)imidazo[1,5-a]pyridine-1- carboxamide | 587.2593 [2M + H]1+ |
| | 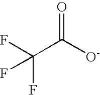 | | |
| 38 | 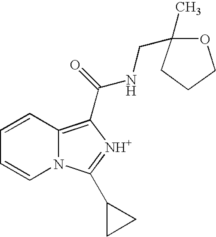 | 3-cyclopropyl-N-[(2- methyltetrahydrofuran-2- yl)methyl]imidazo[1,5-a]pyridine-1- carboxamide | |
| | 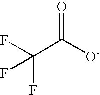 | | |
| 39 | 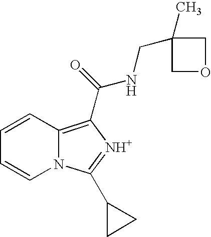 | 3-cyclopropyl-N-[(3-methyloxetan-3- yl)methyl]imidazo[1,5-a]pyridine-1- carboxamide | 286.1568 |
| | 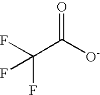 | | |
| 40 | 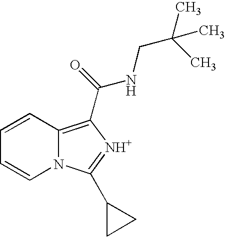 | 3-cyclopropyl-N-(2,2- dimethylpropyl)imidazo[1,5-a]pyridine- 1-carboxamide | 543.3406 [2M + H]1+ |
| | 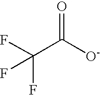 | | |
| 41 | 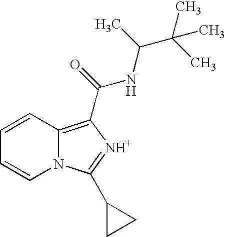 | 3-cyclopropyl-N-(1,2,2- trimethylpropyl)imidazo[1,5-a]pyridine- 1-carboxamide | 571.373 [2M + H]1+ |
| | 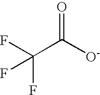 | | |
| 42 | 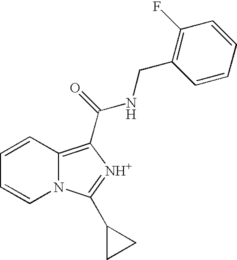 | 3-cyclopropyl-N-(2- fluorobenzyl)imidazo[1,5-a]pyridine-1- carboxamide | 310.1366 |
| | 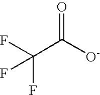 | | |
| 43 | 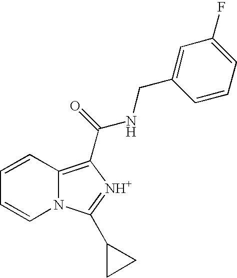 | 3-cyclopropyl-N-(3- fluorobenzyl)imidazo[1,5-a]pyridine-1- carboxamide | 310.1364 |
| | 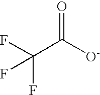 | | |
| 44 | 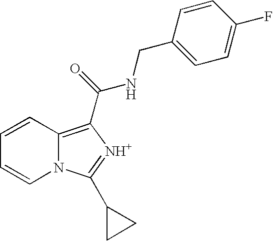 | 3-cyclopropyl-N-(4- fluorobenzyl)imidazo[1,5-a]pyridine-1- carboxamide | 619.2602 [2M + H]1+ |
| | 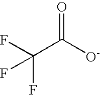 | | |
| 45 | 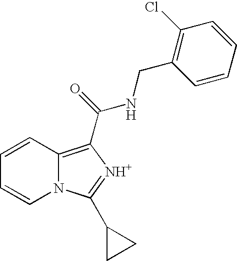 | N-(2-chlorobenzyl)-3- cyclopropylimidazo[1,5-a]pyridine-1- carboxamide | 651.2003 [2M + H]1+ |
| | 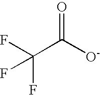 | | |
| 46 | 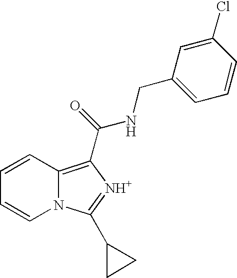 | N-(3-chlorobenzyl)-3- cyclopropylimidazo[1,5-a]pyridine-1- carboxamide | 326.108 |
| | 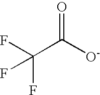 | | |
| 47 | 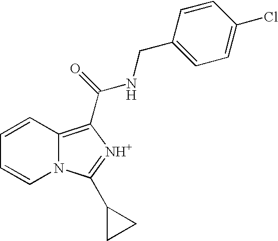 | N-(4-chlorobenzyl)-3- cyclopropylimidazo[1,5-a]pyridine-1- carboxamide | 326.1067 |
| | 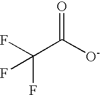 | | |
| 48 | 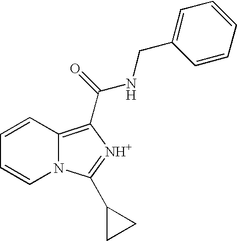 | N-benzyl-3-cyclopropylimidazo[1,5- a]pyridine-1-carboxamide | 292.1462 |
| | 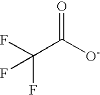 | | |
| 49 | 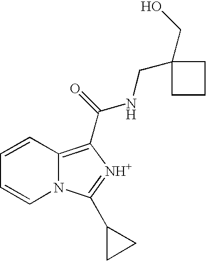 | 3-cyclopropyl-N-{[1- (hydroxymethyl)cyclobutyl]methyl} imidazo[1,5-a]pyridine-1-carboxamide | 599.3318 [2M + H]1+ |
| | 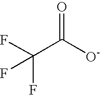 | | |
| 50 | 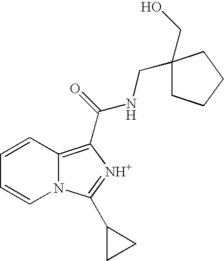 | 3-cyclopropyl-N-{[1- (hydroxymethyl)cyclopentyl]methyl} imidazo[1,5-a]pyridine-1-carboxamide | 314.1879 |
| | 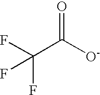 | | |
| 51 | 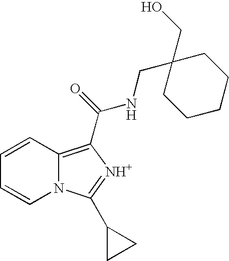 | 3-cyclopropyl-N-{[1- (hydroxymethyl)cyclohexyl]methyl} imidazo[1,5-a]pyridine-1-carboxamide | 328.2032 |
| | 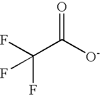 | | |
| 52 | 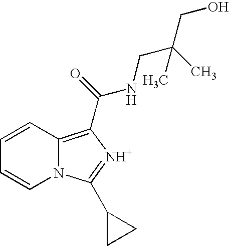 | 3-cyclopropyl-N-(3-hydroxy-2,2- dimethylpropyl)imidazo[1,5-a]pyridine- 1-carboxamide | 575.3312 [2M + H]1+ |
| | 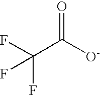 | | |
| 53 | 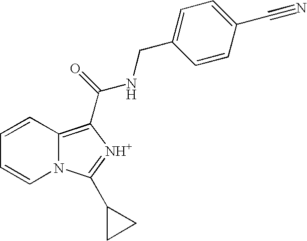 | N-(4-cyanobenzyl)-3- cyclopropylimidazo[1,5-a]pyridine-1- carboxamide | 334.163 [M + NH4]1+ |
| | 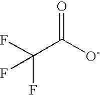 | | |
| 54 | 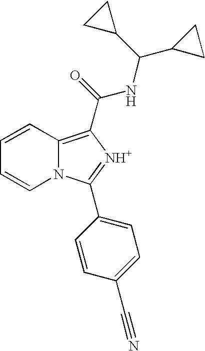 | 3-(4-cyanophenyl)-N- (dicyclopropylmethyl)imidazo[1,5- a]pyridine-1-carboxamide | |
| | 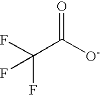 | | |
| 55 | 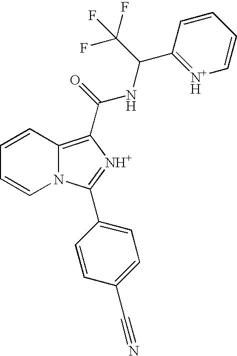 | 3-(4-cyanophenyl)-N-(2,2,2-trifluoro-1- pyridin-2-ylethyl)imidazo[1,5- a]pyridine-1-carboxamide | |
| | 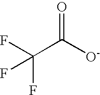 | | |
| 56 | 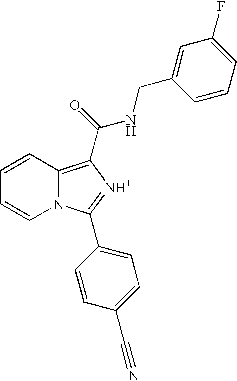 | 3-(4-cyanophenyl)-N-(3- fluorobenzyl)imidazo[1,5-a]pyridine-1- carboxamide | |
| | 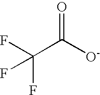 | | |
| 57 | 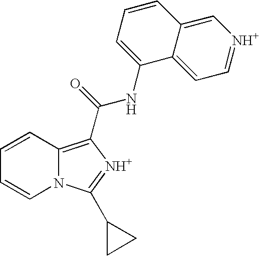 | 3-cyclopropyl-N-isoquinolin-5- ylimidazo[1,5-a]pyridine-1- carboxamide | 329.1414 |
| | 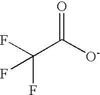 | | |
| 58 | 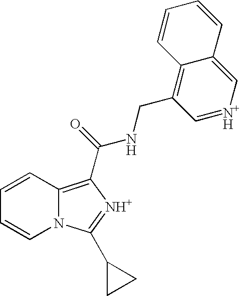 | 3-cyclopropyl-N-(isoquinolin-4- ylmethyl)imidazol[1,5-a]pyridine-1- carboxamide | 343.1581 |
| | 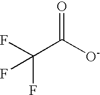 | | |
| 59 | 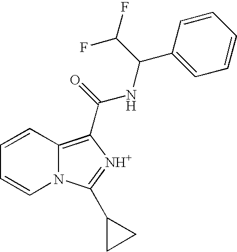 | 3-cyclopropyl-N-(2,2-difluoro-1- phenylethyl)imidazo[1,5-a]pyridine-1- carboxamide | 342.1444 |
| | 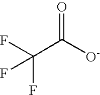 | | |
| 60 | 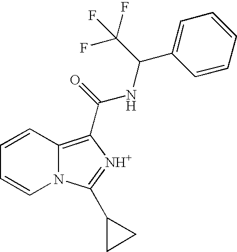 | 3-cyclopropyl-N-(2,2,2-trifluoro-1- phenylethyl)imidazo[1,5-a]pyridine-1- carboxamide | 360.1292 |
| | 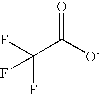 | | |
| 61 | 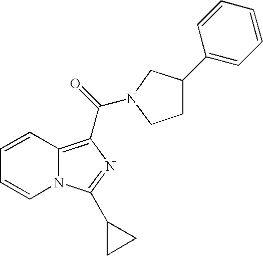 | 3-cyclopropyl-1-[(3-phenylpyrrolidin-1- yl)carbonyl]imidazo[1,5-a]pyridine | 332.1752 |
| 62 | 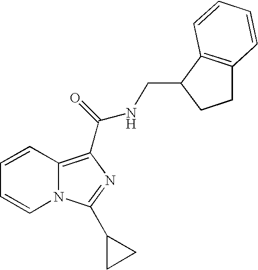 | 3-cyclopropyl-N-(2,3-dihydro-1H- inden-1-ylmethyl)imidazo[1,5- a]pyridine-1-carboxamide | 332.1755 |
| 63 | 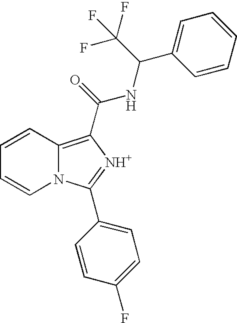 | 3-(4-fluorophenyl)-N-(2,2,2-trifluoro-1- phenylethyl)imidazo[1,5-a]pyridine-1- carboxamide | 414.1210 |
| | 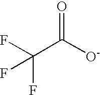 | | |
| 64 | 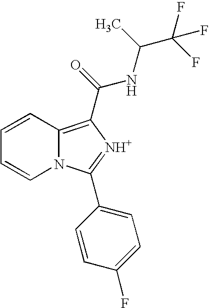 | 3-(4-fluorophenyl)-N-(2,2,2-trifluoro-1- methylethyl)imidazo[1,5-a]pyridine-1- carboxamide | 352.1050 |
| | 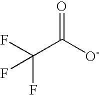 | | |
| 65 | 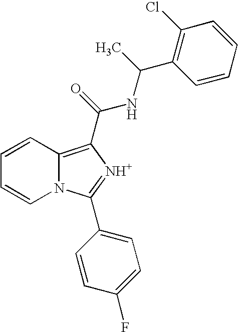 | N-[1-(2-chlorophenyl)ethyl]-3-(4- fluorophenyl)imidazo[1,5-a]pyridine-1- carboxamide | 394.1100 |
| | 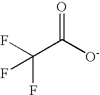 | | |
| 66 | 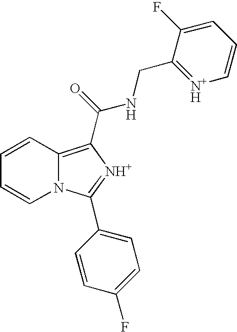 | 3-(4-fluorophenyl)-N-[(3-fluoropyridin- 2-yl)methyl]imidazo[1,5-a]pyridine-1- carboxamide | 365.1194 |
| | 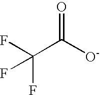 | | |
| 67 | 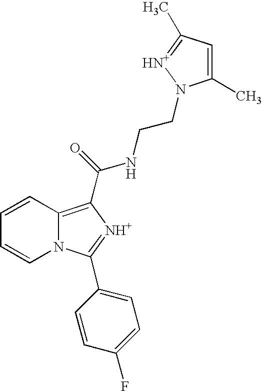 | N-[2-(3,5-dimethyl-1H-pyrazol-1- yl)ethyl]-3-(4- fluorophenyl)imidazo[1,5-a]pyridine-1- carboxamide | 378.1711 |
| | 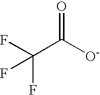 | | |
| 68 | 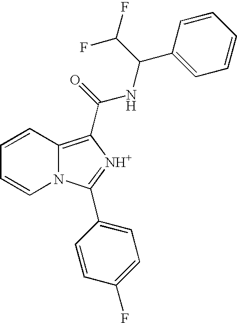 | N-(2,2-difluoro-1-phenylethyl)-3-(4- fluorophenyl)imidazo[1,5-a]pyridine-1- carboxamide | 396.1301 |
| | 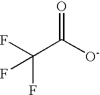 | | |
| 69 | 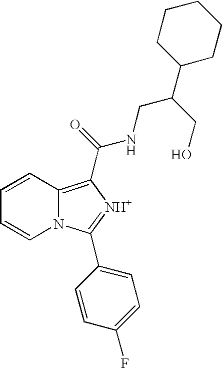 | N-(2-cyclohexyl-3-hydroxypropyl)-3-(4- fluorophenyl)imidazo[1,5-a]pyridine-1- carboxamide | 396.2064 |
| | 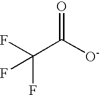 | | |
| 70 | 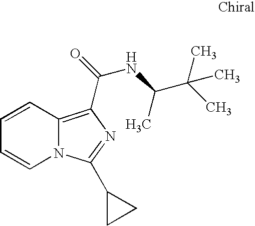 | 3-cyclopropyl-N-[(1R)-1,2,2- trimethylpropyl]imidazo[1,5-a]pyridine- 1-carboxamide | 286.1909 |
| 71 | 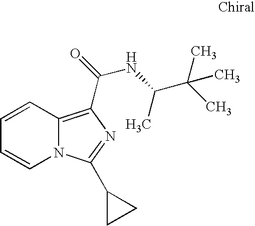 | 3-cyclopropyl-N-[(1S)-1,2,2- trimethylpropyl]imidazo[1,5-a]pyridine- 1-carboxamide | 286.1909 |
| 72 | 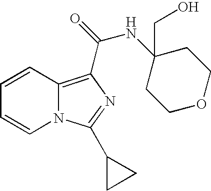 | 3-cyclopropyl-N-[4- (hydroxymethyl)tetrahydro-2H-pyran-4- yl]imidazo[1,5-a]pyridine-1- carboxamide | 316.163 |
| 73 | 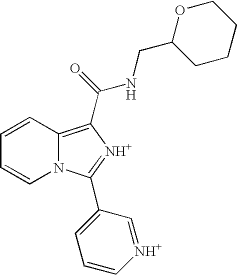 | 3-pyridin-3-yl-N-(tetrahydro-2H-pyran- 2-ylmethyl)imidazo[1,5-a]pyridine-1- carboxamide | 337.1655 |
| | 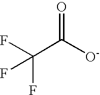 | | |
| 74 | 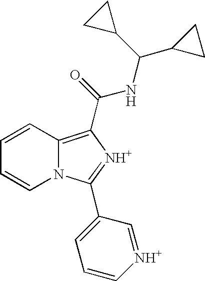 | N-(dicyclopropylmethyl)-3-pyridin-3- ylimidazo[1,5-a]pyridine-1- carboxamide | 333.1706 |
| | 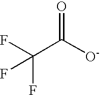 | | |
| 75 | 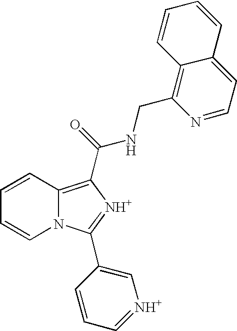 | N-(isoquinolin-1-ylmethyl)-3-pyridin- 3-ylimidazo[1,5-a]pyridine-1- carboxamide | 380.15 |
| | 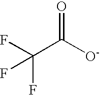 | | |
| 76 | 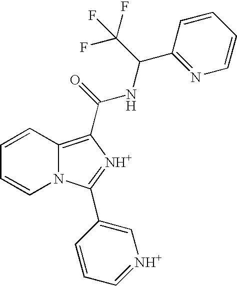 | 3-pyridin-3-yl-N-(2,2,2-trifluoro-1- pyridin-2-ylethyl)imidazo[1,5- a]pyridine-1-carboxamide | 398.1218 |
| | 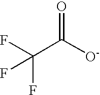 | | |
| 77 | 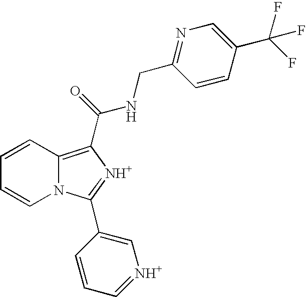 | 3-pyridin-3-yl-N-{[5- (trifluoromethyl)pyridin-2- yl]methyl}imidazo[1,5-a]pyridine-1- carboxamide | 398.1217 |
| | 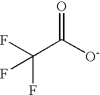 | | |
| 78 | 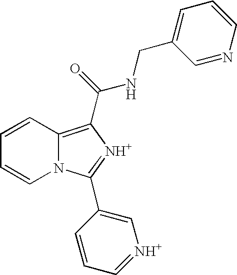 | 3-pyridin-3-yl-N-(pyridin-3- ylmethyl)imidazo[1,5-a]pyridine-1- carboxamide | 330.1347 |
| | 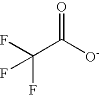 | | |
| 79 | 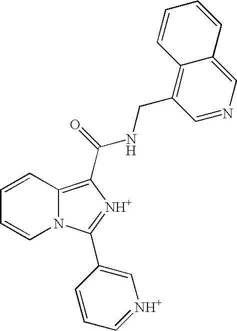 | N-(isoquinolin-4-ylmethyl)-3-pyridin-3- ylimidazo[1,5-a]pyridine-1- carboxamide | 380.1506 |
| | 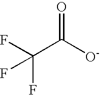 | | |
| 80 | 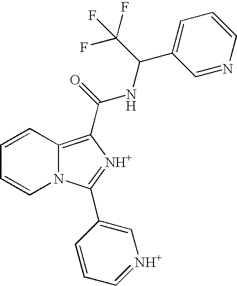 | 3-pyridin-3-yl-N-(2,2,2-trifluoro-1- pyridin-3-ylethyl)imidazo[1,5- a]pyridine-1-carboxamide | 398.122 |
| | 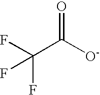 | | |
| 81 | 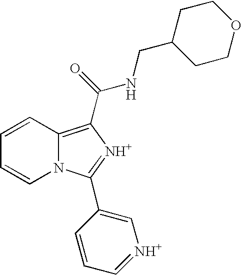 | 3-pyridin-3-yl-N-(tetrahydro-2H-pyran- 4-ylmethyl)imidazo[1,5-a]pyridine-1- carboxamide | 337.1658 |
| | 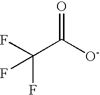 | | |
| 82 | 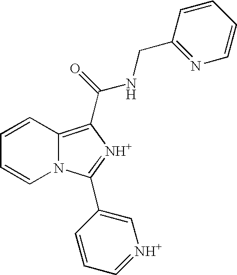 | 3-pyridin-3-yl-N-(pyridin-2- ylmethyl)imidazo[1,5-a]pyridine-1- carboxamide | 330.1346 |
| | 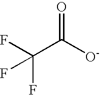 | | |
| 83 | 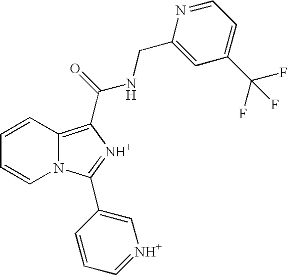 | 3-pyridin-3-yl-N-{[4- (trifluoromethyl)pyridin-2- yl]methyl}imidazo[1,5-a]pyridine-1- carboxamide | 398.1218 |
| | 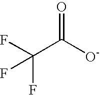 | | |
| 84 | 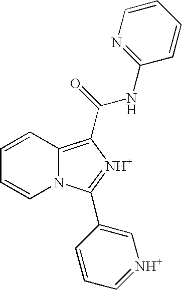 | N-pyridin-2-yl-3-pyridin-3- ylimidazo[1,5-a]pyridine-1- carboxamide | 316.1189 |
| | 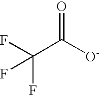 | | |
| 85 | 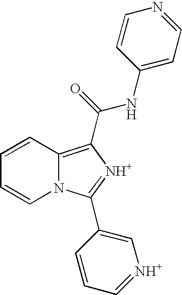 | 3-pyridin-3-yl-N-pyridin-4- ylimidazo[1,5-a]pyridine-1- carboxamide | 316.1189 |
| | 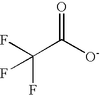 | | |
| 86 | 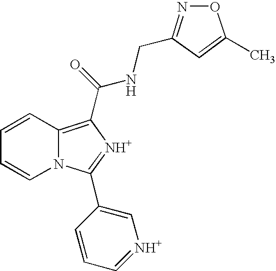 | N-[(5-methylisoxazol-3-yl)methyl]-3- pyridin-3-ylimidazo[1,5-a]pyridine-1- carboxamide | 334.1297 |
| | 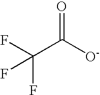 | | |
| 87 | 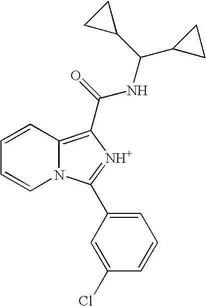 | 3-(3-chlorophenyl)-N- (dicyclopropylmethyl)imidazo[1,5- a]pyridine-1-carboxamide | 366.1362 |
| | 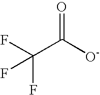 | | |
| 88 | 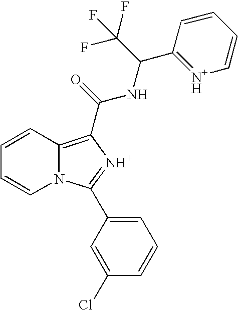 | 3-(3-chlorophenyl)-N-(2,2,2-trifluoro-1- pyridin-2-ylethyl)imidazo[1,5- a]pyridine-1-carboxamide | 431.0854 |
| | 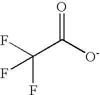 | | |
| 89 | 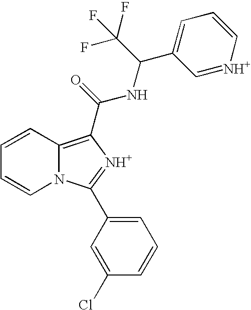 | 3-(3-chlorophenyl)-N-(2,2,2-trifluoro-1- pyridin-3-ylethyl)imidazo[1,5- a]pyridine-1-carboxamide | 431.086 |
| | 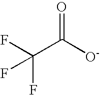 | | |
| 90 | 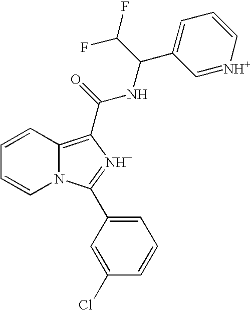 | 3-(3-chlorophenyl)-N-(2,2-difluoro-1- pyridin-3-ylethyl)imidazo[1,5- a]pyridine-1-carboxamide | 413.0986 |
| | 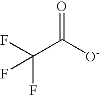 | | |
| 91 | 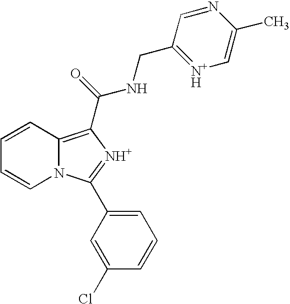 | 3-(3-chlorophenyl)-N-[(5- methylpyrazin-2-yl)methyl]imidazo[1,5- a]pyridine-1-carboxamide | 378.1107 |
| | 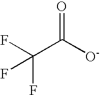 | | |
| 92 | 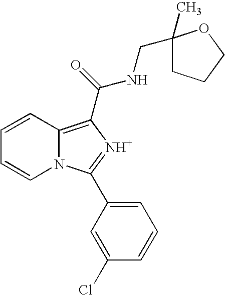 | 3-(3-chlorophenyl)-N-[(2- methyltetrahydrofuran-2- yl)methyl]imidazo [1,5-a]pyridine-1- carboxamide | 370.1313 |
| | 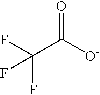 | | |
| 93 | 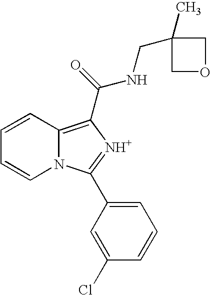 | 3-(3-chlorophenyl)-N-[(3-methyloxetan- 3-yl)methyl]imidazo[1,5-a]pyridine-1- carboxamide | 356.1152 |
| | 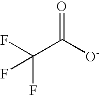 | | |
| 94 | 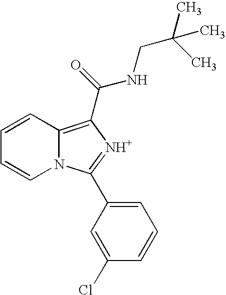 | 3-(3-chlorophenyl)-N-(2,2- dimethylpropyl)imidazo[1,5-a]pyridine- 1-carboxamide | 342.1359 |
| | 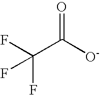 | | |
| 95 | 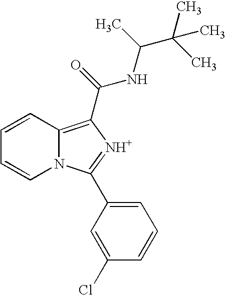 | 3-(3-chlorophenyl)-N-(1,2,2- trimethylpropyl)imidazo[1,5-a]pyridine- 1-carboxamide | 356.1515 |
| | 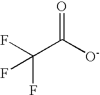 | | |
| 96 | 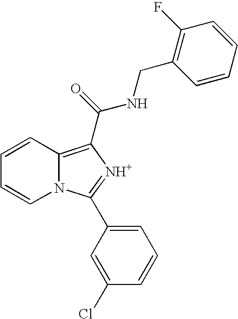 | 3-(3-chlorophenyl)-N-(2- fluorobenzyl)imidazo[1,5-a]pyridine-1- carboxamide | 380.0949 |
| | 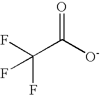 | | |
| 97 | 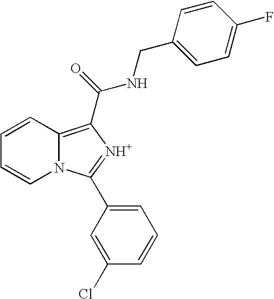 | 3-(3-chlorophenyl)-N-(4- fluorobenzyl)imidazo[1,5-a]pyridine-1- carboxamide | 380.0947 |
| | 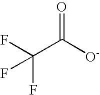 | | |
| 98 | 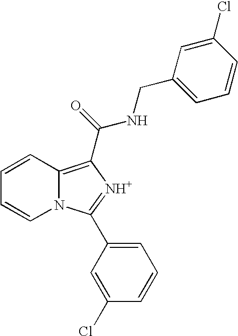 | N-(3-chlorobenzyl)-3-(3- chlorophenyl)imidazo[1,5-a]pyridine-1- carboxamide | 396.066 |
| | 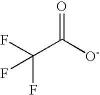 | | |
| 99 | 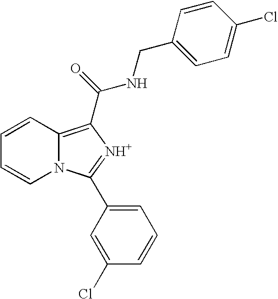 | N-(4-chlorobenzyl)-3-(3- chlorophenyl)imidazo[1,5-a]pyridine-1- carboxamide | 396.0652 |
| | 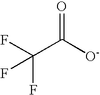 | | |
| 100 | 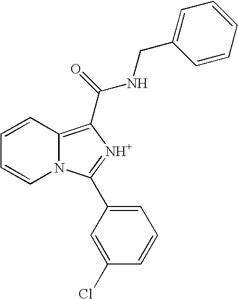 | N-benzyl-3-(3- chlorophenyl)imidazo[1,5-a]pyridine-1- carboxamide | 362.1046 |
| | 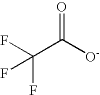 | | |
| 101 | 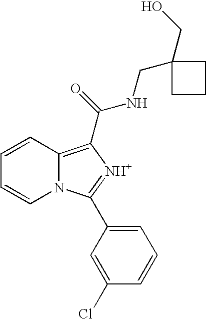 | 3-(3-chlorophenyl)-N-{[1- (hydroxymethyl)cyclobutyl]methyl} imidazo[1,5-a]pyridine-1-carboxamide | 370.1301 |
| | 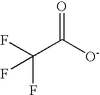 | | |
| 102 | 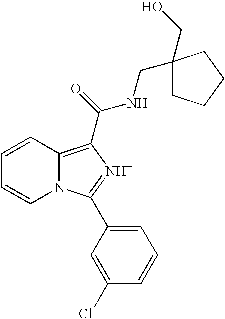 | 3-(3-chlorophenyl)-N-{[1- (hydroxymethyl)cyclopentyl]methyl} imidazo[1,5-a]pyridine-1-carboxamide | 384.1454 |
| | 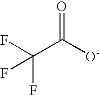 | | |
| 103 | 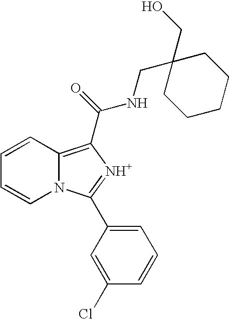 | 3-(3-chlorophenyl)-N-{[1- (hydroxymethyl)cyclohexyl]methyl} imidazo[1,5-a]pyridine-1-carboxamide | 398.1614 |
| | 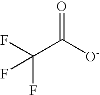 | | |
| 104 | 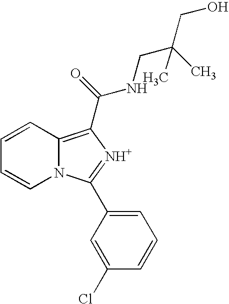 | 3-(3-chlorophenyl)-N-(3-hydroxy-2,2- dimethylpropyl)imidazo[1,5-a]pyridine- 1-carboxamide | 358.1302 |
| | 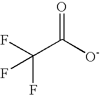 | | |
| 105 | 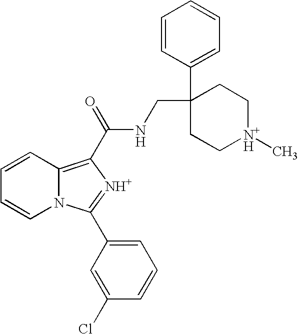 | 3-(3-chlorophenyl)-N-[(1-methyl-4- phenylpiperidin-4- yl)methyl]imidazo[1,5-a]pyridine-1- carboxamide | 459.1931 |
| | 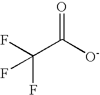 | | |
| 106 | 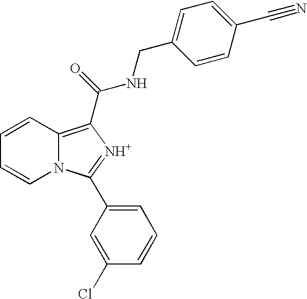 | 3-(3-chlorophenyl)-N-(4- cyanobenzyl)imidazo[1,5-a]pyridine-1- carboxamide | 387.0993 |
| | 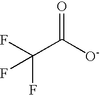 | | |
| 107 | 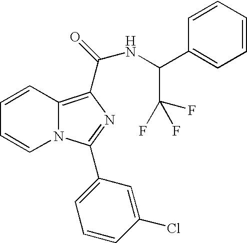 | 3-(3-chlorophenyl)-N-(2,2,2-trifluoro-1- phenylethyl)imidazo[1,5-a]pyridine-1- carboxamide | 430.1 |
| 108 | 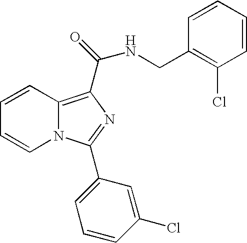 | N-(2-chlorobenzyl)-3-(3- chlorophenyl)imidazo[1,5-a]pyridine-1- carboxamide | 397.1 |
| 109 | 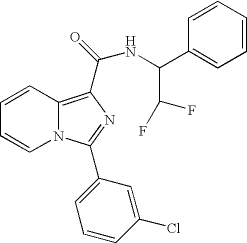 | 3-(3-chlorophenyl)-N-(2,2-difluoro-1- phenylethyl)imidazo[1,5-a]pyridine-1- carboxamide | 412.1 |
| 110 | 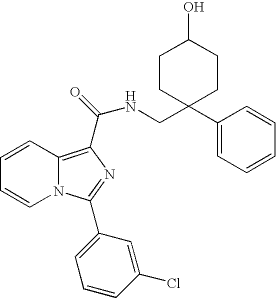 | 3-(3-chlorophenyl)-N-[(4-hydroxy-1- phenylcyclohexyl)methyl]imidazo[1,5- a]pyridine-1-carboxamide | 460.1 |
| 111 | 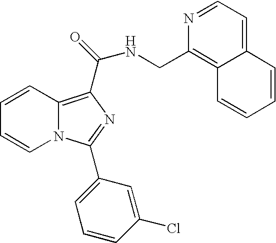 | 3-(3-chlorophenyl)-N-(isoquinolin-1- ylmethyl)imidazo[1,5-a]pyridine-1- carboxamide | 413.1 |
| 112 | 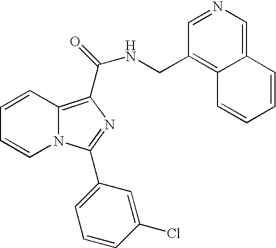 | 3-(3-chlorophenyl)-N-(isoquinolin-4- ylmethyl)imidazo[1,5-a]pyridine-1- carboxamide | 413.1 |
| 113 | 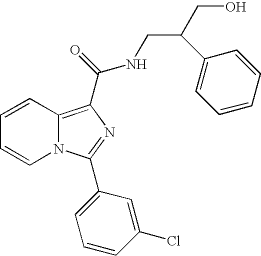 | 3-(3-chlorophenyl)-N-(3-hydroxy-2- phenylpropyl)imidazo[1,5-a]pyridine-1- carboxamide | 406.1 |
| 114 | 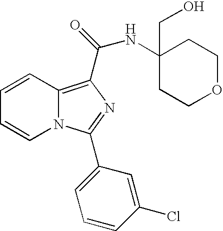 | 3-(3-chlorophenyl)-N-[4- (hydroxymethyl)tetrahydro-2H-pyran-4- yl]imidazo[1,5-a]pyridine-1- carboxamide | 386.1 |
| 115 | 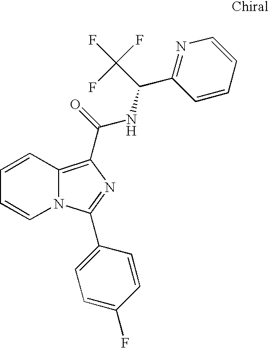 | 3-(4-fluorophenyl)-n-[(1s)-2,2,2- trifluoro-1-pyridin-2- ylethyl]imidazo[1,5-a]pyridine-1- carboxamide | 415.1158 |
| 116 | 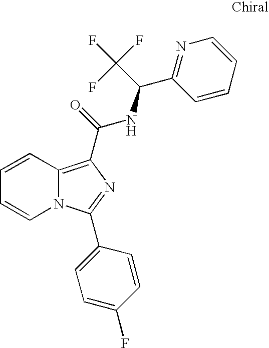 | 3-(4-fluorophenyl)-n-[(1r)-2,2,2- trifluoro-1-pyridin-2- ylethyl]imidazo[1,5-a]pyridine-1- carboxamide | 415.116 |
| 117 | 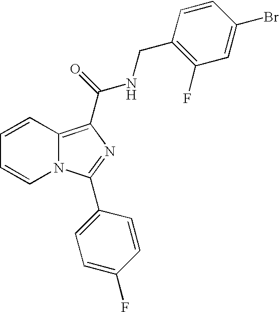 | N-(4-bromo-2-fluorobenzyl)-3-(4- fluorophenyl)imidazo[1,5-a]pyridine-1- carboxamide | 442.0336 |
| 118 | 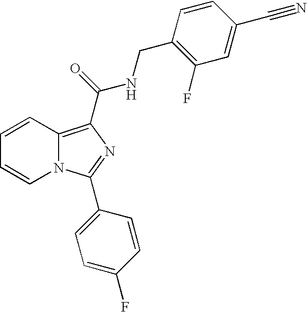 | N-(4-cyano-2-fluorobenzyl)-3-(4- fluorophenyl)imidazo[1,5-a]pyridine-1- carboxamide | 389.1212 |
| 119 | 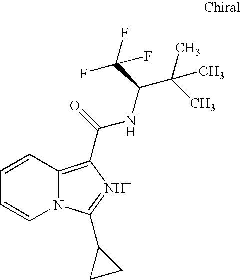 | 3-cyclopropyl-1-({[(1r)-2,2-dimethyl-1- (trifluoromethyl)propyl]amino}carbonyl) imidazo[1,5-a]pyridin-2-ium trifluoroacetate | 340.1626 |
| | 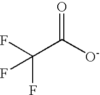 | | |
| 120 | 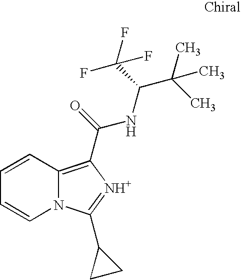 | 3-cyclopropyl-1-({[(1s)-2,2-dimethyl-1- (trifluoromethyl)propyl]amino}carbonyl) imidazo[1,5-a]pyridin-2-ium trifluoroacetate | 340.1626 |
| | 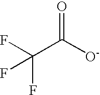 | | |
| 121 | 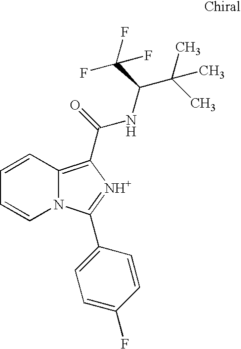 | 1-({[(1r)-2,2-dimethyl-1- (trifluoromethyl)propyl]amino}carbonyl)- 3-(4-fluorophenyl)imidazo[1,5- a]pyridin-2-ium trifluoroacetate | 394.1532 |
| | 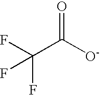 | | |
| 122 | 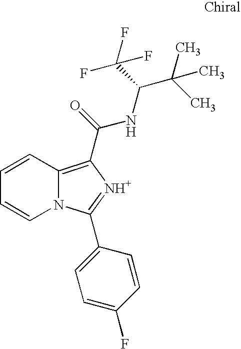 | 1-({[(1s)-2,2-dimethyl-1- (trifluoromethyl)propyl]amino}carbonyl)- 3-(4-fluorophenyl)imidazo[1,5- a]pyridin-2-ium trifluoroacetate | 394.1533 |
| | 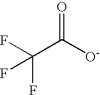 | | |
| 123 | 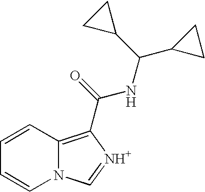 | 1-{[(dicyclopropylmethyl)amino]carbonyl} imidazo[1,5-a]pyridin-2-ium trifluoroacetate | 256.1436 |
| | 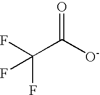 | | |
| 124 | 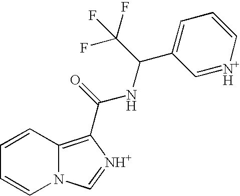 | 1-{[(2,2,2-trifluoro-1-pyridinium-3- ylethyl)amino]carbonyl}imidazo[1,5- a]pyridin-2-ium bis(trifluoroacetate) | 321.0948 |
| | 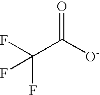 | | |
| 125 | 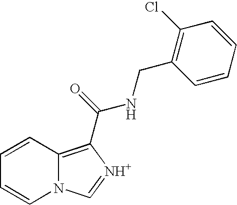 | 1-{[(2- chlorobenzyl)amino]carbonyl}imidazo [1,5-a]pyridin-2-ium trifluoroacetate | 286.0734 |
| | 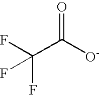 | | |
| 126 | 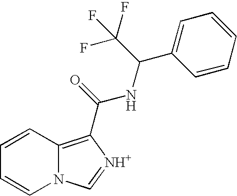 | 1-{[(2,2,2-trifluoro-1- phenylethyl)amino]carbonyl}imidazo [1,5-a]pyridin-2-ium trifluoroacetate | 320.0997 |
| | 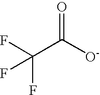 | | |
| 127 | 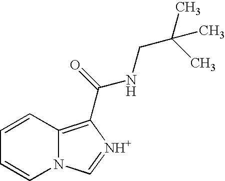 | 1-{[(2,2- dimethylpropyl)amino]carbonyl}imi- dazo[1,5-a]pyridin-2-ium trifluoroacetate | 232.1437 |
| | 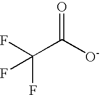 | | |
| 128 | 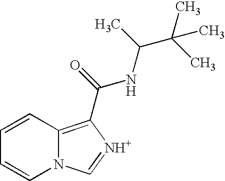 | 1-{[(1,2,2- trimethylpropyl)amino]carbonyl}imi- dazo[1,5-a]pyridin-2-ium trifluoroacetate | 246.1593 |
| | 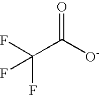 | | |
| 129 | 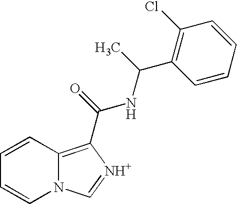 | 1-({[1-(2- chlorophenyl)ethyl]amino}carbonyl)imi dazo[1,5-a]pyridin-2-ium trifluoroacetate | 300.0891 |
| | 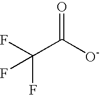 | | |
| 130 | 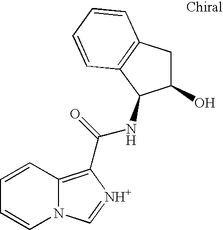 | 1-({[(1S,2R)-2-hydroxy-2,3-dihydro- 1H-inden-1- yl]amino}carbonyl)imidazo[1,5- a]pyridin-2-ium trifluoroacetate | 294.1231 |
| | 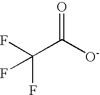 | | |
| 131 | 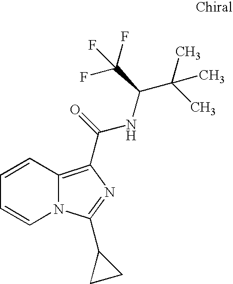 | 3-cyclopropyl-n-[(1r)-2,2-dimethyl-1- (trifluoromethyl)propyl]imidazo[1,5- a]pyridine-1-carboxamide | See TFA- salt above |
| 132 | 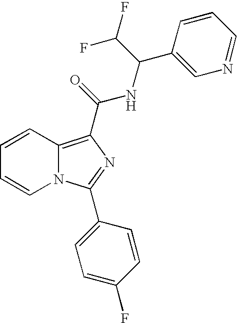 | N-(2,2-difluoro-1-pyridin-3-ylethyl)-3- (4-fluorophenyl)imidazo[1,5-a]pyridine- 1-carboxamide | 397.1256 |
| 133 | 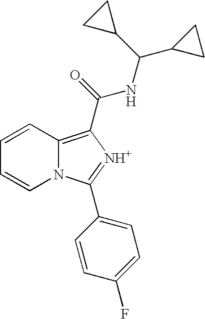 | 1-{[(dicyclopropylmethyl)amino]carbonyl}- 3-(4-fluorophenyl)imidazo[1,5- a]pyridin-2-ium trifluoroacetate | 350.165 |
| | 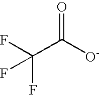 | | |
| 134 | 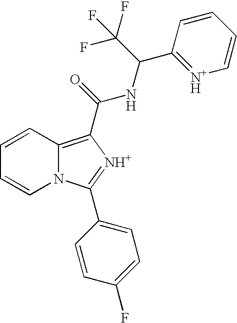 | 3-(4-fluorophenyl)-1-{[(2,2,2-trifluoro- 1-pyridinium-2- ylethyl)amino]carbonyl}imidazo[1,5- a]pyridin-2-ium bis(trifluoroacetate) | 416.1415 |
| | 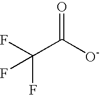 | | |
| 135 | 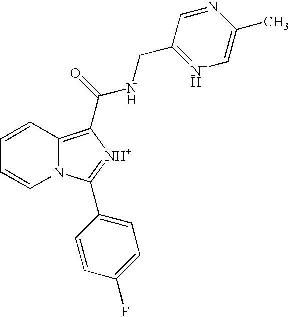 | 3-(4-fluorophenyl)-1-({[(5- methylpyrazin-1-ium-2- yl)methyl]amino}carbonyl)imidazo[1,5- a]pyridin-2-ium bis(trifluoroacetate) | 362.1404 |
| | 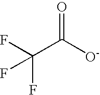 | | |
| 136 | 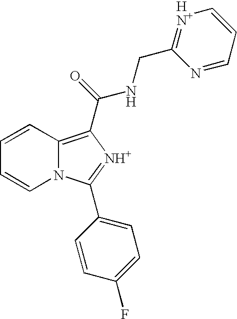 | 3-(4-fluorophenyl)-1-{[(pyrimidin-1- ium-2- ylmethyl)amino]carbonyl}imidazo[1,5- a]pyridin-2-ium bis(trifluoroacetate) | 348.1246 |
| | 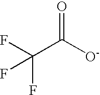 | | |
| 137 | 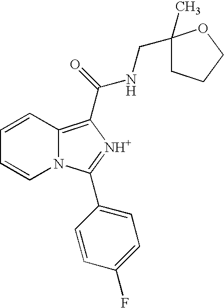 | 3-(4-fluorophenyl)-1-({[(2- methyltetrahydrofuran-2- yl)methyl]amino}carbonyl)imidazo[1,5- a]pyridin-2-ium trifluoroacetate | 354.1607 |
| | 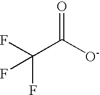 | | |
| 138 | 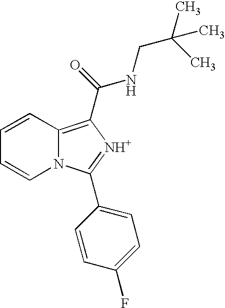 | 1-{[(2,2- dimethylpropyl)amino]carbonyl}-3-(4- fluorophenyl)imidazo[1,5-a]pyridin-2- ium trifluoroacetate | 326.1656 |
| | 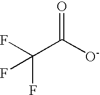 | | |
| 139 | 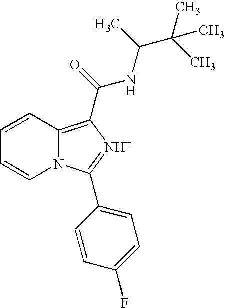 | 3-(4-fluorophenyl)-1-{[(1,2,2- trimethylpropyl)amino]carbonyl}imidazo [1,5-a]pyridin-2-ium trifluoroacetate | 340.1813 |
| | 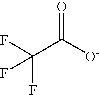 | | |
| 140 | 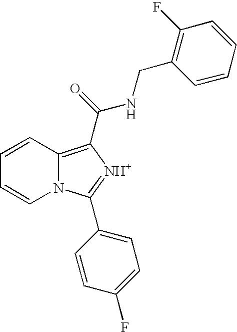 | 1-{[(2-fluorobenzyl)amino]carbonyl}-3- (4-fluorophenyl)imidazo[1,5-a]pyridin- 2-ium trifluoroacetate | 364.125 |
| | 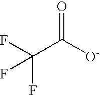 | | |
| 141 | 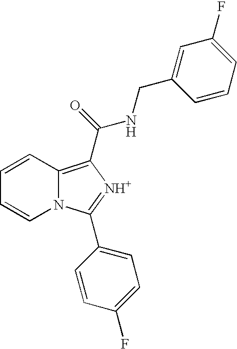 | 1-{[(3-fluorobenzyl)amino]carbonyl}-3- (4-fluorophenyl)imidazo[1,5-a]pyridin- 2-ium trifluoroacetate | 364.1249 |
| | 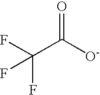 | | |
| 142 | 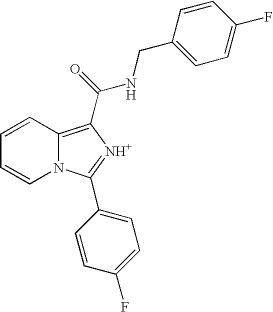 | 1-{[(4-fluorobenzyl)amino]carbonyl}-3- (4-fluorophenyl)imidazo[1,5-a]pyridin- 2-ium trifluoroacetate | 364.125 |
| | 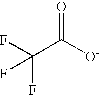 | | |
| 143 | 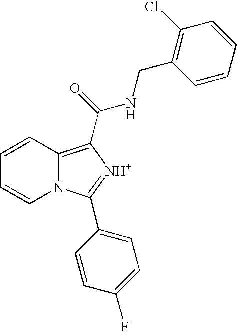 | 1-{[(2-chlorobenzyl)amino]carbonyl}- 3-(4-fluorophenyl)imidazo[1,5- a]pyridin-2-ium trifluoroacetate | 380.0952 |
| | 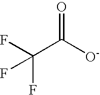 | | |
| 144 | 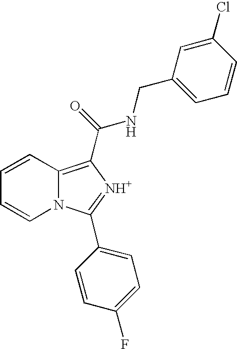 | 1-{[(3-chlorobenzyl)amino]carbonyl}- 3-(4-fluorophenyl)imidazo [1,5- a]pyridin-2-ium trifluoroacetate | 380.0953 |
| | 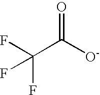 | | |
| 145 | 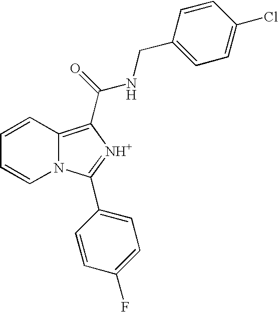 | 1-{[(4-chlorobenzyl)amino]carbonyl}- 3-(4-fluorophenyl)imidazo[1,5- a]pyridin-2-ium trifluoroacetate | 380.0953 |
| | 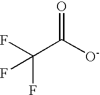 | | |
| 146 | 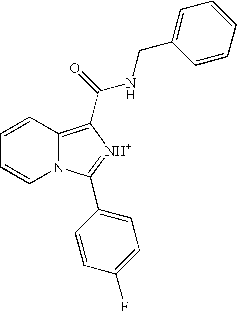 | 1-[(benzylamino)carbonyl]-3-(4- fluorophenyl)imidazo[1,5-a]pyridin-2- ium trifluoroacetate | 346.1343 |
| | 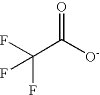 | | |
| 147 | 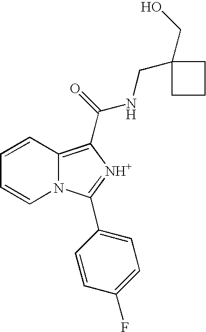 | 3-(4-fluorophenyl)-1-[({[1- (hydroxymethyl)cyclobutyl]methyl} amino)carbonyl]imidazo[1,5-a]pyridin-2- ium trifluoroacetate | 354.1608 |
| | 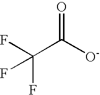 | | |
| 148 | 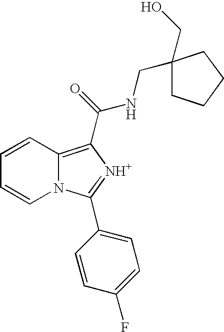 | 3-(4-fluorophenyl)-1-[({[1- (hydroxymethyl)cyclopentyl]methyl} amino)carbonyl]imidazo[1,5-a]pyridin-2- ium trifluoroacetate | 368.1765 |
| | 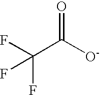 | | |
| 149 | 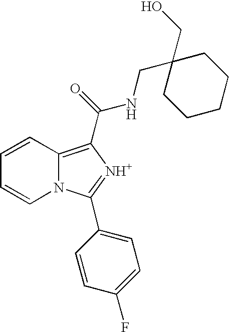 | 3-(4-fluorophenyl)-1-[({[1- (hydroxymethyl)cyclohexyl]methyl} amino)carbonyl]imidazo[1,5-a]pyridin-2- ium trifluoroacetate | 382.1919 |
| | 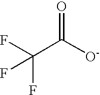 | | |
| 150 | 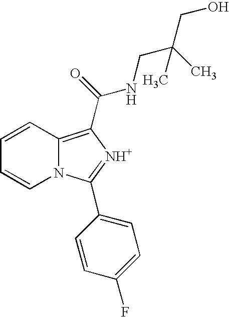 | 3-(4-fluorophenyl)-1-{[(3-hydroxy-2,2- dimethylpropyl)amino]carbonyl}imidazo [1,5-a]pyridin-2-ium trifluoroacetate | 342.1606 |
| | 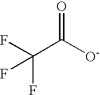 | | |
| 151 | 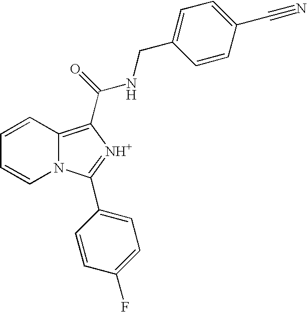 | 1-{[(4-cyanobenzyl)aminolcarbonyl}-3- (4-fluorophenyl)imidazo[1,5-a]pyridin- 2-ium trifluoroacetate | 371.1296 |
| | 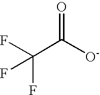 | | |
| 152 | 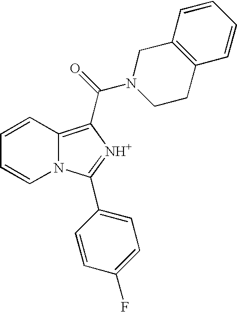 | 1-(3,4-dihydroisoquinolin-2(1H)- ylcarbonyl)-3-(4- fluorophenyl)imidazo[1,5-a]pyridin-2- ium trifluoroacetate | 372.1497 |
| | 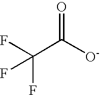 | | |
| 153 | 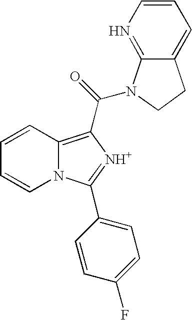 | 1-{[3-(4-fluorophenyl)imidazo[1,5- a]pyridin-2-ium-1-yl]carbonyl}-2,3- dihydro-1H-pyrrolo[2,3-b]pyridin-7-ium bis(trifluoroacetate) | 359.1293 |
| | 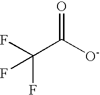 | | |
| 154 | 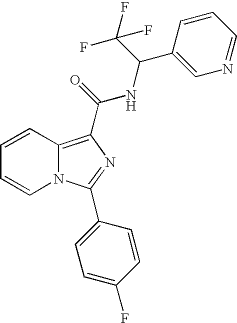 | 3-(4-Fluorophenyl)-N-(2,2,2-trifluoro-1- pyridyl-3-ylethyl)imidazo1,5- a]pyridine-1-carboxamide | 415.1159 |
| 155 | 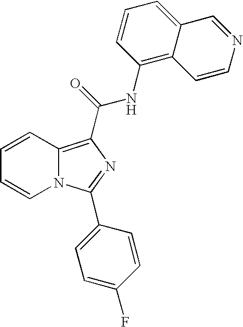 | 3-(4-fluorophenyl)-N-isoquinolin-5- ylamidazo1,5-a]pyridine-1-carboxamide | |
| 156 | 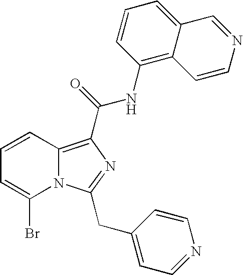 | 5-bromo-N-isoquinolin-5-yl-3-(pyridin- 4-ylmethyl)imidazo[1,5-a]pyridine-1- carboxamide | 458.062 |
| | | | Mass Spec |
| Example | Structure | Name | (M + H)+ |
| 631 | 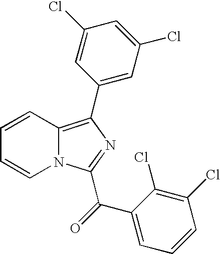 | (2,3-Dichlorophenyl)[1-(3,5- Dichlorophenyl)Imidazo[1,5-A]pyridin-3- YL]methanone | 434.9592 |
| 632 | 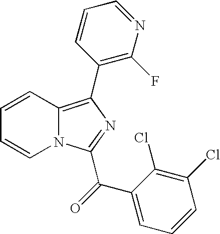 | (2,3-Dichlorophenyl)[1-(2-fluoropyridin-3- yl)imidazo[1,5-a]pyridin-3-yl]methanone | 386.0235 |
| 633 | 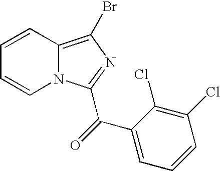 | (1-Bromoimidazo[1,5-a]pyridin-3-yl)(2,3- dichlorophenyl)methanone | 368.7 |
| 634 | 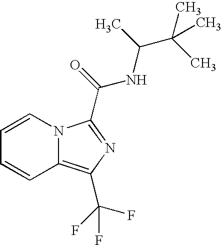 | 1-(trifluoromethyl)-N-(1,2,2- trimethylpropyl)imidazo[1 5-a]pyridine-3- carboxamide | 314.1484 |
| 635 | 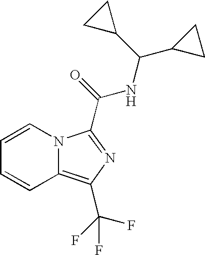 | N-(dicyclopropylmethyl)-1- (trifluoromethyl)imidazo[1,5-a]pyridine-3- carboxamide | 324.1329 |
| 636 | 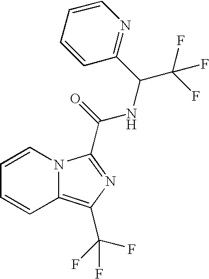 | 1-(trifluoromethyl)-N-(2,2,2-trifluoro-1-pyridin-2- ylethyl)imidazo[1,5-a]pyridine-3-carboxamide | 389.0842 |
| 637 | 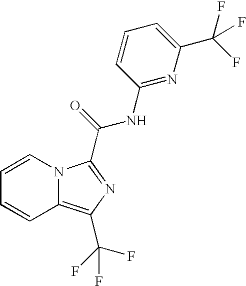 | 1-(trifluoromethyl)-N-[6-(trifluoromethyl)pyridin- 2-yl]imidazo[1,5-a]pyridine-3-carboxamide | 375.0681 |
| 638 | 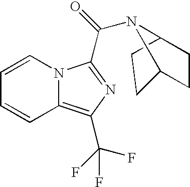 | 3-(7-azabicyclo[2.2.1]hept-7-ylcarbonyl)-1- (trifluoromethyl)imidazo[1,5-a]pyridine | 310.0 |
| 639 | 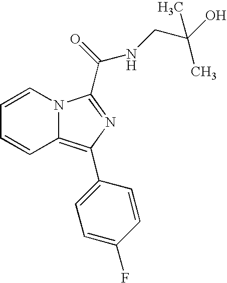 | 1-(4-fluorophenyl)-N-(2-hydroxy-2- methylpropyl)imidazo[1,5-a]pyridine-3- carboxamide | 328.1456 |
| 640 | 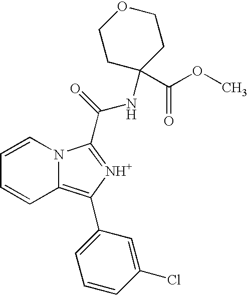 | 1-(3-chlorophenyl)-3-({[4- (methoxycarbonyl)tetrahydro-2H-pyran-4- yl]amino}carbonyl)imidazo[1,5-a]pyridin-2-ium trifluoroacetate | 414.1222 |
| | 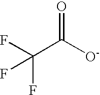 | | |
| 641 | 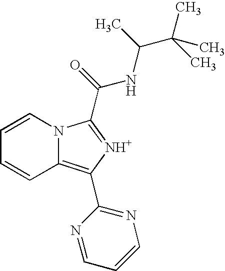 | 1-pyrimidin-2-yl-3-{[(1,2,2- trimethylpropyl)amino]carbonyl}imidazo[1,5- a]pyridin-2-ium trifluoroacetate | 342.3 |
| | 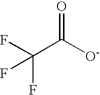 | | |

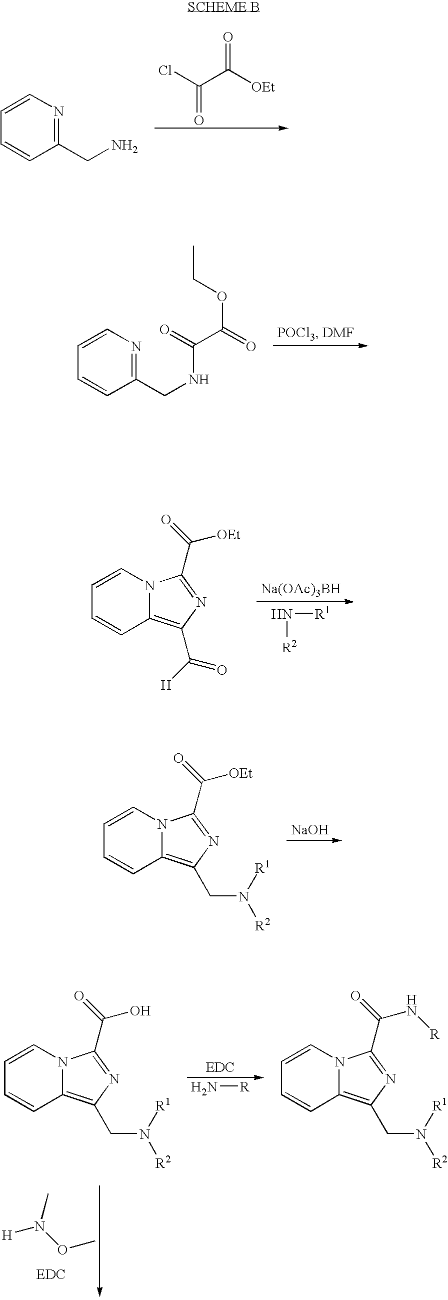

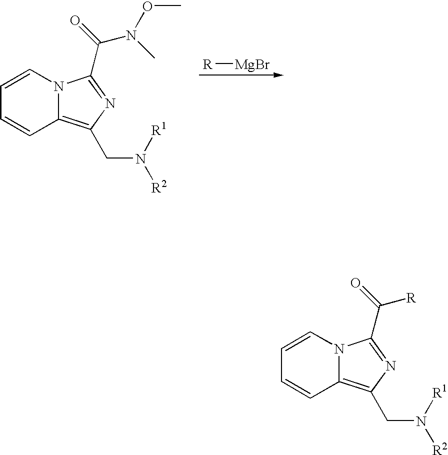

### Intermediate

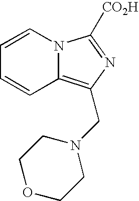

### 1-(Morpholin-4-ylmethyl)imidazo[1,5-a]pyridine-3-carboxylic acid

### Step A: Ethyl oxo[(pyridin-2-ylmethyl)amino]acetate

To a solution of 1-pyridin-2-ylmethanamine (10.0 g, 92.5 mmol) in anhydrous THF (300 mL) at 0° C. was added ethyl chloro(oxo)acetate (11.4 mL, 102 mmol) followed by TEA (19.3 mL, 139 mmol). The reaction mixture was slowly warmed to ambient temperature. After 18 h, the mixture was concentrated. The residue was dilute with saturated aqueous NaHCO3 and extracted with EtOAc. The combined organic extracts were washed with saturated brine, dried over MgSO4, and concentrated to give the title compound (18.7 g, 97%). MS 209.1 (M+1).

### Step B: Ethyl 1-formylimidazo[1,5-a]pyridine-3-carboxylate

A solution of ethyl oxo[(pyridin-2-ylmethyl)amino]acetate (18.7 g, 89.8 mmol) in POCl₃ (150 mL) was heated at reflux for 18 h. DMF (7.96 mL, 103 mmol) was added and the mixture was refluxed for additional 2 h. The mixture was concentrated and saturated aqueous NaHCO₃ was added. The mixture was extracted with EtOAc (3×) and the combined organic extracts were washed with saturated brine, dried over Na₂SO₄, filtered and concentrated to give the title compound (12.8 g, 65%). MS 219.1 (M+1). ¹H NMR (400 MHz, CDCl₃) δ10.19 (s, 1H), 9.46 (d, J=7.6 Hz, 1H), 8.45 (d, J=8.8 Hz, 1H), 7.49-7.47 (m, 1H), 7.19-7.16 (m, 1H), 4.58-4.52 (m, 2H), 1.49 (dd, J=14.0, 7.6 Hz, 3H).

### Step C: Ethyl 1-(morpholin-4-ylmethyl)imidazo[1,5-a]pyridine-3-carboxylate

To a solution of ethyl 1-formylimidazo[1,5-a]pyridine-3-carboxylate (5.0 g, 22.9 mmol), morpholine (2.2 g, 25.2 mmol) in 1,2-dichloroethane (200 mL) was added NaBH(OAc)₃ (9.71 g, 45.8 mmol). The mixture was stirred at ambient temperature for 18 h. The reaction mixture was diluted with saturated aqueous NaHCO₃ and extracted with DCM. The combined organic extracts were dried over MgSO₄, filtered and concentrated. The residue was purified by silica gel chromatography (0%→10% MeOH/DCM) to give the title compound (5.71 g, 86%). MS 290.1 (M+1). ¹H NMR (400 MHz, CDCl₃) δ 9.31 (d, J=9.6 Hz, 1H), 7.88 (d, J=12.0 Hz, 1H), 7.07-7.02 (m, 1H), 6.93-6.88 (m, 1H), 4.49 (q, J=9.6 Hz, 2H), 3.90 (s, 2H), 3.69 (t, J=6.4 Hz, 4H), 2.52 (t, J=6.4 Hz, 4H), 1.46 (t, J=4.8 Hz, 3H).

### Step D: 1-(Morpholin-4-ylmethyl)imidazo[1,5-a]pyridine-3-carboxylic acid

To a solution of ethyl 1-(morpholin-4-ylmethyl)imidazo[1,5-a]pyridine-3-carboxylate (5.71 g, 19.7 mmol) in methanol (30 mL) was added an aqueous solution of 1 M NaOH solution (59.2 mL). The reaction mixture was stirred at ambient temperature for 18 h. The mixture was quenched with an aqueous solution of 6 M HCl (10.2 mL) and concentrated under reduced pressure to give title compound along with NaCl. MS 262.2 (M+1).

### Example 157

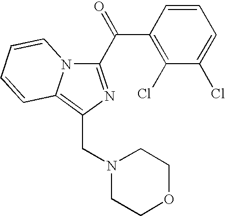

### (2,3-Dichlorophenyl)[1-(morpholin-4-ylmethyl)imidazo[1,5-a]pyridin-3-yl]methanone

### Step A: N-Methoxy-N-methyl-1-(morpholin-4-ylmethyl)imidazo[1,5-a]pyridine-3-carboxamide

To a solution of 1-(morpholin-4-ylmethyl)imidazo[1,5-a]pyridine-3-carboxylic acid (0.39 g, 1.51 mmol) and methoxy(methyl)ammonium chloride (0.18 g, 1.89 mmol) in DMF (10 mL) were added EDC (0.43 g, 2.26 mmol), HOAT (0.31 g, 2.26 mmol) and diisopropylethylamine (1.45 mL, 8.29 mmol). The reaction mixture was stirred at ambient temperature for 18 h. The mixture was concentrated and saturated aqueous NaHCO₃ was added. The mixture was extracted with DCM. The combined organic extracts were dried over Na₂SO₄, filtered and concentrated. Purification by silica gel chromatography (0%→10% MeOH/DCM) to give the title compound (0.32 g). MS 218.1 (M-morpholine+1). ¹H NMR (500 MHz, CDCl₃) δ 9.27 (d, J=7.3 Hz, 1H), 7.74 (d, J=9.0 Hz, 1H), 6.98 (dd, J=9.0, 6.6 Hz, 1H), 6.81-6.78 (m, 1H), 3.91 (s, 3H), 3.87 (s, 2H), 3.72-3.69 (m, 7H), 2.54 (t, J=4.4 Hz, 4H).

### Step B: (2,3-Dichlorophenyl)[1-(morpholin-4-ylmethyl)imidazo[1,5-a]pyridin-3-yl]methanone

To a solution of N-methoxy-N-methyl-1-(morpholin-4-ylmethyl)imidazo[1,5-a]pyridine-3-carboxamide (1.2 g, 3.94 mmol) in THF (5 mL) at −4° C. was added a solution of (2,3-dichlorophenyl)(iodo)magnesium (0.56 M in ether; 21.1 mL, 11.8 mmol). The reaction mixture was slowly warmed to ambient temperature. After 18 h, the mixture was quenched with saturated aqueous NH₄Cl. Saturated aqueous NaHCO₃ was added and the mixture was extracted with EtOAc. The combined organic extracts were washed with saturated brine, dried over Na₂SO₄, filtered and concentrated. Purification by silica gel chromatography (100% DCM→20% EtOAc/DCM→10%/20%/70% MeOH/EtOAc/DCM) gave the title compound (1.3 g). HRMS: m/z found=390.0780 (M+1). ¹H NMR (500 MHz, CDCl₃) δ 9.81 (d, J=7.1 Hz, 1H), 8.01 (d, J=9.0 Hz, 1H), 7.57 (dd, J=8.1, 1.5.Hz, 1H), 7.47-7.45 (m, 1H), 7.33-7.27 (m, 2H), 7.15-7.12 (m, 1H), 3.85 (s, 2H), 3.71-3.69 (m, 4H), 2.51 (t, J=4.2 Hz, 4H).

The following compounds were synthesized according to the procedures detailed above:

TABLE B

| Example | Structure | Name | MS(MH)* |
|---|---|---|---|
| 158 | 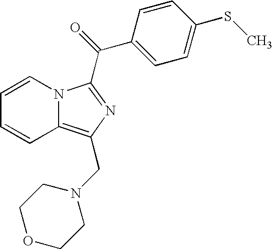 | [4-(methylthio)phenyl][1-(morpholin-4- ylmethyl)imidazo[1,5-a]pyridin-3- yl]methanone | 368.1428 |
| 159 | 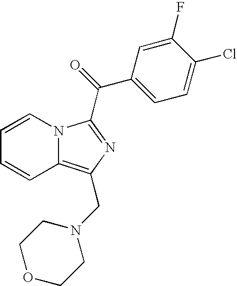 | (4-chloro-3-fluorophenyl)[1-(morpholin- 4-ylmethyl)imidazo[1,5-a]pyridin-3- yl]methanone | 374.1068 |
| 160 | 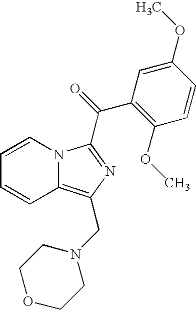 | (2,5-dimethoxyphenyl)[1-(morpholin-4- ylmethyl)imidazo[1,5-a]pyridin-3- yl]methanone | 382.1763 |
| 161 | 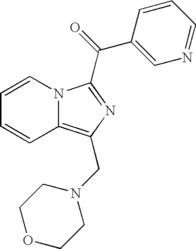 | [1-(morpholin-4-ylmethyl)imidazo[1,5- a]pyridin-3-yl])pyridin-3-yl)methanone | 323.1505 |
| 162 | 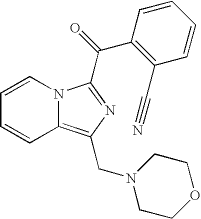 | 2-{[1-(morpholin-4- ylmethyl)imidazo[1,5-a]pyridin-3- yl]carbonyl}benzonitrile | 347.1502 |
| 163 | 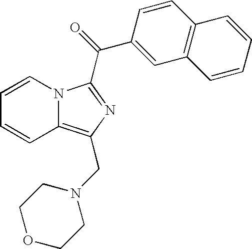 | [1-(morpholin-4-ylmethyl)imidazo[1,5- a]pyridin-3-yl](2-naphthyl)methanone | 372.1705 |
| 164 | 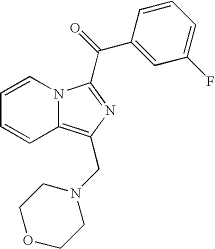 | 3-fluorophenyl)[1-(morpholin-4- ylmethyl)imidazo[1,5-a]pyridin-3- yl]methanone | 340.164 |
| 165 | 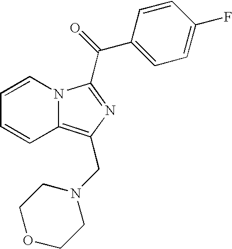 | (4-fluorophenyl)[1-(morpholin-4- ylmethyl)imidazo[1,5-a]pyridin-3- yl]methanone | 340.1458 |
| 166 | 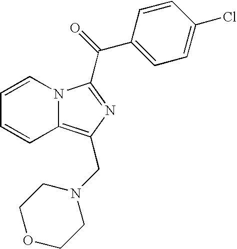 | (4-chlorophenyl)[1-(morpholin-4- ylmethyl)imidazo[1,5-a]pyridin-3- yl]methanone | 356.1164 |
| 167 | 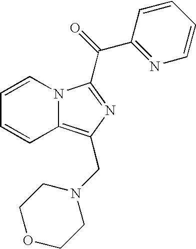 | [1-(morpholin-4-ylmethyl)imidazo[1,5- a]pyridin-3-yl])pyridin-2-yl)methanone | 323.1523 |
| 168 | 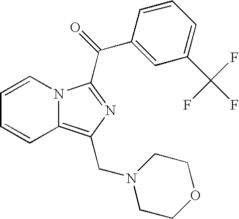 | [1-(morpholin-4-ylmethyl)imidazo[1,5- a]pyridin-3-yl][3- (trifluoromethyl)phenyl]methanone | 390.1412 |
| 169 | 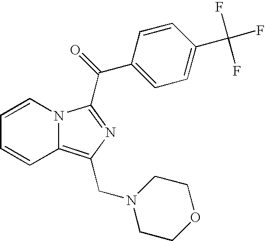 | [1-(morpholin-4-ylmethyl)imidazo[1,5- a]pyridin-3-yl][4- (trifluoromethyl)phenyl]methanone | 390.141 |
| 170 | 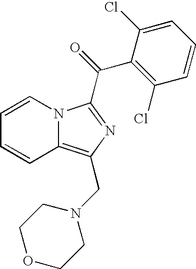 | (2,6-dichlorophenyl)[1-(morpholin-4- ylmethyl)imidazo[1,5-a]pyridin-3- yl]methanone | 390.0752 |
| 171 | 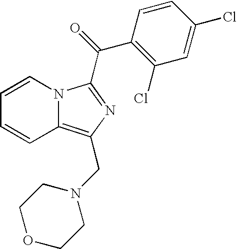 | (2,4-dichlorophenyl)[1-(morpholin-4- ylmethyl)imidazo[1,5-a]pyridin-3- yl]methanone | 390.0738 |
| 172 | 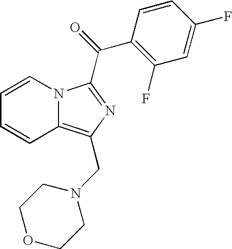 | (2,4-difluorophenyl)[1-(morpholin-4- ylmethyl)imidazo[1,5-a]pyridin-3- yl]methanone | 358.1326 |
| 173 | 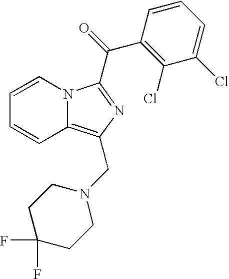 | (2,3-dichlorophenyl){1-[(4,4- difluoropiperidin-1- yl)methyl]imidazo[1,5-a]pyridin-3- yl}methanone | 424.077 |
| 174 | 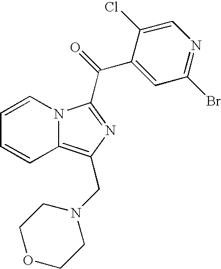 | (2-bromo-5-chloropyridin-4-yl)[1- (morpholin-4-ylmethyl)imidazo[1,5- a]pyridin-3-yl]methanone | 435.0195 |
| 175 | 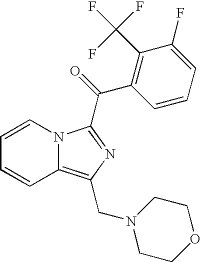 | [3-fluoro-2-(trifluoromethyl)phenyl][1- (morpholin-4-ylmethyl)imidazo[1,5- a]pyridin-3-yl]methanone | 408.1298 |
| 176 | 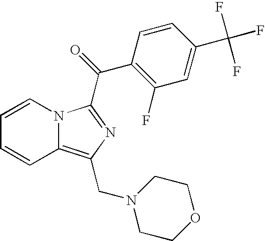 | [2-fluoro-4-(trifluoromethyl)phenyl][1- (morpholin-4-ylmethyl)imidazo[1,5- a]pyridin-3-yl]methanone | 408.1302 |
| 177 | 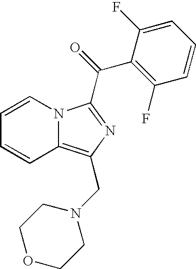 | (2,6-difluorophenyl)[1-(morpholin-4- ylmethyl)imidazo[1,5-a]pyridin-3- yl]methanone | 358.1326 |
| 178 | 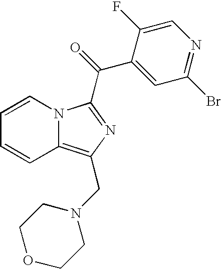 | (2-bromo-5-fluoropyridin-4-yl)[1- (morpholin-4-ylmethyl)imidazo[1,5- a]pyridin-3-yl]methanone | 419.0487 |
| 179 | 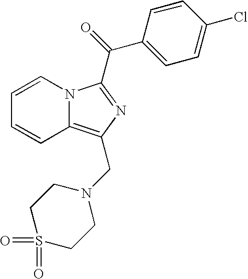 | (4-chlorophenyl){1-[(1,1- dioxidothiomorpholin-4- yl)methyl]imidazo[1,5-a]pyridin-3- yl}methanone | 426.0645 (M + Na) |
| 180 | 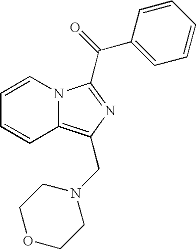 | [1-(morpholin-4-ylmethyl)imidazo[1,5- a]pyridin-3-yl])phenyl)methanone | 322.1524 |
| 181 | 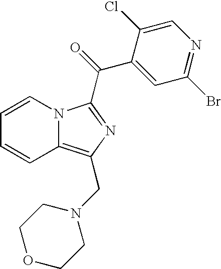 | (2-bromo-5-chloropyridin-4-yl)[1- (morpholin-4-ylmethyl)imidazo[1,5- a]pyridin-3-yl]methanone | 435.0195 |
| 182 | 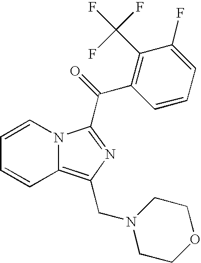 | [3-fluoro-2-(trifluoromethyl)phenyl][1- (morpholin-4-ylmethyl)imidazo[1,5- a]pyridin-3-yl]methanone | 408.1298 |
| | | | Mass Spec |
| Example | Structure | Name | (M + H)+ |
| 642 | 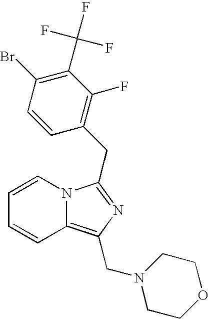 | [4-Bromo-2-fluoro-3- (trifluoromethyl)phenyl][1-(morpholin-4- ylmethyl)imidazo[1,5-a]pyridin-3- yl]methanone | 486.0449 |

### Scheme B Peptide Couplings

### Example 183

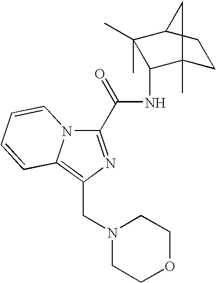

### 1-(Morpholin-4-ylmethyl)-N-(1,3,3-trimethylbicyclo[2.2.1]hept-2-yl)imidazo[1,5-a]pyridine-3-carboxamide

To a solution of 1-(morpholin-4-ylmethyl)imidazo[1,5-a]pyridine-3-carboxylic acid (30 mg, 0.12 mmol) in DMF (5 mL) were added 1,3,3-trimethylbicyclo[2.2.1]heptan-2-amine (21 mg, 0.14 mmol), EDC (33 mg, 0.17 mmol), HOAT (23 mg, 0.17 mmol) and diisopropylethylamine (0.11 mL, 0.63 mmol). The reaction mixture was stirred at ambient temperature for 18 h. The mixture was concentrated and saturated aqueous NaHCO₃ was added. The mixture was extracted with DCM. The combined organic extracts were dried over Na₂SO₄, filtered and concentrated. Purification by silica gel chromatography (0%→8% MeOH/DCM) to give the title compound (30 mg). HRMS: m/z found=397.2604 (M+1). ¹H NMR (500 MHz, CDCl₃) δ 9.46 (d, J=7.3 Hz, 1H), 7.71 (d, J=9.3 Hz, 1H), 7.41 (d, J=9.8 Hz, 1H), 6.93-6.90 (m, 1H), 6.78-6.75 (m, 1H), 3.84-3.80 (m, 3H), 3.73 (t, J=4.5 Hz, 4H), 2.55 (d, J=3.9 Hz, 4H), 1.81-1.72 (m, 3H), 1.54-1.52 (m, 2H), 1.29-1.24 (m, 2H), 1.18 (s, 3H), 1.12 (s, 3H), 0.89 (s, 3H).

### Example 183 (Alternative)

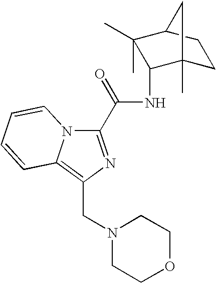

### 1-(Morpholin-4-ylmethyl)imidazo[1,5-a]pyridine-3-carboxylic acid

### Step A: Ethyl oxo[(pyridin-2-ylmethyl)amino]acetate

To a solution of 1-pyridin-2-ylmethanamine (10.0 g, 92.5 mmol) in anhydrous THF (300 mL) at 0° C. was added ethyl chloro(oxo)acetate (11.4 mL, 102 mmol) followed by TEA (19.3 mL, 139 mmol). The reaction mixture was slowly warmed to ambient temperature. After 18 h, the mixture was concentrated. The residue was dilute with saturated aqueous NaHCO3 and extracted with EtOAc. The combined organic extracts were washed with saturated brine, dried over MgSO4, and concentrated to give the title compound (18.7 g, 97%). MS 209.1 (M+1).

### Step B: Ethyl 1-formylimidazo[1,5-a]pyridine-3-carboxylate

A solution of ethyl oxo[(pyridin-2-ylmethyl)amino]acetate (18.7 g, 89.8 mmol) in POCl₃ (150 mL) was heated at reflux for 18 h. DMF (7.96 mL, 103 mmol) was added and the mixture was refluxed for additional 2 h. The mixture was concentrated and saturated aqueous NaHCO₃ was added. The mixture was extracted with EtOAc (3×) and the combined organic extracts were washed with saturated brine, dried over Na₂SO₄, filtered and concentrated to give the title compound (12.8 g, 65%). MS 219.1 (M+1). ¹H NMR (400 MHz, CDCl₃) δ 10.19 (s, 1H), 9.46 (d, J=7.6 Hz, 1H), 8.45 (d, J=8.8 Hz, 1H), 7.49-7.47 (m, 1H), 7.19-7.16 (m, 1H), 4.58-4.52 (m, 2H), 1.49 (dd, J=14.0, 7.6 Hz, 3H).

### Step C: Ethyl 1-(morpholin-4-ylmethyl)imidazo[1,5-a]pyridine-3-carboxylate

To a solution of ethyl 1-formylimidazo[1,5-a]pyridine-3-carboxylate (5.0 g, 22.9 mmol), morpholine (2.2 g, 25.2 mmol) in 1,2-dichloroethane (200 mL) was added NaBH(OAc)₃ (9.71 g, 45.8 mmol). The mixture was stirred at ambient temperature for 18 h. The reaction mixture was diluted with saturated aqueous NaHCO₃ and extracted with DCM. The combined organic extracts were dried over MgSO₄, filtered and concentrated. The residue was purified by silica gel chromatography (0%→10% MeOH/DCM) to give the title compound (5.71 g, 86%). MS 290.1 (M+1). ¹H NMR (400 MHz, CDCl₃) δ 9.31 (d, J=9.6 Hz, 1H), 7.88 (d, J=12.0 Hz, 1H), 7.07-7.02 (m, 1H), 6.93-6.88 (m, 1H), 4.49 (q, J=9.6 Hz, 2H), 3.90 (s, 2H), 3.69 (t, J=6.4 Hz, 4H), 2.52 (t, J=6.4 Hz, 4H), 1.46 (t, J=4.8 Hz, 3H).

### Step D: 1-(Morpholin-4-ylmethyl)imidazo[1,5-a]pyridine-3-carboxylic acid

To a solution of ethyl 1-(morpholin-4-ylmethyl)imidazo[1,5-a]pyridine-3-carboxylate (5.71 g, 19.7 mmol) in methanol (30 mL) was added an aqueous solution of 1 M NaOH solution (59.2 mL). The reaction mixture was stirred at ambient temperature for 18 h. The mixture was quenched with an aqueous solution of 6 M HCl (10.2 mL) and concentrated under reduced pressure to give title compound along with NaCl. MS 262.2 (M+1).

### Step F: 1-(Morpholin-4-ylmethyl)-N-(1,3,3-trimethylbicyclo[2.2.1]hept-2-yl)imidazo[1,5-a]pyridine-3-carboxamide

To a solution of 1-(morpholin-4-ylmethyl)imidazo[1,5-a]pyridine-3-carboxylic acid (30 mg, 0.12 mmol) in DMF (5 mL) were added 1,3,3-trimethylbicyclo[2.2.1]heptan-2-amine (21 mg, 0.14 mmol), EDC (33 mg, 0.17 mmol), HOAT (23 mg, 0.17 mmol) and diisopropylethylamine (0.11 mL, 0.63 mmol). The reaction mixture was stirred at ambient temperature for 18 h. The mixture was concentrated and saturated aqueous NaHCO₃ was added. The mixture was extracted with DCM. The combined organic extracts were dried over Na₂SO₄, filtered and concentrated. Purification by silica gel chromatography (0%→8% MeOH/DCM) to give the title compound (30 mg). HRMS: m/z found=397.2604 (M+1). ¹H NMR (500 MHz, CDCl3) δ 9.46 (d, J=7.3 Hz, 1H), 7.71 (d, J=9.3 Hz, 1H), 7.41 (d, J=9.8 Hz, 1H), 6.93-6.90 (m, 1H), 6.78-6.75 (m, 1H), 3.84-3.80 (m, 3H), 3.73 (t, J=4.5 Hz, 4H), 2.55 (d, J=3.9 Hz, 4H), 1.81-1.72 (m, 3H), 1.54-1.52 (m, 2H), 1.29-1.24 (m, 2H), 1.18 (s, 3H), 1.12 (s, 3H), 0.89 (s, 3H).

The following compounds were prepared according to the procedures above.

TABLE B′

| Example | Structure | Name | MS(MH) |
|---|---|---|---|
| 184 | 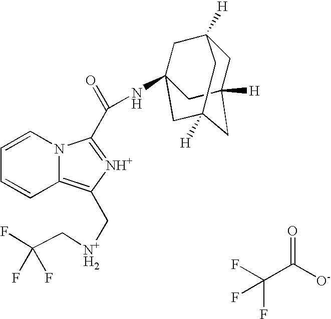 | N-[(3S,5S,7S)-adamantan-1-yl]-1- {[(2,2,2- trifluoroethyl)amino]methyl}imidazo [1,5-a]pyridine-3-carboxamide | 308.2 (fragment) |
| 185 | 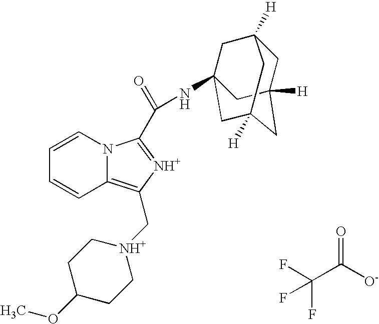 | N-[(3S,5S,7S)-adamantan-1-yl]-1- [(4-methoxypiperidin-1- yl)methyl]imidazo[1,5-a]pyridine-3- carboxamide | 423.2755 |
| 186 | 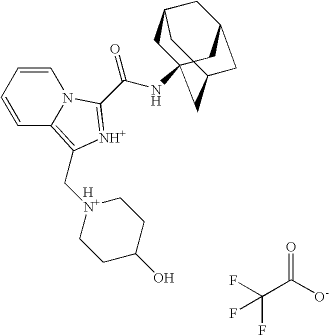 | N-[(3S,5S,7S)-adamantan-1-yl]-1- [(4-hydroxypiperidin-1- yl)methyl]imidazo[1,5-a]pyridine-3- carboxamide | 409.261 |
| 187 | 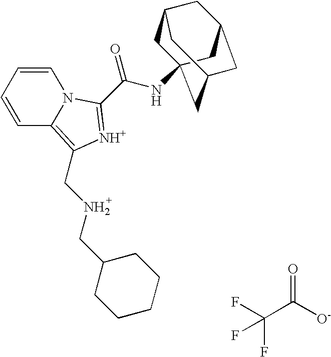 | N-[(3S,5S,7S)-adamantan-1-yl]-1- {[(cyclohexylmethyl)amino]methyl} imidazo[1,5-a]pyridine-3- carboxamide | 421.2971 |
| 188 | 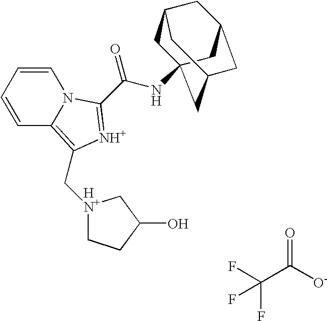 | N-[(3S,5S,7S)-adamantan-1-yl]-1- [(3-hydroxypyrrolidin-1- yl)methyl]imidazo[1,5-a]pyridine-3- carboxamide | 395.2452 |
| 189 | 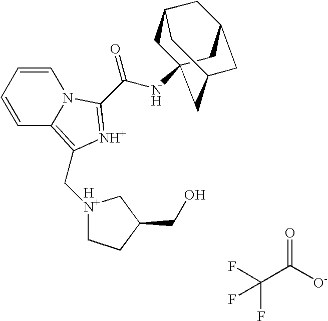 | N-[(3S,5S,7S)-adamantan-1-yl]-1- {[(3S)-3-(hydroxymethyl)pyrrolidin- 1-yl]methyl}imidazo[1,5- a]pyridine-3-carboxamide | 409.2609 |
| 190 | 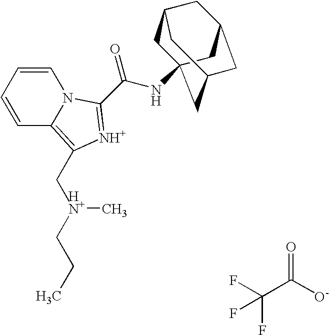 | N-[(3S,5S,7S)-adamantan-1-yl]-1- {[methyl(propyl)amino]methyl} imidazo[1,5-a]pyridine-3- carboxamide | 381.2657 |
| 191 | 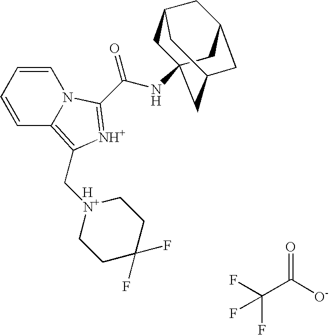 | N-[(3S,5S,7S)-adamantan-1-yl]-1- [(4,4-difluoropiperidin-1- yl)methyl]imidazo[1,5-a]pyridine-3- carboxamide | 429.2466 |
| 192 | 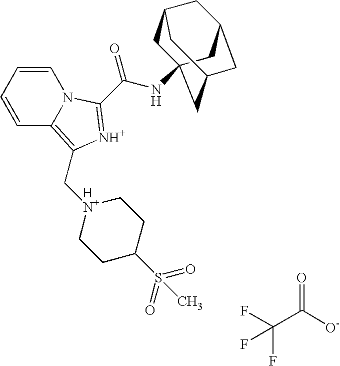 | N-[(3S,5S,7S)-adamantan-1-yl]-1- {[4-(methylsulfonyl)piperidin-1- yl]methyl}imidazo[1,5-a]pyridine- 3-carboxamide | 471.2457 |
| 193 | 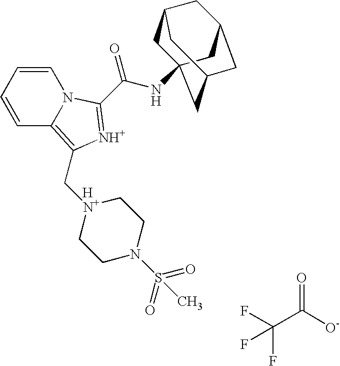 | N-[(3S,5S,7S)-adamantan-1-yl]-1- {[4-(methylsulfonyl)piperazin-1- yl]methyl}imidazo[1,5-a]pyridine- 3-carboxamide | 472.2393 |
| 194 | 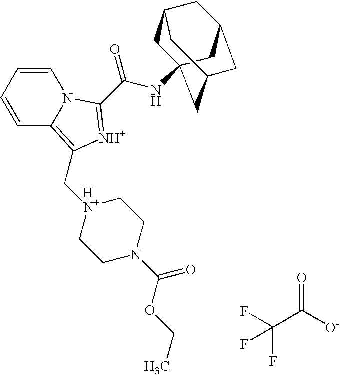 | ethyl 4-({3-(3S,5S,7S)-adamantan- 1-ylcarbamoyl]imidazo[1,5- a]pyridin-1-yl}methyl)piperazine-1- carboxylate | 466.2827 |
| 195 | 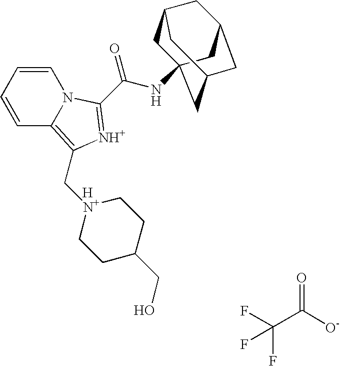 | N-[(3S,5S,7S)-adamantan-1-yl]-1- {[4-(hydroxymethyl)piperidin-1- yl]methyl}imidazo[1,5-a]pyridine- 3-carboxamide | 423.2767 |
| 196 | 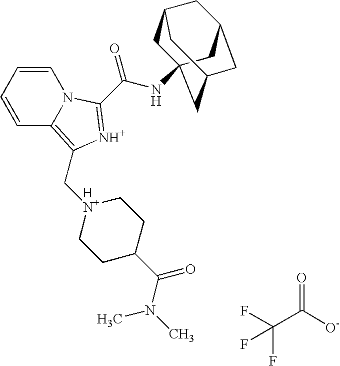 | N-[(3S,5S,7S)-adamantan-1-yl]-1- {[4-(dimethylcarbamoyl)piperidin- 1-yl]methyl}imidazo[1,5- a]pyridine-3-carboxamide | 464.3037 |
| 197 | 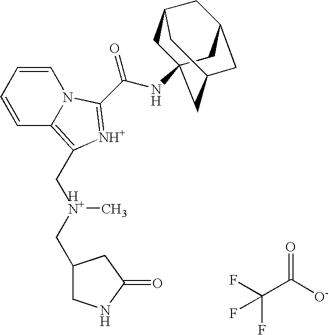 | N-[(3S,5S,7S)-adamantan-1-yl]-1- ({methyl[(5-oxopyrrolidin-3- yl)methyl]amino}methyl)imidazo[1, 5-a]pyridine-3-carboxamide | 436.2722 |
| 198 | 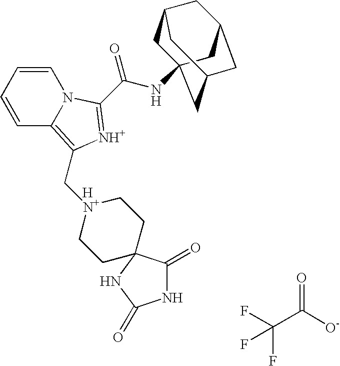 | N-[(3S,5S,7S)-adamantan-1-yl]-1- [(2,4-dioxo-1,3,8- triazaspiro[4.5]dec-8- yl)methyl]imidazo[1,5-a]pyridine-3- carboxamide | 477.2619 |
| 199 | 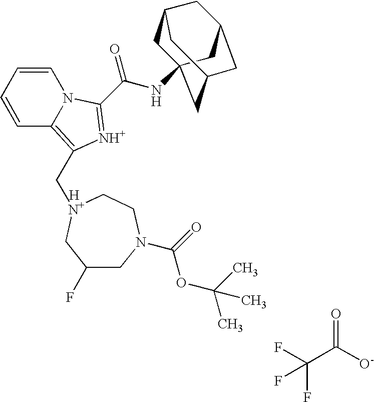 | tert-butyl 4-({3-[(3S,5S,7S)- adamantan-1- ylcarbamoyl]imidazo[1,5-a]pyridin- 1-yl}methyl)-6-fluoro-1,4- diazepane-1-carboxylate | 524.2358 |
| 200 | 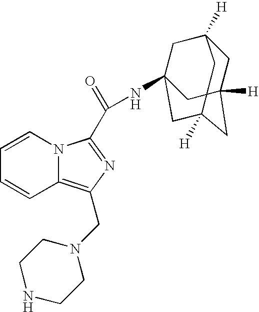 | N-[(3S,5S,7S)-adamantan-1-yl]-1- (piperazin-1-ylmethyl)imidazo[1,5- a]pyridine-3-carboxamide | 394.2615 |
| 201 | 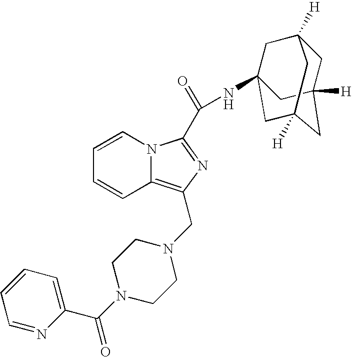 | N-[(3S,5S,7S)-adamantan-1-yl]-1- {[4-(pyridin-2-ylcarbonyl)piperazin- 1-yl]methyl}imidazo[1,5- a]pyridine-3-carboxamide | 499.2836 |
| 202 | 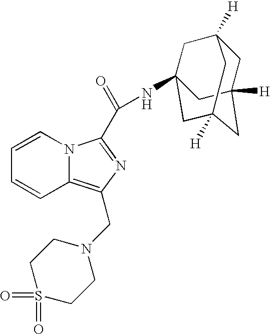 | N-[(3S,5S,7S)-adamantan-1-yl]-1- [(1,1-dioxidothiomorpholin-4- yl)methyl]imidazo[1,5-a]pyridine-3- carboxamide | 443.2121 |
| 203 | 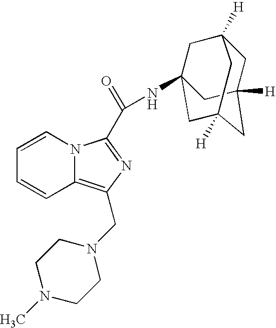 | N-[(3S,5S,7S)-adamantan-1-yl]-1- [(4-methylpiperazin-1- yl)methyl]imidazo[1,5-a]pyridine-3- carboxamide | 408.2759 |
| 204 | 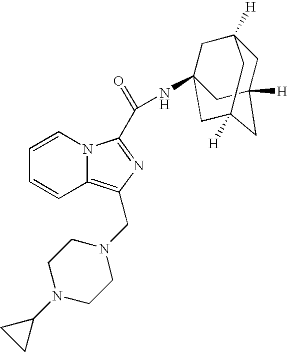 | N-[(3S,5S,7S)-adamantan-1-yl]-1- [(4-cyclopropylpiperazin-1- yl)methyl]imidazo[1,5-a]pyridine-3- carboxamide | 308.2 (fragment) |
| 205 | 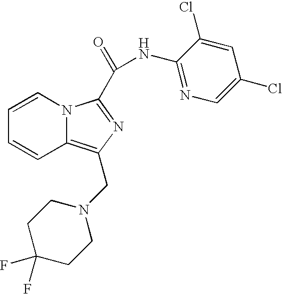 | N-(3,5-dichloropyridin-2-yl)-1- [(4,4-difluoropiperidin-1- yl)methyl]imidazo[1,5-a]pyridine-3- carboxamide | 440.0865 |
| 206 | 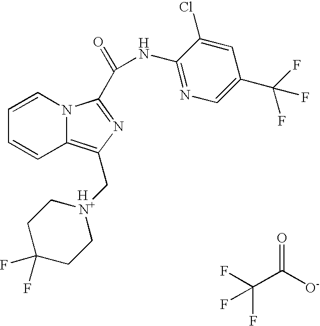 | N-[3-chloro-5- (trifluoromethyl)pyridin-2-yl]-1- [(4,4-difluoropiperidin-1- yl)methyl]imidazo[1,5-a]pyridine-3- carboxamide | 474.1129 |
| 207 | 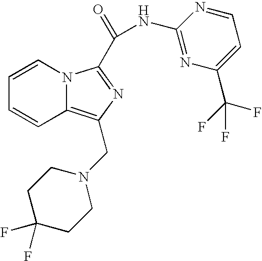 | 1-[(4,4-difluoropiperidin-1- yl)methyl]-N-[4- (trifluoromethyl)pyrimidin-2- yl]imidazo[1,5-a]pyridine-3- carboxamide | 441.1464 |
| 208 | 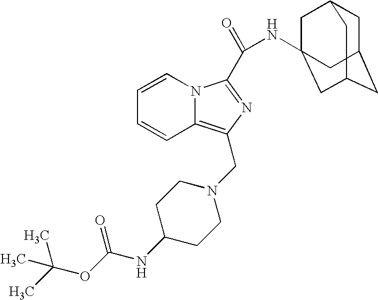 | tert-butyl (1-{[3-(adamantan-1- ylcarbamoyl)imidazo[1,5-a]pyridin- 1-yl]methyl}piperidin-4- yl)carbamate | 508.3 (fragment) |
| 209 | 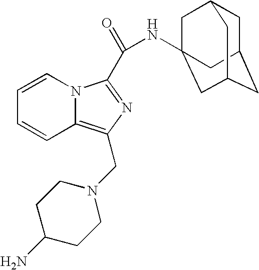 | N-adamantan-1-yl-1-[(4- aminopiperidin-1- yl)methyl]imidazo[1,5-a]pyridine-3- carboxamide | 408.2 (fragment) |
| 210 | 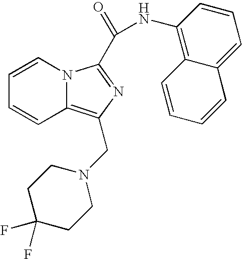 | 1-[(4,4-difluoropiperidin-1- yl)methyl]-N-1- naphthylimidazo[1,5-a]pyridine-3- carboxamide | 421.1851 |
| 211 | 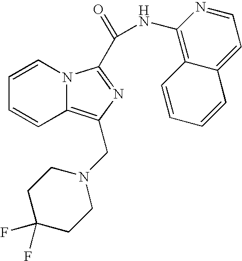 | 1-[(4,4-difluoropiperidin-1- yl)methyl]-N-isoquinolin-1- ylimidazo[1,5-a]pyridine-3- carboxamide | 422.1787 |
| 212 | 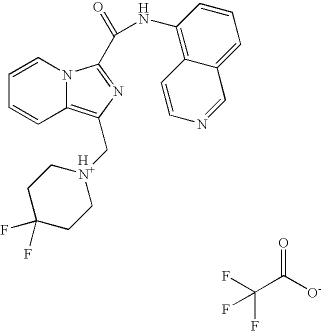 | 1-[(4,4-difluoropiperidin-1- yl)methyl]-N-isoquinolin-5- ylimidazo[1,5-a]pyridine-3- carboxamide | 422.1795 |
| 213 | 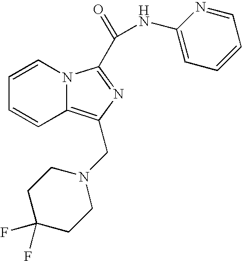 | 1-[(4,4-difluoropiperidin-1- yl)methyl]-N-pyridin-2- ylimidazo[1,5-a]pyridine-3- carboxamide | 372.1639 |
| 214 | 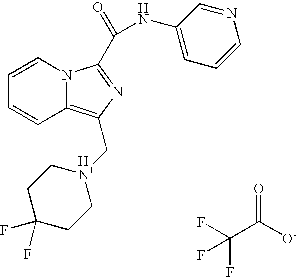 | 1-[(4,4-difluoropiperidin-1- yl)methyl]-N-pyridin-3- ylimidazo[1,5-a]pyridine-3- carboxamide | 372.1636 |
| 215 | 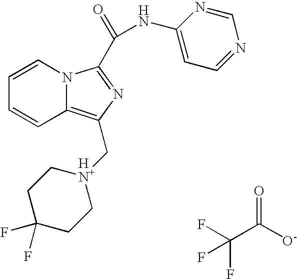 | 1-[(4,4-difluoropiperidin-1- yl)methyl]-N-pyrimidin-4- ylimidazo[1,5-a]pyridine-3- carboxamide | 373.1589 |
| 216 | 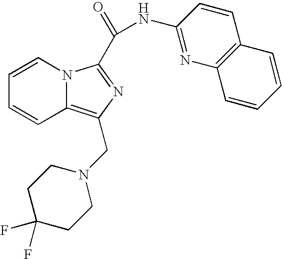 | 1-[(4,4-difluoropiperidin-1- yl)methyl]-N-quinolin-2- ylimidazo[1,5-a]pyridine-3- carboxamide | 422.18 |
| 217 | 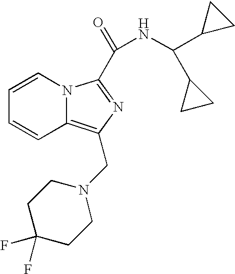 | N-(dicyclopropylmethyl)-1-[(4,4- difluoropiperidin-1- yl)methyl]imidazo[1,5-a]pyridine-3- carboxamide | 268.1 (fragment) |
| 218 | 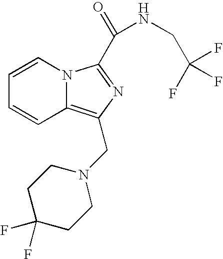 | 1-[(4,4-difluoropiperidin-1- yl)methyl]-N-(2,2,2- trifluoroethyl)imidazo[1,5- a]pyridine-3-carboxamide | 377.1407 |
| 219 | 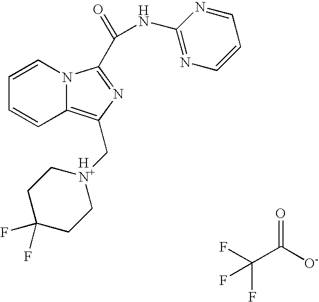 | 1-[(4,4-difluoropiperidin-1- yl)methyl]-N-pyrimidin-2- ylimidazo[1,5-a]pyridine-3- carboxamide | 373.1589 |
| 220 | 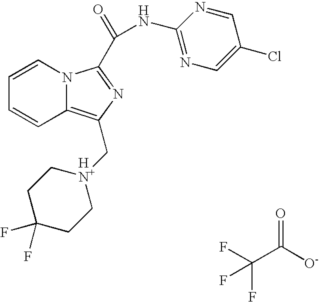 | N-(5-chloropyrimidin-2-yl)-1-[(4,4- difluoropiperidin-1- yl)methyl]imidazo[1,5-a]pyridine-3- carboxamide | 407.1204 |
| 221 | 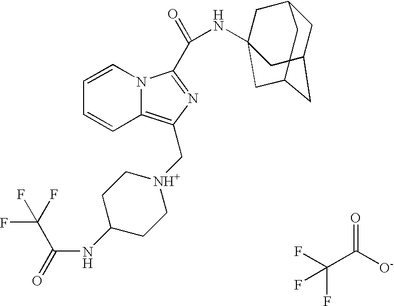 | N-adamantan-1-yl-1-({4- [(trifluoroacetyl)amino]piperidin-1- yl}methyl)imidazo[1,5-a]pyridine- 3-carboxamide | 504.2599 |
| 222 | 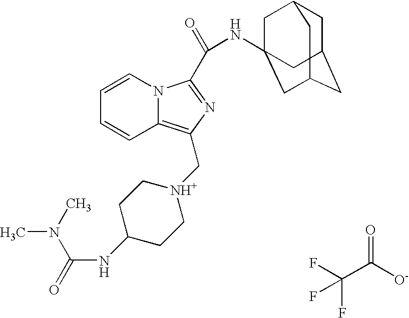 | N-adamantan-1-yl-1-({4- [(dimethylcarbamoyl)amino]piperidin- 1-yl}methyl)imidazo[1,5- a]pyridine-3-carboxamide | 479.3138 |
| 223 | 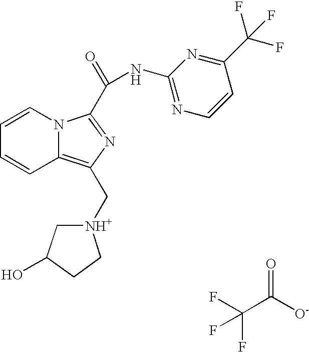 | 1-[(3-hydroxypyrrolidin-1- yl)methyl]-N-[4- (trifluoromethyl)pyrimidin-2- yl]imidazo[1,5-a]pyridine-3- carboxamide | 320.0 (fragment) |
| 224 | 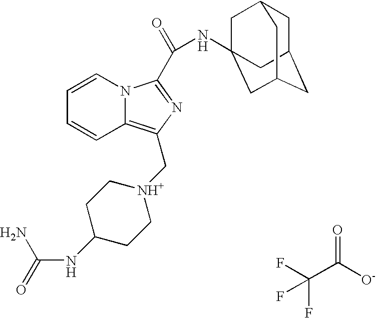 | N-adamantan-1-yl-1-{[4- (carbamoylamino)piperidin-1- yl]methyl}imidazo[1,5-a]pyridine- 3-carboxamide | 451.2827 |
| 225 | 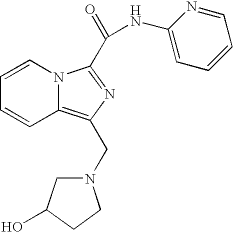 | 1-[(3-hydroxypyrrolidin-1- yl)methyl]-N-pyridin-2- ylimidazo[1,5-a]pyridine-3- carboxamide | 338.158 |
| 226 | 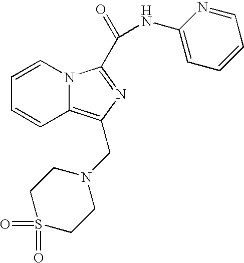 | 1-[(1,1-dioxidothiomorpholin-4- yl)methyl]-N-pyridin-2- ylimidazo[1,5-a]pyridine-3- carboxamide | 386.1254 |
| 227 | 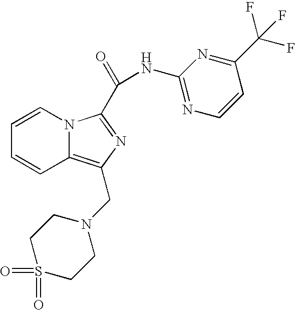 | 1-[(1,1-dioxidothiomorpholin-4- yl)methyl]-N-[4- (trifluoromethyl)pyrimidin-2- yl]imidazo[1,5-a]pyridine-3- carboxamide | 451.1088 |
| 228 | 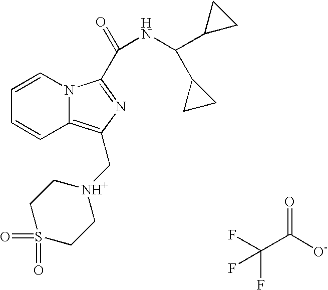 | N-(dicyclopropylmethyl)-1-[(1,1- dioxidothiomorpholin-4- yl)methyl]imidazo[1,5-a]pyridine-3- carboxamide | 403.1776 |
| 229 | 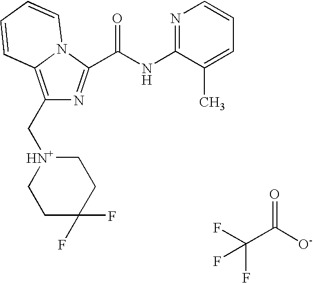 | 1-[(4,4-difluoropiperidin-1- yl)methyl]-N-(3-methylpyridin-2- yl)imidazo[1,5-a]pyridine-3- carboxamide | 386.1786 |
| 230 | 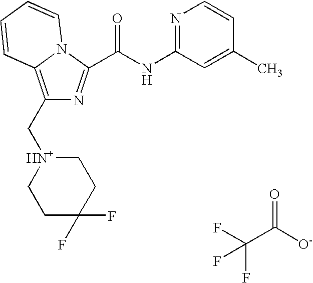 | 1-[(4,4-difluoropiperidin-1- yl)methyl]-N-(4-methylpyridin-2- yl)imidazo[1,5-a]pyridine-3- carboxamide | 386.1785 |
| 231 | 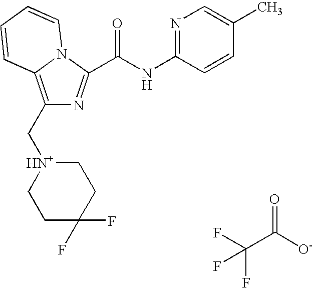 | 1-[(4,4-difluoropiperidin-1- yl)methyl]-N-(5-methylpyridin-2- yl)imidazo[1,5-a]pyridine-3- carboxamide | 386.1786 |
| 232 | 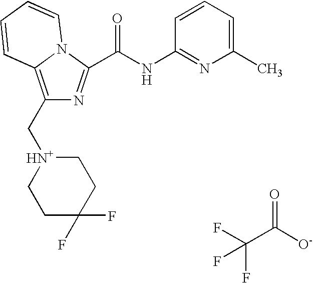 | 1-[(4,4-difluoropiperidin-1- yl)methyl]-N-(6-methylpyridin-2- yl)imidazo[1,5-a]pyridine-3- carboxamide | 386.1786 |
| 233 | 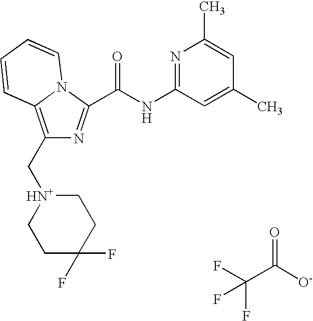 | 1-[(4,4-difluoropiperidin-1- yl)methyl]-N-(4,6-dimethylpyridin- 2-yl)imidazo[1,5-a]pyridine-3- carboxamide | 400.1939 |
| 234 | 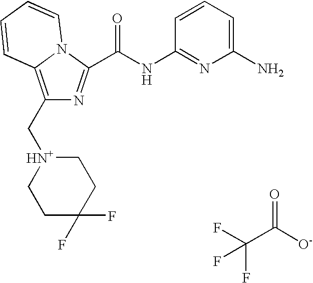 | N-(6-aminopyridin-2-yl)-1-[(4,4- difluoropiperidin-1- yl)methyl]imidazo[1,5-a]pyridine-3- carboxamide | 387.1739 |
| 235 | 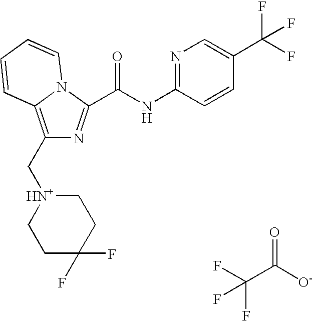 | 1-[(4,4-difluoropiperidin-1- yl)methyl]-N-[5- (trifluoromethyl)pyridin-2- yl]imidazo[1,5-a]pyridine-3- carboxamide | 440.1494 |
| 236 | 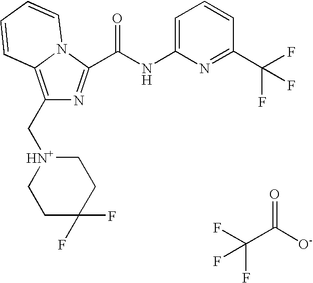 | 1-[(4,4-difluoropiperidin-1- yl)methyl]-N-[6- (trifluoromethyl)pyridin-2- yl]imidazo[1,5-a]pyridine-3- carboxamide | 440.1494 |
| 237 | 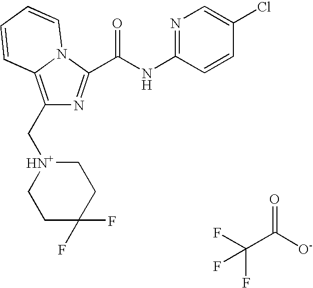 | N-(5-chloropyridin-2-yl)-1-[(4,4- difluoropiperidin-1- yl)methyl]imidazo[1,5-a]pyridine-3- carboxamide | 406.1238 |
| 238 | 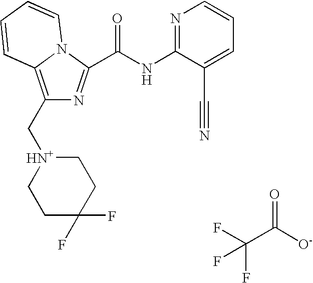 | N-(3-cyanopyridin-2-yl)-1-[(4,4- difluoropiperidin-1- yl)methyl]imidazo[1,5-a]pyridine-3- carboxamide | 397.1582 |
| 239 | 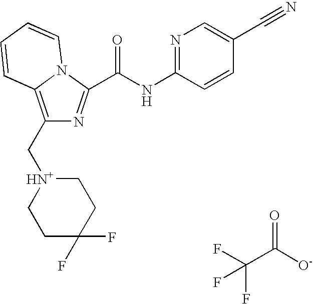 | N-(5-cyanopyridin-2-yl)-1-[(4,4- difluoropiperidin-1- yl)methyl]imidazo[1,5-a]pyridine-3- carboxamide | 397.1582 |
| 240 | 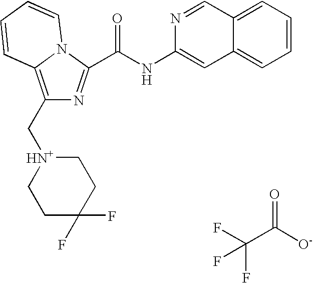 | 1-[(4,4-difluoropiperidin-1- yl)methyl]-N-isoquinolin-3- ylimidazo[1,5-alpyridine-3- carboxamide | 422.1785 |
| 241 | 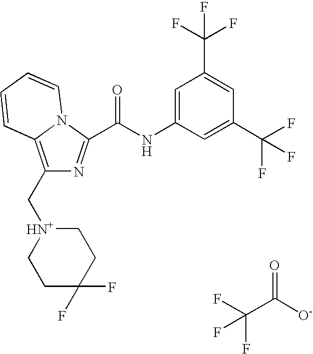 | N-[3,5-bis(trifluoromethyl)phenyl]- 1-[(4,4-difluoropiperidin-1- yl)methyl]imidazo[1,5-a]pyridine-3- carboxamide | 507.1411 |
| 242 | 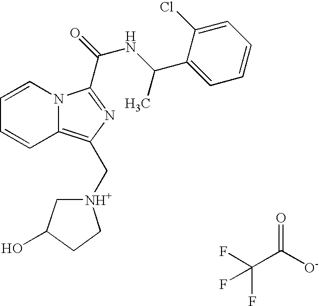 | N-[1-(2-chlorophenyl)ethyl]-1-[(3- hydroxypyrrolidin-1- yl)methyl]imidazo[1,5-a]pyridine-3- carboxamide | 399.158 |
| 243 | 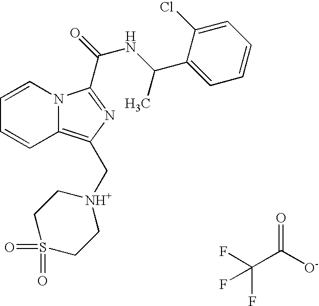 | N-[1-(2-chlorophenyl)ethyl]-1-[(1,1- dioxidothiomorpholin-4- yl)methyl]imidazo[1,5-a]pyridine-3- carboxamide | 469.1065 (M + Na) |
| 244 | 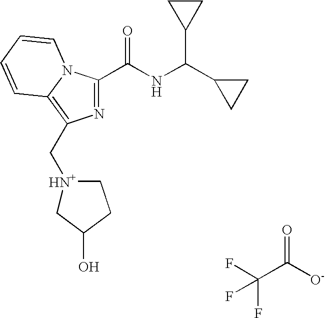 | N-(dicyclopropymethyl)-1-[(3- hydroxypyrrolidin-1- yl)methyl]imidazo[1,5-a]pyridine-3- carboxamide | 377.1948 (M + Na) |
| 245 | 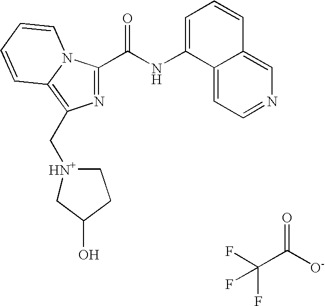 | 1-[(3-hydroxypyrrolidin-1- yl)methyl]-N-isoquinolin-5- ylimidazo[1,5-a]pyridine-3- carboxamide | 388.1768 |
| 246 | 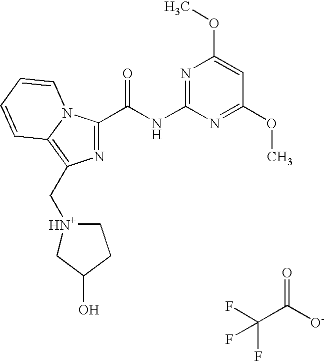 | N-(4,6-dimethoxypyrimidin-2-yl)-1- [(3-hydroxypyrrolidin-1- yl)methyl]imidazo[1,5-a]pyridine-3- carboxamide | 399.1776 |
| 247 | 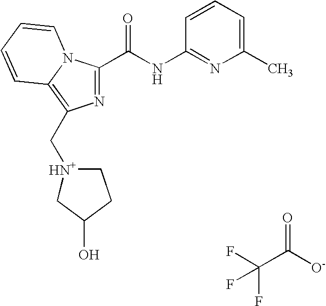 | 1-[(3-hydroxypyrrolidin-1- yl)methyl]-N-(6-methylpyridin-2- ylimidazo[1,5-a]pyridine-3- carboxamide | 352.1772 |
| 248 | 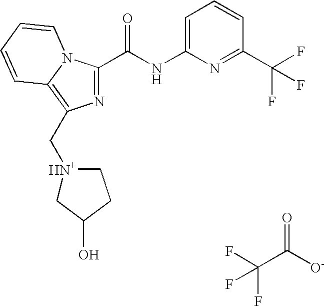 | 1-[(3-hydroxypyrrolidin-1- yl)methyl]-N-[6- (trifluoromethyl)pyridin-2- yl]imidazo[1,5-a]pyridine-3- carboxamide | 406.1483 |
| 249 | 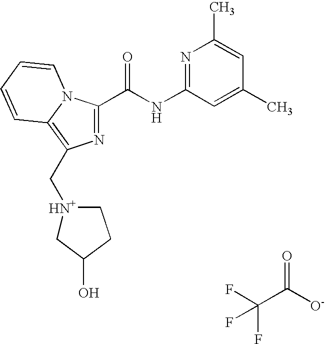 | N-(4,6-dimethylpyridin-2-yl)-1-[(3- hydroxypyrrolidin-1- yl)methyl]imidazo[1,5-a]pyridine-3- carboxamide | 366.1927 |
| 250 | 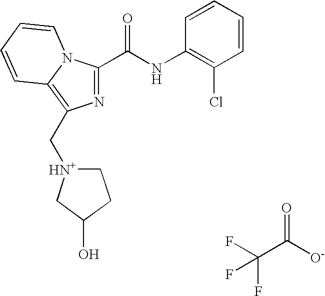 | N-(2-chlorophenyl)-1-[(3- hydroxypyrrolidin-1- yl)methyl]imidazo[1,5-a]pyridine-3- carboxamide | 371.1273 |
| 251 | 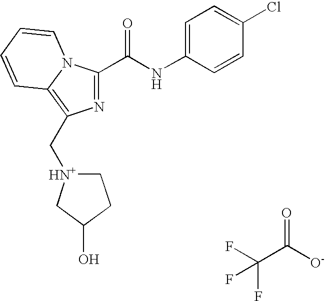 | N-(4-chlorophenyl)-1-[(3- hydroxypyrrolidin-1- yl)methyl]imidazo[1,5-a]pyridine-3- carboxamide | 371.1273 |
| 252 | 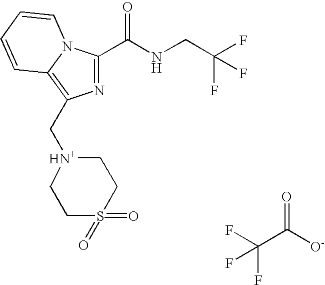 | 1-[(1,1-dioxidothiomorpholin-4- yl)methyl]-N-(2,2,2- trifluoroethyl)imidazo[1,5- a]pyridine-3-carboxamide | 281.1049 |
| 253 | 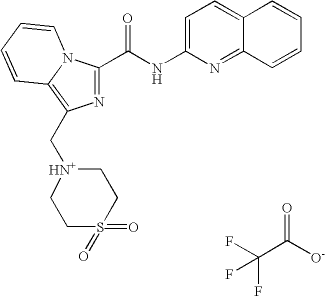 | 1-[(1,1-dioxidothiomorpholin-4- yl)methyl]-N-quinolin-2- ylimidazo[1,5-a]pyridine-3- carboxamide | 436.1419 |
| 254 | 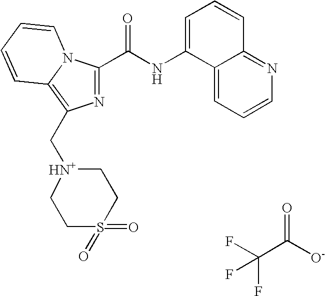 | 1-[(1,1-dioxidothiomorpholin-4- yl)methyl]-N-quinolin-5- ylimidazo[1,5-a]pyridine-3- carboxamide | 436.1426 |
| 255 | 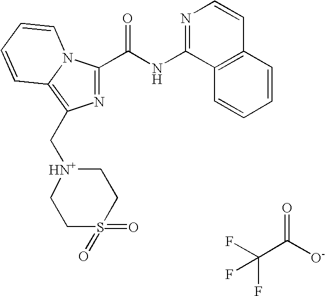 | 1-[(1,1-dioxidothiomorpholin-4- yl)methyl]-N-isoquinolin-1- ylimidazo[1,5-a]pyridine-3- carboxamide | 436.1424 |
| 256 | 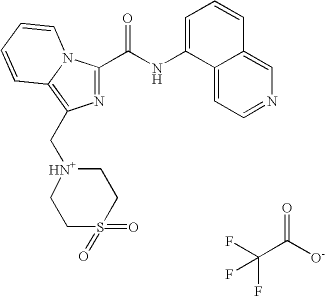 | 1-[(1,1-dioxidothiomorpholin-4- yl)methyl]-N-isoquinolin-5- ylimidazo[1,5-a]pyridine-3- carboxamide | 436.1419 |
| 257 | 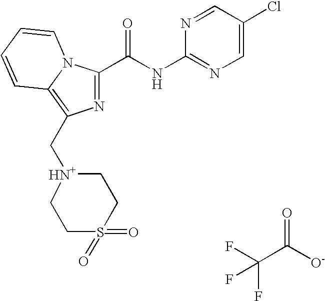 | N-(5-chloropyrimidin-2-yl)-1-[(1,1- dioxidothiomorpholin-4- yl)methyl]imidazo[1,5-a]pyridine-3- carboxamide | 421.0834 |
| 258 | 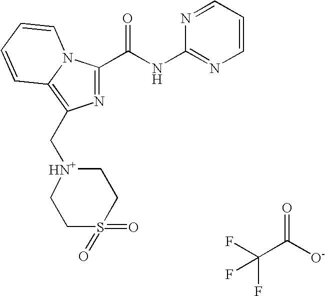 | 1-[(1,1-dioxidothiomorpholin-4- yl)methyl]-N-pyrimidin-2- ylimidazo[1,5-a]pyridine-3- carboxamide | 409.104 |
| 259 | 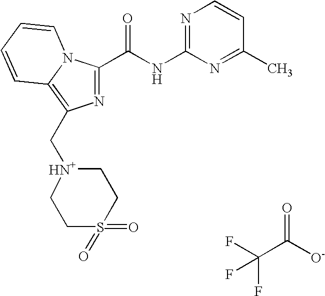 | 1-[(1,1-dioxidothiomorpholin-4- yl)methyl]-N-(4-methylpyrimidin-2- ylimidazo[1,5-a]pyridine-3- carboxamide | 401.1386 |
| 260 | 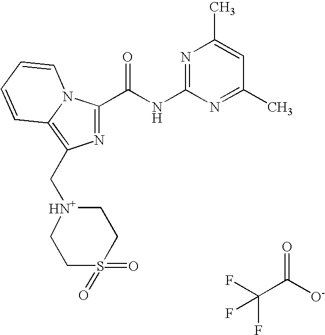 | N-(4,6-dimethylpyrimidin-2-yl)-1- [(1,2-dioxidothiomorpholin-4- yl)methyl]imidazo[1,5-a]pyridine-3- carboxamide | 415.1535 |
| 261 | 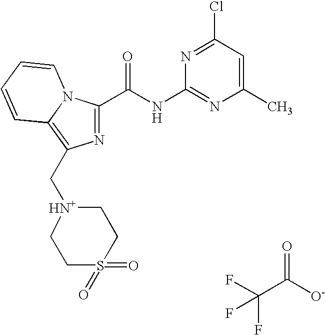 | N-(4-chloro-6-methylpyrimidin-2- yl)-1-[(1,1-dioxidothiomorpholin-4- yl)methyl]imidazo[1,5-a]pyridine-3- carboxamide | 435.0992 |
| 262 | 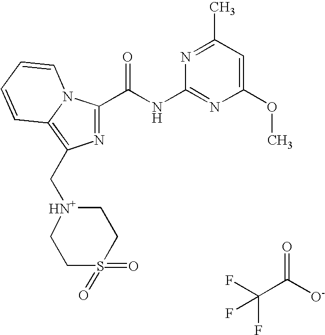 | 1-[(1,1-dioxidothiomorpholin-4- yl)methyl]-N-(4-methoxy-6- methylpyrimidin-2-yl)imidazo[1,5- a]pyridine-3-carboxamide | 431.1479 |
| 263 | 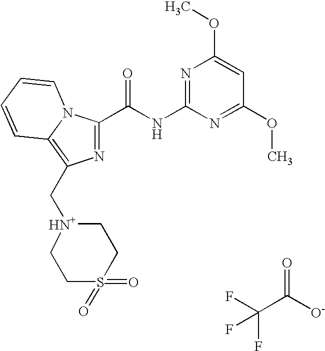 | N-(4,6-dimethylpyrimidin-2-yl)-1- [(1,1-dioxidothiomorpholin-4- yl)methyl]imidazo[1,5-a]pyridine-3- carboxamide | 447.1425 |
| 264 | 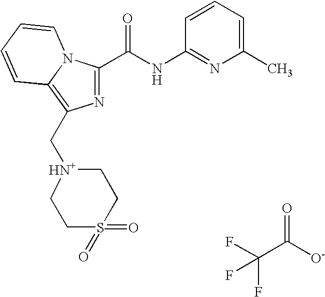 | 1-[(1,1-dioxidothiomorpholin-4- yl)methyl]-N-(6-methylpyridin-2- yl)imidazo[1,5-a]pyridine-3- carboxamide | 400.1428 |
| 265 | 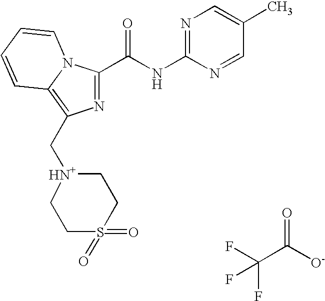 | 1-[(1,1-dioxidothiomorpholin-4- yl)methyl]-N-(5-methylpyridin-2- yl)imidazo[1,5-a]pyridine-3- carboxamide | 400.1427 |
| 266 | 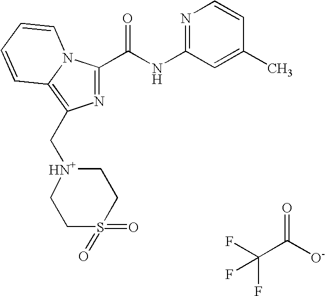 | 1-[(1,1-dioxidothiomorpholin-4- yl)methyl]-N-(4-methylpyridin-2- yl)imidazo[1,5-a]pyridine-3- carboxamide | 400.1429 |
| 267 | 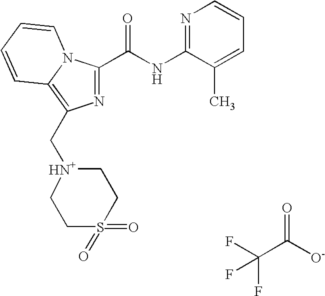 | 1-[(1,1-dioxidothiomorpholin-4- yl)methyl]-N-(3-methylpyridin-2- yl)imidazo[1,5-a]pyridine-3- carboxamide | 400.1428 |
| 268 | 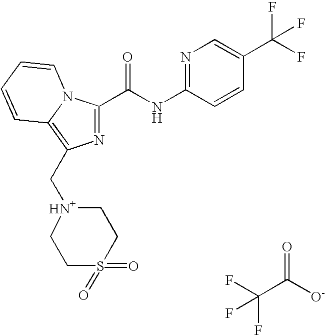 | 1-[(1,1-dioxidothiomorpholin-4- yl)methyl]-N-[5- (trifluoromethyl)pyridin-2- yl]imidazo[1,5-a]pyridine-3- carboxamide | 454.1135 |
| 269 | 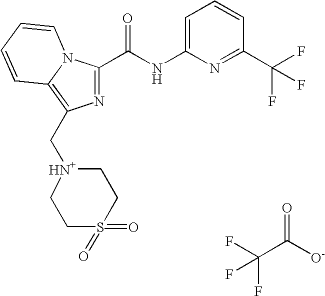 | 1-[(1,1-dioxidothiomorpholin-4- yl)methyl]-N-[6- (trifluoromethyl)pyridin-2- yl]imidazo[1,5-a]pyridine-3- carboxamide | 454.115 |
| 270 | 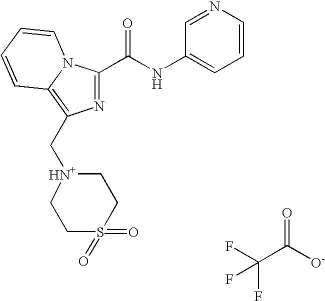 | 1-[(1,1-dioxidothiomorpholin-4- yl)methyl]-N-pyridin-3- ylimidazo[1,5-a]pyridine-3- carboxamide | 386.1275 |
| 271 | 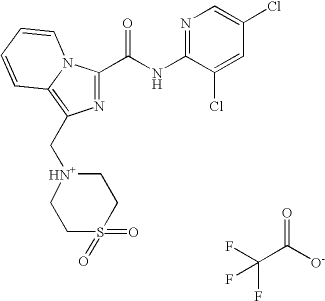 | N-(3,5-dichloropyridin-2-yl)-1- [(1,1-dioxidothiomorpholin-4- yl)methyl]imidazo[1,5-a]pyridine-3- carboxamide | 454.0483 |
| 272 | 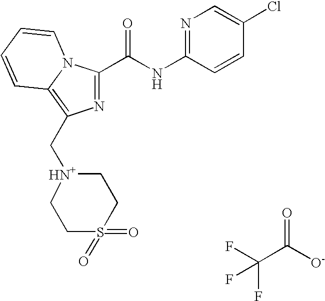 | N-(5-chloropyridin-2-yl)-1-[(1,1- dioxidothiomorpholin-4- yl)methyl]imidazo[1,5-a]pyridine-3- carboxamide | 420.0904 |
| 273 | 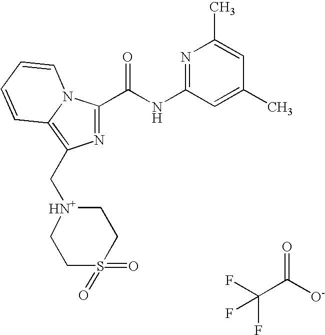 | N-(4,6-dimethylpyridin-2-yl)-1- [(1,1-dioxidothiomorpholin-4- yl)methyl]imidazo[1,5-a]pyridine-3- carboxamide | 414.1582 |
| 274 | 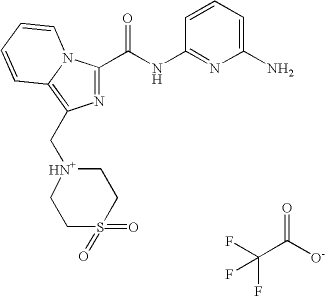 | N-(6-aminopyridin-2-yl)-1-[(1,1- dioxidothiomorpholin-4- yl)methyl]imidazo[1,5-a]pyridine-3- carboxamide | 401.1386 |
| 275 | 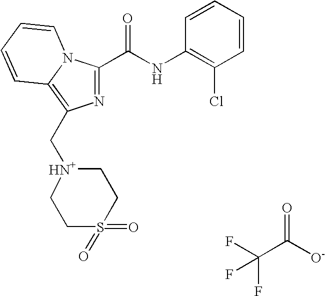 | N-(2-chlorophenyl)-1-[(1,1- dioxidothiomorpholin-4- yl)methyl]imidazo[1,5-a]pyridine-3- carboxamide | 441.0743 (M + Na) |
| 276 | 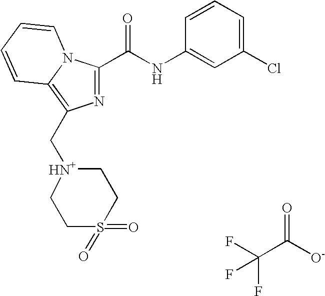 | N-(3-chlorophenyl)-1-[(1,1- dioxidothiomorpholin-4- yl)methyl]imidazo[1,5-a]pyridine-3- carboxamide | 419.0933 |
| 277 | 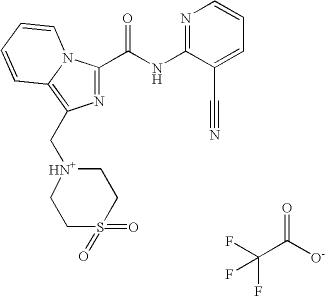 | N-(3-cyanopyridin-2-yl)-1-[(1,1- dioxidothiomorpholin-4- yl)methyl]imidazo[1,5-a]pyridine-3- carboxamide | 411.1221 |
| 278 | 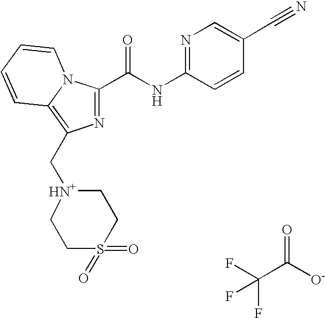 | N-(5-cyanopyridin-2-yl)-1-[(1,1- dioxidothiomorpholin-4- yl)methyl]imidazo[1,5-a]pyridine-3- carboxamide | |
| 279 | 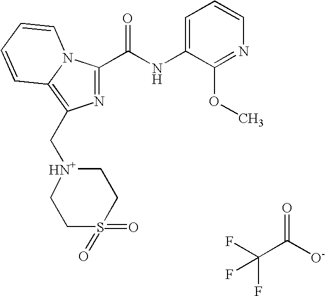 | 1-[(1,1-dioxidothiomorpholin-4- yl)methyl]-N-(2-methoxypyridin-3- yl)imidazo[1,5-a]pyridine-3- carboxamide | 416.1375 |
| 280 | 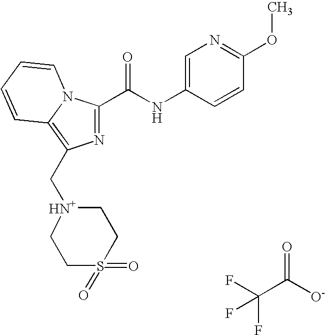 | 1-[(1,1-dioxidothiomorpholin-4- yl)methyl]-N-(6-methoxypyridin-3- yl)imidazo[1,5-a]pyridine-3- carboxamide | 416.1374 |
| 281 | 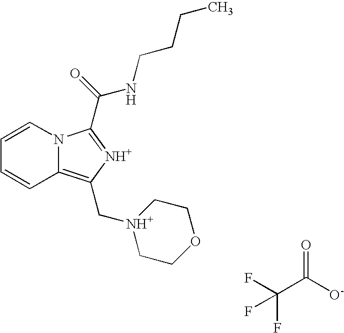 | 3-[(butylamino)carbonyl]-1- (morpholin-4-ium-4- ylmethyl)imidazo[1,5-a]pyridin-2- ium bis(trifluoroacetate) | 229.2384 |
| 282 | 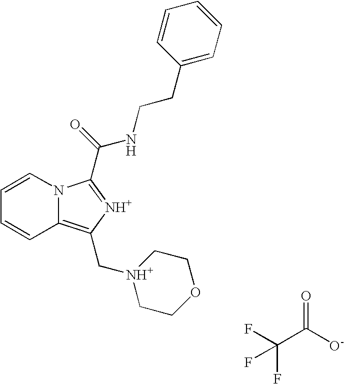 | 1-(morpholin-4-ium-4-ylmethyl)-3- {[(2- phenylethyl)amino]carbonyl}imidazo [1,5-a]pyridin-2-ium bis(trifluoroacetate) | 277.2379 |
| 283 | 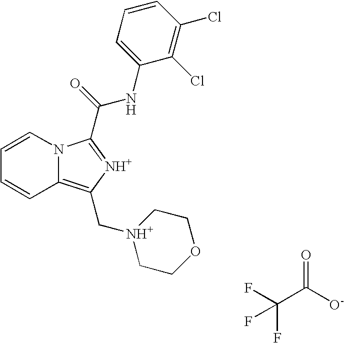 | 3-{[(2,3- dichlorophenyl)amino]carbonyl}-1- (morpholin-4-ium-4- ylmethyl)imidazo[1,5-a]pyridin-2- ium bis(trifluoroacetate) | 405.0868 |
| 284 | 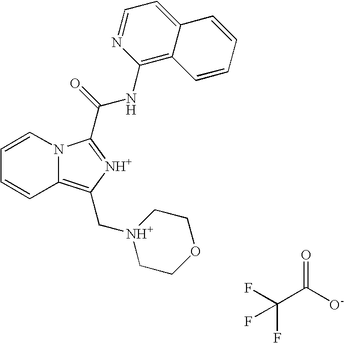 | 3-[(isoquinolin-1- ylamino)carbonyl]-1-(morpholin-4- ium-4-ylmethyl)imidazo[1,5- a]pyridin-2-ium bis(trifluoroacetate) | 388.1758 |
| 285 | 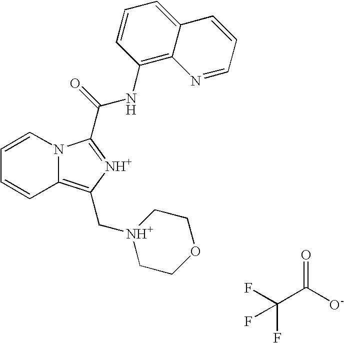 | 1-(morpholin-4-ium-4-ylmethyl)-3- [(quinolin-8- ylamino)carbonyl]imidazo[1,5- a]pyridin-2-ium bis(trifluoroacetate) | 388.1755 |
| 286 | 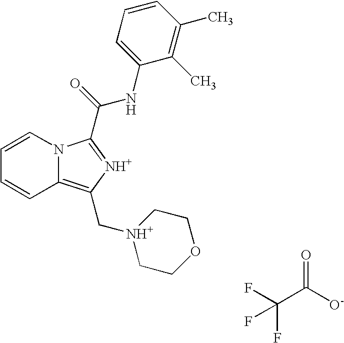 | 3-{[(2,3- dimethylphenyl)amino]carbonyl}-1- (morpholin-4-ium-4- ylmethyl)imidazo[1,5-a]pyridin-2- ium bis(trifluoroacetate) | 365.1965 |
| 287 | 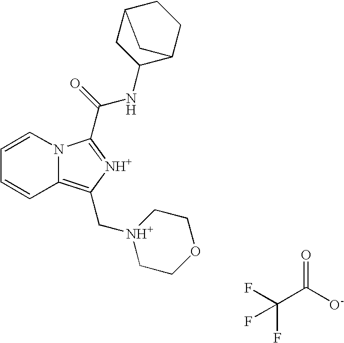 | 3-[(bicyclo[2.2.1]hept-2- ylamino)carbonyl]-1-(morpholin-4- ium-4-ylmethyl)imidazo[1,5- a]pyridin-2-ium bis(trifluoroacetate) | 267.2537 |
| 288 | 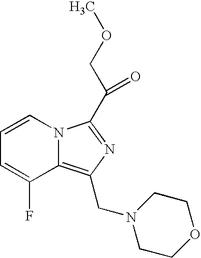 | ethyl 8-fluoro-1-(morpholin-4- ylmethyl)imidazo[1,5-a]pyridine-3- carboxylate | 308.1 |
| 289 | 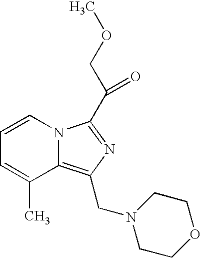 | ethyl 8-methyl-1-(morpholin-4- ylmethyl)imidazo[1,5-a]pyridine-3- carboxylate | 217.1 (m-C4H8NO) |
| 290 | 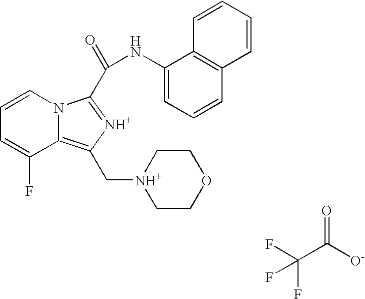 | 8-fluoro-1-(morpholin-4-ium-4- ylmethyl)-3-[(1- naphthylamino)carbonyl]imidazo[1, 5-a]pyridin-2-ium bis(trifluoroacetate) | 405.1 |
| 291 | 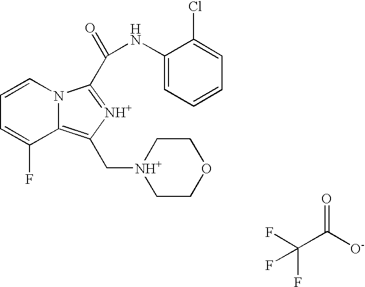 | 3-{[(2- chlorophenyl)amino]carbonyl}-8- fluoro-1-(morpholin-4-ium-4- ylmethyl)imidazo[1,5-a]pyridin-2- ium bis(trifluoroacetate) | 389.1 |
| 292 | 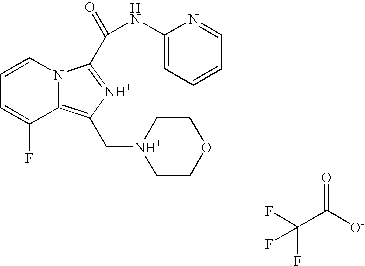 | 8-fluoro-1-(morpholin-4-ium-4- ylmethyl)-3-[(pyridin-2- ylamino)carbonyl]imidazo[1,5- a]pyridin-2-ium bis(trifluoroacetate) | 356.1 |
| 293 | 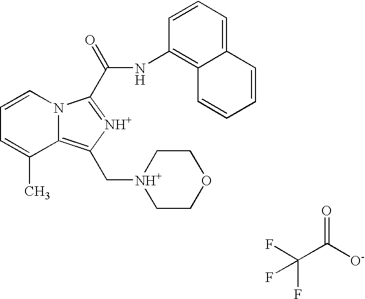 | 8-methyl-1-(morpholin-4-ium-4- ylmethyl)-3-[(1- naphthylamino)carbonyl]imidazo[1, 5-a]pyridin-2-ium bis(trifluoroacetate) | 401.1 |
| 294 | 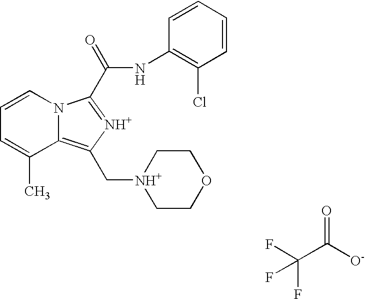 | 3-{[(2- chlorophenyl)amino]carbonyl}-8- methyl-1-(morpholin-4-ium-4- ylmethyl)imidazo[1,5-a]pyridin-2- ium bis(trifluoroacetate) | 298.1 (m-C4H8NO) |
| 295 | 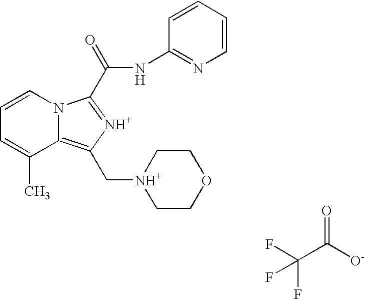 | 8-methyl-1-(morpholin-4-ium-4- ylmethyl)-3-[(pyridin-2- ylamino)carbonyl]imidazo[1,5- a]pyridin-2-ium bis(trifluoroacetate) | 265.1 (m-C4H8NO) |
| 296 | 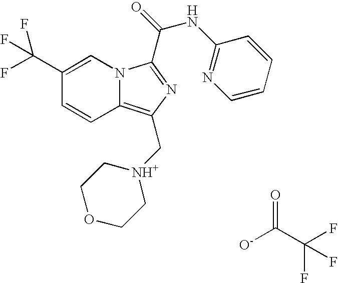 | 4-{[3-[(pyridin-2- ylamino)carbonyl]-6- (trifluoromethyl)iinidazo[1,5- a]pyridin-1-yl]methyl}morpholin-4- ium trifluoroacetate | MS(M + 1) = 406.1 |
| 643 | 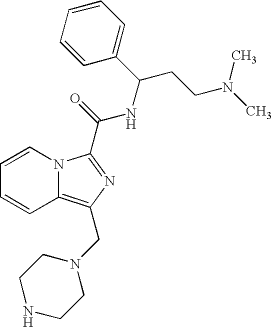 | N-[3-(dimethylamino)-1- phenylpropyl]-1-(piperazin-1- ylmethyl)imidazo[1,5-a]pyridine-3- carboxamide | 421.0 |

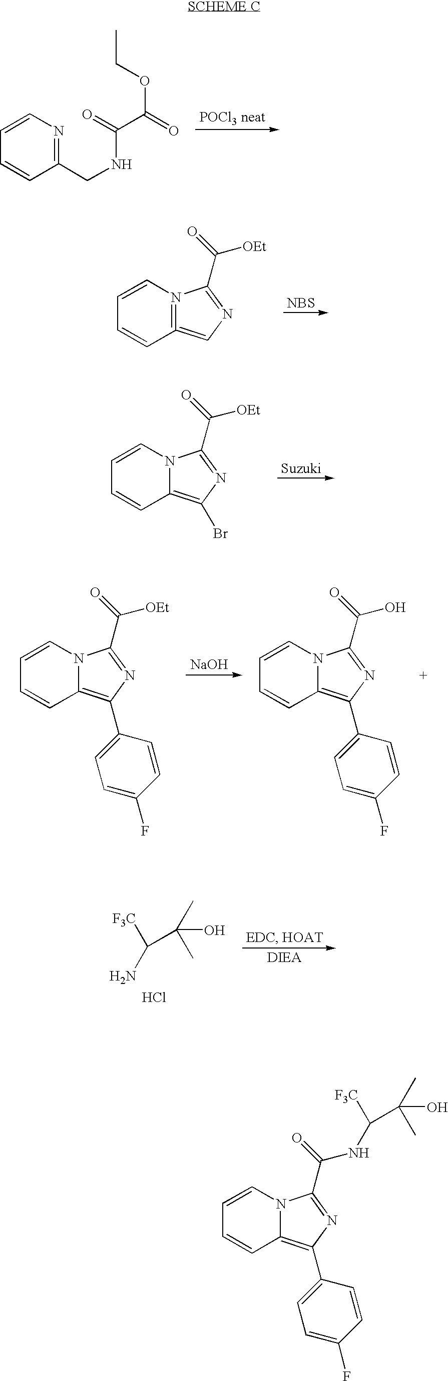

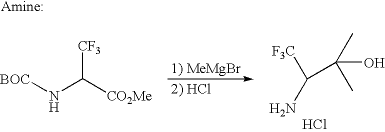

### Example 297

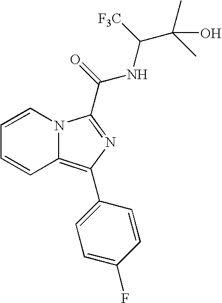

### 1-(4-Fluorophenyl)-N-[2-hydroxy-2-methyl-1-(trifluoromethyl)propyl]imidazo[1,5-a]pyridine-3-carboxamide

### Step A: Ethyl oxo[(pyridin-2-ylmethyl)amino]acetate

Reaction was done as previously described for intermediate X.

### Step B: Ethyl imidazo[1,5-a]pyridine-3-carboxylate

A solution of ethyl oxo[(pyridin-2-ylmethyl)amino]acetate (40 g, 192 mmol) in POCl₃ (300 mL) was heated to reflux for 18 h. The mixture was concentrated and saturated aqueous NaHCO₃ was added. The mixture was extracted with EtOAc (3×, 500 mL) and the combined organic extracts were washed with saturated brine, dried over Na₂SO₄, filtered, and concentrated to give a black oily solid. The residue was purified by silica gel chromatography (12%-70% EtOAc/Hexanes) to give the title compound (16 g, 44%). MS 191.1 (M+1).

### Step C: Ethyl 1-bromoimidazo[1,5-a]pyridine-3-carboxylate

To an ice cold solution of ethyl imidazo[1,5-a]pyridine-3-carboxylate (10 g, 52.6 mmol) in acetonitrile (500 mL) was added N-bromo succinimide (9.36 g, 52.6 mmol). The mixture was allowed to stir for 1 h at which time triethylamine (10 mL) was added to the deep red-violet solution. The color dissipated and the solution was concentrated. Purification of the crude solid by silica gel chromatography (0-20% EtOAc/Hexanes) provided a white crystalline solid (13.5 g 95%). MS 268.9 (M+1)

### Step D: Ethyl 1-(4-fluorophenyl)imidazo[1,5-a]pyridine-3-carboxylate

To a solution of ethyl 1-bromoimidazo[1,5-a]pyridine-3-carboxylate (1.0 g, 3.72 mmol) and (4-fluorophenyl)boronic acid (0.78 g, 5.56 mmol) in dioxane (25 ml) was added PdCl2(dppf) (0.272 g, 0.372 mmol) and potassium phosphate tribasic (2.4 g, 11.2 mmol) dissolved in 3 mL of water. The mixture was heated to reflux for 4 h and then cooled and concentrated. Purification of the crude product by silica gel chromatography (20-50% EtOAc/hexanes) provided a white solid (0.91 g, 86%). MS 285.1 (M+1).

### Step E: 1-(4-Fluorophenyl)imidazo[1,5-a]pyridine-3-carboxylic acid

To a solution of ethyl 1-(4-fluorophenyl)imidazo[1,5-a]pyridine-3-carboxylate (1.35 g, 4.75 mmol) in THF (20 mL) was added sodium hydroxide (2.0 mL, 2.0 M). The reaction was stirred overnight and then hydrochloric acid (12 M) was added until the pH was 3. The mixture was then concentrated to give a white solid as the sodium chloride salt (1.2 g, 99%). MS 257.1 (M+1)

### Step F: tert-Butyl [2-hydroxy-2-methyl-1-(trifluoromethyl)propyl]carbamate

To a solution of methyl N-(tert-butoxycarbonyl)-3,3,3-trifluoroalaninate (2 g, 7.78 mmol) in dry THF (50 ml) was added methyl magnesium bromide in ether (10.63 ml, 31.9 mmol) drop wise at 0° C. under a nitrogen atmosphere. After 3 h at 0° C. the mixture was quenched with a saturated aqueous NH₄Cl solution. Ethyl acetate was added and the layers were separated. The resulting aqueous layer was extracted with EtOAc (2×, 100 mL). The combined organic layers were dried over sodium sulfate, filtered and concentrated. Purification of the crude product by silica gel chromatography (20-50% EtOAc/hexanes) provided a yellow oil. MS 184.1 (M-tBuOH).

### Step G: 3-amino-4,4,4-trifluoro-2-methylbutan-2-ol hydrochloride

To a solution of 4 M dioxane/HCl at 0° C. was added tert-Butyl [2-hydroxy-2-methyl-1-(trifluoromethyl)propyl]carbamate (0.20 g, 0.777 mmol). After 1 h the solution was concentrated to give an off white solid hydrochloride salt. MS 258.1 (M+1)

### Step H: 1-(4-Fluorophenyl)-N-[2-hydroxy-2-methyl-1-(trifluoromethyl)propyl]imidazo[1,5-a]pyridine-3-carboxamide

To a solution of 1-(4-fluorophenyl)imidazo[1,5-a]pyridine-3-carboxylic acid (0.10 g, 0.29 mmol) was added 3-amino-4,4,4-trifluoro-2-methylbutan-2-ol hydrochloride (0.11 g, 0.57 mmol), DIEA (0.256 ml, 1.42 mmol), EDC (0.164 g, 0.854 mmol), and HOAT (0.044 g, 0.285 mmol) in DMF (3.0 ml). After stirring overnight the crude mixture was filtered thru a fiberglass fiber and purified by reverse phase chromatography (5%-95% water/CH3CN) to give the desired product. HRMS 396.1312 (M+1); ¹H NMR (500 MHz, CDCl3) δ 9.49 (d, J=7.2 Hz, 1H), 8.02 (d, J=10.3 Hz, 1H), 7.87 (m, 3H), 7.19 (m, 2H), 7.09 (m, 1H), 6.90 (t, J=6.8 Hz, 1H), 4.70 (m, 1H), 1.92 (bs, 1H), 1.54 (s, 3H), 1.40 (s, 3H).

The following compounds were prepared using the procedures described above:

TABLE C

| Example | Structure | Name | MS(MH) |
|---|---|---|---|
| 297 | 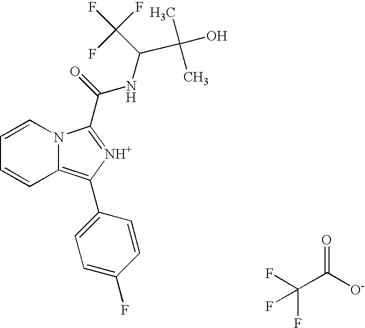 | 1-(4-fluorophenyl)-3-({[2-hydroxy- 2-methyl-1- (trifluoromethyl)propyl]amino} carbonyl)imidazo[1,5-a]pyridin- 2-ium trifluoroacetate | 396.1 |
| 298 | 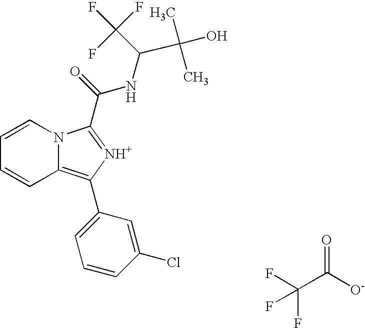 | 1-(3-fluorophenyl)-3-({[2-hydroxy- 2-methyl-1- (trifluoromethyl)propyl]amino} carbonyl)imidazo[1,5-a]pyridin- 2-ium trifluoroacetate | 412.1 |
| 299 | 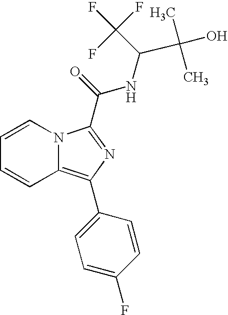 | 1-(4-fluorophenyl)-N-[2-hydroxy- 2-methyl-1- (trifluoromethyl)propyl]imidazo[1, 5-a]pyridine-3-carboxamide | 396.1 |
| 300 | 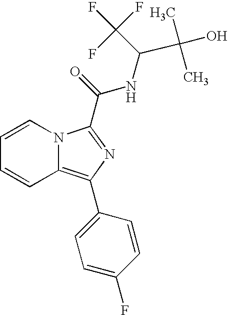 | 1-(4-fluorophenyl)-N-[2-hydroxy- 2-methyl-1- (trifluoromethyl)propyl]imidazo[1, 5-a]pyridine-3-carboxamide | 396.1 |
| 301 | 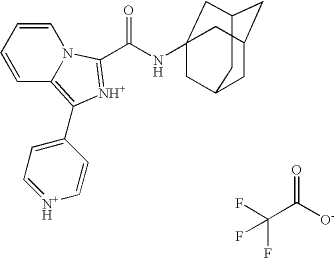 | 3-[(1-adamantylamino)carbonyl]-1- pyridinium-4-ylimidazo[1,5- a]pyridin-2-ium bis(trifluoroacetate) | HRMS(M + 1) = 373.1987 |
| 302 | 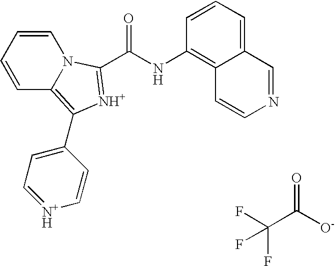 | 3-[(isoquinolin-5- ylamino)carbonyl]-1-pyridinium-4- ylimidazo[1,5-a]pyridin-2-ium bis(trifluoroacetate) | HRMS(M + 1) = 366.1314 |
| 303 | 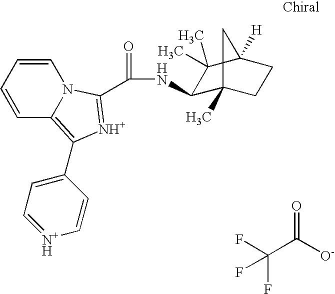 | 1-pyridinium-4-yl-3-({[(1S,2R,4R)- 1,3,3-trimethylbicyclo[2.2.1]hept- 2-yl]amino}carbonyl)imidazo[1,5- a]pyridin-2-ium bis(trifluoroacetate) | HRMS(M + 1) = 375.2147 |
| 304 | 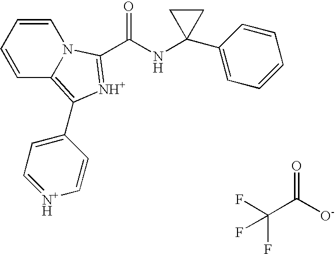 | 3-{[(1- phenylcyclopropyl)amino]carbonyl}- 1-pyridinium-4-ylimidazo[1,5- a]pyridin-2-ium bis(trifluoroacetate) | HRMS(M + 1) = 355.1513 |
| 305 | 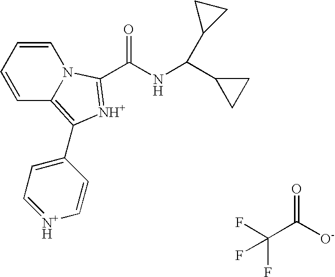 | 3- {[(dicyclopropylmethyl)amino] carbonyl}-1-pyridinium-4- ylimidazo[1,5-a]pyridin-2-ium bis(trifluoroacetate) | HRMS(M + 1) = 333.1666 |
| 306 | 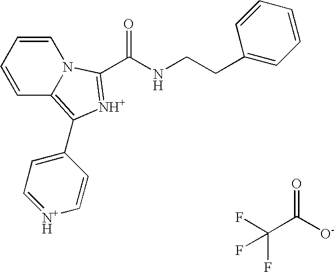 | 3-{[(2- phenylethyl)amino]carbonyl}- 1-pyridinium-4-ylimidazo[1,5- a]pyridin-2-ium bis(trifluoroacetate) | HRMS(M + 1) = 343.1512 |
| 307 | 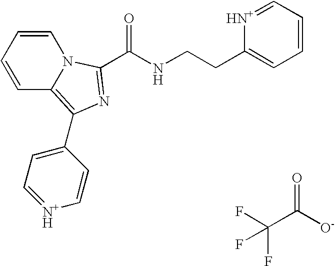 | 2-(2-{[(1-pyridinium-4- ylimidazo[1,5-a]pyridin-3- yl)carbonyl]amino}ethyl)pyridinium bis(trifluoroacetate) | HRMS(M + 1) = 344.1463 |
| 308 | 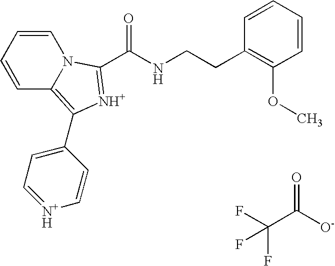 | 3-({[2-(2- methoxyphenyl)ethyl]amino} carbonyl)-1-pyridinium-4-ylimidazo [1,5-a]pyridin-2-ium bis(trifluoroacetate) | HRMS(M + 1) = 373.1623 |
| 309 | 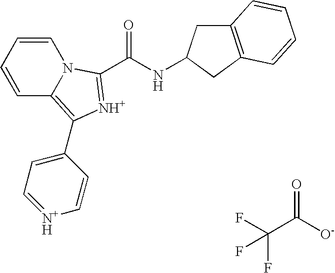 | 3-[(2,3-dihydro-1H-inden-2- ylamino)carbonyl]-1-pyridinium-4- ylimidazo[1,5-a]pyridin-2-ium bis(trifluoroacetate) | HRMS(M + 1) = 355.1513 |
| 310 | 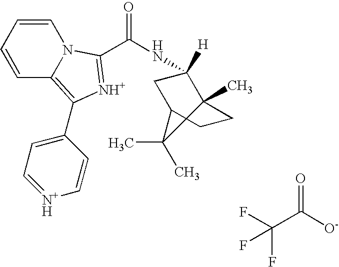 | 1-pyridinium-4-yl-3-({[(1R,2S)- 1,7,7-trimethylbicyclo[2.2.1]hept- 2-yl]amino}carbonyl)imidazo[1,5- a]pyridin-2-ium bis(trifluoroacetate) | HRMS(M + 1) = 375.2147 |
| 311 | 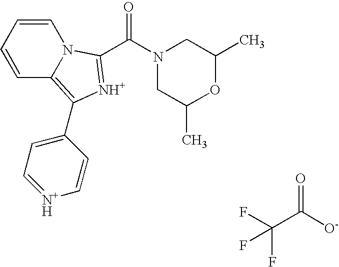 | 3-[(2,6-dimethylmorpholin-4- yl)carbonyl]-1-pyridinium-4- ylimidazo[1,5-a]pyridin-2-ium bis(trifluoroacetate) | HRMS(M + 1) = 337.1615 |
| 312 | 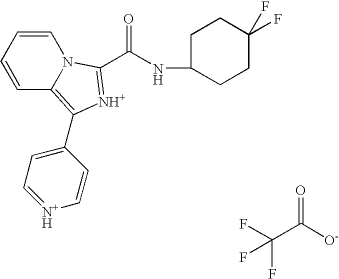 | 3-{[(4,4- difluorocyclohexyl)amino]carbonyl}- 1-pyridinium-4-ylimidazo[1,5- a]pyridin-2-ium bis(trifluoroacetate) | HRMS(M + 1) = 357.1484 |
| 313 | 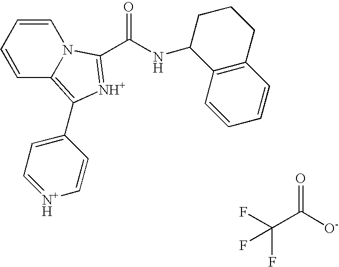 | 1-pyridinium-4-yl-3-[(1,2,3,4- tetrahydronaphthalen-1- ylamino)carbonyl]imidazo[1,5- a]pyridin-2-ium bis(trifluoroacetate) | HRMS(M + 1) = 369.1673 |
| 314 | 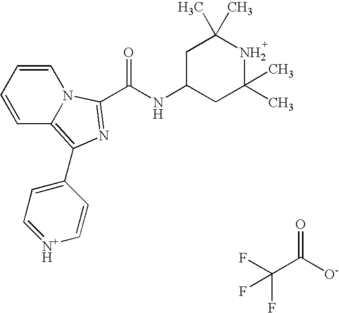 | 4-(3-{[(2,2,6,6- tetramethylpiperidinium-4- yl)amino]carbonyl}imidazo[1,5- a]pyridin-1-yl)pyridinium bis(trifluoroacetate) | HRMS(M + 1) = 378.2251 |
| 315 | 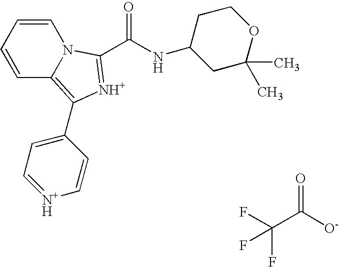 | 3-{[(2,2-dimethyltetrahydro-2H- pyran-4-yl)amino]carbonyl}-1- pyridinium-4-ylimidazo[1,5- a]pyridin-2-ium bis(trifluoroacetate) | HRMS(M + 1) = 351.1775 |
| 316 | 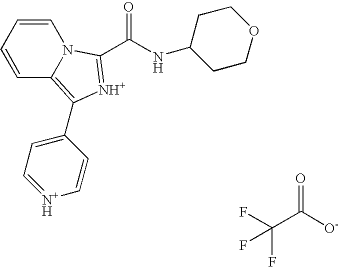 | 1-pyridinium-4-yl-3-[(tetrahydro- 2H-pyran-4- ylamino)carbonyl]imidazo[1,5- a]pyridin-2-ium bis(trifluoroacetate) | HRMS(M + 1) = 323.1458 |
| 317 | 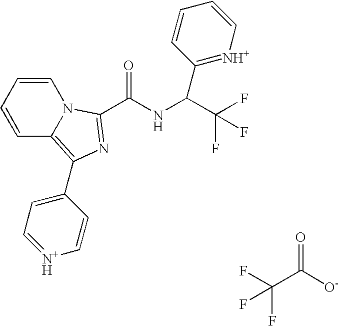 | 2-(2,2,2-trifluoro-1-{[(1- pyridinium-4-ylimidazo[1,5- a]pyridin-3- yl)carbonyl]amino}ethyl)pyridinium bis(trifluoroacetate) | HRMS(M + 1) = 398.1771 |
| 318 | 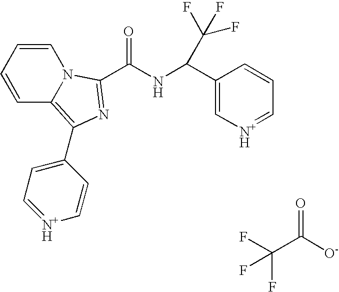 | 3-(2,2,2-trifluoro-1-{[(1- pyridinium-4-ylimidazo[1,5- a]pyridin-3- yl)carbonyl]amino}ethyl)pyridinium bis(trifluoroacetate) | HRMS(M + 1) = 398.1187 |
| 319 | 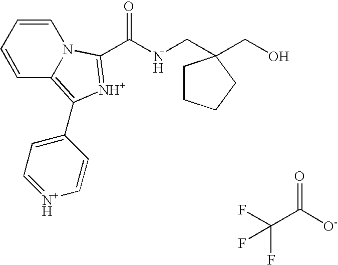 | 3-[({[1- (hydroxymethyl)cyclopentyl]methyl} amino)carbonyl]-1-pyridinium-4- ylimidazo[1,5-a]pyridin-2-ium bis(trifluoroacetate) | MS(M + 1) = 351.2 |
| 320 | 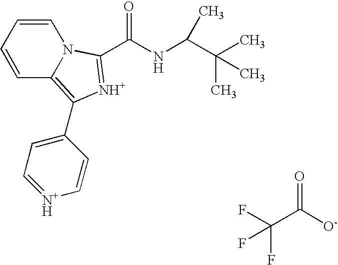 | 1-pyridinium-4-yl-3-{[(1,2,2- trimethylpropyl)amino]carbonyl} imidazo[1,5-a]pyridin-2-ium bis(trifluoroacetate) | HRMS(M + 1) = 323.1820 |
| 321 | 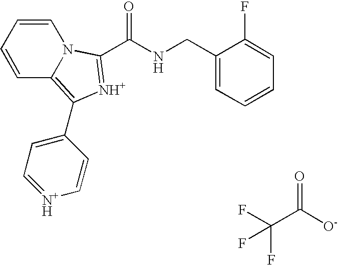 | 3-{[(2- fluorobenzyl)amino]carbonyl}-1- pyridinium-4-ylimidazo[1,5- a]pyridin-2-ium bis(trifluoroacetate) | HRMS(M + 1) = 347.1260 |
| 322 | 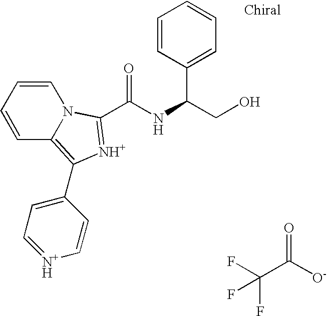 | 3-{[(2- fluorobenzyl)amino]carbonyl}-1- pyridinium-4-ylimidazo[1,5- a]pyridin-2-ium bis(trifluoroacetate) | HRMS(M + 1) = 359.1467 |
| 323 | 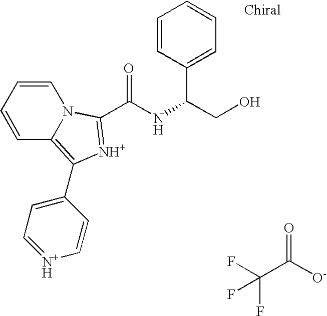 | 3-{[(2- fluorobenzyl)amino]carbonyl}-1- pyridinium-4-ylimidazo[1,5- a]pyridin-2-ium bis(trifluoroacetate) | HRMS(M + 1) = 359.1466 |
| 324 | 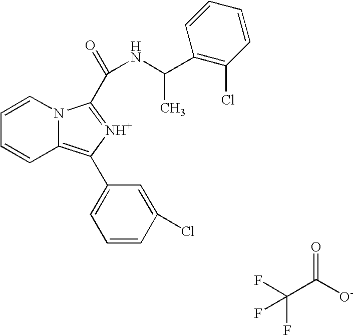 | 1-(3-chlorophenyl)-3-({[1-(2- chlorophenyl)ethyl]amino}carbonyl) imidazo[1,5-a]pyridin-2-ium trifluoroacetate | MS(M + 1) = 411.1 |
| 325 | 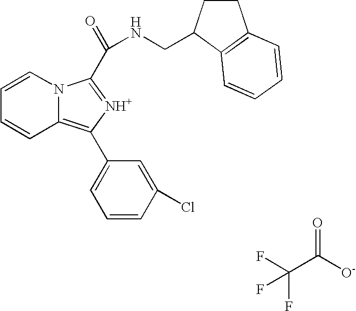 | 1-(3-chlorophenyl)-3-{[(2,3- dihydro-1H-inden-1- ylmethyl)amino]carbonyl}imidazo [1,5-a]pyridin-2-ium trifluoroacetate | MS(M + 1) = 402.1 |
| 326 | 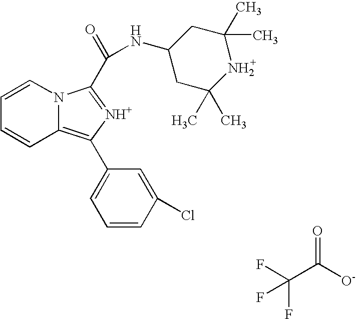 | 1-(3-chlorophenyl)-3-{[(2,2,6,6- tetramethylpiperidinium-4- yl)amino]carbonyl}imidazo[1,5- a]pyridin-2-ium bis(trifluoroacetate) | MS(M + 1) = 411.1 |
| 327 | 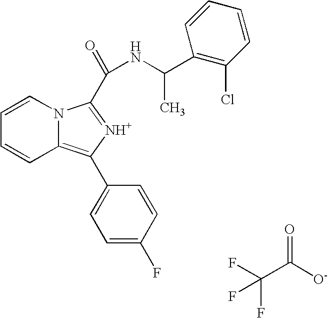 | 3-({[1-(2- chlorophenyl)ethyl]amino}carbonyl)- 1-(4-fluorophenyl)imidazo[1,5- a]pyridin-2-ium trifluoroacetate | MS(M + 1) = 394.0 |
| 328 | 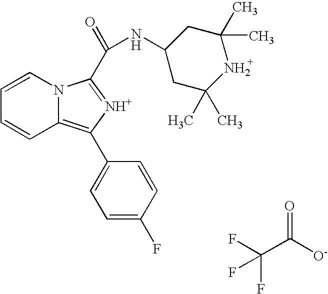 | 1-(4-fluorophenyl)-3-{[(2,2,6,6- tetramethylpiperidinium-4- yl)amino]carbonyl}imidazo[1,5- a]pyridin-2-ium bis(trifluoroacetate) | MS(M + 1) = 395.1 |
| 329 | 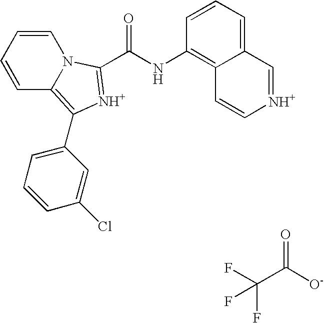 | 5-({[1-(3- chlorophenyl)imidazo[1,5- a]pyridin-2-ium-3- yl]carbonyl}amino)isoquinolinium bis(trifluoroacetate) | HRMS(M + 1) = 399.1007 |
| 330 | 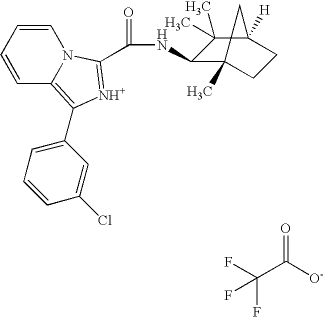 | 1-(3-chlorophenyl)-3- ({[(1S,2R,4R)-1,3,3- trimethylbicyclo[2.2.1]hept-2- yl]amino}carbonyl)imidazo[1,5- a]pyridin-2-ium trifluoroacetate | HRMS(M + 1) = 408.1837 |
| 331 | 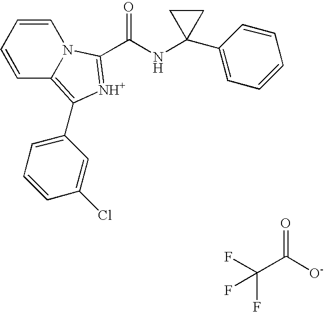 | 1-(3-chlorophenyl)-3-{[(1- phenylcyclopropyl)amino]carbonyl} imidazo[1,5-a]pyridin-2-ium trifluoroacetate | HRMS(M + 1) = 388.1215 |
| 332 | 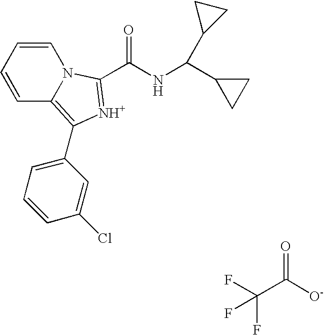 | 1-(3-chlorophenyl)-3- {[(dicyclopropylmethyl)amino] carbonyl}imidazo[1,5-a]pyridin- 2-ium trifluoroacetate | HRMS(M + 1) = 388.1214 |
| 333 | 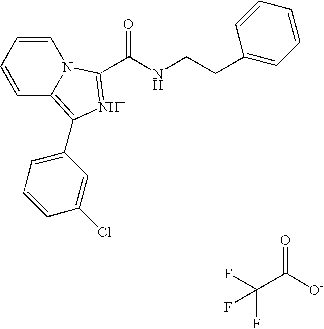 | 1-(3-chlorophenyl)-3-{[(2- phenylethyl)amino]carbonyl} imidazo[1,5-a]pyridin-2-ium trifluoroacetate | HRMS(M + 1) = 376.1213 |
| 334 | 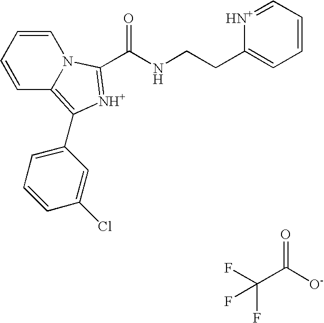 | 1-(3-chlorophenyl)-3-{[(2- pyridinium-2- ylethyl)amino]carbonyl}imidazo[1, 5-a]pyridin-2-ium bis(trifluoroacetate) | HRMS(M + 1) = 377.1165 |
| 335 | 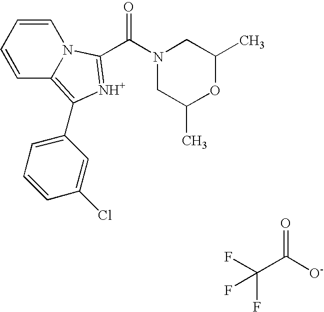 | 1-(3-chlorophenyl)-3-[(2,6- dimethylmorpholin-4- yl)carbonyl]imidazo[1,5-a]pyridin- 2-ium trifluoroacetate | HRMS(M + 1) = 370.1319 |
| 336 | 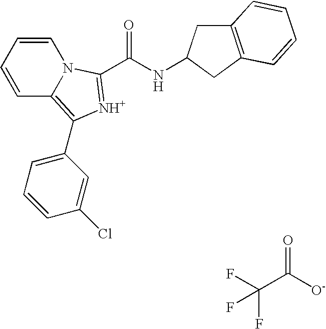 | 1-(3-chlorophenyl)-3-[(2,3-dihydro- 1H-inden-2- ylamino)carbonyl]imidazo[1,5- a]pyridin-2-ium trifluoroacetate | HRMS(M + 1) = 388.1215 |
| 337 | 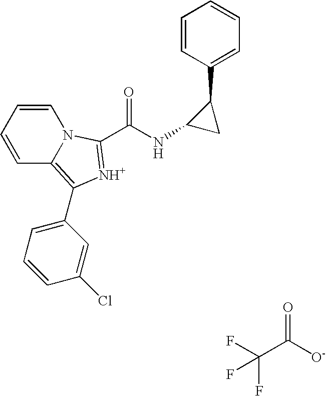 | 1-(3-chlorophenyl)-3-({[(1S,2R)-2- phenylcyclopropyl]amino}carbonyl) imidazo[1,5-a]pyridin-2-ium trifluoroacetate | HRMS(M + 1) = 388.1211 |
| 338 | 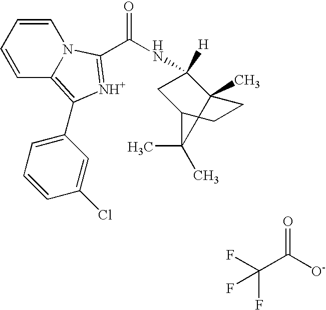 | 1-(3-chlorophenyl)-3-({[(1R,2S)- 1,7,-trimethylbicyclo[2.2.1]hept- 2-yl]amino}carbonyl)imidazo[1,5- a]pyridin-2-ium trifluoroacetate | HRMS(M + 1) = 408.1836 |
| 339 | 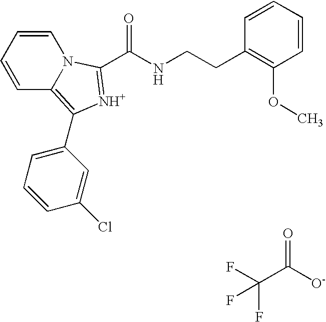 | 1-(3-chlorophenyl)-3-({[2-(2- methoxyphenyl)ethyl]amino} carbonyl)imidazo[1,5-a]pyridin-2-ium trifluoroacetate | HRMS(M + 1) = 406.1317 |
| 340 | 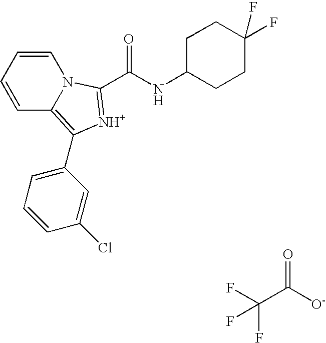 | 1-(3-chlorophenyl)-3-{[(4,4- difluorocyclohexyl)amino] carbonyl}imidazo[1,5-a]pyridin-2-ium trifluoroacetate | HRMS(M + 1) = 390.1180 |
| 341 | 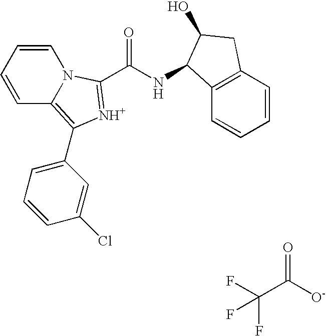 | 1-(3-chlorophenyl)-3-({[(1R,2S)-2- hydroxy-2,3-dihydro-1H-inden-1- yl]amino}carbonyl)imidazo[1,5- a]pyridin-2-ium trifluoroacetate | HRMS(M + 1) = 404.116 |
| 342 | 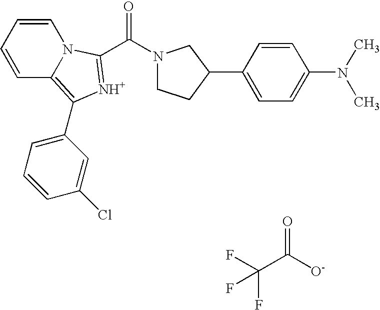 | 1-(3-chlorophenyl)-3-({3-[4- (dimethylamino)phenyl]pyrrolidin- 1-yl}carbonyl)imidazo[1,5- a]pyridin-2-ium trifluoroacetate | HRMS(M + 1) = 445.1788 |
| 343 | 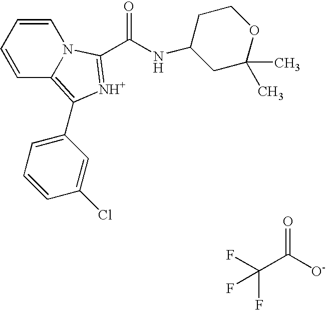 | 1-(3-chlorophenyl)-3-{[(2,2- dimethyltetrahydro-2H-pyran-4- yl)amino]carbonyl}imidazo[1,5- a]pyridin-2-ium trifluoroacetate | HRMS(M + 1) = 384.1474 |
| 344 | 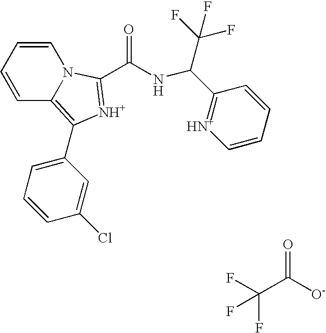 | 1-(3-chlorophenyl)-3-{[(2,2,2- trifluoro-1-pyridinium-2- ylethyl)amino]carbonyl}imidazo[1, 5-a]pyridin-2-ium bis(trifluoroacetate) | HRMS(M + 1) = 431.0876 |
| 345 | 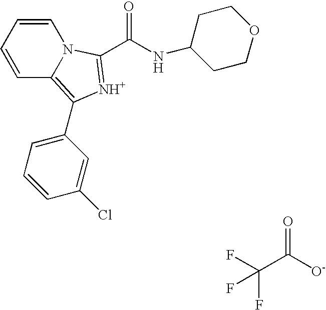 | 1-(3-chlorophenyl)-3-[(tetrahydro- 2H-pyran-4- ylamino)carbonyl]imidazo[1,5- a]pyridin-2-ium trifluoroacetate | HRMS(M + 1) = 356.1165 |
| 346 | 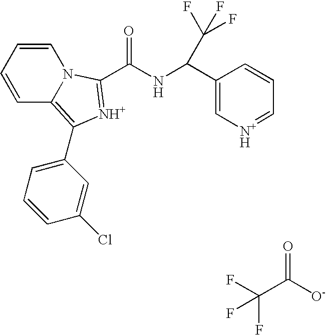 | 1-(3-chlorophenyl)-3-{[(2,2,2- trifluoro-1-pyridinium-3- ylethyl)amino]carbonyl}imidazo[1, 5-a]pyridin-2-ium bis(trifluoroacetate) | HRMS(M + 1) = 431.0878 |
| 347 | 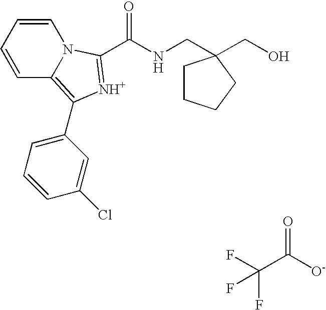 | 1-(3-chlorophenyl)-3-[({[1- (hydroxymethyl)cyclopentyl]methyl} amino)carbonyl]imidazo[1,5- a]pyridin-2-ium trifluoroacetate | HRMS(M + 1) = 384.1474 |
| 348 | 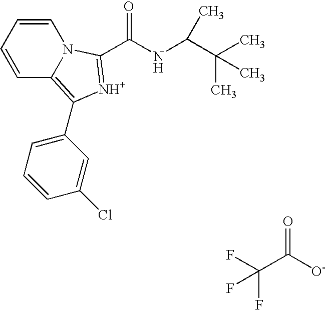 | 1-(3-chlorophenyl)-3-{[(1,2,2- trimethylpropyl)amino]carbonyl} imidazo[1,5-a]pyridin-2-ium trifluoroacetate | HRMS(M + 1) = 356.1165 |
| 349 | 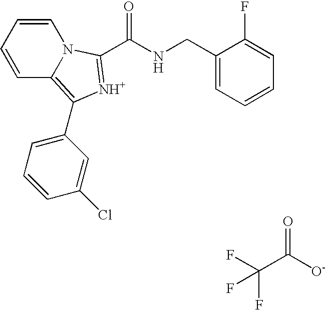 | 1-(3-chlorophenyl)-3-{[(1,2,2- trimethylpropyl)amino]carbonyl} imidazo[1,5-a]pyridin-2-ium trifluoroacetate | HRMS(M + 1) = 380.0962 |
| 350 | 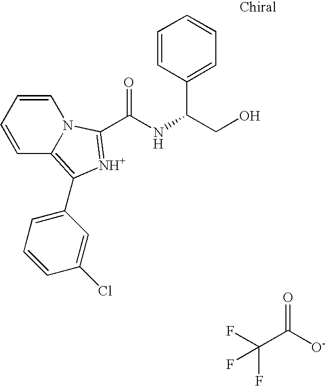 | 1-(3-chlorophenyl)-3-({[(1R)-2- hydroxy-1- phenylethyl]amino}carbonyl) imidazo[1,5-a]pyridin-2-ium trifluoroacetate | HRMS(M + 1) = 392.1162 |
| 351 | 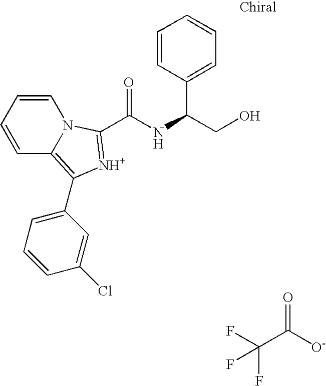 | 1-(3-chlorophenyl)-3-({[(1S)-2- hydroxy-1- phenylethyl]amino}carbonyl) imidazo[1,5-a]pyridin-2-ium trifluoroacetate | HRMS(M + 1) = 392.1161 |
| 352 | 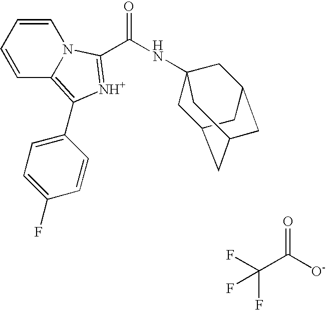 | 3-[(1-adamantylamino)carbonyl]-1- (4-fluorophenyl)imidazo[1,5- a]pyridin-2-ium trifluoroacetate | HRMS(M + 1) = 390.1969 |
| 353 | 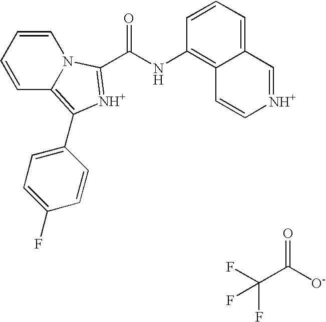 | 5-({[1-(4- fluorophenyl)imidazo[1,5- a]pyridin-2-ium-3- yl]carbonyl}amino)isoquinolinium bis(trifluoroacetate) | HRMS(M + 1) = 383.1290 |
| 354 | 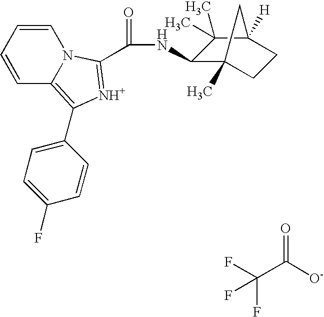 | 1-(4-fluorophenyl)-3- ({[(1S,2R,4R)-1,3,3- trimethylbicyclo[2.2.1 ]hept-2- yl]amino}carbonyl)imidazo[1,5- a]pyridin-2-ium trifluoroacetate | HRMS(M + 1) = 392.2120 |
| 355 | 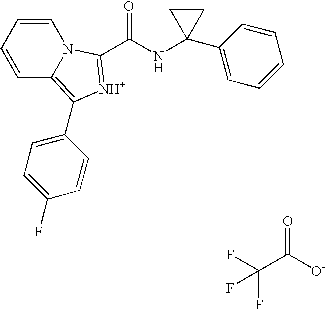 | 1-(4-fluorophenyl)-3-{[(1- phenylcyclopropyl)amino]carbonyl} imidazo[1,5-a]pyridin-2-ium trifluoroacetate | HRMS(M + 1) = 372.1495 |
| 356 | 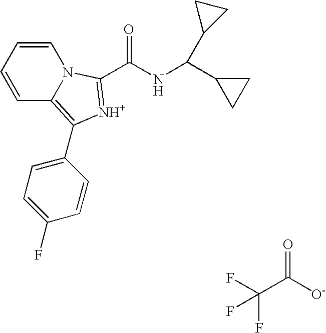 | 3- {[(dicyclopropylmethyl)amino] carbonyl}-1-(4- fluorophenyl)imidazo[1,5- a]pyridin-2-ium trifluoroacetate | HRMS(M + 1) = 350.1655 |
| 357 | 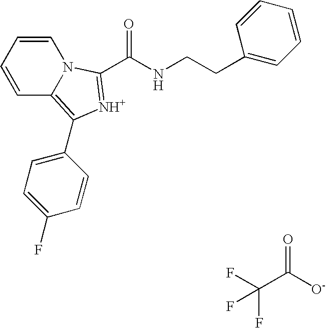 | 1-(4-fluorophenyl)-3-{[(2- phenylethyl)amino]carbonyl} imidazo[1,5-a]pyridin-2-ium trifluoroacetate | HRMS(M + 1) = 360.1497 |
| 358 | 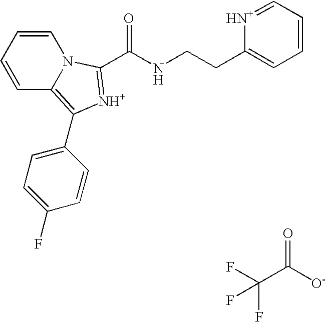 | 1-(4-fluorophenyl)-3-{[(2- pyridinium-2- ylethyl)amino]carbonyl}imidazo[1, 5-a]pyridin-2-ium bis(trifluoroacetate) | HRMS(M + 1) = 361.1450 |
| 359 | 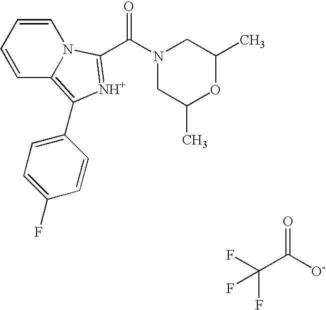 | 3-[(2,6-dimethylmorpholin-4- yl)carbonyl]-1-(4- fluorophenyl)imidazo[1,5- a]pyridin-2-ium trifluoroacetate | HRMS(M + 1) = 354.1603 |
| 360 | 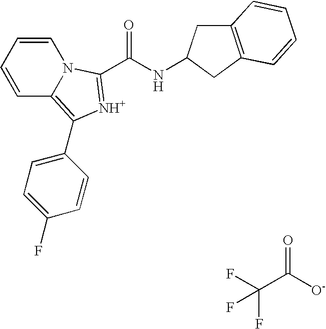 | 3-[(2,3-dihydro-1H-inden-2- ylamino)carbonyl]-1-(4- fluorophenyl)imidazo[1,5- a]pyridin-2-ium trifluoroacetate | HRMS(M + 1) = 372.1499 |
| 361 | 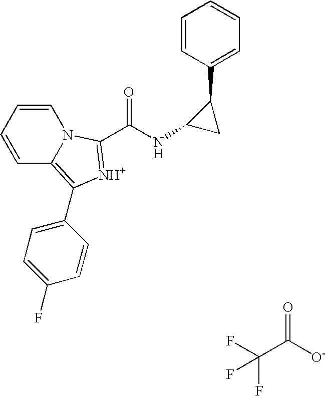 | 1-(4-fluorophenyl)-3-({[(1S,2R)-2- phenylcyclopropyl]amino}carbonyl) imidazo[1,5-a]pyridin-2-ium trifluoroacetate | HRMS(M + 1) = 372.1507 |
| 362 | 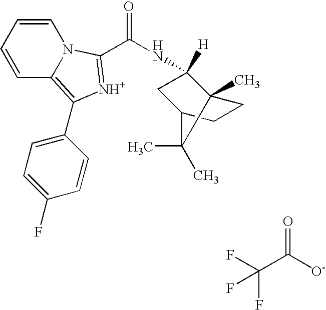 | 1-(4-fluorophenyl)-3-({[(1R,2S)- 1,7,7-trimethylbicyclo[2.2.1]hept- 2-yl]amino}carbonyl)imidazo[1,5- a]pyridin-2-ium trifluoroacetate | HRMS(M + 1) = 392.2128 |
| 363 | 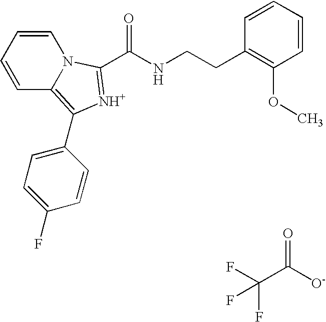 | 1-(4-fluorophenyl)-3-({[2-(2- methoxyphenyl)ethyl]amino} carbnyl)imidazo[1,5-a]pyridin-2- ium trifluoroacetate | HRMS(M + 1) = 390.1602 |
| 364 | 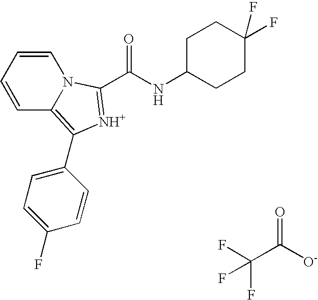 | 3-{[(4,4- difluorocyclohexyl)amino]carbonyl}- 1-(4-fluorophenyl)imidazo[1,5- a]pyridin-2-ium trifluoroacetate | HRMS(M + 1) = 374.1465 |
| 365 | 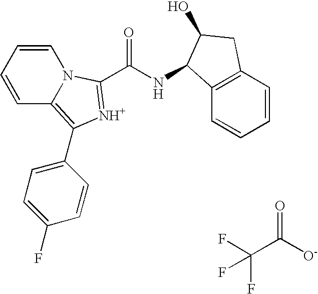 | 1-(4-fluorophenyl)-3-({[(1R,2S)-2- hydroxy-2,3-dihydro-1H-inden-1- yl]amino}carbonyl)imidazo[1,5- a]pyridin-2-ium trifluoroacetate | HRMS(M + 1) = 388.1451 |
| 366 | 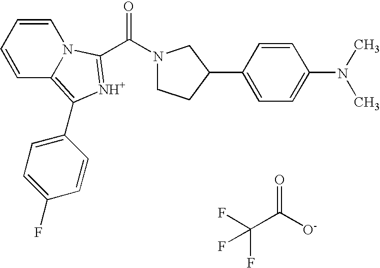 | 3-({3-[4- (dimethylamino)phenyl]pyrrolidin- 1-yl}carbonyl)-1-(4- fluorophenyl)imidazo[1,5- a]pyridin-2-ium trifluoroacetate | HRMS(M + 1) = 429.2080 |
| 367 | 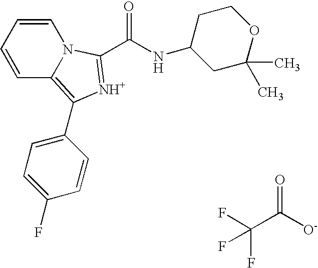 | 3-{[(2,2-dimethyltetrahydro-2H- pyran-4-yl)amino]carbonyl}-1-(4- fluorophenyl)imidazo[1,5- a]pyridin-2-ium trifluoroacetate | HRMS(M + 1) = 368.1764 |
| 368 | 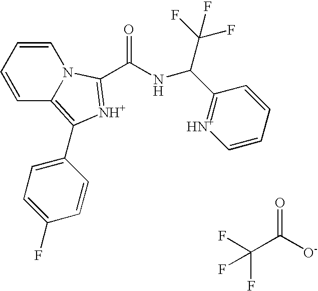 | 1-(4-fluorophenyl)-3-{[(2,2,2- trifluoro-1-pyridinium-2- ylethyl)amino]carbonyl}imidazo[1, 5-a]pyridin-2-ium bis(trifluoroacetate) | HRMS(M + 1) = 415.1165 |
| 369 | 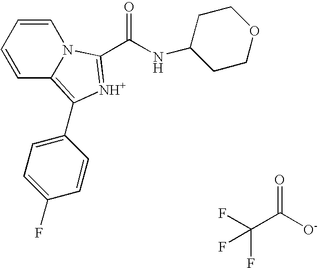 | 1-(4-fluorophenyl)-3-[(tetrahydro- 2H-pyran-4- ylamino)carbonyl]imidazo[1,5- a]pyridin-2-ium trifluoroacetate | HRMS(M + 1) = 340.1449 |
| 370 | 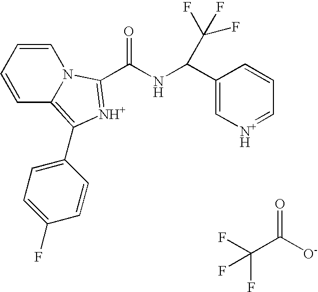 | 1-(4-fluorophenyl)-3-{[(2,2,2- trifluoro-1-pyridinium-3- ylethyl)amino]carbonyl}imidazo[1, 5-a]pyridin-2-ium bis(trifluoroacetate) | HRMS(M + 1) = 415.1172 |
| 371 | 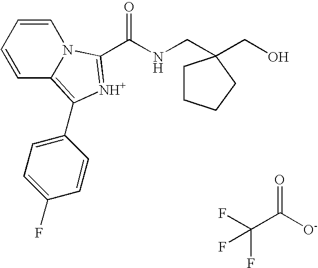 | 1-(4-fluorophenyl)-3-[({[1- (hydroxymethyl)cyclopentyl]methyl} amino)carbonyl]imidazo[1,5- a]pyridin-2-ium trifluoroacetate | HRMS(M + 1) = 368.1761 |
| 372 | 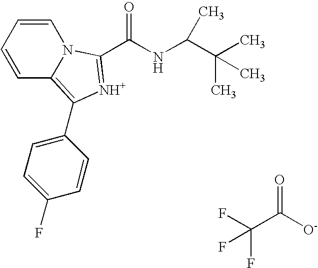 | 1-(4-fluorophenyl)-3-{[(1,2,2- trimethylpropyl)amino]carbonyl} imidazo[1,5-a]pyridin-2-ium trifluoroacetate | HRMS(M + 1) = 340.1813 |
| 373 | 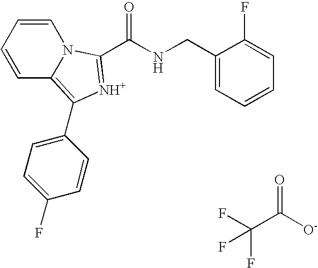 | 3-{[(2- fluorobenzyl)amino]carbonyl}-1- (4-fluorophenyl)imidazo[1,5- a]pyridin-2-ium trifluoroacetate | HRMS(M + 1) = 364.1246 |
| 374 | 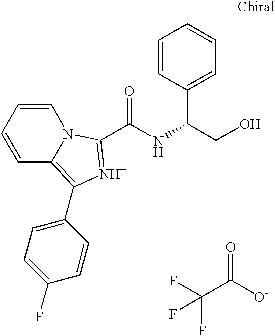 | 1-(4-fluorophenyl)-3-({[(1R)-2- hydroxy-1- phenylethyl]amino}carbonyl) imidazo[1,5-a]pyridin-2-ium trifluoroacetate | HRMS(M + 1) = 376.1445 |
| 375 | 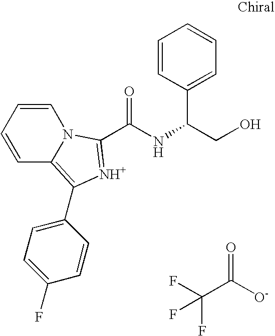 | 1-(4-fluorophenyl)-3-({[(1R)-2- hydroxy-1- phenylethyl]amino}carbonyl) imidazo[1,5-a]pyridin-2-ium trifluoroacetate | HRMS(M + 1) = 376.1446 |
| 376 | 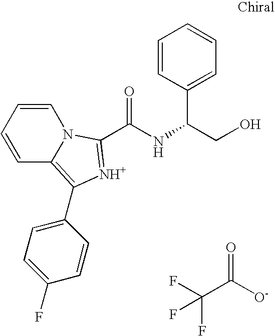 | 1-(4-fluorophenyl)-3-({[(1R)-2- hydroxy-1- phenylethyl]amino}carbonyl) imidazo[1,5-a]pyridin-2-ium trifluoroacetate | HRMS(M + 1) = 376.2 |
| 377 | 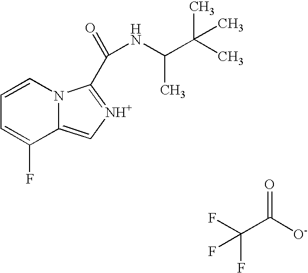 | 8-fluoro-3-{[(1,2,2- trimethylpropyl)amino]carbonyl} imidazo[1,5-a]pyridin-2-ium trifluoroacetate | MS(M + 1) = 264.2 |
| 378 | 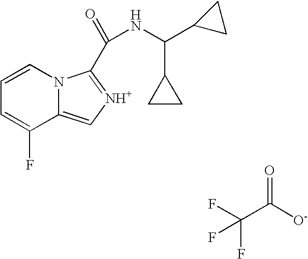 | 3- {[(dicyclopropylmethyl)amino] carbonyl}-8-fluoroimidazo[1,5- a]pyridin-2-ium trifluoroacetate | MS(M + 1) = 274.1 |
| 379 | 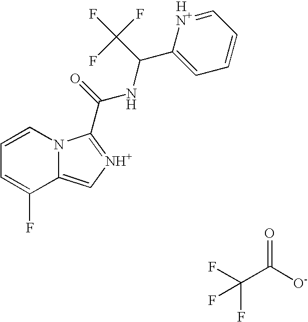 | 8-fluoro-3-{[(2,2,2-trifluoro-1- pyridinium-2- ylethyl)amino]carbonyl}imidazo[1, 5-a]pyridin-2-ium bis(trifluoroacetate) | MS(M + 1) = 339.1. |
| 380 | 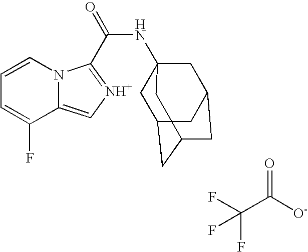 | 3-[(1-adamantylamino)carbonyl]-8- fluoroimidazo[1,5-a]pyridin-2-ium trifluoroacetate | MS(M + 1) = 314.2 |
| 381 | 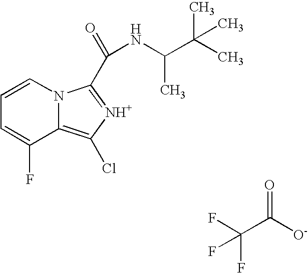 | 1-chloro-8-fluoro-3-{[(1,2,2- trimethylpropyl)amino]carbonyl} imidazo[1,5-a]pyridin-2-ium trifluoroacetate | MS(M + 1) = 298.1 |
| 382 | 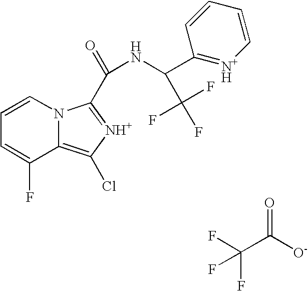 | 1-chloro-8-fluoro-3-{[(2,2,2- trifluoro-1-pyridinium-2- ylethyl)amino]carbonyl}imidazo[1, 5-a]pyridin-2-ium bis(trifluoroacetate) | MS(M + 1) = 373.0 |
| 383 | 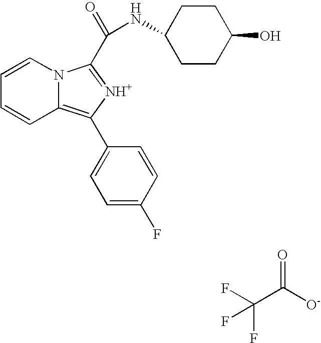 | 1-(4-fluorophenyl)-3-{[(trans-4- hydroxycyclohexyl)amino]carbonyl} imidazo[1,5-a]pyridin-2-ium trifluoroacetate | MS(M + 1) = 354.1 |
| 384 | 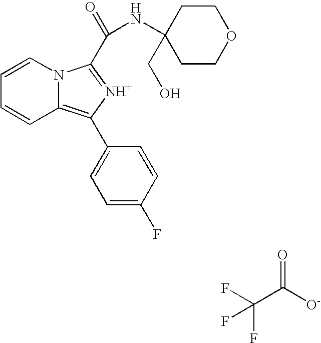 | 1-(4-fluorophenyl)-3-({[4- (hydroxymethyl)tetrahydro-2H- pyran-4- yl]amino}carbonyl)imidazo[1,5- a]pyridin-2-ium trifluoroacetate | MS(M + 1) = 370.2 |
| 385 | 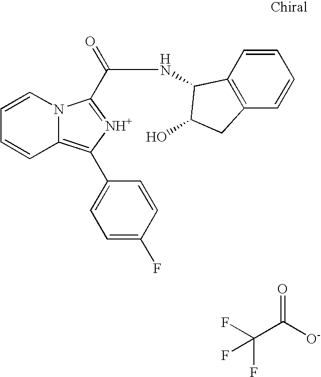 | 1-(4-fluorophenyl)-3-({[(1R,2S)-2- hydroxy-2,3-dihydro-1H-inden-1- yl]amino}carbonyl)imidazo[1,5- a]pyndin-2-ium trifluoroacetate | MS(M + 1) = 388.1 |
| 386 | 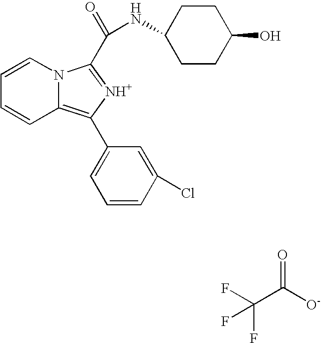 | 1-(3-chlorophenyl)-3-{[(trans-4- hydroxycyclohexyl)amino]carbonyl} imidazo[1,5-a]pyridin-2-ium trifluoroacetate | MS(M + 1) = 370.1 |
| 387 | 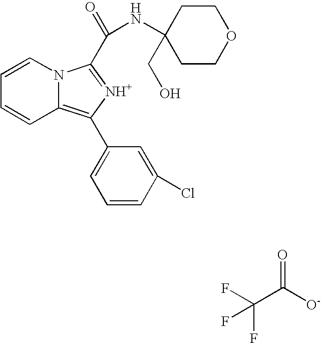 | 1-(3-chlorophenyl)-3-({[4- (hydroxymethyl)tetrahydro-2H- pyran-4- yl]amino}carbonyl)imidazo[1,5- a]pyridin-2-ium trifluoroacetate | MS(M + 1) = 386.1 |
| 388 | 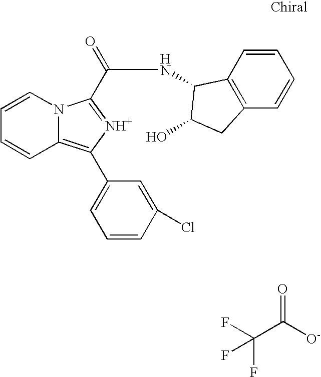 | 1-(3-chlorophenyl)-3-({[(1R,2S)-2- hydroxy-2,3-dihydro-1H-inden-1- yl]amino}carbonyl)imidazo[1,5- a]pyridin-2-ium trifluoroacetate | MS(M + 1) = 386.1 |
| 389 | 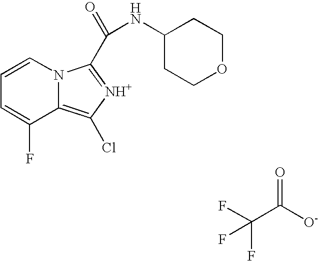 | 1-chloro-8-fluoro-3-[(tetrahydro- 2H-pyran-4- ylamino)carbonyl]imidazo[1,5- a]pyridin-2-ium trifluoroacetate | MS(M + 1) = 298.1 |
| 390 | 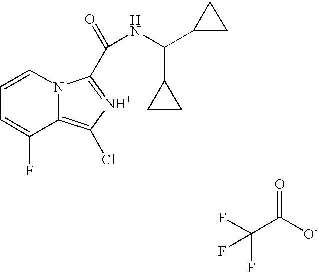 | 1-chloro-3- {[(dicyclopropylmethyl)amino] carbonyl}-8-fluoroimidazo[1,5- a]pyridin-2-ium trifluoroacetate | MS(M + 1) = 308.1 |
| 391 | 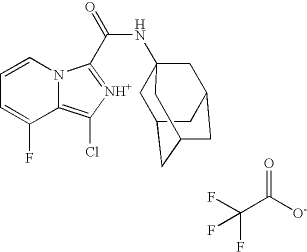 | 3-[(1-adamantylamino)carbonyl]-1- chloro-8-fluoroimidazo[1,5- a]pyridin-2-ium trifluoroacetate | MS(M + 1) = 348.1 |
| 392 | 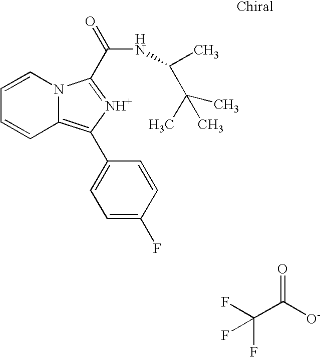 | 1-(4-fluorophenyl)-3-({[(1R)-1,2,2- trimethylpropyl]amino}carbonyl) imidazo[1,5-a]pyridin-2-ium trifluoroacetate | MS(M + 1) = 340.1 |
| 393 | 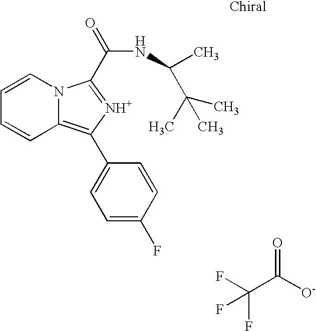 | 1-(4-fluorophenyl)-3-({[(1S)-1,2,2- trimethylpropyl]amino}carbonyl) imidazo[1,5-a]pyridin-2-ium trifluoroacetate | MS(M + 1) = 340.1 |
| 394 | 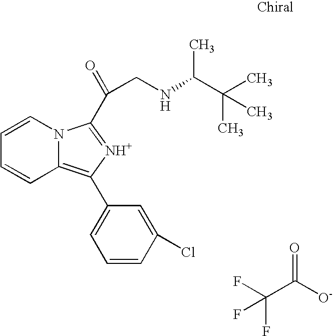 | 1-(3-chlorophenyl)-3-({[(1R)-1,2,2- trimethylpropyl]amino}carbonyl) imidazo[1,5-a]pyridin-2-ium trifluoroacetate | MS(M + 1) = 356.1 |
| 395 | 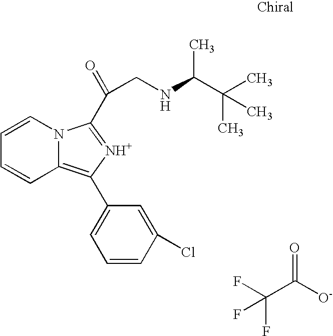 | 1-(3-chlorophenyl)-3-({[(1S)-1,2,2- trimethylpropyl]amino}carbonyl) imidazo[1,5-a]pyridin-2-ium trifluoroacetate | MS(M + 1) = 356.1 |
| 396 | 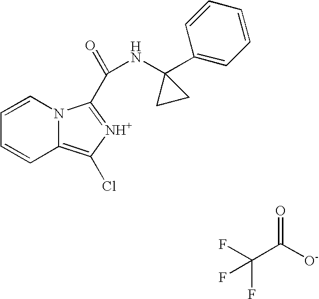 | 1-chloro-3-{[(1- phenylcyclopropyl)amino]carbonyl} imidazo[1,5-a]pyridin-2-ium trifluoroacetate | MS(M + 1) = 312.1 |
| 397 | 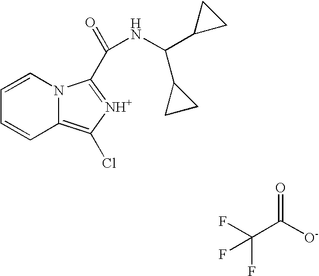 | 1-chloro-3- {[(dicyclopropylmethyl)amino] carbonyl}imidazo[1,5-a]pyridin- 2-ium trifluoroacetate | MS(M + 1) = 290.1 |
| 398 | 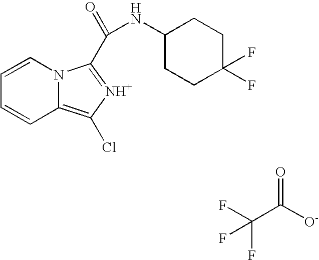 | 1-chloro-3-{[(4,4- difluorocyclohexyl)amino] carbonyl}imidazo[1,5-a]pyridin- 2-ium trifluoroacetate | MS(M + 1) = 314.1 |
| 399 | 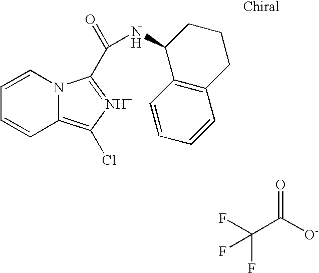 | 1-chloro-3-{[(1S)-1,2,3,4- tetrahydronaphthalen-1- ylamino]carbonyl}imidazo[1,5- a]pyridin-2-ium trifluoroacetate | MS(M + 1) = 326.1 |
| 400 | 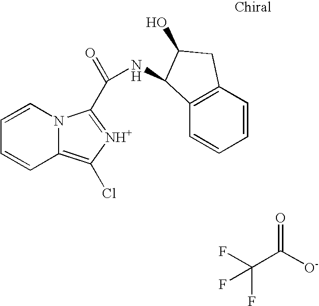 | 1-chloro-3-({[(1R,2S)-2-hydroxy- 2,3-dihydro-1H-inden-1- yl]amino}carbonyl)imidazo[1,5- a]pyridin-2-ium trifluoroacetate | MS(M + 1) = 328.1 |
| 401 | 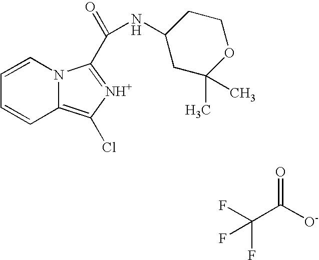 | 1-chloro-3-{[(2,2- dimethyltetrahydro-2H-pyran-4- yl)amino]carbonyl}imidazo[1,5- a]pyridin-2-ium trifluoroacetate | MS(M + 1) = 308.1 |
| 402 | 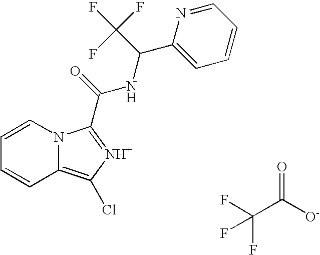 | 1-chloro-3-{[(2,2,2-trifluoro-1- pyridin-2- ylethyl)amino]carbonyl}imidazo[1, 5-a]pyridin-2-ium trifluoroacetate | MS(M + 1) = 355.1 |
| 403 | 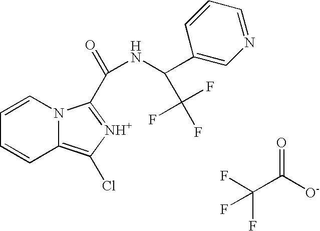 | 1-chloro-3-{[(2,2,2-trifluoro-1- pyridin-3- ylethyl)amino]carbonyl}imidazo[1, 5-a]pyridin-2-ium trifluoroacetate | MS(M + 1) = 355.0 |
| 404 | 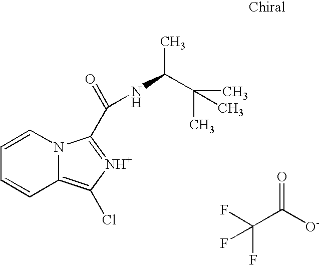 | 1-chloro-3-({[(1S)-1,2,2- trimethylpropyl]amino}carbonyl) imidazo[1,5-a]pyridin-2-ium trifluoroacetate | MS(M + 1) = 280.1 |
| 405 | 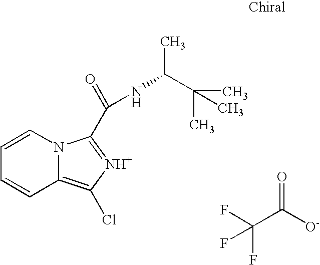 | 1-chloro-3-({[(1R)-1,2,2- trimethylpropyl]amino}carbonyl) imidazo[1,5-a]pyridin-2-ium trifluoroacetate | MS(M + 1) = 280.1 |
| 406 | 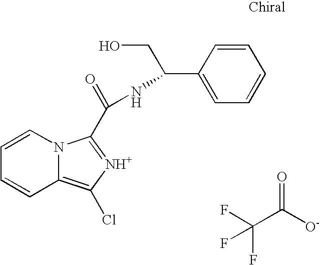 | 1-chloro-3-({[(1S)-2-hydroxy-1- phenylethyl]amino}carbonyl) imidazo[1,5-a]pyridin-2-ium trifluoroacetate | MS(M + 1) = 316.0 |
| 407 | 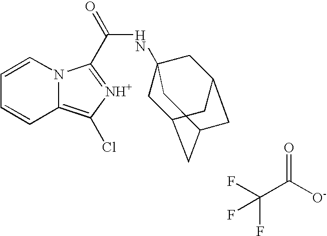 | 3-[(1-adamantylamino)carbonyl]-1- chloroimidazo[1,5-a]pyridin-2-ium trifluoroacetate | MS(M + 1) = 330.1 |
| 408 | 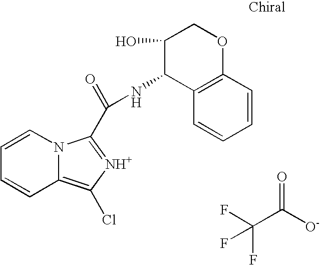 | 1-chloro-3-({[(3S,4S)-3-hydroxy- 3,4-dihydro-2H-chromen-4- yl]amino}carbonyl)imidazo[1,5- a]pyridin-2-ium trifluoroacetate | MS(M + 1) = 344.0 |
| 409 | 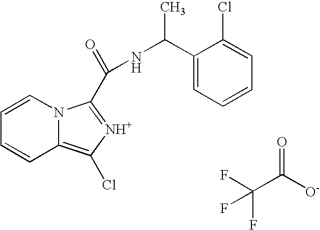 | 1-chloro-3-({[1-(2- chlorophenyl)ethyl]amino}carbonyl) imidazo[1,5-a]pyridin-2-ium trifluoroacetate | MS(M + 1) = 335.1 |
| 410 | 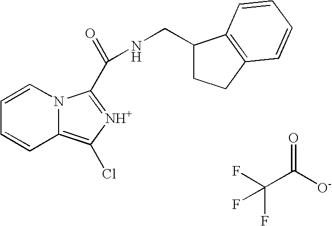 | 1-chloro-3-{[(2,3-dihydro-1H- inden-1- ylmethyl)amino]carbonyl}imidazo [1,5-a]pyridin-2-ium trifluoroacetate | MS(M + 1) = 326.1 |
| 411 | 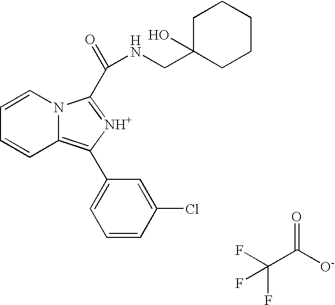 | 1-(3-chlorophenyl)-3-({[(1- hydroxycyclohexyl)methyl]amino} carbonyl)imidazo[1,5-a]pyridin-2- ium trifluoroacetate | MS(M + 1) = 384.1 |
| 412 | 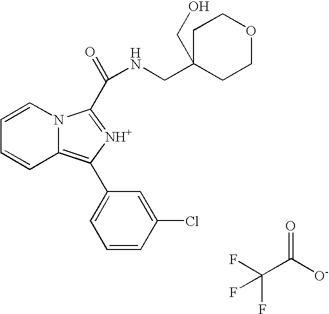 | 1-(3-chlorophenyl)-3-[({[4- (hydroxymethyl)tetrahydro-2H- pyran-4- yl]methyl}amino)carbonyl]imidazo [1,5-a]pyridin-2-ium trifluoroacetate | MS(M + 1)- 400.2 |
| 413 | 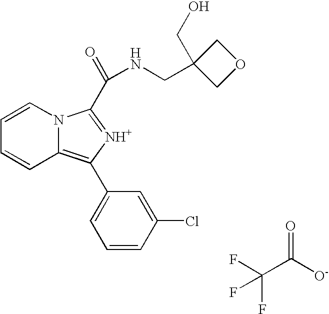 | 1-(3-chlorophenyl)-3-[({[3- (hydroxymethyl)oxetan-3- yl]methyl}amino)carbonyl]imidazo [1,5-a]pyridin-2-ium trifluoroacetate | MS(M + 1) = 372.1 |
| 414 | 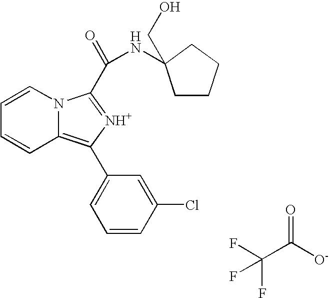 | 1-(3-chlorophenyl)-3-({[1- (hydroxymethyl)cyclopentyl]amino} carbonyl)imidazo[1,5-a]pyridin-2- ium trifluoroacetate | MS(M + 1) = 370.2 |
| 415 | 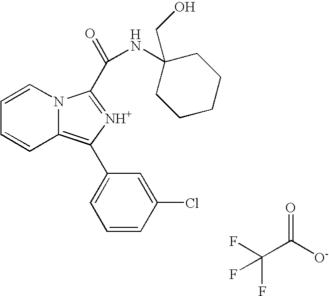 | 1-(3-chlorophenyl)-3-({[1- (hydroxymethyl)cyclohexyl]amino} carbonyl)imidazo[1,5-a]pyridin-2- ium trifluoroacetate | MS(M + 1) = 384.1 |
| 416 | 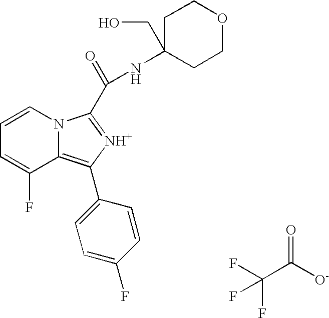 | 8-fluoro-1-(4-fluorophenyl)-3-({[4- (hydroxymethyl)tetrahydro-2H- pyran-4- yl]amino}carbonyl)imidazo[1,5- a]pyridin-2-ium trifluoroacetate | MS(M + 1) = 388.1 |
| 417 | 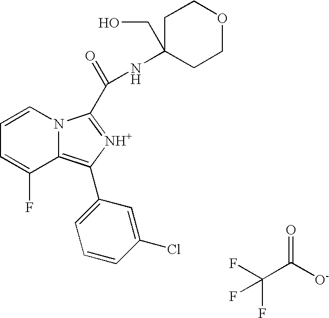 | 1-(3-chlorophenyl)-8-fluoro-3-({[4- (hydroxymethyl)tetrahydro-2H- pyran-4- yl]amino}carbonyl)imidazo[1,5- a]pyridin-2-ium trifluoroacetate | MS(M + 1) = 404.1 |
| 418 | 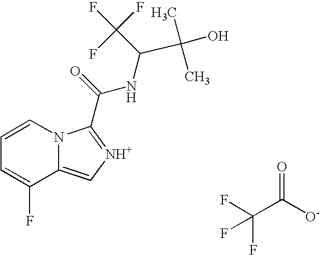 | 8-fluoro-3-({[2-hydroxy-2-methyl- 1- (trifluoromethyl)propyl]amino} carbonyl)imidazo[1,5-a]pyridin- 2-ium trifluoroacetate | 320.1 |
| 419 | 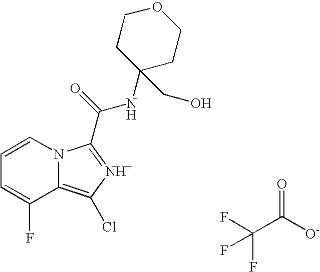 | 1-chloro-8-fluoro-3-({[4- (hydroxymethyl)tetrahydro-2H- pyran-4- yl]amino}carbonyl)imidazo[1,5- a]pyridin-2-ium trifluoroacetate | 328.1 |
| 420 | 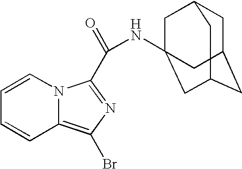 | N-1-adamantyl-1- bromoimidazo[1,5-a]pyridine-3- carboxamide | 376.0, 374.0 |
| 421 | 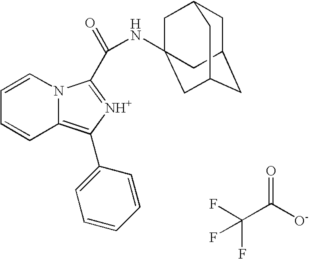 | N-1-adamantyl-1- phenylimidazo[1,5-a]pyridine-3- carboxamide trifluoroacetate | 372.2 |
| 422 | 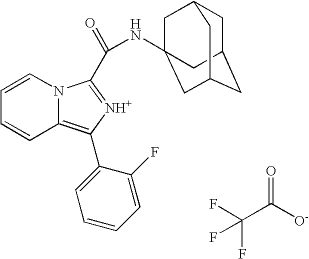 | N-1-adamantyl-1-(2- fluorophenyl)imidazo[1,5- a]pyridine-3-carboxamide trifluoroacetate | 390.1 |
| 423 | 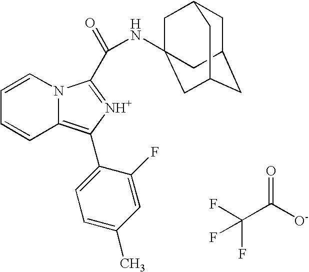 | N-1-adamantyl-1-(2-fluoro-4- methylphenyl)imidazo[1,5- a]pyridine-3-carboxamide trifluoroacetate. | 404.1 |
| 424 | 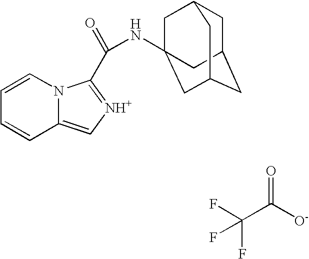 | N-1-adamantylimidazo[1,5- a]pyridine-3-carboxamide trifluoroacetate | 296.1 |
| 425 | 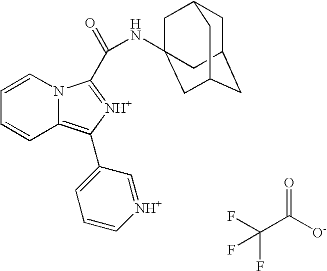 | N-1-adamantyl-1-pyridin-3- ylimidazo[1,5-a]pyridine-3- carboxamide bis-trifluoroacetate | 373.2 |
| 426 | 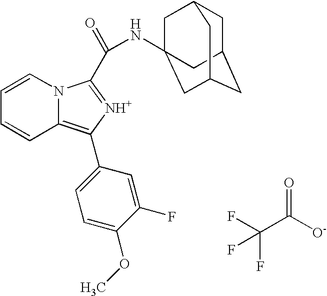 | N-1-adamantyl-1-(3-fluoro-4- methoxyphenyl)imidazo[1,5- a]pyridine-3-carboxamide trifluoroacetate | 420.1 |
| 427 | 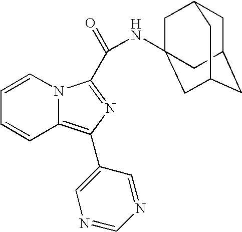 | N-1-adamantyl-1-pyrimidin-5- ylimidazo[1,5-a]pyridine-3- carboxamide | 374.1 |
| 428 | 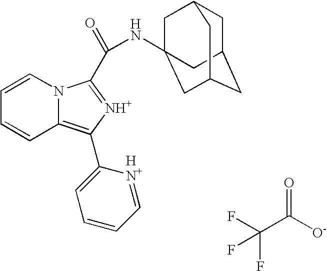 | N-1-adamantyl-1-pyridin-2- ylimidazo[1,5-a]pyridine-3- carboxamide bis-trifluoroacetate | 373.2 |
| 644 | 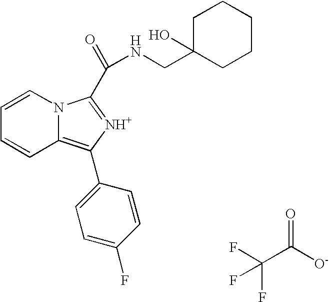 | N′-cyclohexyl-N-(2,3-dihydro-1,4-benzodioxin-6- ylmethyl)-N-(2,2-diphenylethyl)urea | 471.3 |
| 645 | 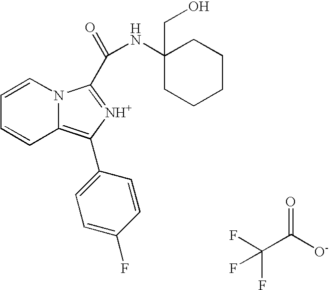 | 1-(4-fluorophenyl)-3-({[1- (hydroxymethyl)cyclohexyl]amino}carbonyl) imidazo[1,5-a]pyridin-2-ium trifluoroacetate | 368.2 |
| 646 | 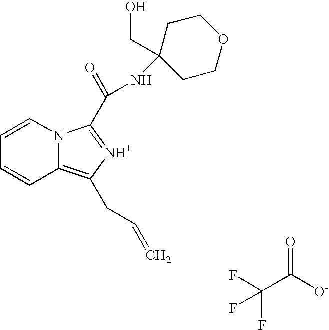 | 1-allyl-n-[4-(hydroxymethyl)tetrahydro-2h-pyran- 4-yl]imidazo[1,5-a]pyridine-3-carboxamide | 316.1656 |
| 647 | 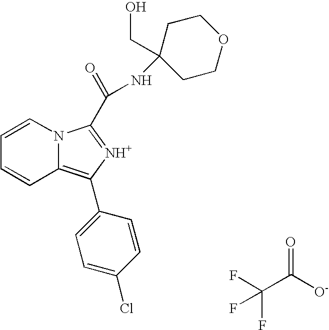 | 1-(4-chlorophenyl)-n-[4- (hydroxymethyl)tetrahydro-2h-pyran-4- yl]imidazo[1,5-A]pyridine-3-carboxamide | 386.1265 |
| 648 | 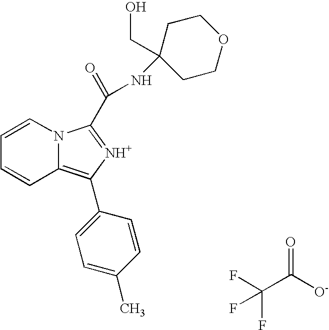 | N-[4-(hydroxymethyl)tetrahydro-2h-pyran-4-yl]-1- (4-methylphenyl)imidazo[1,5-a]pyridine-3- carboxamide | 366.1812 |
| 649 | 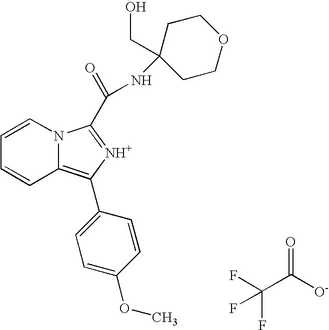 | N-[4-(hydroxymethyl)tetrahydro-2h-pyran-4-yl]-1- (4-methoxyphenyl)imidazo[1,5-a]pyridine-3- carboxamide | 382.1781 |
| 650 | 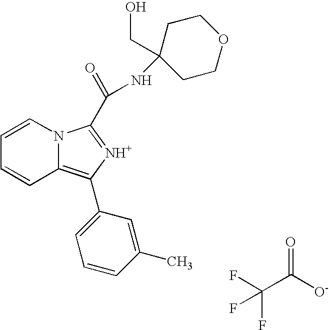 | N-[4-(hydroxymethyl)tetrahydro-2h-pyran-4-yl]-1- (3-methylphenyl)imidazo[1,5-a]pyridine-3- carboxamide | 366.1836 |
| 651 | 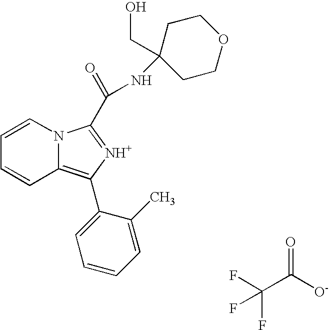 | N-[4-(hydroxymethyl)tetrahydro-2h-pyran-4-yl]-1- (2-methylphenyl)imidazo[1,5-a]pyridine-3- carboxamide | 366.1833 |
| 652 | 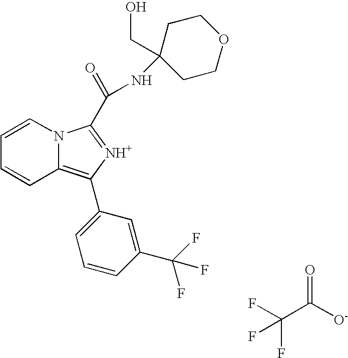 | N-[4-(hydroxymethyl)tetrahydro-2h-pyran-4-yl]-1- [3-(trifluoromethyl)phenyl]imidazo[1,5-a]pyridine- 3-carboxamide | 420.2 |
| 653 | 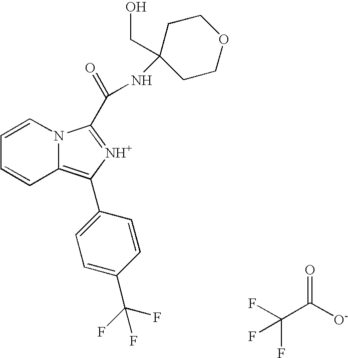 | N-[4-(hydroxymethyl)tetrahydro-2h-pyran-4-yl]-1- [4-(trifluoromethyl)phenyl]imidazo[1,5-a]pyridine- 3-carboxamide | 420.1535 |
| 654 | 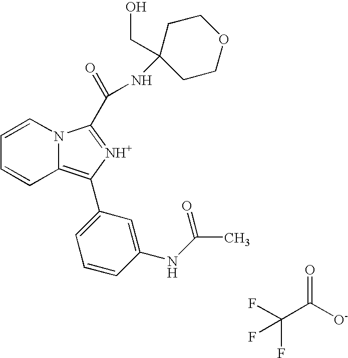 | 1-[3-(acetylamino)phenyl]-n-[4- (hydroxymethyl)tetrahydro-2h-pyran-4- yl]imidazo[1,5-a]pyridine-3-carboxamide | 409.1865 |
| 655 | 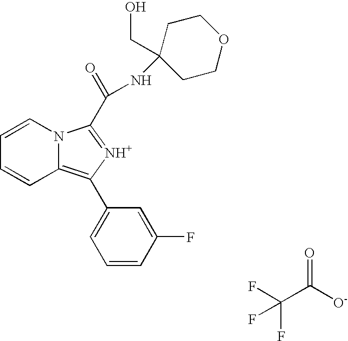 | 1-(3-fluorophenyl)-n-[4- (hydroxymethyl)tetrahydro-2h-pyran-4- yl]imidazo[1,5-a]pyridine-3-carboxamide | 370.1562 |
| 656 | 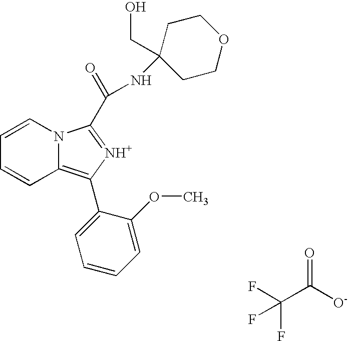 | N-[4-(hydroxymethyl)tetrahydro-2h-pyran-4-yl]-1- (2-methoxyphenyl)imidazo[1,5-a]pyridine-3- carboxamide | 382.1768 |
| 657 | 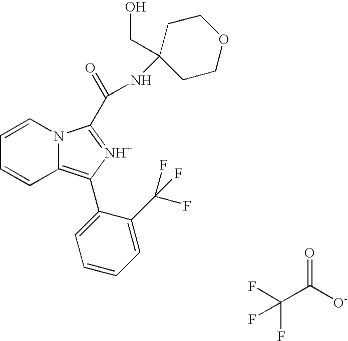 | N-[4-(hydroxymethyl)tetrahydro-2h-pyran-4-yl]-1- [2-(trifluoromethyl)phenyl]imidazo[1,5-a]pyridine- 3-carboxamide | 420.1527 |
| 658 | 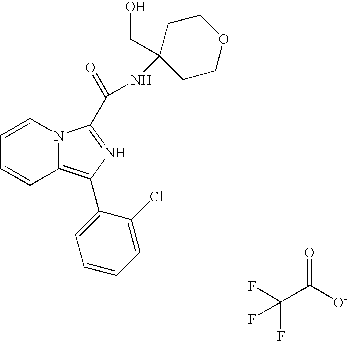 | 1-(2-chlorophenyl)-n-[4- (hydroxymethyl)tetrahydro-2h-pyran-4- yl]imidazo[1,5-a]pyridine-3-carboxamide | 386.1264 |
| 659 | 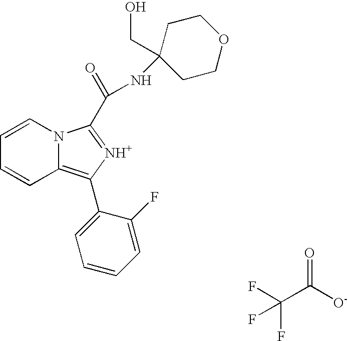 | 1-(2-fluorophenyl)-n-[4- (hydroxymethyl)tetrahydro-2h-pyran-4- yl]imidazo[1,5-a]pyridine-3-carboxamide | 386.1264 |
| 660 | 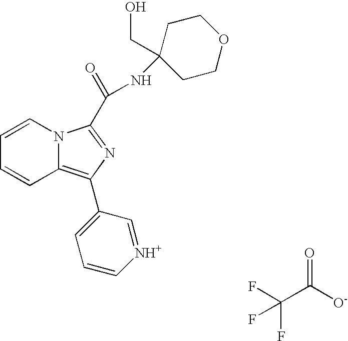 | N-[4-(hydroxymethyl)tetrahydro-2h-pyran-4-yl]-1- pyridin-3-ylimidazo[1,5-a]pyridine-3-carboxamide | 353.1608 |
| 661 | 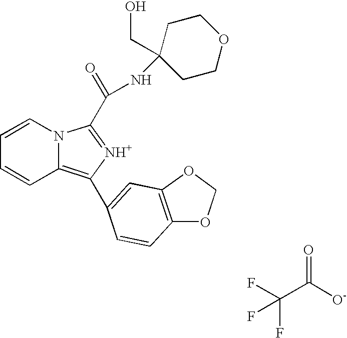 | 1-(1,3-benzodioxol-5-yl)-n-[4- (hydroxymethyl)tetrahydro-2h-pyran-4- yl]imidazo[1,5-a]pyridine-3-carboxamide | 396.155 |
| 662 | 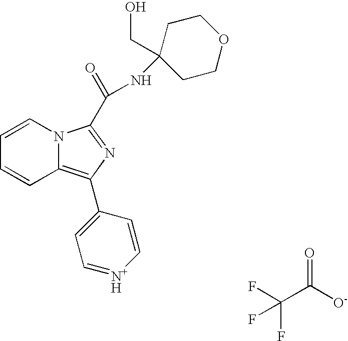 | N-[4-(hydroxymethyl)tetrahydro-2h-pyran-4-yl]-1- pyridin-4-ylimidazo[1,5-a]pyridine-3-carboxamide | 353.1606 |
| 663 | 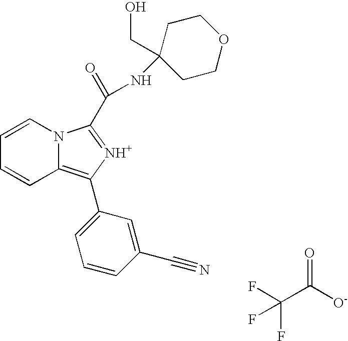 | 1-(3-cyanophenyl)-n-[4- (hydroxymethyl)tetrahydro-2h-pyran-4- yl]imidazo[1,5-a]pyridine-3-carboxamide | 436.1485 |
| 664 | 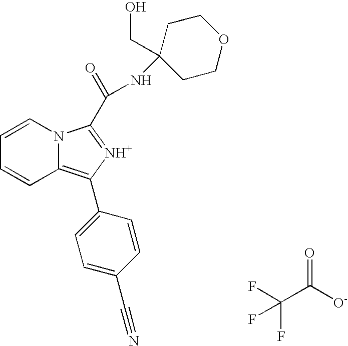 | 1-(4-cyanophenyl)-n-[4- (hydroxymethyl)tetrahydro-2h-pyran-4- yl]imidazo[1,5-a]pyridine-3-carboxamide | 377.1605 |
| 665 | 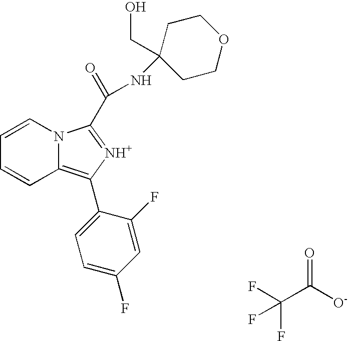 | 1-(2,4-difluorophenyl)-n-[4- (hydroxymethyl)tetrahydro-2h-pyran-4- yl]imidazo[1,5-a]pyridine-3-carboxamide | 388.2 |
| 666 | 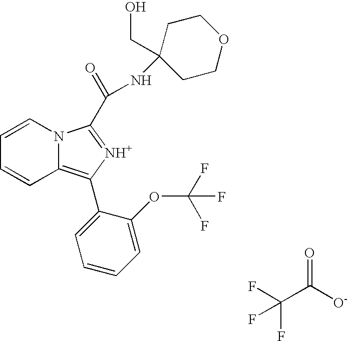 | N-[4-(hydroxymethyl)tetrahydro-2h-pyran-4-yl]-1- [2-(trifluoromethoxy)phenyl]imidazo[1,5- a]pyridine-3-carboxamide | 436.1373 |
| 667 | 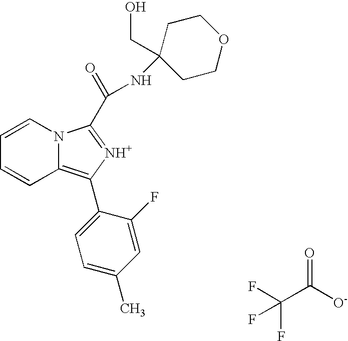 | 1-(2-fluoro-4-methylphenyl)-n-[4- (hydroxymethyl)tetrahydro-2h-pyran-4- yl]imidazo[1,5-a]pyridine-3-carboxamide | 384.1723 |
| 668 | 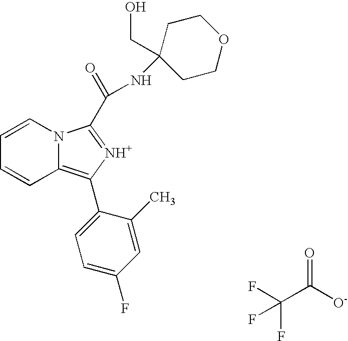 | 1-(4-fluoro-2-methylphenyl)-n-[4- (hydroxymethyl)tetrahydro-2h-pyran-4- yl]imidazo[1,5-a]pyridine-3-carboxamide | 384.1721 |
| 669 | 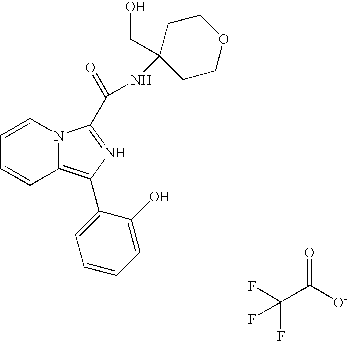 | N-[4-(hydroxymethyl)tetrahydro-2h-pyran-4-yl]-1- (2-hydroxyphenyl)imidazo[1,5-a]pyridine-3- carboxamide | 368.1623 |
| 670 | 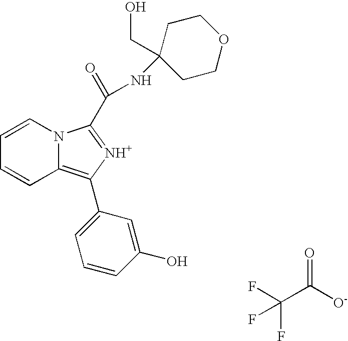 | N-[4-(hydroxymethyl)tetrahydro-2h-pyran-4-yl]-1- (3-hydroxyphenyl)imidazo[1,5-a]pyridine-3- carboxamide | 368.1604 |
| 671 | 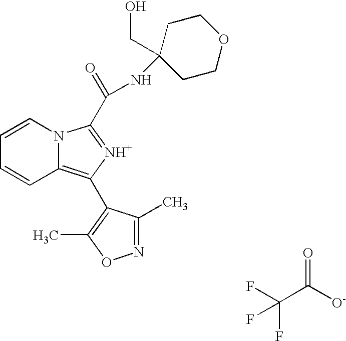 | 1-(3,5-dimethylisoxazol-4-yl)-n-[4- (hydroxymethyl)tetrahydro-2h-pyran-4- yl]imidazo[1,5-a]pyridine-3-carboxamide | 371.1714 |
| 672 | 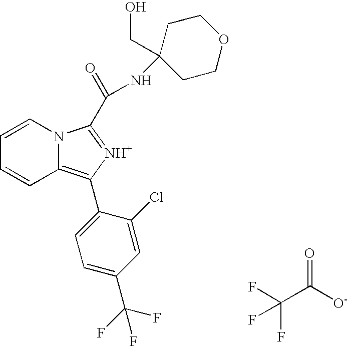 | 1-[2-chloro-4-(trifluoromethyl)phenyl]-n-[4- (hydroxymethyl)tetrahydro-2h-pyran-4- yl]imidazo[1,5-a]pyridine-3-carboxamide | 454.1133 |
| 673 | 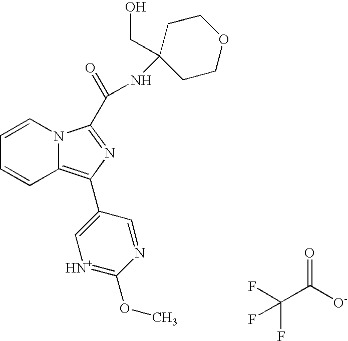 | N-[4-(hydroxymethyl)tetrahydro-2h-pyran-4-yl]-1- (2-methoxypyrimidin-5-yl)imidazo[1,5-a]pyridine- 3-carboxamide | 384.1663 |
| 674 | 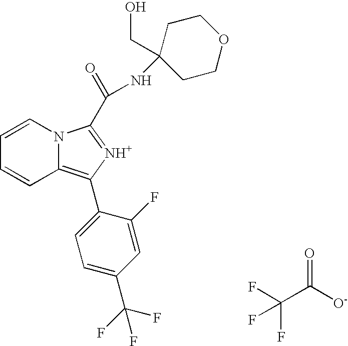 | 1-[2-fluoro-4-(trifluoromethyl)phenyl]-n-[4- (hydroxymethyl)tetrahydro-2h-pyran-4- yl]imidazo[1,5-a]pyridine-3-carboxamide | 438.144 |
| 675 | 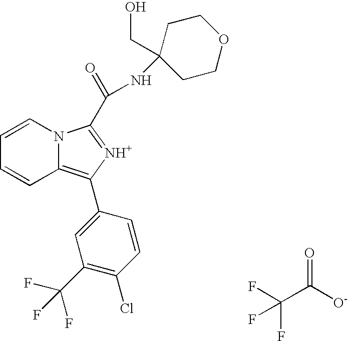 | 1-[4-chloro-3-(trifluoromethyl)phenyl]-n-[4- (hydroxymethyl)tetrahydro-2h-pyran-4- yl]imidazo[1,5-a]pyridine-3-carboxamide | 454.1133 |
| 676 | 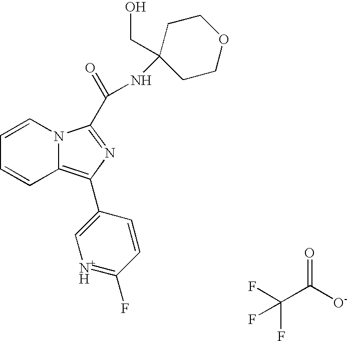 | 1-(6-fluoropyridin-3-yl)-n-[4- (hydroxymethyl)tetrahydro-2h-pyran-4- yl]imidazo[1,5-a]pyridine-3-carboxamide | 371.1516 |
| 677 | 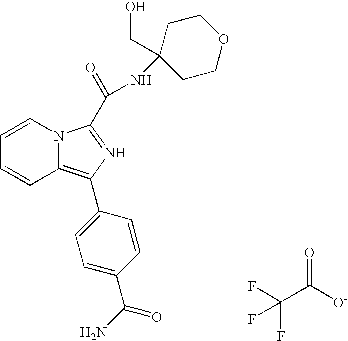 | 1-[4-(aminocarbonyl)phenyl]-n-[4- (hydroxymethyl)tetrahydro-2h-pyran-4- yl]imidazo[1,5-a]pyridine-3-carboxamide | 395.1 |
| 678 | 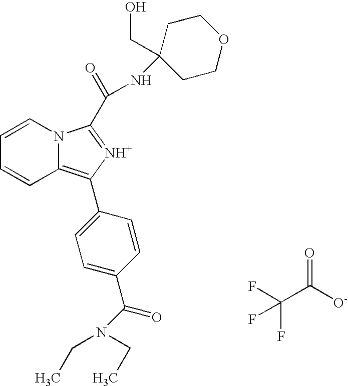 | 1-{4-[(diethylamino)carbonyl]phenyl}-n-[4- (hydroxymethyl)tetrahydro-2h-pyran-4- yl]imidazo[1,5-a]pyridine-3-carboxamide | 451.2342 |
| 679 | 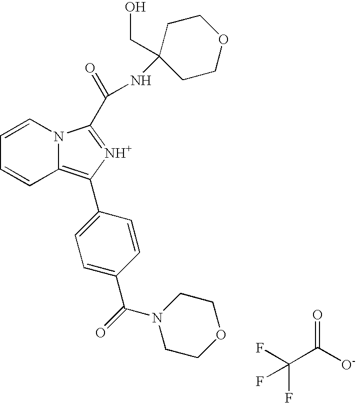 | N-[4-(hydroxymethyl)tetrahydro-2h-pyran-4-yl]-1- [4-(morpholin-4-ylcarbonyl)phenyl]imidazo[1,5- a]pyridine-3-carboxamide | 465.2137 |
| 680 | 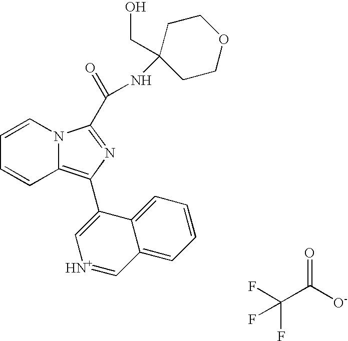 | N-[4-(hydroxymethyl)tetrahydro-2h-pyran-4-yl]-1- isoquinolin-4-ylimidazo[1,5-a]pyridine-3- carboxamide | 403.1768 |
| 681 | 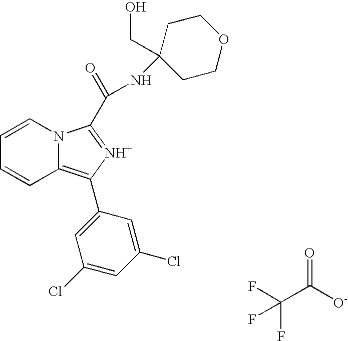 | 1-(3,5-dichlorophenyl)-n-[4- (hydroxymethyl)tetrahydro-2h-pyran-4- yl]imidazo[1,5-a]pyridine-3-carboxamide | 420.0876 |
| 682 | 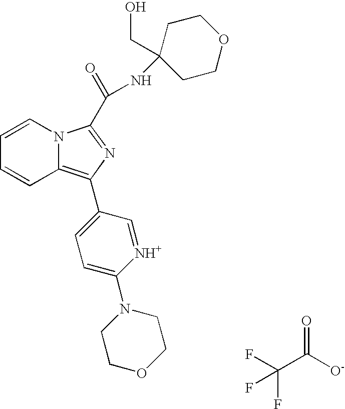 | N-[4-(hydroxymethyl)tetrahydro-2h-pyran-4-yl]-1- (6-morpholin-4-ylpyridin-3-yl)imidazo[1,5- a]pyridine-3-carboxamide | 438.2143 |
| 683 | 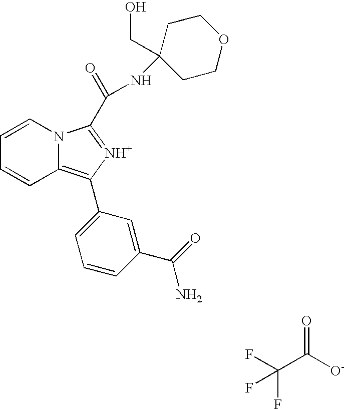 | 1-[3-(aminocarbonyl)phenyl]-n-[4- (hydroxymethyl)tetrahydro-2h-pyran-4- yl]imidazo[1,5-a]pyridine-3-carboxamide | 395.1717 |
| 684 | 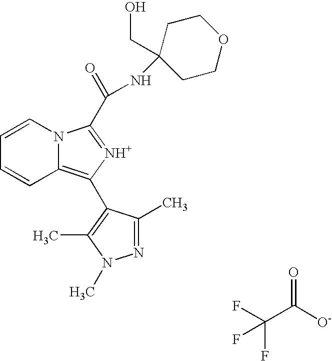 | n-[4-(hydroxymethyl)tetrahydro-2h-pyran-4-yl]-1- (1,3,5-trimethyl-1h-pyrazol-4-yl)imidazo[1,5- a]pyridine-3-carboxamide | 384.2034 |
| 685 | 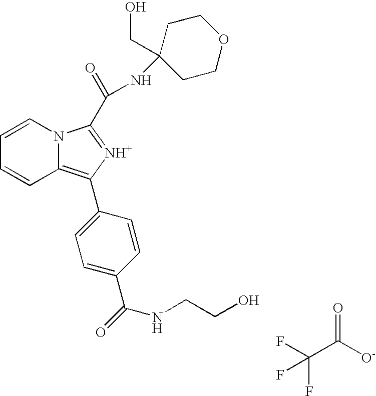 | 1-(4-{[(2-hydroxyethyl)amino]carbonyl}phenyl)-n- [4-(hydroxymethyl)tetrahydro-2h-pyran-4- yl]imidazo[1,5-a]pyridine-3-carboxamide | 439.2 |
| 686 | 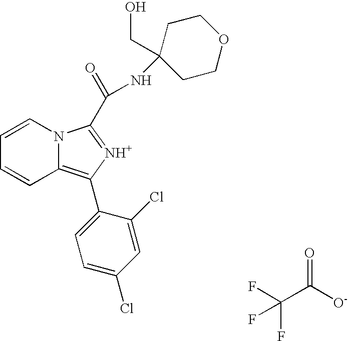 | 1-(2,4-dichlorophenyl)-n-[4- (hydroxymethyl)tetrahydro-2h-pyran-4- yl]imidazo[1,5-a]pyridine-3-carboxamide | 420.0882 |
| 687 | 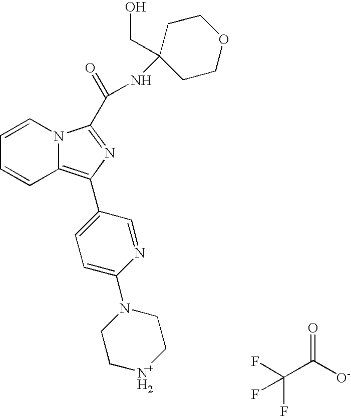 | n-[4-(hydroxymethyl)tetrahydro-2h-pyran-4-yl]-1- (6-piperazin-1-ylpyridin-3-yl)imidazo[1,5- a]pyridine-3-carboxamide | 437.2298 |
| 688 | 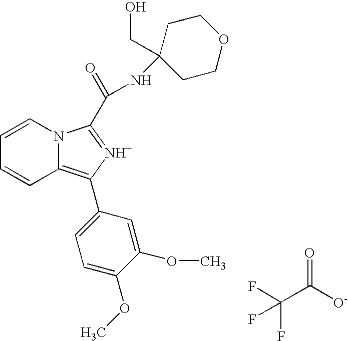 | 1-(3,4-dimethoxyphenyl)-n-[4- (hydroxymethyl)tetrahydro-2h-pyran-4- yl]imidazo[1,5-a]pyridine-3-carboxamide | 412.2 |
| 689 | 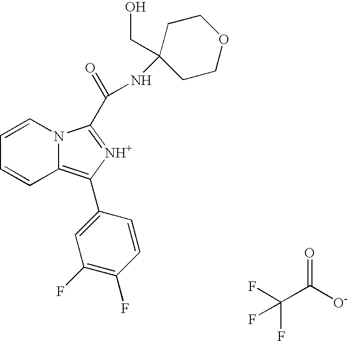 | 1-(3,4-difluorophenyl)-n-[4- (hydroxymethyl)tetrahydro-2h-pyran-4- yl]imidazo[1,5-a]pyridine-3-carboxamide | 388.1473 |
| 690 | 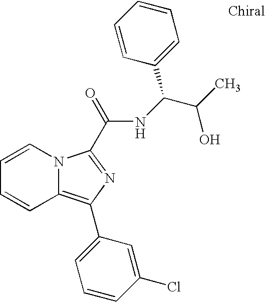 | 1-(3-chlorophenyl)-n-[(1r)-2-hydroxy-1- phenylpropyl]imidazo[1,5-a]pyridine-3- carboxamide | 406.2 |
| 691 | 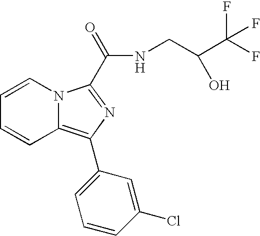 | 1-(3-chlorophenyl)-N-(3,3,3-trifluoro-2- hydroxypropyl)imidazo[1,5-a]pyridine-3- carboxamide | 384.1 |
| 692 | 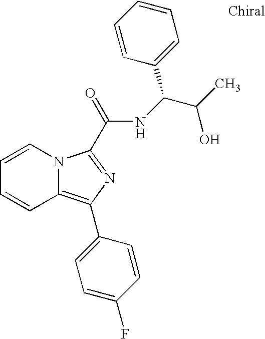 | 1-(4-fluorophenyl)-N-[(1R)-2-hydroxy-1- phenylpropyl]imidazo[1,5-a]pyridine-3- carboxamide | 390.2 |
| 693 | 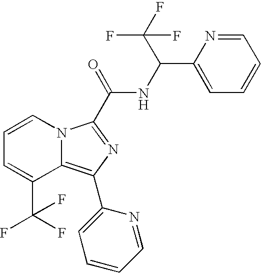 | 1-pyridin-2-yl-8-(trifluoromethyl)-N-(2,2,2-trifluoro- 1-pyridin-2-ylethyl)imidazo[1,5-a]pyridine-3- carboxamide | 466.2 |
| 694 | 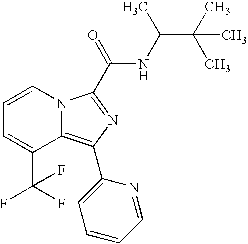 | 1-pyridin-2-yl-8-(trifluoromethyl)-N-(1,2,2- trimethylpropyl)imidazo[1,5-a]pyridine-3- carboxamide | 391.2 |

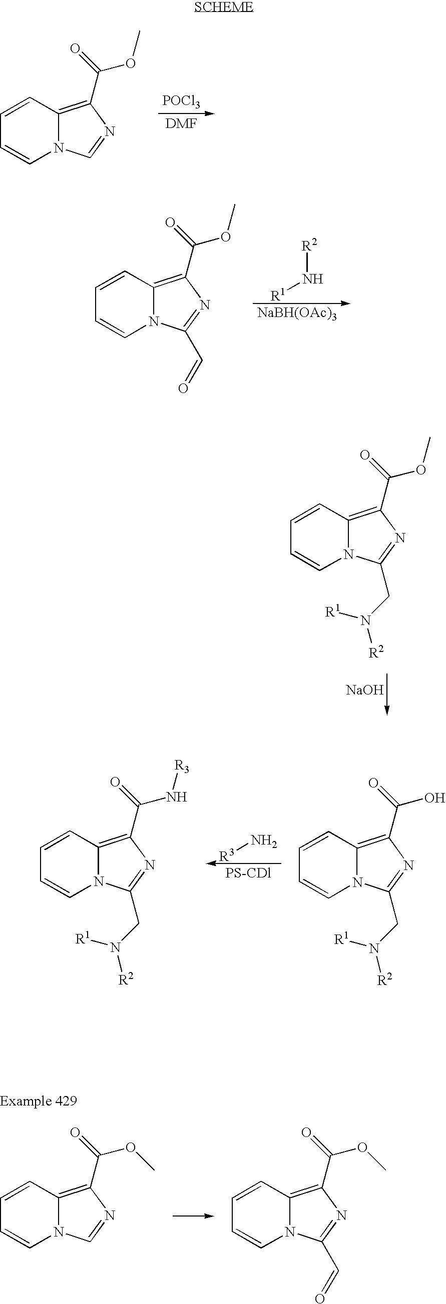

Methyl 3-formylimidazo[1,5-a]pyridine-1-carboxylate. To a suspension of methyl imidazo[1,5-a]pyridine-1-carboxylate (J. Heterocyclic Chem., 1991, 28, 1715; 1 g, 5.68 mmol) in 30 mL POCl₃ at 50° C. was added DMF (498 mg, 6.81 mmol) dropwise and then heated 115° C. After 1.5 h, the reaction mixture was cooled to room temperature and concentrated. CH₂Cl₂ was added to the resulting brown residue, cooled to 0° C. Saturated aqueous NaHCO₃ was added slowly to the cool mixture until the aqueous layer became basic. Layers were separated. Aqueous layer was extracted with CH₂Cl₂ (3×). Combined organic layers were dried over Na₂SO₄ and concentrated. The solid residue was recrystallized from CH₂Cl₂-heanes. Remaining desired product in the mother liquor from recrystallization was separated by passing it through a short silica column (elution with 2% MeOH—NH₃ in CH₂Cl₂). The combined yield was 1.158 g (tan solid, 100%).

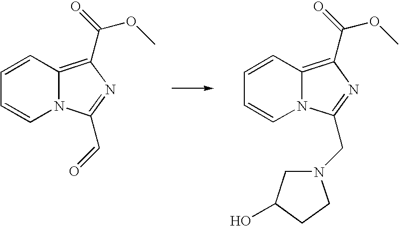

Methyl 3-[(3-hydroxypyrrolidin-1-yl)methyl]imidazo[1,5-a]pyridine-1-carboxylate. To a solution of methyl 3-formylimidazo[1,5-a]pyridine-1-carboxylate (400 mg, 1.959 mmol) in CH₂Cl₂—AcOH (98:2, v/v) was added 3-pyrrolidinol (205 mg, 2.353 mmol). The resulting mixture was stirred at room temperature for 45 min and then solid NaBH(OAc)₃ (498 mg, 2.351 mmol) was added. Stirred for 4 h. The reaction mixture was partitioned between satd. aqueous NaHCO₃ and CH₂Cl₂. Layers were separated and the aqueous layer was extracted with CH₂Cl₂ (3×). Combined organic layers were dried over Na₂SO₄ and concentrated. Desired product was separated by flash chromatography (silica gel) using a linear gradient of 0% to 10% MeOH in CH₂Cl₂ (brown glass, 522 mg, 97%).

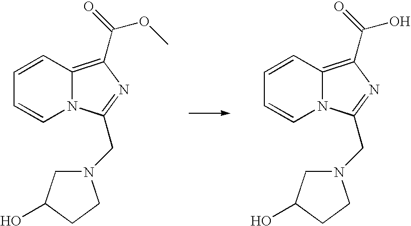

3-[(3-hydroxypyrrolidin-1-yl)methyl]imidazo[1,5-a]pyridine-1-carboxylic acid. To a solution of methyl 3-[(3-hydroxypyrrolidin-1-yl)methyl]imidazo[1,5-a]pyridine-1-carboxylate (522 mg, 1.896 mmol) in 2 mL MeOH was added aqueous NaOH (1 N, 2.275 mL) and was heated at 60° C. for 5 h. Cooled to room temperature and aqueous HCl (1 N, 2.275 mL) was added. The resulting mixture was then concentrated to light brown glass and was used as is in the next step.

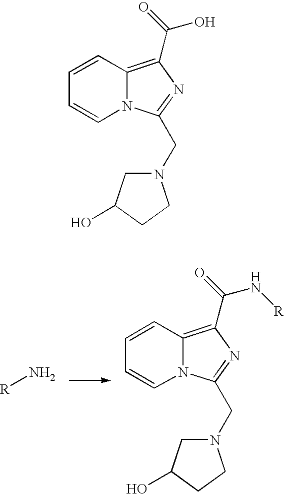

Amide derivatives. To a mixture of amine and polystyrene-CDI resin (PS-CDI) was added a solution containing 3-[(3-hydroxypyrrolidin-1-yl)methyl]imidazo[1,5-a]pyridine-1-carboxylic acid (35 mg, 0.134 mmol), HOAT (40 mg, 0.295 mmol) and Hunig's base (52 mg, 0.402 mmol) in 2 mL DMF. The resulting mixture was heated at 60° C. overnight and then filtered and concentrated. The desired product was separated by reverse phase HPLC. The following compounds were prepared according to this general method:

| | | | |
|---|---|---|---|
| 429 | 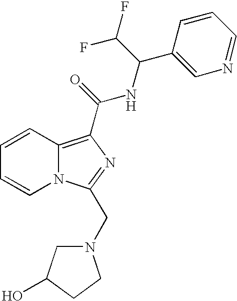 | N-(2,2-difluoro-1-pyridin-3-ylethyl)- 3-[(3-hydroxypyrrolidin-1- yl)methyl]imidazo[1,5-a]pyridine-1- carboxamide | 402.1735 |
| 430 | 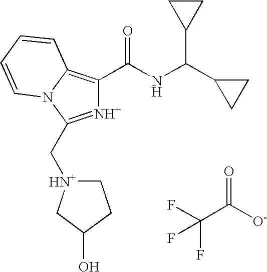 | 1- {[(dicyclopropylmethyl)amino] carbonyl}-3-[(3-hydroxypyrrolidinium-1- yl)methyl]imidazo[1,5-a]pyridin-2- ium bis(trifluoroacetate) | 355.2137 |
| 431 | 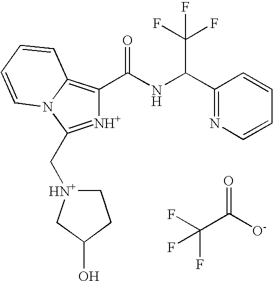 | 3-[(3-hydroxypyrrolidinium-1- yl)methyl]-1-{[(2,2,2-trifluoro-1- pyridin-2- ylethyl)amino]carbonyl}imidazo[1,5- a]pyridin-2-ium bis(trifluoroacetate) | 420.1644 |
| 432 | 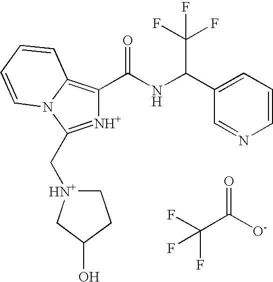 | 3-[(3-hydroxypyrrolidinium-1- yl)methyl]-1-{[2,2,2-trifluoro-1- pyridin-3- ylethyl)amino]carbonyl}imidazo[1,5- a]pyridin-2-ium bis(trifluoroacetate) | 420.1638 |
| 433 | 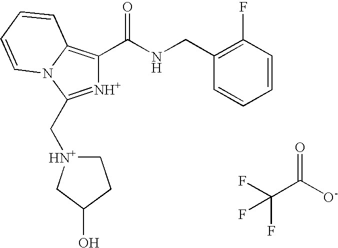 | 1-{[(2- fluorobenzyl)amino]carbonyl}-3-[(3- hydroxypyrrolidinium-1- yl)methyl]imidazo[1,5-a]pyridin-2- ium bis(trifluoroacetate) | 369.1734 |
| 434 | 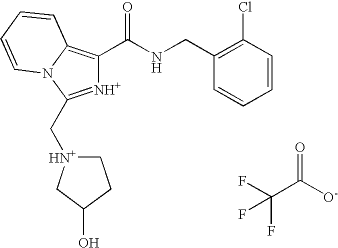 | 1-{[(2- chlorobenzyl)amino]carbonyl}-3-[(3- hydroxypyrrolidinium-1- yl)methyl]imidazo[1,5-a]pyridin-2- ium bis(trifluoroacetate) | 385.1435 |
| 435 | 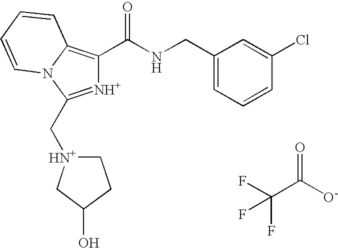 | 1-{[(3- chlorobenzyl)amino]carbonyl}-3-[(3- hydroxypyrrolidinium-1- yl)methyl]imidazo[1,5-a]pyridin-2- ium bis(trifluoroacetate) | 385.1434 |
| 436 | 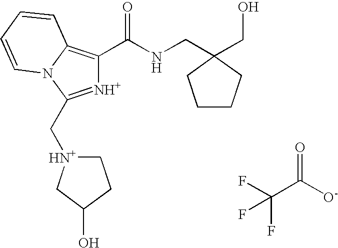 | 1-[({[1- (hydroxymethyl)cyclopentyl]methyl} amino)carbonyl]-3-[(3- hydroxypyrrolidinium-1- yl)methyl]imidazo[1,5-a]pyridin-2- ium bis(trifluoroacetate) | 373.2243 |
| 437 | 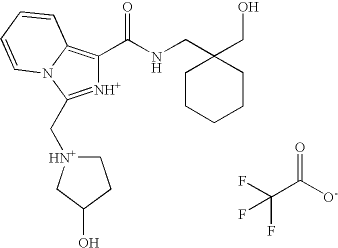 | 1-[({[1- (hydroxymethyl)cyclohexyl]methyl} amino)carbonyl]-3-[(3- hydroxypyrrolidinium-1- yl)methyl]imidazo[1,5-a]pyridin-2- ium bis(trifluoroacetate) | 387.2400 |
| 438 | 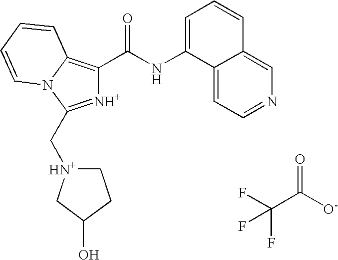 | 3-[(3-hydroxypyrrolidinium-1- yl)methyl]-1-[(isoquinolin-5- ylamino)carbonyl]imidazo[1,5- a]pyridin-2-ium bis(trifluoroacetate | 388.1773 |
| 439 | 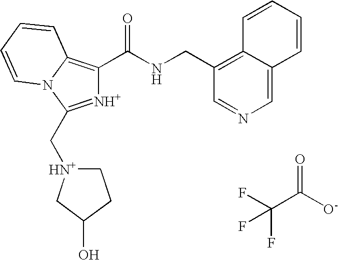 | 3-[(3-hydroxypyrrolidinium-1- yl)methyl]-1-{[(isoquinolin-4- ylmethyl)amino]carbonyl}imidazo[1, 5-a]pyridin-2-ium bis(trifluoroacetate) | 402.1929 |
| 440 | 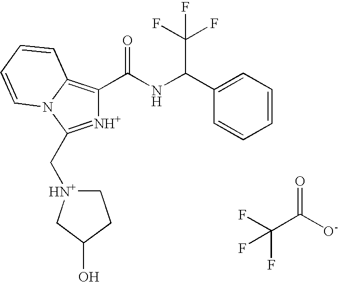 | 3-[(3-hydroxypyrrolidinium-1- yl)methyl]-1-{[(2,2,2-trifluoro-1- phenylethyl)amino]carbonyl}imidazo [1,5-a]pyridin-2-ium bis(trifluoroacetate) | 419.1692 |
| 441 | 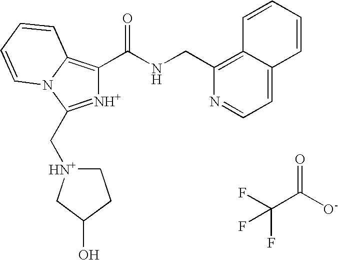 | 3-[(3-hydroxypyrrolidinium-1- yl)methyl]-1-{[(isoquinolin-1- ylmethyl)amino]carbonyl}imidazo[1, 5-a]pyridin-2-ium bis(trifluoroacetate) | 402.1925 |
| 442 | 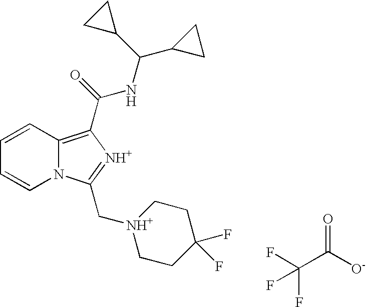 | 1-{[(dicyclopropylmethyl)amino]carbonyl}- 3-[(4,4-difluoropiperidinium-1- yl)methyl]imidazo[1,5-a]pyridin-2-ium bis(trifluoroacetate) | 389.2132 |
| 443 | 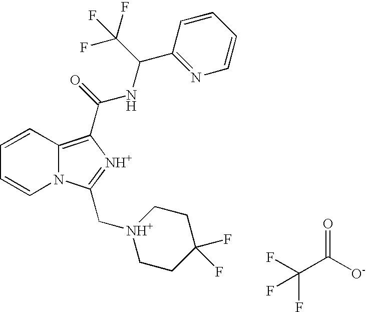 | 3-[(4,4-difluoropiperidinium-1- yl)methyl]-1-{[(2,2,2-trifluoro-1- pyridin-2- ylethyl)amino]carbonyl}imidazo[1,5- a]pyridin-2-ium bis(trifluoroacetate) | 454.1654 |
| 444 | 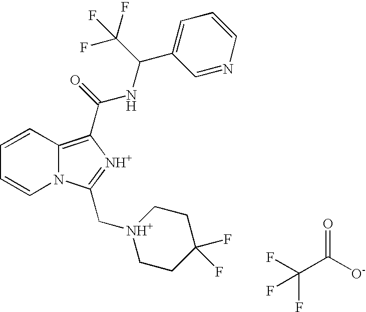 | 3-[(4,4-difluoropiperidinium-1- yl)methyl]-1-{[(2,2,2-trifluoro-1- pyridin-3- ylethyl)amino]carbonyl}imidazo[1,5- a]pyridin-2-ium bis(trifluoroacetate) | 454.1646 |
| 445 | 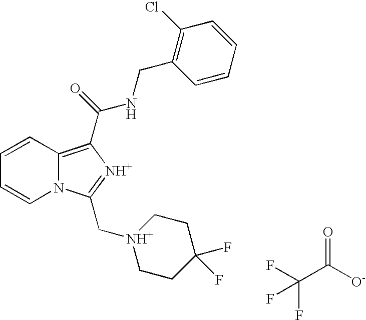 | 1-{[(2- chlorobenzyl)amino]carbonyl}-3- [(4,4-difluoropiperidinium-1- yl)methyl]imidazo[1,5-a]pyridin-2- ium bis(trifluoroacetate) | 419.1435 |
| 446 | 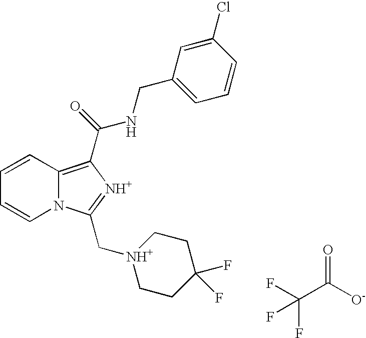 | 1-{[(3- chlorobenzyl)amino]carbonyl}-3- [(4,4-difluoropiperidinium-1- yl)methyl]imidazo[1,5-a]pyridin-2- ium bis(trifluoroacetate) | 419.1436 |
| 447 | 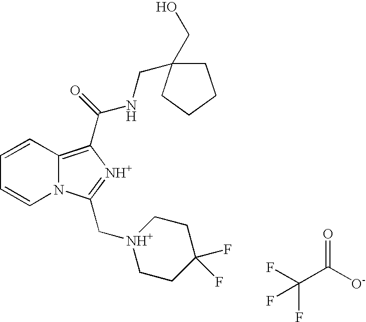 | 3-[(4,4-difluoropiperidinium-1- yl)methyl]-1-[({[1- (hydroxymethyl)cyclopentyl]methyl} amino)carbonyl]imidazo[1,5- a]pyridin-2-ium bis(trifluoroacetate) | 407.2244 |
| 448 | 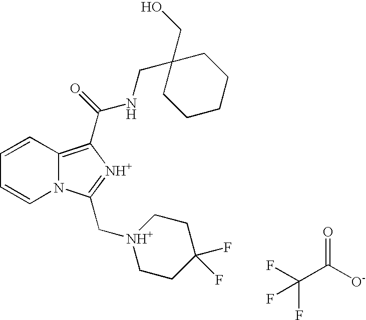 | 3-[(4,4-difluoropiperidinium-1- yl)methyl]-1-[({[1- (hydroxymethyl)cyclohexyl]methyl} amino)carbonyl]imidazo[1,5- a]pyridin-2-ium bis(trifluoroacetate) | 421.2397 |
| 449 | 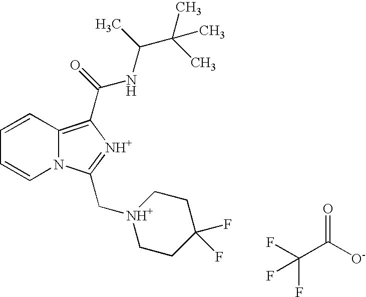 | 3-[(4,4-difluoropiperidinium-1- yl)methyl]-1-{[(1,2,2- trimethylpropyl)amino]carbonyl} imidazo[1,5-a]pyridin-2-ium bis(trifluoroacetate) | 379.2291 |
| 450 | 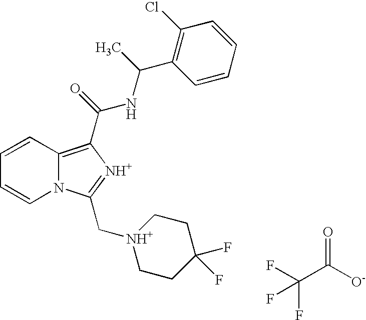 | 1-({[1-(2- chlorophenyl)ethyl]amino}carbonyl)- 3-[(4,4-difluoropiperidinium-1- yl)methyl]imidazo[1,5-a]pyridin-2- ium bis(trifluoroacetate) | 433.1590 |

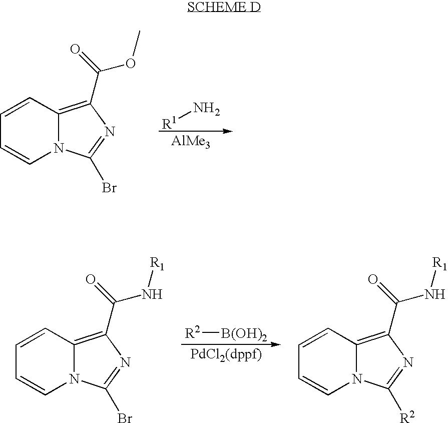

### Example 452

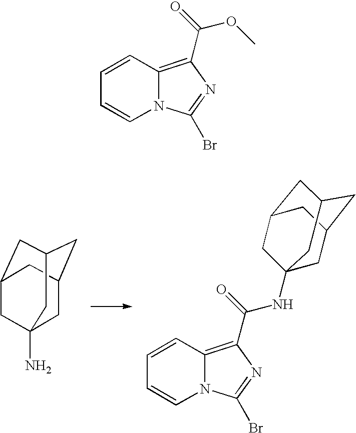

N-1-adamantyl-3-bromoimidazo[1,5-a]pyridine-1-carboxamide. To a solution of methyl 3-bromoimidazo[1,5-a]pyridine-1-carboxylate (J. Heterocyclic Chem., 1991, 28, 1715; 100 mg, 0.392 mmol) and adamantylamine (119 mg, 0.784 mmol) in 2.5 mL anhydrous toluene was added a Me₃Al solution (2 M in toluene, 0.392 mL, 0.784 mmol). The mixture was stirred at room temperature for 30 min and then heated at 85° C. for 10 h. Cooled to room temperature and then quenched by adding 10 drops of satd. aqueous NaHCO₃ and 1 mL ethyl acetate. After adding satd. aqueous K—Na-tartrate the mixture was stirred for 15 min and then partitioned between water and EtOAc. Layers were separated and the aqueous layer was extracted with EtOAc (3×). Combined organic layers were dried over Na₂SO₄ and concentrated. Purified by flash chromatography (silica gel) using a linear gradient of 3% to 40% EtOAc in hexanes. Desired product was isolated as a white solid (147 mg, 74%).

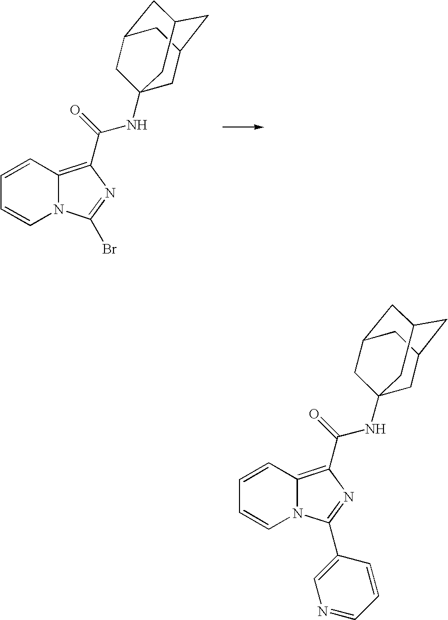

N-1-adamantyl-3-pyridin-3-ylimidazo[1,5-a]pyridine-1-carboxamide. A mixture of N-1-adamantyl-3-bromoimidazo[1,5-a]pyridine-1-carboxamide (30 mg, 0.08 mmol), 3-pyridylboronic acid (22 mg, 0.176 mmol), PdCl₂(dppf)CH₂Cl₂ (7 mg, 0.008 mmol) and aqueous Na₂CO₃ 2 M, 0.12 mL) in THF (2 mL) was heated at 150° C. (microwave) for 20 min. The reaction mixture was then cooled to room temperature and filtered through a pad of celite (celite pad was washed 3× with THF). The combined filtrate and washings were concentrated. The resulting residue was purified by r.p. HPLC. Desired product was isolated as a white solid (free base, 30 mg, 80%). ¹H NMR (500 MHz, CDCl₃): δ 9.06 (d, J=1.9 Hz, 1H), 8.73-8.72 (m, 1H), 8.43-8.41 (m, 1H), 8.22 (d, J=7.3 Hz, 1H), 8.12-8.10 (m, 1H), 7.51-7.47 (m, 1H), 7.10-7.02 (m, 2H), 6.78-6.75 (m, 1H), 2.22 (d, J=2.4 Hz, 6H), 2.14 (s, 3H), 1.75 (dd, J=24.2, 12.2 Hz, 6H).

The following compounds were prepared according to the procedures above:

TABLE D

| | | | |
|---|---|---|---|
| 452 | 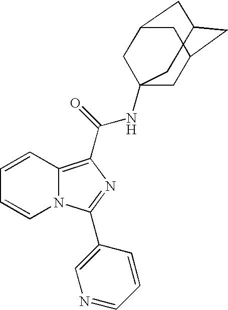 | N-1-adamantyl-3-pyridin-3-ylimidazo[1,5- a]pyridine-1-carboxamide | 373.2052 |
| 453 | 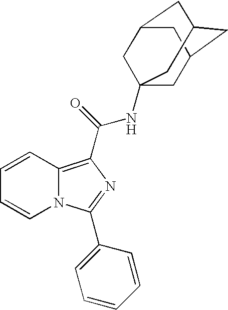 | N-1-adamantyl-3-phenylimidazo[1,5- a]pyridine-1-carboxamide | 372.2099 |
| 454 | 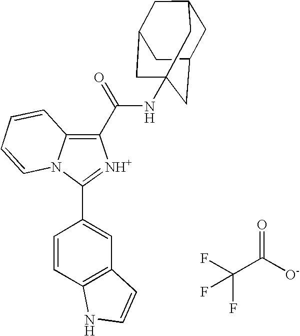 | 1-[(1-adamantylamino)carbonyl]-3-(1H- indol-5-yl)imidazo[1,5-a]pyridin-2-ium trifluoroacetate | 411.2199 |
| 455 | 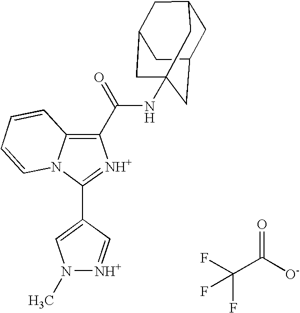 | 1-[(1-adamantylamino)carbonyl]-3-(1- methyl-1H-pyrazol-2-ium-4-yl)imidazo[1,5- a]pyridin-2-ium bis(trifluoroacetate) | 376.2142 |
| 456 | 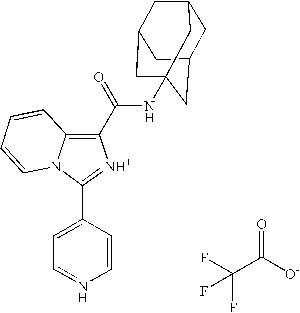 | 1-[(1-adamantylamino)carbonyl]-3- pyridinium-4-ylimidazo[1,5-a]pyridin-2-ium bis(trifluoroacetate) | 373.2026 |
| 457 | 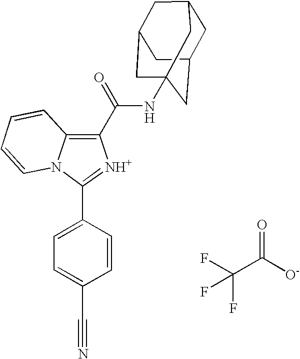 | 1-[(1-adamantylamino)carbonyl]-3-(4- cyanophenyl)imidazo[1,5-a]pyridin-2-ium trifluoroacetate | 397.2031 |
| 458 | 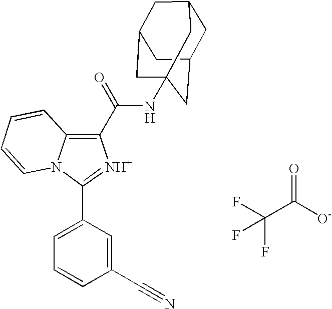 | 1-[(1-adamantylamino)carbonyl]-3-(3- cyanophenyl)imidazo[1,5-a]pyridin-2-ium trifluoroacetate | 397.2033 |
| 459 | 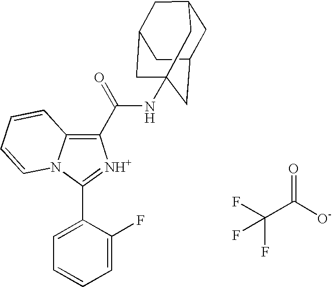 | 1-[(1-adamantylamino)carbonyl]-3-(2- fluorophenyl)imidazo[1,5-a]pyridin-2-ium trifluoroacetate | 390.1985 |
| 460 | 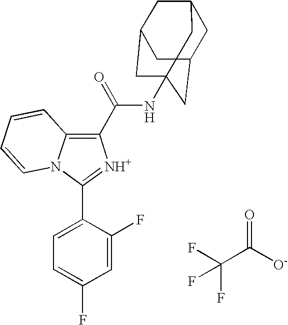 | 1-[(1-adamantylamino)carbonyl]-3-(2,4- difluorophenyl)imidazo[1,5-a]pyridin-2-ium trifluoroacetate | 408.1888 |
| 461 | 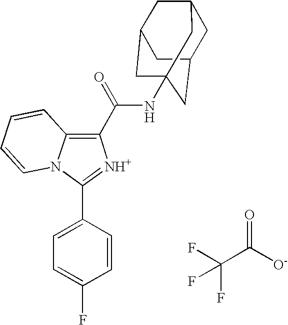 | 1-[(1-adamantylamino)carbonyl]-3-(4- fluorophenyl)imidazo[1,5-a]pyridin-2-ium trifluoroacetate | 390.1987 |
| 462 | 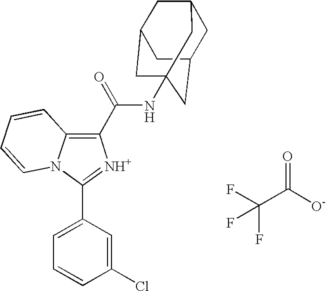 | 1-[(1-adamantylamino)carbonyl]-3-(3- chlorophenyl)imidazo[1,5-a]pyridin-2-ium trifluoroacetate | 406.1692 |
| 463 | 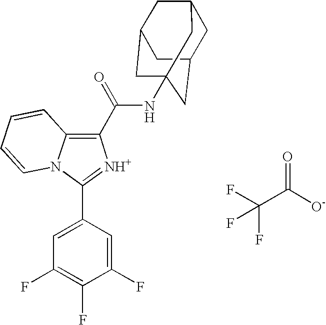 | 1-[(1-adamantylamino)carbonyl]-3-(3,4,5- trifluorophenyl)imidazo[1,5-a]pyridin-2-ium | 426.1793 |
| 464 | 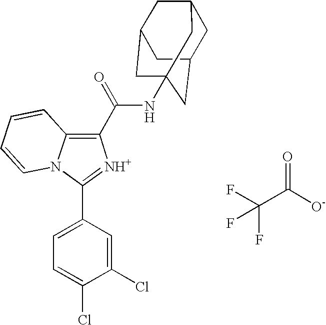 | 1-[(1-adamantylamino)carbonyl]-3-(3,4- dichlorophenyl)imidazo[1,5-a]pyridin-2-ium trifluoroacetate | 440.1298 |
| 465 | 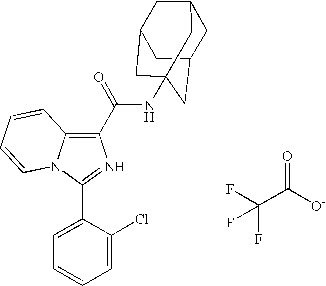 | 1-[(1-adamantylamino)carbonyl]-3-(2- chlorophenyl)imidazo[1,5-a]pyridin-2-ium trifluoroacetate | 406.1701 |
| 466 | 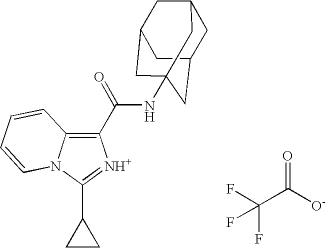 | 1-[(1-adamantylamino)carbonyl]-3- cyclopropylimidazo[1,5-a]pyridin-2-ium trifluoroacetate | 336.2074 |
| 467 | 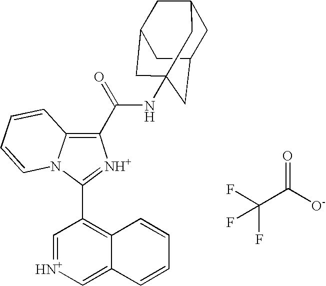 | 4-{1-[(1- adamantylamino)carbonyl]imidazo[1,5- a]pyridin-2-ium-3-yl}isoquinolinium bis(trifluoroacetate) | 423.2193 |
| 468 | 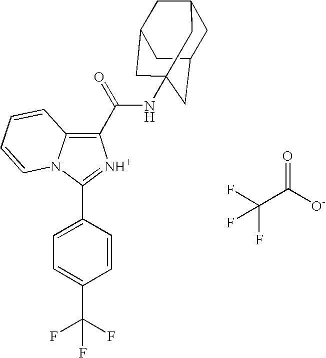 | 1-[(1-adamantylamino)carbonyl]-3-[4- (trifluoromethyl)phenyl]imidazo[1,5- a]pyridin-2-ium trifluoroacetate | 440.1950 |
| 469 | 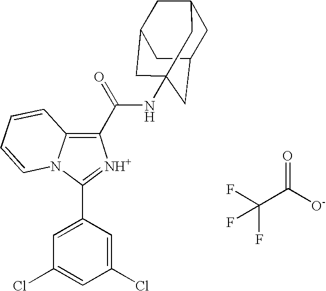 | 1-[(1-adamantylamino)carbonyl]-3-(3,5- dichlorophenyl)imidazo[1,5-a]pyridin-2-ium trifluoroacetate | 440.1303 |
| 470 | 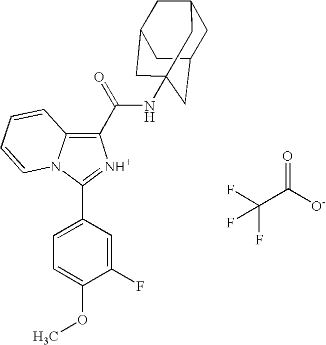 | 1-[(1-adamantylamino)carbonyl]-3-(3- fluoro-4-methoxyphenyl)imidazo[1,5- a]pyridin-2-ium trifluoroacetate | 420.2099 |
| 471 | 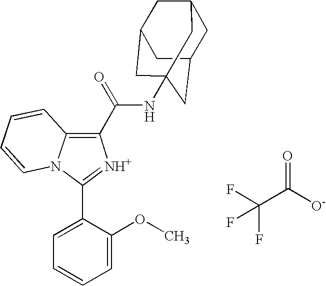 | 1-[(1-adamantylamino)carbonyl]-3-(2- methoxyphenyl)imidazo[1,5-a]pyridin-2- ium trifluoroacetate | 402.2190 |
| 472 | 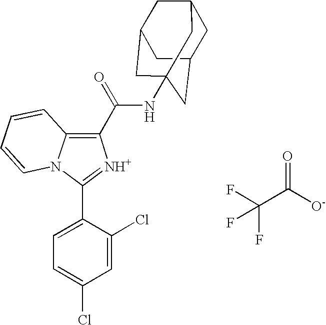 | 1-[(1-adamantylamino)carbonyl]-3-(2,4- dichlorophenyl)imidazo[1,5-a]pyridin-2-ium trifluoroacetate | 440.1303 |
| 473 | 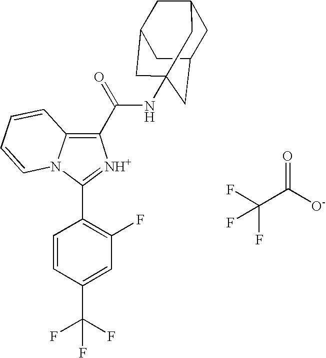 | 1-[(1-adamantylamino)carbonyl]-3-[2- fluoro-4- (trifluoromethyl)phenyl]imidazo[1,5- a]pyridin-2-ium trifluoroacetate | 458.1863 |
| 474 | 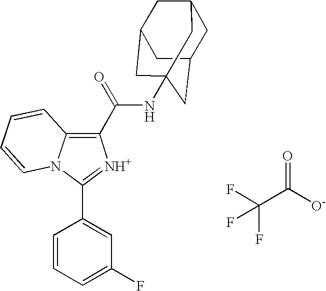 | 1-[(1-adamantylamino)carbonyl]-3-(3- fluorophenyl)imidazo[1,5-a]pyridin-2-ium trifluoroacetate | 390.1988 |
| 475 | 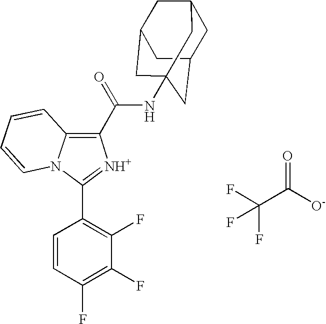 | 1-[(1-adamantylamino)carbonyl]-3-(2,3,4- trifluorophenyl)imidazo[1,5-a]pyridin-2-ium trifluoroacetate | 426.1803 |
| 476 | 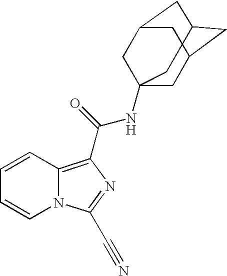 | N-1-adamantyl-3-cyanoimidazo[1,5- a]pyridine-1-carboxamide | 353.1984 |
| 477 | 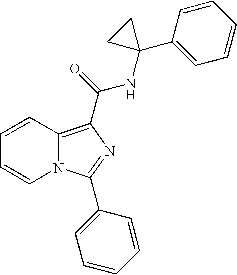 | 3-phenyl-n-(1- phenylcyclopropyl)imidazo[1,5-a]pyridine- 1-carboxamide | 354.1613 |
| 478 | 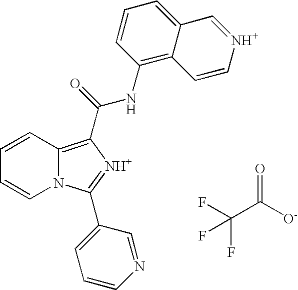 | 5-{[(3-pyridin-3-ylimidazo[1,5-a]pyridin-2- ium-1-yl)carbonyl]amino}isoquinolinium bis(trifluoroacetate) | 366.1349 |
| 479 | 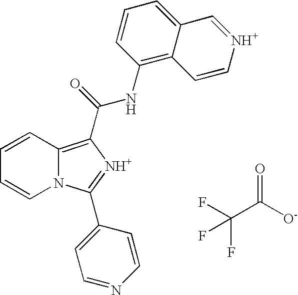 | 5-{[(3-pyridin-4-ylimidazo[1,5-a]pyridin-2- ium-1-yl)carbonyl]amino}isoquinolinium bis(trifluoroacetate) | 366.1349 |
| 480 | 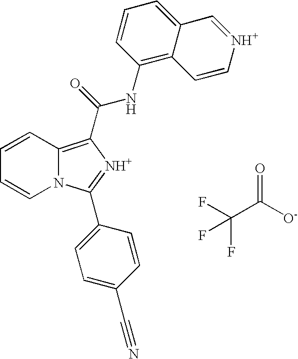 | 5-({[3-(4-cyanophenyl)imidazo[1,5- a]pyridin-2-ium-1- yl]carbonyl}amino)isoquinolinium bis(trifluoroacetate) | 390.1351 |
| 481 | 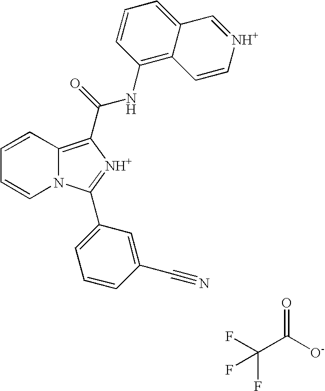 | 5-({[3-(3-cyanophenyl)imidazo[1,5- a]pyridin-2-ium-1- yl]carbonyl}amino)isoquinolinium bis(trifluoroacetate) | 390.1352 |
| 482 | 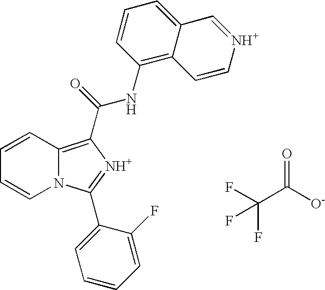 | 5-({[3-(2-fluorophenyl)imidazo[1,5- a]pyridin-2-ium-1- yl]carbonyl}amino)isoquinolinium bis(trifluoroacetate) | 383.1312 |
| 483 | 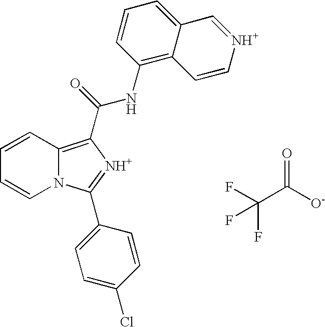 | 5-({[3-(4-chlorophenyl)imidazo[1,5- a]pyridin-2-ium-1- yl]carbonyl}amino)isoquinolinium bis(trifluoroacetate) | 399.102 |
| 484 | 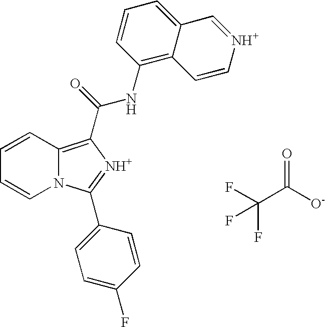 | 5-({[3-(4-fluorophenyl)imidazo[1,5- a]pyridin-2-ium-1- yl]carbonyl}amino)isoquinolinium bis(trifluoroacetate) | 383.1304 |
| 485 | 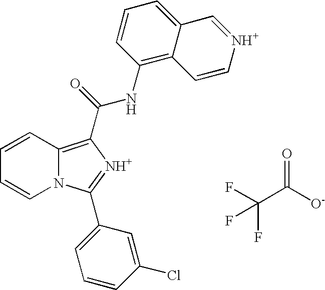 | 5-({[3-(3-chlorophenyl)imidazo[1,5- a]pyridin-2-ium-1- yl]carbonyl}amino)isoquinolinium bis(trifluoroacetate) | 399.1011 |
| 486 | 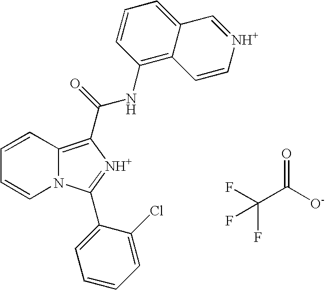 | 5-({[3-(2-chlorophenyl)imidazo[1,5- a]pyridin-2-ium-1- yl]carbonyl}amino)isoquinolinium bis(trifluoroacetate) | 399.1025 |
| 487 | 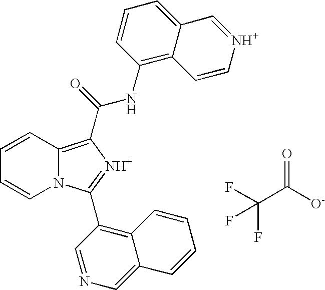 | 5-{[(3-isoquinolinium-4-ylimidazo[1,5- a]pyridin-2-ium-1- yl)carbonyl]amino}isoquinolinium bis(trifluoroacetate) | 416.1509 |
| 488 | 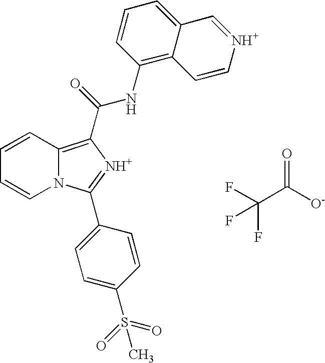 | 5-[({3-[4- (methylsulfonyl)phenyl]imidazo[1,5- a]pyridin-2-ium-1- yl}carbonyl)amino]isoquinolinium bis(trifluoroacetate) | 443.1184 |
| 489 | 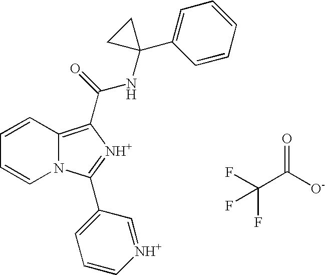 | 1-{[(1-phenylcyclopropyl)amino]carbonyl}- 3-pyridinium-3-ylimidazo[1,5-a]pyridin-2- ium bis(trifluoroacetate) | 355.1560 |
| 490 | 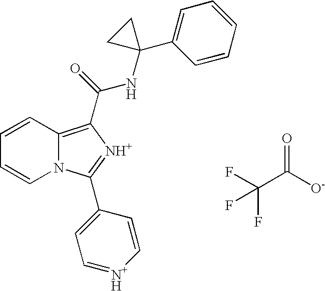 | 1-{[(1-phenylcyclopropyl)amino]carbonyl}- 3-pyridinium-4-ylimidazo[1,5-a]pyridin-2- ium bis(trifluoroacetate) | 355.1559 |
| 491 | 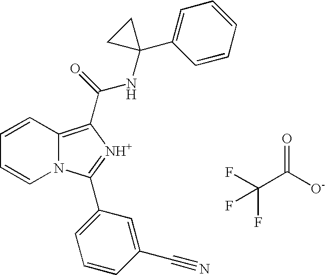 | 3-(3-cyanophenyl)-1-{[(1- phenylcyclopropyl)amino]carbonyl}imidazo [1,5-a]pyridin-2-ium trifluoroacetate | 379.1568 |
| 492 | 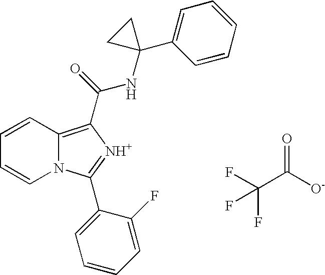 | 3-(2-fluorophenyl)-1-{[(1- phenylcyclopropyl)amino]carbonyl}imidazo [1,5-a]pyridin-2-ium trifluoroacetate | 372.1518 |
| 493 | 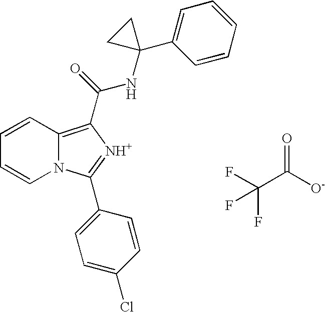 | 3-(4-chlorophenyl)-1-{[(1- phenylcyclopropyl)amino]carbonyl}imidazo [1,5-a]pyridin-2-ium trifluoroacetate | 388.1227 |
| 494 | 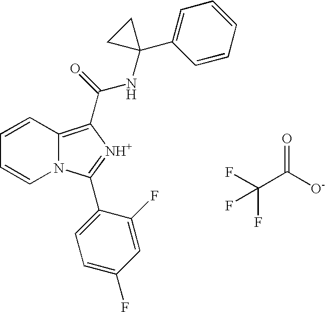 | 3-(2,4-difluorophenyl)-1-{[(1- phenylcyclopropyl)amino]carbonyl}imidazo [1,5-a]pyridin-2-ium trifluoroacetate | 390.1428 |
| 495 | 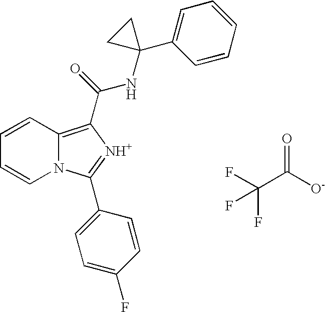 | 3-(4-fluorophenyl)-1-{[(1- phenylcyclopropyl)amino]carbonyl}imidazo [1,5-a]pyridin-2-ium trifluoroacetate | 372.1519 |
| 496 | 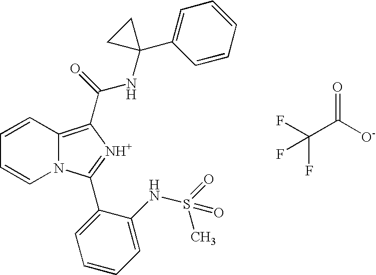 | 3-{2-[(methylsulfonyl)amino]phenyl}-1- {[(1- phenylcyclopropyl)amino]carbonyl}imidazo [1,5-a]pyridin-2-ium trifluoroacetate | 447.1511 |
| 497 | 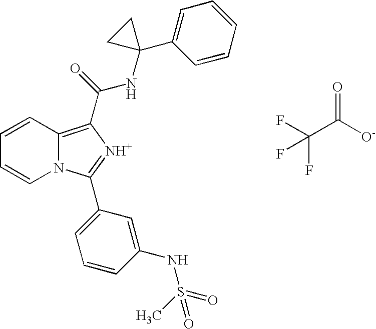 | 3-{3-[(methylsulfonyl)amino]phenyl}-1- {[(1- phenylcyclopropyl)amino]carbonyl}imidazo [1,5-a]pyridin-2-ium trifluoroacetate | 447.1496 |
| 498 | 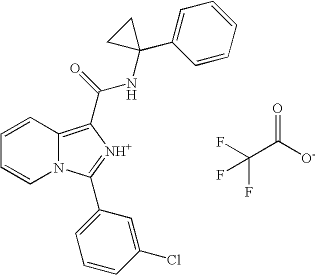 | 3-(3-chlorophenyl)-1-{[(1- phenylcyclopropyl)amino]carbonyl}imidazo [1,5-a]pyridin-2-ium trifluoroacetate | 388.1230 |
| 499 | 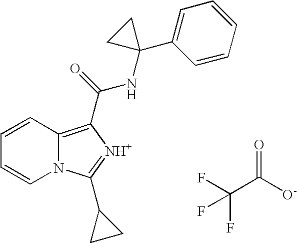 | 3-cyclopropyl-1-{[(1- phenylcyclopropyl)amino]carbonyl}imidazo [1,5-a]pyridin-2-ium trifluoroacetate | 318.1605 |
| 500 | 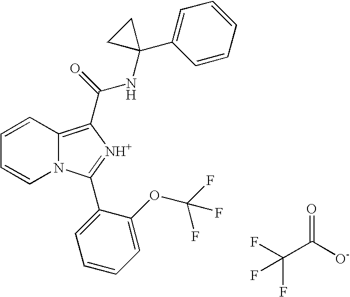 | 1-{[(1-phenylcyclopropyl)amino]carbonyl}- 3-[2-(trifluoromethoxy)phenyl]imidazo[1,5- a]pyridin-2-ium trifluoroacetate | 438.1441 |
| 501 | 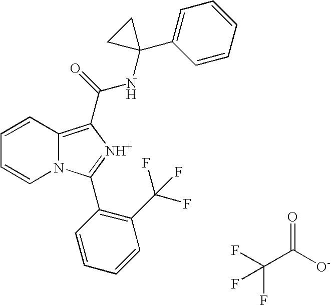 | 1-{[(1-phenylcyclopropyl)amino]carbonyl}- 3-[2-(trifluoromethyl)phenyl]imidazo[1,5- a]pyridin-2-ium trifluoroacetate | 422.1490 |
| 502 | 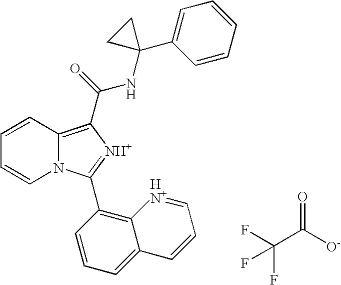 | 8-(1-{[(1- phenylcyclopropyl)amino]carbonyl}imidazo [1,5-a]pyridin-2-ium-3-yl)quinolinium bis(trifluoroacetate) | 405.1723 |
| 503 | 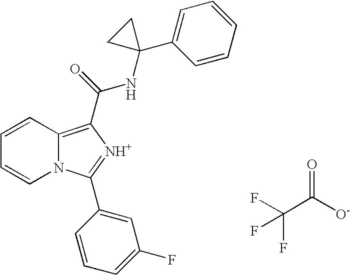 | 3-(3-fluorophenyl)-1-{[(1- phenylcyclopropyl)amino]carbonyl}imidazo [1,5-a]pyridin-2-ium trifluoroacetate | 372.1524 |
| 504 | 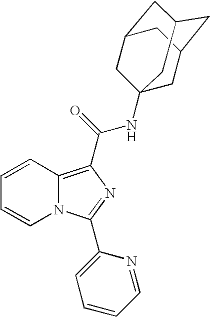 | N-1-adamantyl-3-pyridin-2-ylimidazo[1,5- a]pyridine-1-carboxamide | 373.2042 |
| 505 | 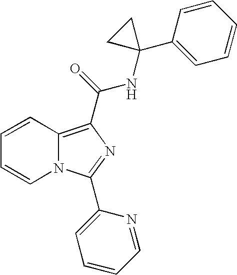 | N-(1-phenylcyclopropyl)-3-pyridin-2- ylimidazo[1,5-a]pyridine-1-carboxamide | 355.1570 |
| 506 | 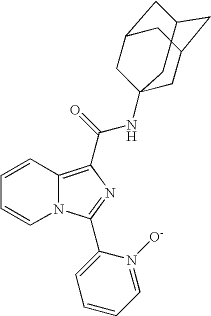 | N-1-adamantyl-3-(1-oxidopyridin-2- yl)imidazo[1,5-a]pyridine-1-carboxamide | 389.1958 |
| 507 | 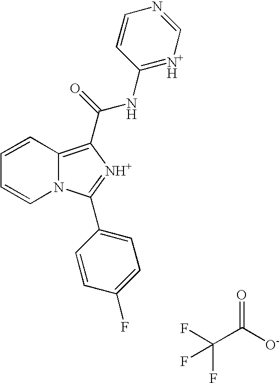 | 3-(4-fluorophenyl)-1-[(pyrimidin-3-ium-4- ylamino)carbonyl]imidazo[1,5-a]pyridin-2- ium bis(trifluoroacetate) | 334.1095 |
| 508 | 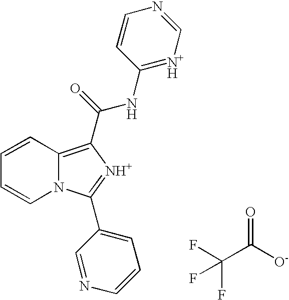 | 3-pyridin-3-yl-1-[(pyrimidin-3-ium-4- ylamino)carbonyl]imidazo[1,5-a]pyridin-2- ium bis(trifluoroacetate) | 317.1142 |
| 509 | 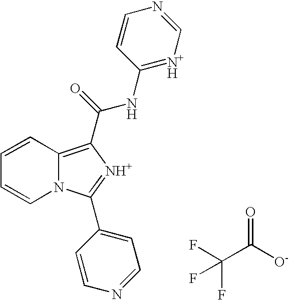 | 3-pyridin-4-yl-1-[(pyrimidin-3-ium-4- ylamino)carbonyl]imidazo[1,5-a]pyridin-2- ium bis(trifluoroacetate) | 317.1143 |
| 510 | 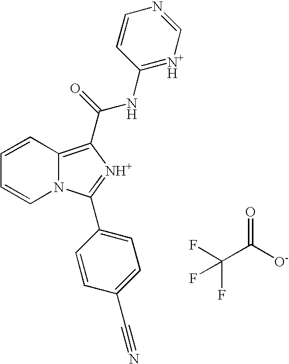 | 3-(4-cyanophenyl)-1-[(pyrimidin-3-ium-4- ylamino)carbonyl]imidazo[1,5-a]pyridin-2- ium bis(trifluoroacetate) | 341.1141 |
| 511 | 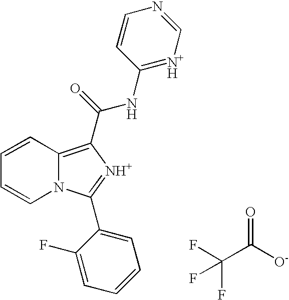 | 3-(2-fluorophenyl)-1-[(pyrimidin-3-ium-4- ylamino)carbonyl]imidazo[1,5-a]pyridin-2- ium bis(trifluoroacetate) | 334.1095 |
| 512 | 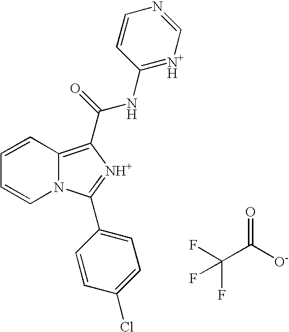 | 3-(4-chlorophenyl)-1-[(pyrimidin-3-ium-4- ylamino)carbonyl]imidazo[1,5-a]pyridin-2- ium bis(trifluoroacetate) | 350.0798 |
| 513 | 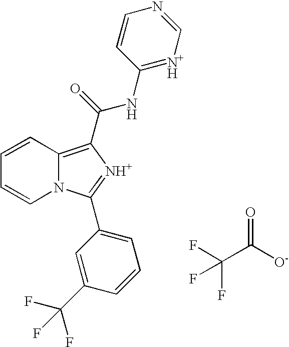 | 1-[(pyrimidin-3-ium-4-ylamino)carbonyl]-3- [3-(trifluoromethyl)phenyl]imidazo[1,5- a]pyridin-2-ium bis(trifluoroacetate) | 384.1062 |
| 514 | 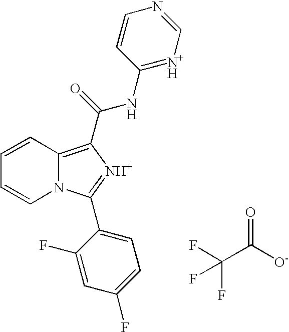 | 3-(2,4-difluorophenyl)-1-[(pyrimidin-3-ium- 4-ylamino)carbonyl]imidazo[1,5-a]pyridin- 2-ium bis(trifluoroacetate) | 352.0999 |
| 515 | 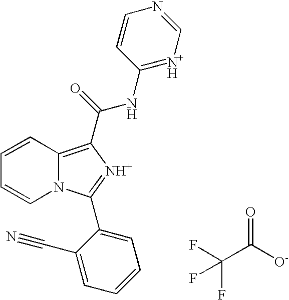 | 3-(2-cyanophenyl)-1-[(pyrimidin-3-ium-4- ylamino)carbonyl]imidazo[1,5-a]pyridin-2- ium bis(trifluoroacetate) | 341.1142 |
| 516 | 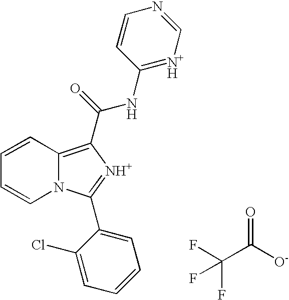 | 3-(2-chlorophenyl)-1-[(pyrimidin-3-ium-4- ylamino)carbonyl]imidazo[1,5-a]pyridin-2- ium bis(trifluoroacetate) | 350.0801 |
| 517 | 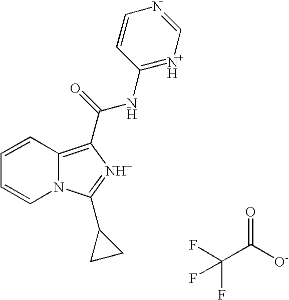 | 3-cyclopropyl-1-[(pyrimidin-3-ium-4- ylamino)carbonyl]imidazo[1,5-a]pyridin-2- ium bis(trifluoroacetate) | 280.1190 |
| 518 | 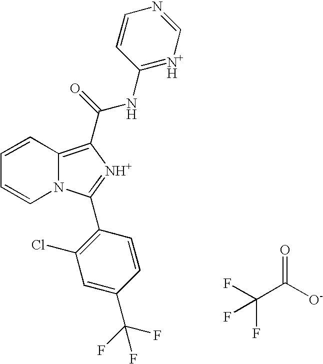 | 3-[2-chloro-4-(trifluoromethyl)phenyl]-1- [(pyrimidin-3-ium-4- ylamino)carbonyl]imidazo[1,5-a]pyridin-2- ium bis(trifluoroacetate) | 418.0672 |
| 519 | 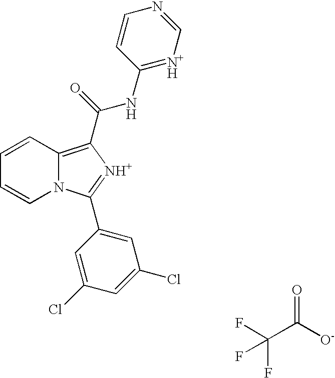 | 3-(3,5-dichlorophenyl)-1-[(pyrimidin-3-ium- 4-ylamino)carbonyl]imidazo[1,5-a]pyridin- 2-ium bis(trifluoroacetate) | 384.0408 |
| 520 | 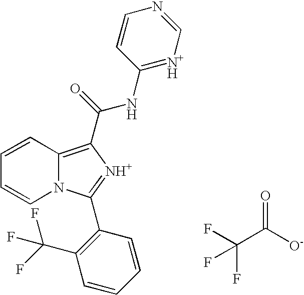 | 1-[(pyrimidin-3-ium-4-ylamino)carbonyl]-3- [2-(trifluoromethyl)phenyl]imidazo[1,5- a]pyridin-2-ium bis(trifluoroacetate) | 384.1063 |
| 521 | 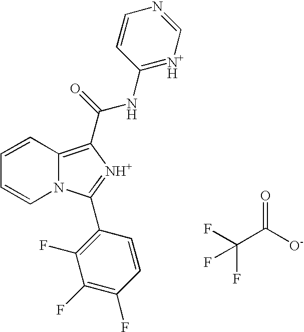 | 1-[(pyrimidin-3-ium-4-ylamino)carbonyl]-3- (2,3,4-trifluorophenyl)imidazo[1,5- a]pyridin-2-ium bis(trifluoroacetate) | 370.0906 |
| 522 | 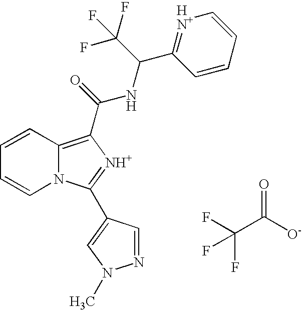 | 3-(1-methyl-1H-pyrazol-4-yl)-1-{[(2,2,2- trifluoro-1-pyridinium-2- ylethyl)amino]carbonyl}imidazo[1,5- a]pyridin-2-ium bis(trifluoroacetate) | 401.1326 |
| 523 | 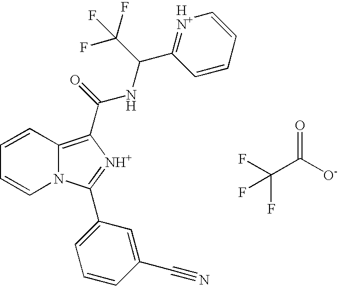 | 3-(3-cyanophenyl)-1-{[(2,2,2-trifluoro-1- pyridinium-2- ylethyl)amino]carbonyl}imidazo[1,5- a]pyridin-2-ium bis(trifluoroacetate) | 422.1212 |
| 524 | 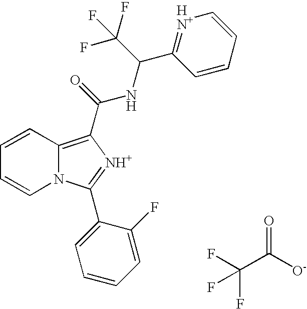 | 3-(2-fluorophenyl)-1-{[(2,2,2-trifluoro-1- pyridinium-2- ylethyl)amino]carbonyl}imidazo[1,5- a]pyridin-2-ium bis(trifluoroacetate) | 415.1172 |
| 525 | 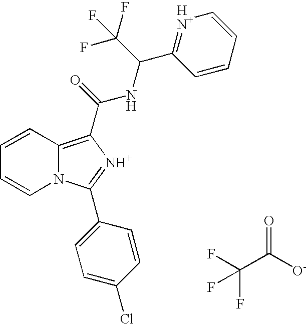 | 3-(4-chlorophenyl)-1-{[(2,2,2-trifluoro-1- pyridinium-2- ylethyl)amino]carbonyl}imidazo[1,5- a]pyridin-2-ium bis(trifluoroacetate) | 431.0868 |
| 526 | 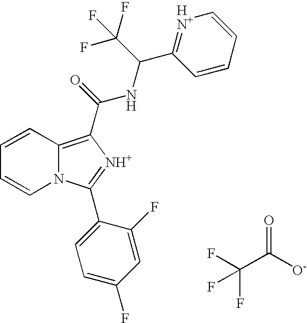 | 3-(2,4-difluorophenyl)-1-{[(2,2,2-trifluoro- 1-pyridinium-2- ylethyl)amino]carbonyl}imidazo[1,5- a]pyridin-2-ium bis(trifluoroacetate) | 433.1071 |
| 527 | 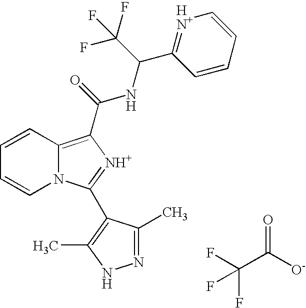 | 3-(3,5-dimethyl-1H-pyrazol-4-yl)-1-{[(2,2,2- trifluoro-1-pyridinium-2- ylethyl)amino]carbonyl}imidazo[1,5- a]pyridin-2-ium bis(trifluoroacetate) | 415.1481 |
| 528 | 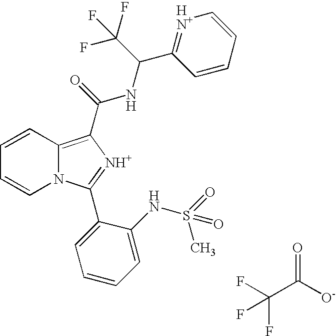 | 3-{2-[(methylsulfonyl)amino]phenyl}-1- {[(2,2,2-trifluoro-1-pyridinium-2- ylethyl)amino]carbonyl}imidazo[1,5- a]pyridin-2-ium bis(trifluoroacetate) | 490.1150 |
| 529 | 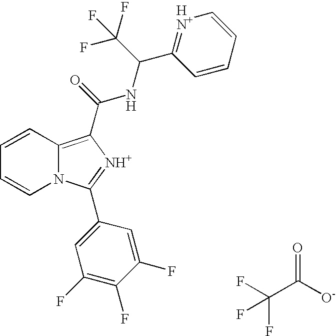 | 3-(3,4,5-trifluorophenyl)-1-{[(2,2,2- trifluoro-1-pyridinium-2- ylethyl)amino]carbonyl}imidazo[1,5- a]pyridin-2-ium bis(trifluoroacetate) | 451.0974 |
| 530 | 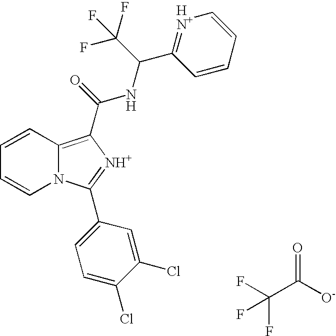 | 3-(3,4-dichlorophenyl)-1-{[(2,2,2-trifluoro- 1-pyridinium-2- ylethyl)amino]carbonyl}imidazo[1,5- a]pyridin-2-ium bis(trifluoroacetate) | 465.0480 |
| 531 | 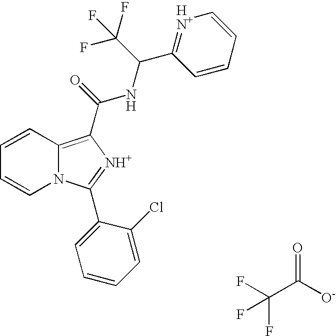 | 3-(2-chlorophenyl)-1-{[(2,2,2-trifluoro-1- pyridinium-2- ylethyl)amino]carbonyl}imidazo[1,5- a]pyridin-2-ium bis(trifluoroacetate) | 431.0867 |
| 532 | 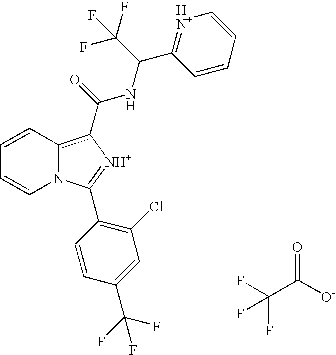 | 3-[2-chloro-4-(trifluoromethyl)phenyl]-1- {[(2,2,2-trifluoro-1-pyridinium-2- ylethyl)amino]carbonyl}imidazo[1,5- a]pyridin-2-ium bis(trifluoroacetate) | 499.0732 |
| 533 | 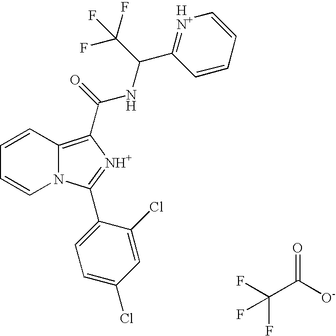 | 3-(2,4-dichlorophenyl)-1-{[(2,2,2-trifluoro- 1-pyridinium-2- ylethyl)amino]carbonyl}imidazo[1,5- a]pyridin-2-ium bis(trifluoroacetate) | 465.0476 |
| 534 | 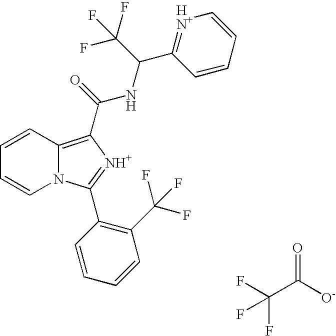 | 3-[2-(trifluoromethyl)phenyl]-1-{[(2,2,2- trifluoro-1-pyridinium-2- ylethyl)amino]carbonyl}imidazo[1,5- a]pyridin-2-ium bis(trifluoroacetate) | 465.1131 |
| 535 | 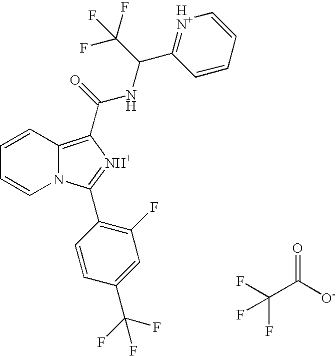 | 3-[2-fluoro-4-(trifluoromethyl)phenyl]-1- {[(2,2,2-trifluoro-1-pyridinium-2- ylethyl)amino]carbonyl}imidazo[1,5- a]pyridin-2-ium bis(trifluoroacetate) | 483.1030 |
| 536 | 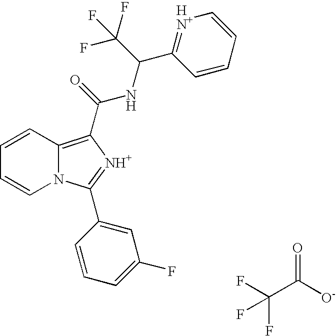 | 3-(3-fluorophenyl)-1-{[(2,2,2-trifluoro-1- pyridinium-2- ylethyl)amino]carbonyl}imidazo[1,5- a]pyridin-2-ium bis(trifluoroacetate) | 415.1169 |

### Example 537

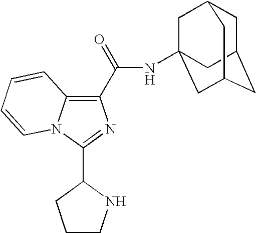

### N-1-Adamantyl-3-pyrrolidin-2-ylimidazo[1,5-a]pyridine-1-carboxamide

### Step A: Benzyl 2-{1-[(1-adamantylamino)carbonyl]imidazo[1,5-a]pyridin-3-yl}pyrrolidine-1-carboxylate

To a solution of 3-{1-[(benzyloxy)carbonyl]pyrrolidin-2-yl}imidazo[1,5-a]pyridine-1-carboxylic acid (0.24 g, 0.66 mmol) in DMF (25 mL) were added adamantan-1-amine (0.12 g, 0.82 mmol), EDC (0.19 g, 0.99 mmol), HOAT (0.13 g, 0.99 mmol) and diisopropylethylamine (0.63 mL, 3.61 mmol). The reaction mixture was stirred at ambient temperature for 18 h. The mixture was concentrated and saturated aqueous NaHCO₃ was added. The mixture was extracted with DCM. The combined organic extracts were dried over Na₂SO₄, filtered and concentrated. Purification by silica gel chromatography (1%→50% EtOAc/hexanes) to give the title compound (0.21 g). MS 499.2 (M+1).

### Step B: N-1-Adamantyl-3-pyrrolidin-2-ylimidazo[1,5-a]pyridine-1-carboxamide

To a solution of benzyl 2-{1-[(1-adamantylamino)carbonyl]imidazo[1,5-a]pyridin-3-yl}pyrrolidine-1-carboxylate (0.11 g, 0.22 mmol) in CH₃CN (2 mL) at 0° C. was added iodo(trimethyl)silane (0.17 mL, 1.18 mmol). The reaction mixture was allowed to warm to ambient temperature. After 20 min, an aqueous solution of 1N HCl and ether were added. The layers were separated and saturated NaHCO₃ was added to the aqueous layer. The basic aqueous layer was extracted with DCM (3×). The combined organic extracts were dried over Na₂SO₄, filtered and concentrated to give the title compound. HRMS: m/z found=365.2344 (M+1). ¹H NMR (500 MHz, CDCl₃) δ 8.26 (d, J=9.3 Hz, 1H), 8.17 (d, J=7.1 Hz, 1H), 6.97-6.94 (m, 2H), 6.66 (t, J=6.8 Hz, 1H), 4.53 (t, J=7.4 Hz, 1H), 3.23-3.21 (m, 1H), 3.04-3.02 (m, 1H), 2.25-2.19 (m, 7H), 2.13-2.00 (m, 3H), 1.99-1.91 (m, 4H), 1.77-1.70 (m, 6H).

| Example | Structure | Name | MS(M + 1)* |
|---|---|---|---|
| 538 | 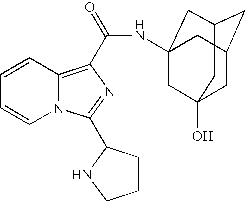 | N-(3-hydroxyadamantan-1-yl)-3- pyrrolidin-2-ylimidazo[1,5-a]pyridine-1- carboxamide | 381.2 |
| 539 | 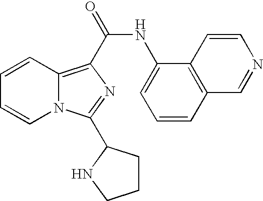 | N-isoquinolin-5-yl-3-pyrrolidin-2- ylimidazo[1,5-a]pyridine-1-carboxamide | 358.1 |
| 540 | 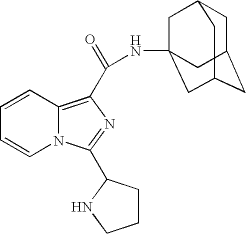 | N-adamantan-1-yl-3-pyrrolidin-2- ylimidazo[1,5-a]pyridine-1-carboxamide | 365.2 |
| 541 | 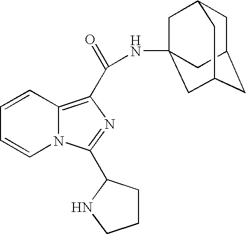 | N-adamantan-1-yl-3-pyrrolidin-2- ylimidazo[1,5-a]pyridine-1-carboxamide | 365.2 |
| 542 | 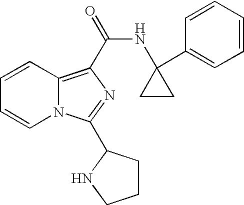 | N-(1-phenylcyclopropyl)-3-pyrrolidin-2- ylimidazo[1,5-a]pyridine-1-carboxamide | 347.1 |
| 695 | 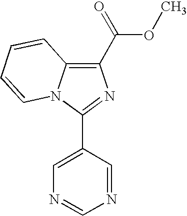 | Methyl 3-pyrimidin-5-ylimidazo[1,5- a]pyridine-1-carboxylate | 223.2 |
| 696 | 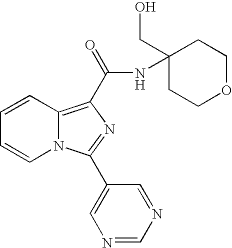 | N-[4-(hydroxymethyl)tetrahydro-2h- pyran-4-yl]-3-pyrimidin-5-ylimidazo[1,5- a]pyridine-1-carboxamide | 354.2 |
| 697 | 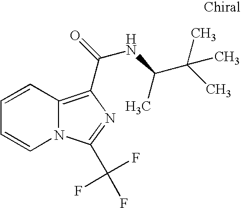 | 3-(Trifluoromethyl)-n-[(1r)-1,2,2- trimethylpropyl]imidazo[1,5-a]pyridine- 1-carboxamide | 314.1497 |
| 698 | 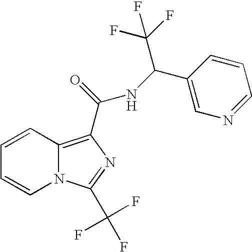 | 3-(Trifluoromethyl)-n-(2,2,2-trifluoro-1- pyridin-3-ylethyl)imidazo[1,5-a]pyridine- 1-carboxamide | 389.0815 |
| 699 | 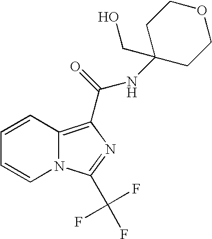 | N-[4-(hydroxymethyl)tetrahydro-2h- pyran-4-yl]-3- (trifluoromethyl)imidazo[1,5-a]pyridine- 1-carboxamide | 344.1197 |
| 700 | 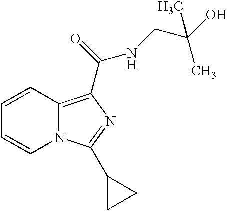 | 3-cyclopropyl-N-(2-hydroxy-2- methylpropyl)imidazo [1,5-a]pyridine-1- carboxamide | 274.1549 |

*Except as otherwise indicated

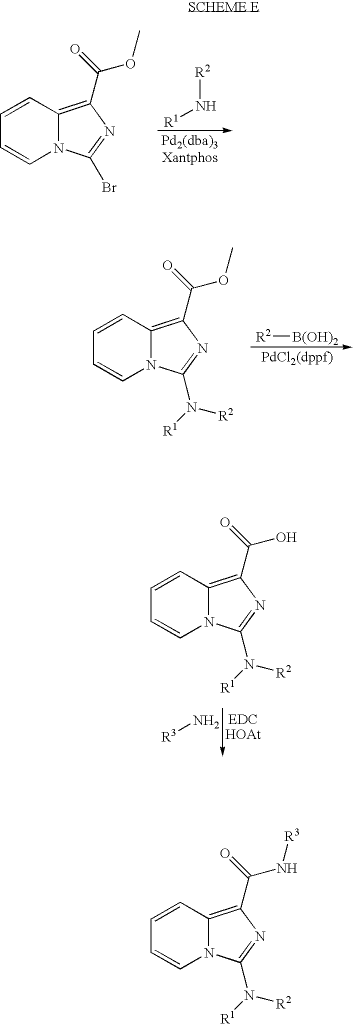

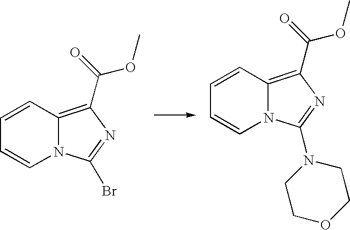

Methyl 3-morpholin-4-ylimidazo[1,5-a]pyridine-1-carboxylate. To a mixture of methyl 3-bromoimidazo[1,5-a]pyridine-1-carboxylate (1 g, 3.92 mmol), morpholine (683 mg, 7.84 mmol), Pd₂(dba)₃ (72 mg, 0.078 mmol), Xantphos (91 mg, 0.157 mmol) and Cs₂CO₃ (1.916 g, 5.88 mmol) was added 30 mL dioxane and heated at 100° C., under N₂. After 10 h, more morpholine, catalyst and ligand were added to the r×n mixture and heating at 100° C. was continued for 10 more hours. Cooled to room temperature and filtered through a pad of celite (celite pad was washed with CH₂Cl₂ for 3×). Combined filtrate and washings were concentrated and the resulting residue was subjected to flash chromatographic separation (silica gel, using a linear gradient of 0% to 8% meOH in CH₂Cl₂). Isolated product was still impure. It was further purified by r.p. HPLC and the desired product was obtained as a light yellow solid (free base, 610 mg, 60%).

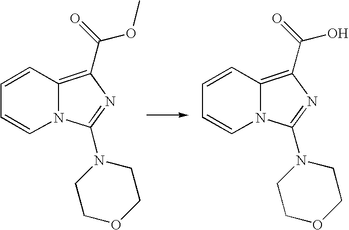

3-morpholin-4-ylimidazo[1,5-a]pyridine-1-carboxylic acid. To a solution of methyl 3-morpholin-4-ylimidazo[1,5-a]pyridine-1-carboxylate (610 mg, 2.335 mmol) in 1 mL MeOH was added aqueous NaOH (1 N, 2.335 mL) followed by H₂O (4 mL). The mixture was heated at 60° C. for 6 h. Then more aqueous NaOH (1 N, 0.235 mL) was added and heated at 60° C. overnight. Cooled to room temperature and aqueous HCl (1 N, 2.57 mL) was added. The resulting mixture was then concentrated to light yellow glass and was used as is in the next step.

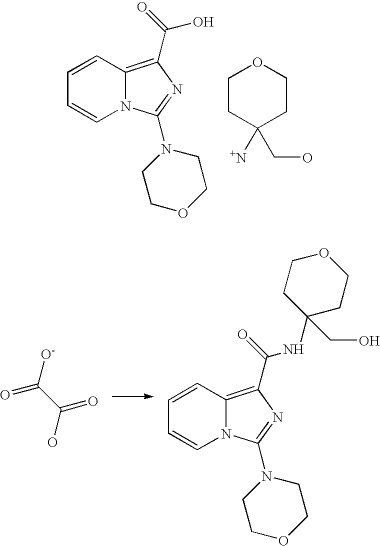

N-[1-(hydroxymethyl)cyclohexyl]-3-morpholin-4-ylimidazo[1,5-a]pyridine-1-carboxamide. To a mixture of 3-morpholin-4-ylimidazo[1,5-a]pyridine-1-carboxylic acid (69 mg, 0.279 mmol), (1-aminocyclohexyl)methanol (oxalic acid salt; 68 mg, 0.307 mmol), HOAt (49 mg, 0.363 mmol) and EDC (70 mg, 0.363 mmol) in DMF (1.3 mL) was added Hunig's base (180 mg, 1.395 mmol). The reaction mixture was then heated at 80° C. After 8 h, the desired product was separated by r.p. HPLC and obtained as a tan solid (free base, 62 mg, 62%). ¹H NMR (500 MHz, CDCl₃): δ 8.15 (d, J=9.3 Hz, 1H), 7.81 (d, J=7.3 Hz, 1H), 7.28 (s, 1H), 6.97 (dd, J=9.0, 6.4 Hz, 1H), 6.69 (t, J=6.6 Hz, 1H), 5.51 (br, 1H), 3.94-3.92 (m, 4H), 3.86-3.73 (m, 6H), 3.21-3.19 (m, 4H), 2.08-2.05 (m, 2H), 1.95-1.89 (m, 2H).

The following compounds were prepared using the procedures above:

TABLE E

| Example | Structure | Name | MS(M + 1)* |
|---|---|---|---|
| 543 | 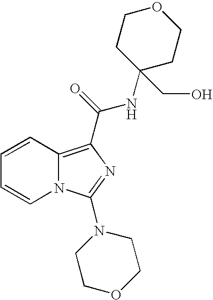 | N-[4-(hydroxymethyl)tetrahydro-2h-pyran- 4-yl]-3-morpholin-4-ylimidazo[1,5- a]pyridine-1-carboxamide | 361.1855 |
| 544 | 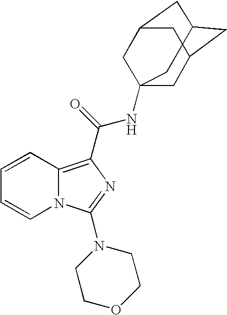 | N-1-adamantyl-3-morpholin-4- ylimidazo[1,5-a]pyridine-1-carboxamide | 381.2292 |
| 545 | 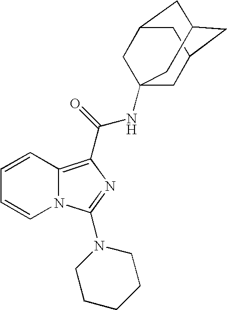 | N-1-adamantyl-3-piperidin-1- ylimidazo[1,5-a]pyridine-1-carboxamide | 379.2501 |
| 545 | 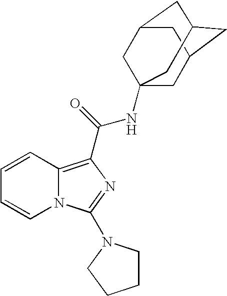 | N-1-adamantyl-3-pyrrolidin-1- ylimidazo[1,5-a]pyridine-1-carboxamide | 365.2318 |
| 546 | 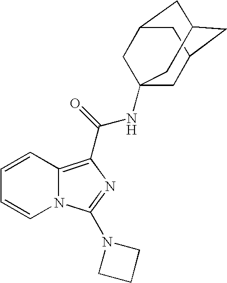 | N-1-adamantyl-3-azetidin-1- ylimidazo[1,5-a]pyridine-1-carboxamide | 351.2170 |
| 547 | 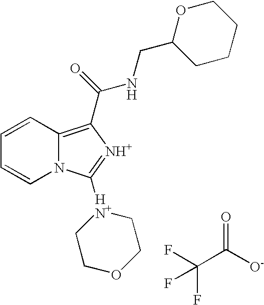 | 3-morpholin-4-ium-4-yl-1-{[(tetrahydro- 2H-pyran-2- ylmethyl)amino]carbonyl}imidazo[1,5- a]pyridin-2-ium bis(trifluoroacetate) | 345.192 |
| 548 | 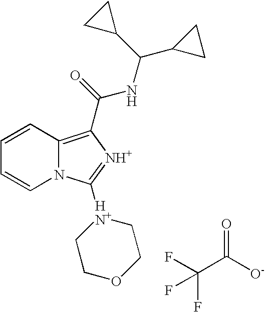 | 1- {[(dicyclopropylmethyl)amino]carbonyl}- 3-morpholin-4-ium-4-ylimidazo[1,5- a]pyridin-2-ium bis(trifluoroacetate) | 341.1971 |
| 549 | 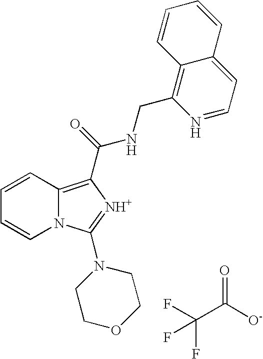 | 1-({[(3-morpholin-4-ylimidazo[1,5- a]pyridin-2-ium-1- yl)carbonyl]amino}methyl)isoquinolinium bis(trifluoroacetate) | 388.1766 |
| 550 | 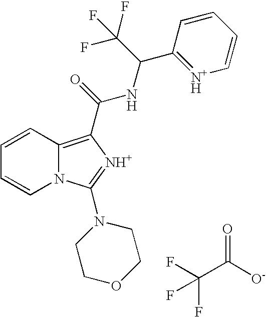 | 3-morpholin-4-yl-1-{[(2,2,2-trifluoro-1- pyridinium-2- ylethyl)amino]carbonyl}imidazo[1,5- a]pyridin-2-ium bis(trifluoroacetate) | 406.1484 |
| 551 | 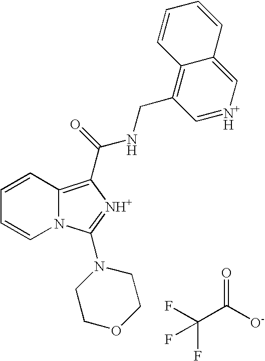 | 4-({[(3-morpholin-4-ylimidazo[1,5- a]pyridin-2-ium-1- yl)carbonyl]amino}methyl)isoquinolinium bis(trifluoroacetate) | 388.177 |
| 552 | 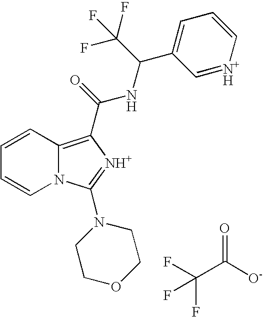 | 3-morpholin-4-yl-1-{[(2,2,2-trifluoro-1- pyridinium-3- ylethyl)amino]carbonyl}imidazo[1,5- a]pyridin-2-ium bis(trifluoroacetate) | 406.1482 |
| 553 | 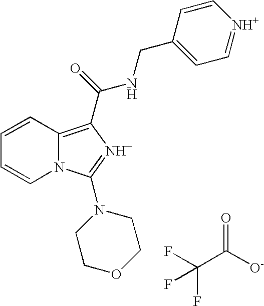 | 3-morpholin-4-yl-1-{[(pyridinium-4- ylmethyl)amino]carbonyl}imidazo[1,5- a]pyridin-2-ium bis(trifluoroacetate) | 338.161 |
| 554 | 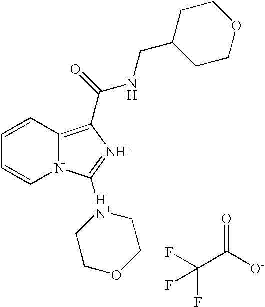 | 3-morpholin-4-ium-4-yl-1-{[(tetrahydro- 2H-pyran-4- ylmethyl)amino]carbonyl}imidazo[1,5- a]pyridin-2-ium bis(trifluoroacetate) | 345.1921 |
| 555 | 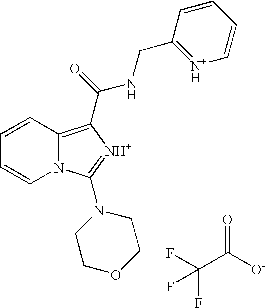 | 3-morpholin-4-yl-1-{[(pyridinium-2- ylmethyl)amino]carbonyl}imidazo [1,5- a]pyridin-2-ium bis(trifluoroacetate) | 338.161 |
| 556 | 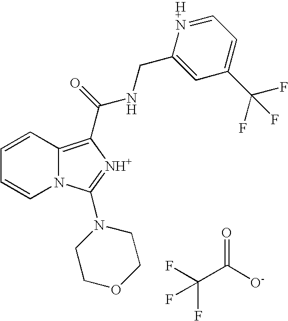 | 3-morpholin-4-yl-1-[({[4- (trifluoromethyl)pyridinium-2- yl]methyl}amino)carbonyl]imidazo[1,5- a]pyridin-2-ium bis(trifluoroacetate) | 406.1481 |
| 557 | 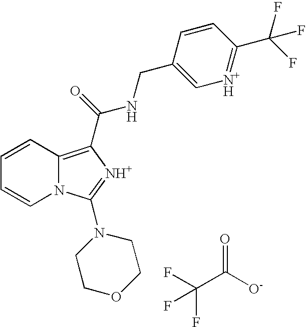 | 3-morpholin-4-yl-1-[({[6- (trifluoromethyl)pyridinium-3- yl]methyl}amino)carbonyl]imidazo[1,5- a]pyridin-2-ium bis(trifluoroacetate) | 406.1485 |
| 558 | 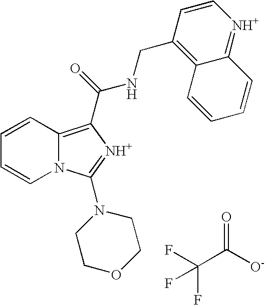 | 4-({[(3-morpholin-4-ylmidazo[1,5- a]pyridin-2-ium-1- yl)carbonyl]amino}methyl)quinolinium bis(trifluoroacetate) | 388.1772 |
| 559 | 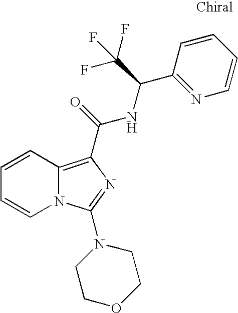 | 3-morpholin-4-yl-n-[(1r)-2,2,2-trifluoro-1- pyridin-2-ylethyl]imidazo[1,5-a]pyridine- 1-carboxamide | 406.1481 |
| 560 | 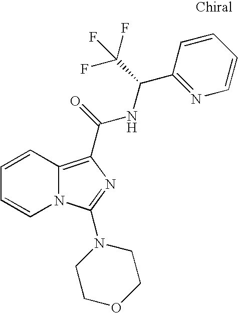 | 3-morpholin-4-yl-n-[(1s)-2,2,2-trifluoro-1- pyridin-2-ylethyl]imidazo[1,5-a]pyridine- 1-carboxamide | 406.1483 |
| 561 | 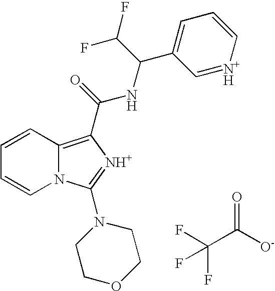 | 1-{[(2,2-difluoro-1-pyridinium-3- ylethyl)amino]carbonyl}-3-morpholin-4- ylimidazo[1,5-a]pyridin-2-ium bis(trifluoroacetate) | 388.1584 |
| 562 | 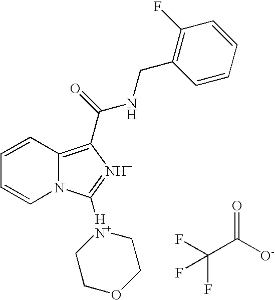 | 1-{[(2-fluorobenzyl)amino]carbonyl}-3- morpholin-4-ium-4-ylimidazo[1,5- a]pyridin-2-ium bis(trifluoroacetate) | 355.1574 |
| 563 | 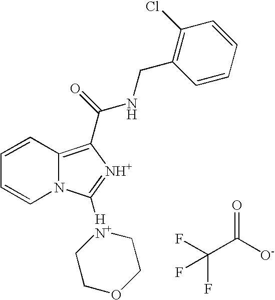 | 1-{[(2-chlorobenzyl)amino]carbonyl}-3- morpholin-4-ium-4-ylimidazo[1,5- a]pyridin-2-ium bis(trifluoroacetate) | 371.1275 |
| 564 | 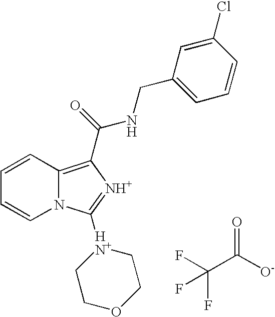 | 1-{[(3-chlorobenzyl)amino]carbonyl}-3- morpholin-4-ium-4-ylimidazo[1,5- a]pyridin-2-ium bis(trifluoroacetate) | 371.1274 |
| 565 | 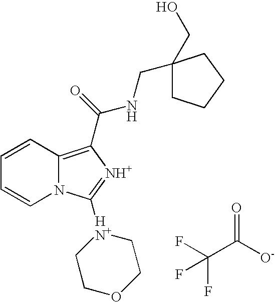 | 1-[({[1- (hydroxymethyl)cyclopentyl]methyl}amino) carbonyl]-3-morpholin-4-ium-4- ylimidazo[1,5-a]pyridin-2-ium bis(trifluoroacetate) | 359.2087 |
| 566 | 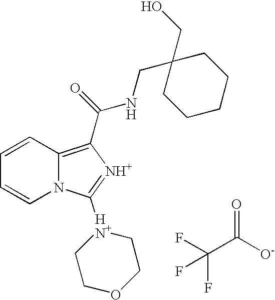 | 1-[({[1- (hydroxymethyl)cyclohexyl]methyl}amino) carbonyl]-3 -morpholin-4-ium-4- ylimidazo[1,5-a]pyridin-2-ium bis(trifluoroacetate) | 373.2242 |
| 567 | 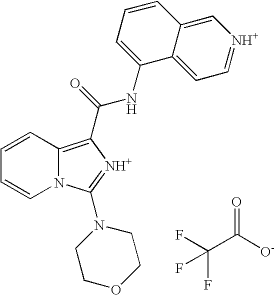 | 5-{[(3-morpholin-4-ylimidazo[1,5- a]pyridin-2-ium-1- yl)carbonyl]amino}isoquinolinium bis(trifluoroacetate) | 374.1617 |
| 568 | 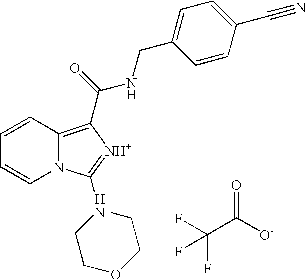 | 1-{[(4-cyanobenzyl)amino]carbonyl}-3- morpholin-4-ium-4-ylimidazo[1,5- a]pyridin-2-ium bis(trifluoroacetate) | 362.1633 |
| 569 | 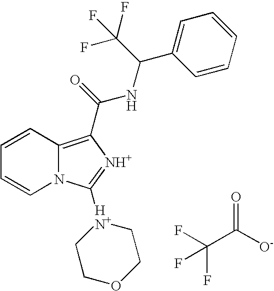 | 3-morpholin-4-ium-4-yl-1-{[(2,2,2- trifluoro-1- phenylethyl)amino]carbonyl}imidazo[1,5- a]pyridin-2-ium bis(trifluoroacetate) | 405.1535 |
| 570 | 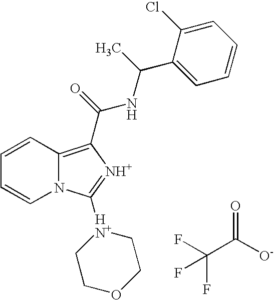 | 1-({[1-(2- chlorophenyl)ethyl]amino}carbonyl)-3- morpholin-4-ium-4-ylimidazo[1,5- a]pyridin-2-ium bis(trifluoroacetate) | 385.1430 |
| 571 | 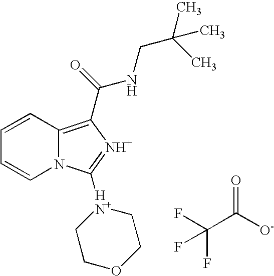 | 1-{[(2,2-dimethylpropyl)amino]carbonyl}- 3-morpholin-4-ium-4-ylimidazo[1,5- a]pyridin-2-ium bis(trifluoroacetate) | 317.1989 |
| 572 | 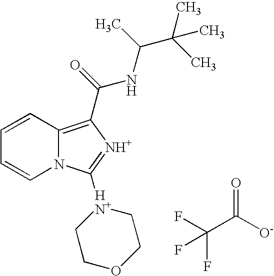 | 3-morpholin-4-ium-4-yl-1-{[(1,2,2- trimethylpropyl)amino]carbonyl}imidazo [1,5-a]pyridin-2-ium bis(trifluoroacetate) | 331.2142 |
| 573 | 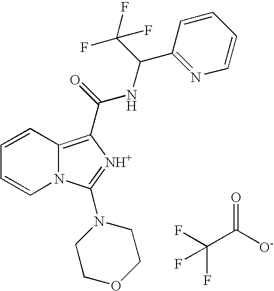 | 3-piperidin-1-yl-n-(2,2,2-trifluoro-1- pyridin-2-ylethyl)imidazo[1,5-a]pyridine- 1-carboxamide | 404.1694 |
| 574 | 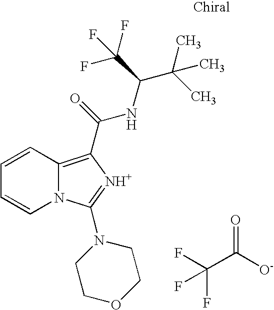 | 4-[1-({[(1r)-2,2-dimethyl-1- (trifluoromethyl)propyl]amino}carbonyl) imidazo[1,5-a]pyridin-3-yl]morpholin-4- ium trifluoroacetate | 385.1842 |
| 575 | 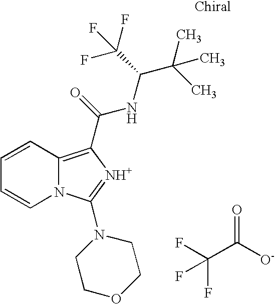 | 4-[1-({[(1s)-2,2-dimethyl-1- (trifluoromethyl)propyl]amino}carbonyl) imidazo[1,5-a]pyridin-3-yl]morpholin-4- ium trifluoroacetate | 385.1842 |
| 576 | 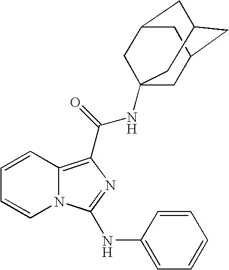 | N-1-adamantyl-3-anilinoimidazo[1,5- a]pyridine-1-carboxamide | 387.2211 |
| 701 | 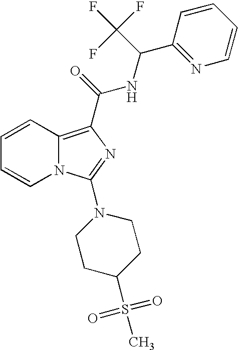 | 3-[4-(methylsulfony1)piperidin-1-yl]-N- (2,2,2-trifluoro-1-pyridin-2- ylethyl)imidazo[1,5-a]pyridine-1- carboxamide | 482.1475 |
| 702 | 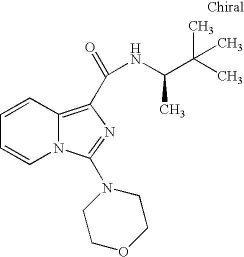 | 3-morpholin-4-yl-n-[(1r)-1,2,2- trimethylpropyl]imidazo[1,5-a]pyridine-1- carboxamide | 331.2117 |

### Example 577

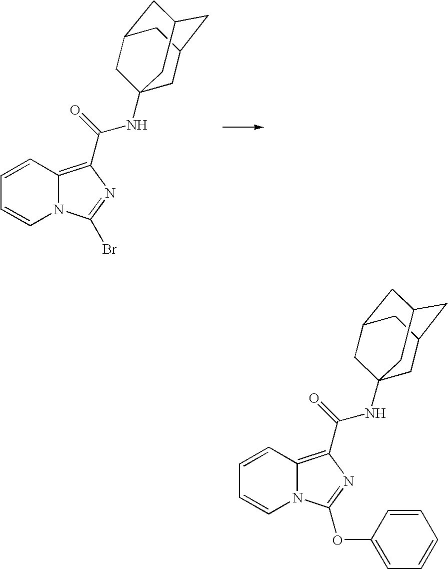

N-1-adamantyl-3-phenoxyimidazo[1,5-a]pyridine-1-carboxamide. A mixture of N-1-adamantyl-3-bromoimidazo[1,5-a]pyridine-1-carboxamide (200 mg, 0.534 mmol), phenol (75 mg, 0.802 mmol), N,N-dimethylglycine hydrochloride (67 mg, 0.481 mmol), CuI (31 mg, 0.160 mmol) and Cs₂CO₃ (522 mg, 1.603 mmol) in dioxane (3 mL) was heated at 110° C. overnight. The cooled reaction mixture was the filtered through a pad of celite (washing with CH₂Cl₂ 3×). The combined filtrate and washings were concentrated and purified by r.p. HPLC. Desired product was obtained as a white solid (free base, 100 mg, 48%). ¹H NMR (500 MHz, CDCl₃): δ 8.24-8.21 (m, 1H), 7.77-7.75 (m, 1H), 7.47-7.37 (m, 2H), 7.21-7.18 (m, 3H), 6.92-6.89 (m, 1H), 6.71 (br, 1H), 6.64-6.61 (m, 1H), 2.16 (d, J=2.4 Hz, 6H), 2.11 (s, 3H), 1.72 (dd, J=22.9, 12.2 Hz, 6H).

The following compounds were prepared according to this procedure:

| | | | |
|---|---|---|---|
| 577 | 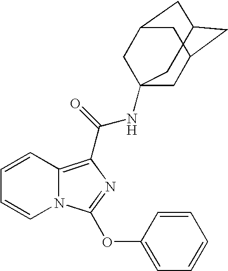 | N-1-adamantyl-3- phenoxylmidazo[1,5-a]pyridine- 1-carboxamide | 388.2033 |
| 578 | 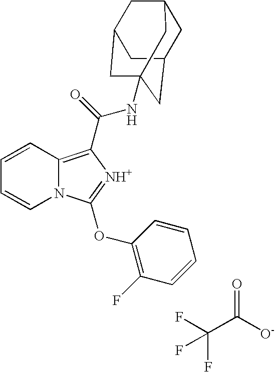 | 1-[(1- adamantylamino)carbonyl]-3-(2- fluorophenoxy)imidazo[1,5- a]pyridin-2-ium trifluoroacetate | 406.1943 |
| 579 | 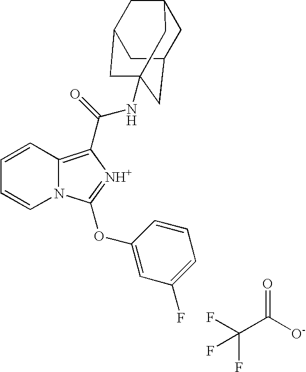 | 1-[(1- adamantylamino)carbonyl]-3-(3- fluorophenoxy)imidazo[1,5- a]pyridin-2-ium trifluoroacetate | 406.1944 |
| 580 | 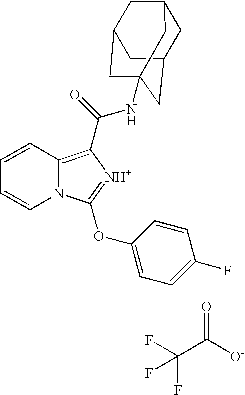 | 1-[(1- adamantylamino)carbonyl]-3-(4- fluorophenoxy)imidazo[1,5- a]pyridin-2-ium trifluoroacetate | 406.1936 |
| 581 | 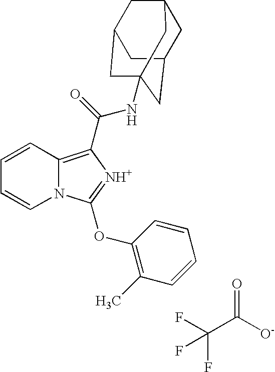 | 1-[(1- adamantylamino)carbonyl]-3-(2- methylphenoxy)imidazo[1,5- a]pyridin-2-ium trifluoroacetate | 402.2203 |
| 582 | 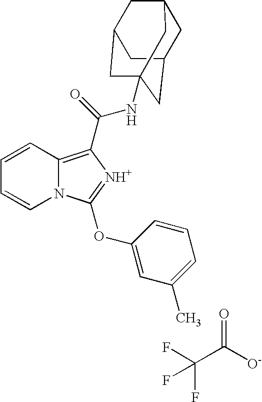 | 1-[(1- adamantylamino)carbonyl]-3-(3- methylphenoxy)imidazo[1,5- a]pyridin-2-ium trifluoroacetate | 402.2197 |
| 583 | 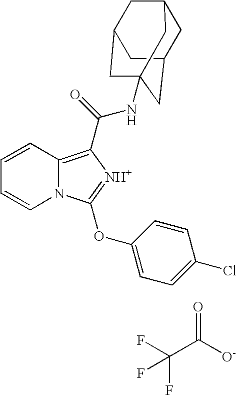 | 1-[(1- adamantylamino)carbonyl]-3-(4- chlorophenoxy)imidazo[1,5- a]pyridin-2-ium trifluoroacetate | 422.1641 |
| 584 | 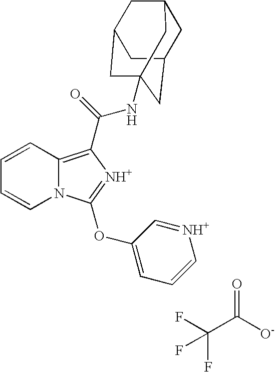 | 1-[(1- adamantylamino)carbonyl]-3- (pyridinium-3- yloxy)imidazo[1,5-a]pyridin-2- ium bis(trifluoroacetate) | 389.1983 |
| 585 | 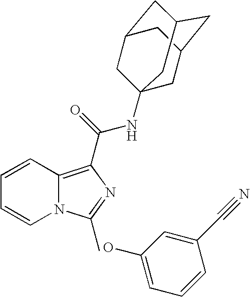 | N-1-adamantyl-3-(3- cyanophenoxy)imidazo[1,5- a]pyridine-1-carboxamide | 413.1983 |

### Example 586

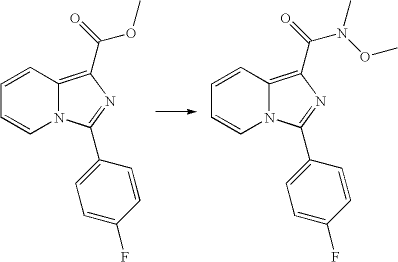

To a mixture of sm (1 g, 3.7 mmol) and N,O-dimethylhydroxylamine hydrochloride (794 mg, 8.14 mmol) in 50 mL CH₂Cl₂ at 0° C. was added a solution of Me₃Al (2 M in toluene, 4.07 mL). The cooling bath was then removed and the reaction mixture was stirred at room temperature for 3.5 h. Quenched by adding satd. aqueous NaHCO₃. Then satd. aqueous K—Na-tartrate (50 mL) was added. After stirring overnight layers were separated. Aqueous layer was extracted with CH₂Cl₂ (3×). Combined organic layers were dried over Na₂SO₄ and concentrated. Desired product was separated by flash chromatography (silica gel) using a linear gradient of 3% to 90% EtOAc in hexanes (white sticky solid, 980 mg, 88%).

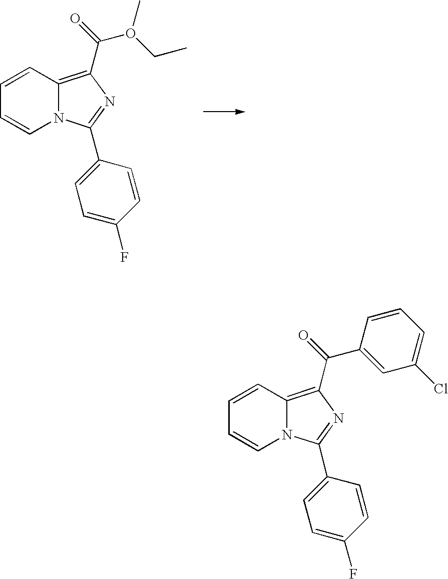

To a solution of 3-bromochlorobenzene (576 mg, 3.01 mmol) in THF (8 mL) at −78° C. was added n-BuLi solution (2.5 M, 1.203 mL) dropwise and stirred for 15 min. To this mixture a solution of sm (300 mg, 1.002 mmol) in 2 mL THF was added via canula (rinse with 1 mL THF) slowly. The resulting light red solution was stirred at −78° C. for 30 min and the quenched by adding satd. aqueous NH₄Cl. Partitioned between satd. aqueous NaHCO₃ and CH₂Cl₂. Layers were separated. Aqueous layer was extracted with CH₂Cl₂ (3×). Combined organic layers were dried over Na₂SO₄ and concentrated. Desired product was separated by flash chromatography (silica gel) using a linear gradient of 3% to 40% EtOAc in hexanes (yellow solid, 324 mg, 92%).

| | | | |
|---|---|---|---|
| 586 | 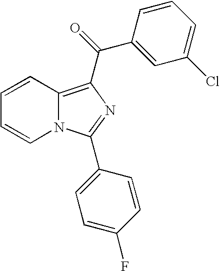 | (3-chlorophenyl)[3-(4- fluorophenyl)imidazo[1,5- a]pyridin-1-yl]methanone | 351.0678 |

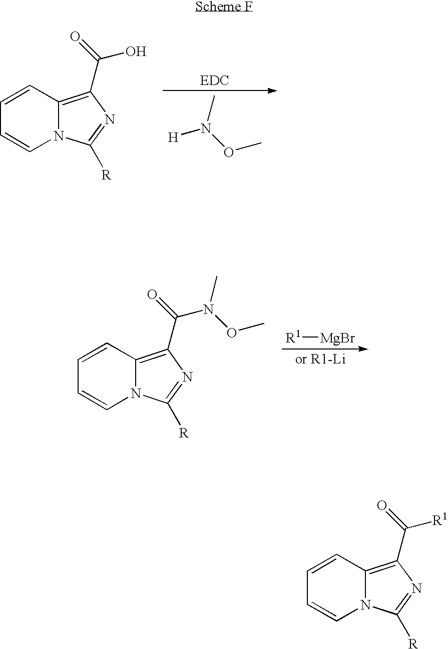

| | | | |
|---|---|---|---|
| 587 | 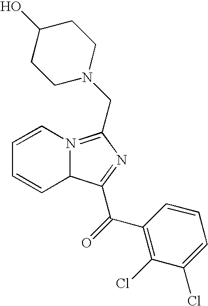 | (2,3-dichlorophenyl){3-[(4- hydroxypiperidin-1- yl)methyl]imidazo[1,5-a]pyridin-1- yl}methanone | 404.0941 |
| 588 | 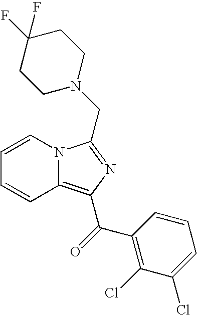 | (2,3-dichlorophenyl){3-[(4,4- difluoropiperidin-1- yl)methyl]imidazo[1,5-a]pyridin-1- yl}methanone | 424.0799 |
| 589 | 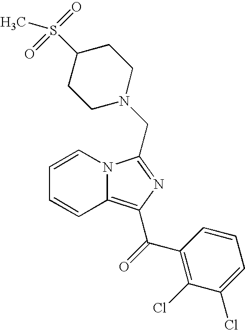 | (2,3-dichlorophenyl)(3-{[4- (methylsulfonyl)piperidin-1- yl]methyl}imidazo [1,5-a]pyridin-1- yl)methanone | 466.075 |
| 590 | 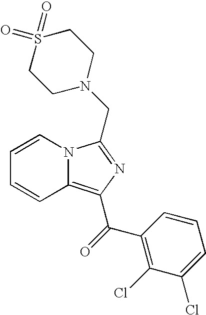 | (2,3-dichlorophenyl){3-[(1,1- dioxidothiomorpholin-4- yl)methyl]imidazo [1,5-a]pyridin-1- yl}methanone | 438.043 |
| 591 | 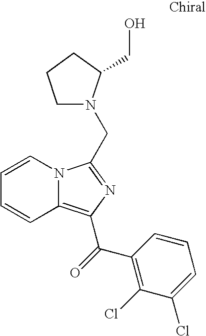 | (2,3-dichlorophenyl)(3-{[(2r)-2- (hydroxymethyl)pyrrolidin-1- yl]methyl}imidazo[1,5-a]pyridin-1- yl)methanone | 404.0913 |
| 592 | 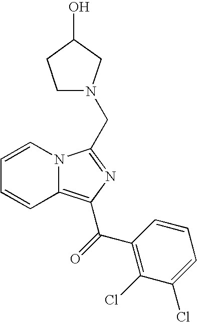 | (2,3-dichlorophenyl){3-[(3- hydroxypyrrolidin-1- yl)methyl]imidazo[1,5-a]pyridin-1- yl}methanone | 390.0755 |
| 593 | 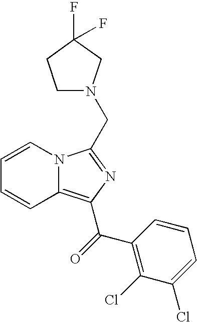 | (2,3-dichlorophenyl){3-[(3,3- difluoropyrrolidin-1- yl)methyl]imidazo[1,5-a]pyridin-1- yl}methanone | 410.0619 |
| 594 | 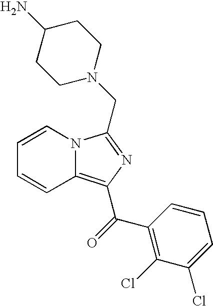 | {3-[(4-aminopiperidin-1- yl)methyl]imidazo[1,5-a]pyridin-1- yl}(2,3-dichlorophenyl)methanone | 403.1018 |
| 595 | 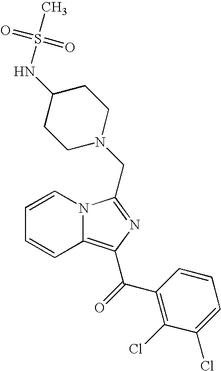 | N-(1-{[1-(2,3- dichlorobenzoyl)imidazo[1,5-a]pyridin- 3-yl]methyl}piperidin-4- yl)methanesulfonamide | 481.0853 |
| 596 | 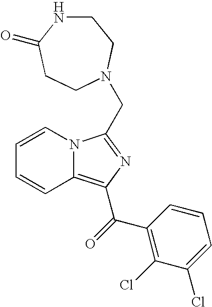 | 1-{[1-(2,3-dichlorobenzoyl)imidazo[1,5- a]pyridin-3-yl]methyl}-1,4-diazepan-5- one | 417.0873 |
| 597 | 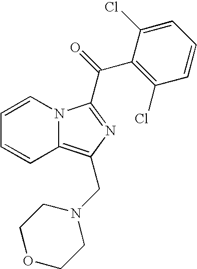 | (2,6-dichlorophenyl)[1-(morpholin-4- ylmethyl)imidazo[1,5-a]pyridin-3- yl]methanone | 390.0752 |
| 598 | 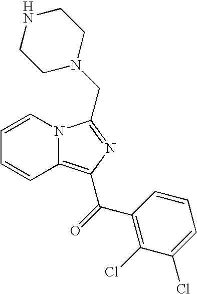 | (2,3-dichlorophenyl)[3-(piperazin-1- ylmethyl)imidazo[1,5-a]pyridin-1- yl]methanone | 389.0917 |
| 599 | 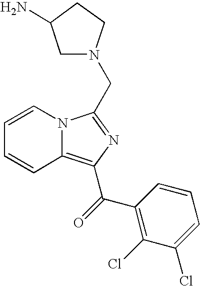 | {3-[(3-aminopyrrolidin-1- yl)methyl]imidazo[1,5-a]pyridin-1- yl}(2,3-dichlorophenyl)methanone | 389.092 |
| 600 | 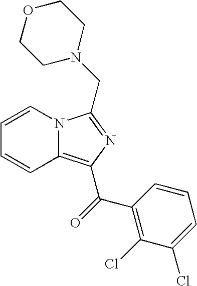 | (2,3-dichlorophenyl)[3-(morpholin-4- ylmethyl)imidazo[1,5-a]pyridin-1- yl]methanone | 390.0743 |
| 601 | 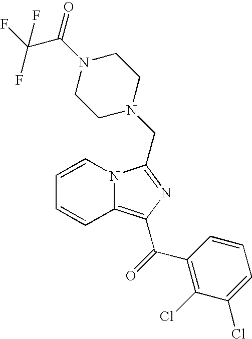 | (2,3-dichlorophenyl)(3-{[4- (trifluoroacetyl)piperazin-1- yl]methyl}imidazo[1,5-a]pyridin-1- yl)methanone | 485.0738 |
| 602 | 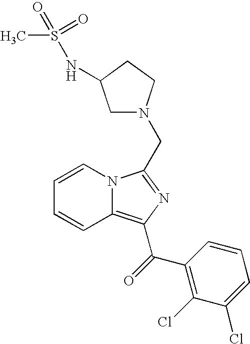 | N-(1-{[1-(2,3- dichlorobenzoyl)imidazo[1,5-a]pyridin- 3-yl]methyl}pyrrolidin-3- yl)methanesulfonamide | 467.0689 |

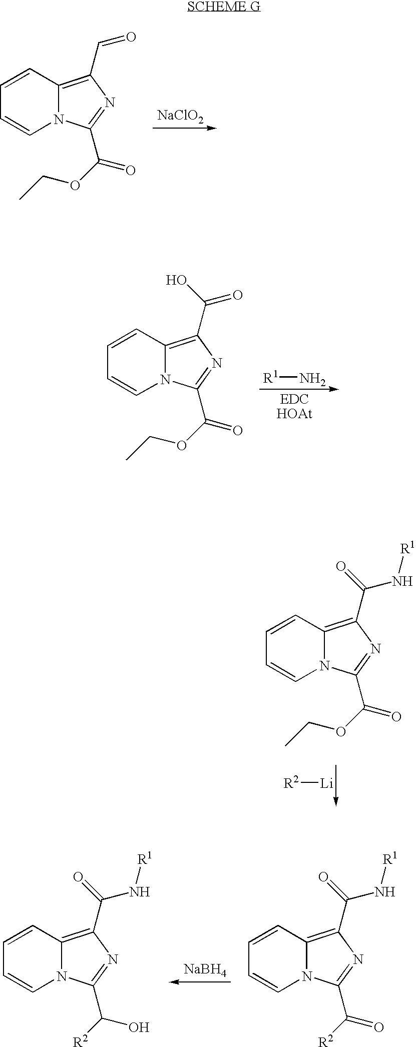

### Example 603

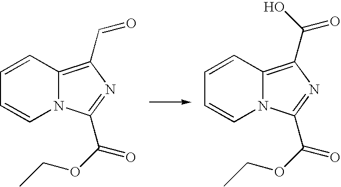

3-(ethoxycarbonyl)imidazo[1,5-a]pyridine-1-carboxylic acid. To a suspension of ethyl 1-formylimidazo[1,5-a]pyridine-3-carboxylate (100 mg, 0.458 mmol) in t-BuOH (4 mL) was added 2-methyl-2-butene (2 mL). To this resulting mixture was added a solution of NaClO₂ (108 mg, 1.192 mmol)/NaHPO₄.H₂O (164 mg, 1.192 mmol) in 2 mL H₂O. After stirring for 7 h a solution of NaClO₂ (108 mg, 1.192 mmol)/NaHPO₄.H₂O (164 mg, 1.192 mmol) in 1 mL H₂O was added to the reaction mixture and stirring was continued overnight. The resulting turbid solution was then partitioned between water and EtOAc. Layers were separated. Aqueous layer was saturated with NaCl and then extracted with EtOAc (3×). Combined organic layers were dried over Na2SO4 and concentrated to yield the desired product as a yellow solid (89 mg, 83%).

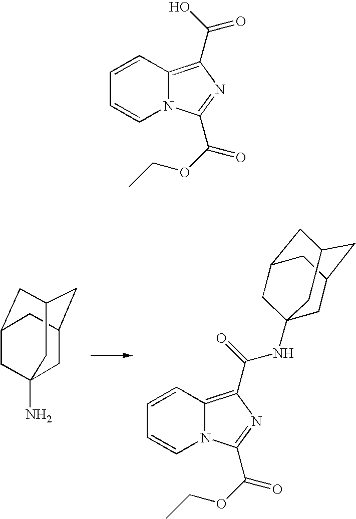

Ethyl1-[(1-adamantylamino)carbonyl]imidazo[1,5-a]pyridine-3-carboxylate. To a mixture of 3-(ethoxycarbonyl)imidazo[1,5-a]pyridine-1-carboxylic acid (140 mg, 0.598 mmol), adamantyl amine (136 mg, 1.196 mmol), EDC (229 mg, 1.196 mmol) and HOAT (81 mg, 0.598 mmol) in 4 mL DMF was added Et₃N (242 mg, 2.391 mmol) and then heated at 60° C. for 6 h. Cooled to room temperature and partitioned between water and CH₂Cl₂. Layers were separated and the aqueous layer was extracted with CH₂Cl₂ (3×). Combined organic layers were dried over Na₂SO₄ and concentrated. Desired product was then separated by flash chromatography (silica gel, using a linear gradient of 3% to 40% EtOAc in hexanes) and obtained as a light yellow solid (167 mg, 76%).

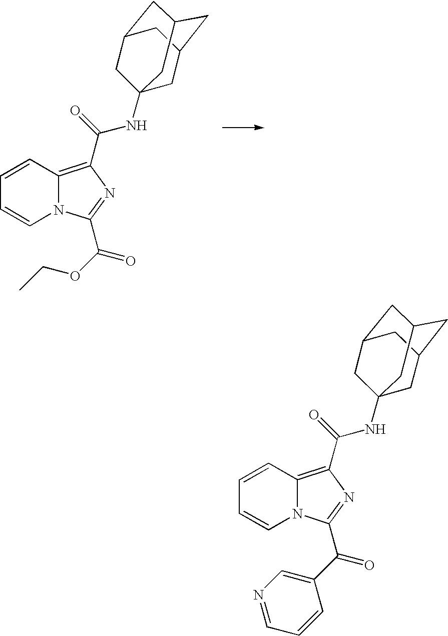

N-1-adamantyl-3-(pyridin-3-ylcarbonyl)imidazo[1,5-a]pyridine-1-carboxamide. A solution of 3-bromopyridine (144 mg, 0.909 mmol) in ether (5 mL) was treated with n-BuLi at −78° C. and stirred at −78° C. for 50 min. Light yellow precipitate of 3-lithiopyridine appeared. Then add a solution of ethyl1-[(1-adamantylamino)carbonyl]imidazo[1,5-a]pyridine-3-carboxylate (167 mg, 0.454 mmol), precooled to −78° C., to the 3-lithiopyridine suspension via canula. After stirring at −78° C. for 1.5 h, the cooling bath was removed and the reaction mixture was allowed to warm up to room temperature. The reaction mixture was then quenched by adding satd. aqueous NH₄Cl and partitioned between satd. aqueous NaHCO₃ and CH₂Cl₂. Layers were separated and the aqueous layer was extracted with CH₂Cl₂ (3×). Combined organic layers were dried over Na₂SO₄ and concentrated. Desired product was separated by r.p. HPLC and obtained as a yellow solid (free base, 324 mg, 92%). ¹H NMR (500 MHz, CDCl₃): δ 9.83 (d, J=7.1 Hz, 1H), 9.63-9.62 (m, 1H) 8.82 (dd, J=4.9, 1.7 Hz, 1H), 8.64 (dd, J=9.0, 7.8 Hz, 1H), 8.57-8.55 (m, 1H), 7.51-7.44 (m, 2H), 7.19-7.05 (m, 1H), 6.97 (s, 1H), 2.19, (d, J=2.2 Hz, 6H), 2.15 (s, 3H), 1.75 (dd, J=20.0, 12.7 Hz, 6H).

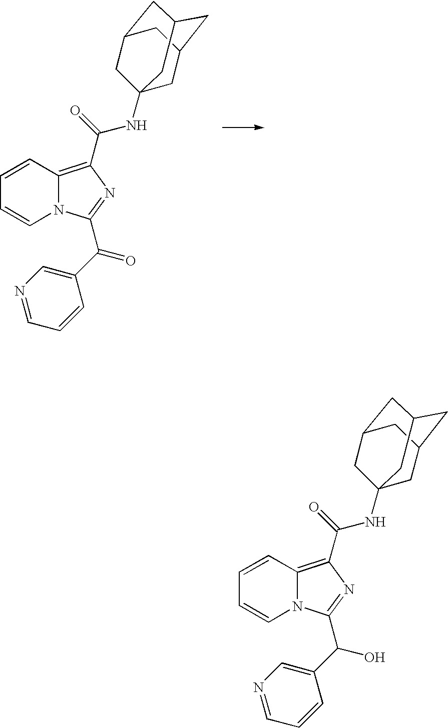

N-1-adamantyl-3-[hydroxy(pyridin-3-yl)methyl]imidazo[1,5-a]pyridine-1-carboxamide. Solid NaBH₄ (13 mg, 0.33 mmol) was added to a stirring suspension of N-1-adamantyl-3-(pyridin-3-ylcarbonyl)imidazo[1,5-a]pyridine-1-carboxamide (66 mg, 0.165 mmol) in 5 mL MeOH at room temperature. Stirred for 30 min. To the resulting clear solution was added aqueous 1 N HCl (1 mL) and water (4 mL). Desired product was isolated as free base by passing the acidic solution through a column containing cation exchange resin (Strata column) (white solid, 66 mg, 99%). ¹H NMR (500 MHz, CDCl₃): δ 8.66 (d, J=1.2 Hz, 1H), 8.56 (d, J=4.6 Hz, 1H), 8.10-8.09 (m, 1H), 7.77 (dd, J=7.3, 1.0 Hz, 1H), 7.68 (d, J=8.1 Hz, 1H), 7.00 (s, 1H), 6.85 (dd, J=9.3, 6.6 Hz, 1H), 6.47 (t, J=7.1 Hz, 1H), 6.28 (d, J=4.2 Hz, 1H), 4.33 (d, J=4.2 Hz, 1H), 2.23 (s, 6H), 2.16 (s, 3H), 1.76 (dd, J=22.7, 12.2, Hz, 6H).

| | | | |
|---|---|---|---|
| 603 | 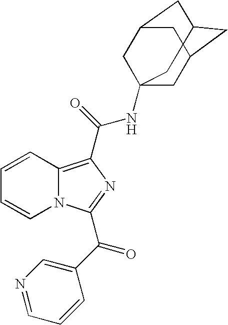 | N-1-adamantyl-3-(pyridin-3- ylcarbonyl)imidazo[1,5-a]pyridine- 1-carboxamide | 401.2011 |
| 604 | 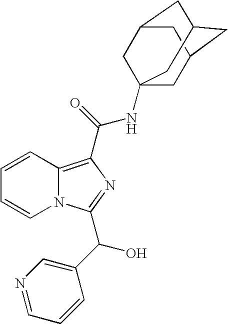 | N-1-adamantyl-3-[hydroxy(pyridin- 3-yl)methyl]imidazo[1,5-a]pyridine- 1-carboxamide | 403.2174 |
| 605 | 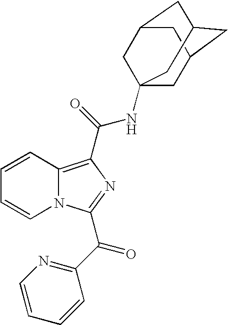 | N-1-adamantyl-3-(pyridin-2- ylcarbonyl)imidazo[1,5-a]pyridine- 1-carboxamide | 401.1997 |
| 606 | 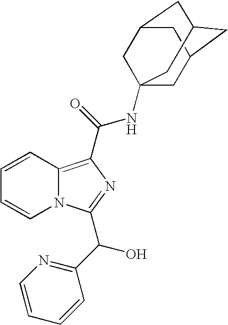 | N-1-adamantyl-3-[hydroxy(pyridin- 2-yl)methyl]imidazo[1,5-a]pyridine- 1-carboxamide | 403.2156 |
| 607 | 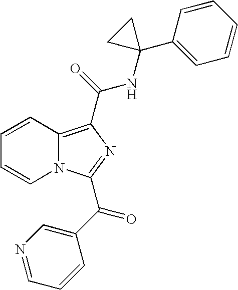 | N-(1-phenylcyclopropyl)-3-(pyridin- 3-ylcarbonyl)imidazo[1,5- a]pyridine-1-carboxamide | 383.1523 |
| 608 | 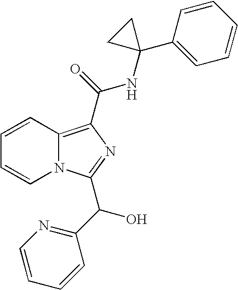 | 3-[hydroxy(pyridin-2-yl)methyl]-n- (1-phenylcyclopropyl)imidazo[1,5- a]pyridine-1-carboxamide | 385.1674 |
| 609 | 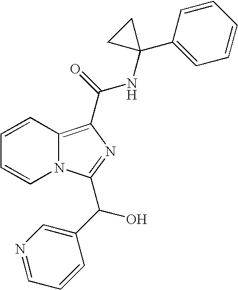 | 3-[hydroxy(pyridin-3-yl)methyl]-n- (1-phenylcyclopropyl)imidazo[1,5- a]pyridine-1-carboxamide | 385.1673 |
| 610 | 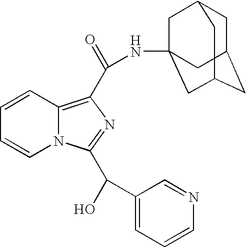 | N-adamantan-1-yl-1- [hydroxy(pyridin-3- yl)methyl]imidazo[1,5-a]pyridine-3- carboxamide | 403.2 |

The following compounds were synthesized from intermediates analogous to X using standard ester hydrolysis and peptide coupling procedures

| | | | |
|---|---|---|---|
| 611 | 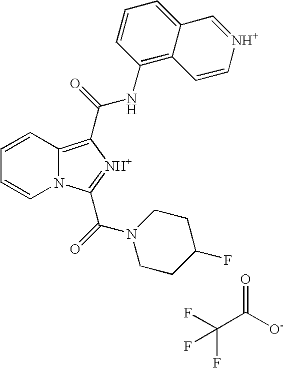 | 3-[(4-fluoropiperidin-1-yl)carbonyl]- N-isoquinolin-5-ylimidazo[1,5- a]pyridine-1-carboxamide | 418.21 |
| 612 | 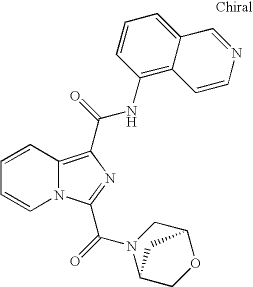 | N-isoquinolin-5-yl-3 -[(1S,4S)-2-oxa- 5-azabicyclo[2.2.1]hept-5- ylcarbonyl]imidazo[1,5-a]pyridine-1- carboxamide | 414.2 |
| 613 | 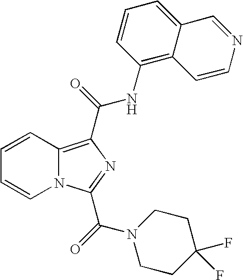 | 3-[(4,4-difluoropiperidin-1- yl)carbonyl]-N-isoquinolin-5- ylimidazo[1,5-a]pyridine-1- carboxamide | 436.19 |
| 614 | 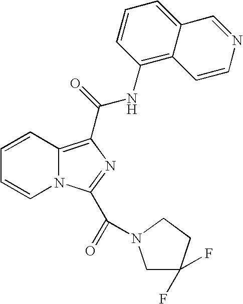 | 3-[(3,3-difluoropyrrolidin-1- yl)carbonyl]-N-isoquinolin-5- carboxamide | 422.18 |
| 615 | 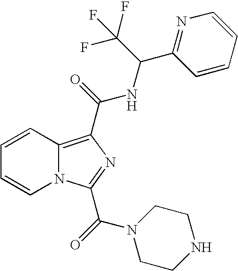 | 3-(piperazin-1-ylcarbonyl)-N-(2,2,2- trifluoro-1-pyridin-2- ylethyl)imidazo[1,5-a]pyridine-1- carboxamide | 433.158 |
| 616 | 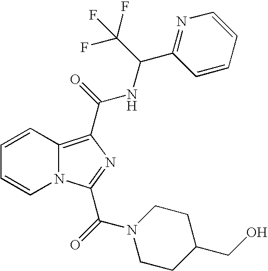 | 3-{[4-(hydroxymethyl)piperidin-1- yl]carbonyl}-N-(2,2,2-trifluoro-1- pyridin-2-ylethyl)imidazo[1,5- a]pyridine-1-carboxamide | 462.1724 |
| 617 | 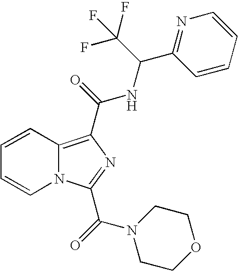 | 3-(morpholin-4-ylcarbonyl)-N- (2,2,2-trifluoro-1-pyridin-2- ylethyl)imidazo[1,5-a]pyridine-1- carboxamide | 434.1418 |
| 618 | 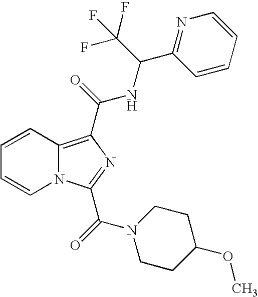 | 3-[(4-methoxypiperidin-1- yl)carbonyl]-N-(2,2,2-trifluoro-1- pyridin-2-ylethyl)imidazo[1,5- a]pyridine-1-carboxamide | 462.1734 |
| 619 | 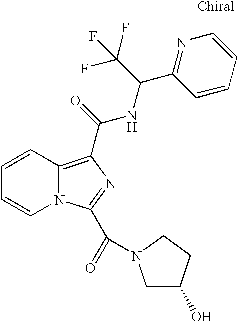 | 3-{[(3S)-3-hydroxypyrrolidin-1- yl]carbonyl}-N-(2,2,2-trifluoro-1- pyridin-2-ylethyl)imidazo[1,5- a]pyridine-1-carboxamide | 434.142 |
| 620 | 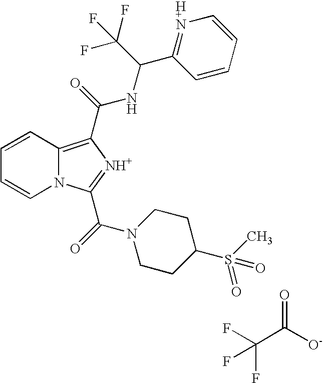 | 3-{[4-(methylsulfonyl)piperidin-1- yl]carbonyl}-N-(2,2,2-trifluoro-1- pyridin-2-ylethyl)imidazo[1,5- a]pyridine-1-carboxamide | 510.1400 |
| 621 | 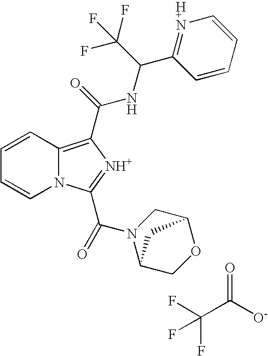 | 3-[(1S,4S)-2-oxa-5- azabicyclo[2.2.1]hept-5-ylcarbonyl]- N-(2,2,2-trifluoro-1-pyridin-2- ylethyl)imidazo[1,5-a]pyridine-1- carboxamide | 446.1418 |
| 622 | 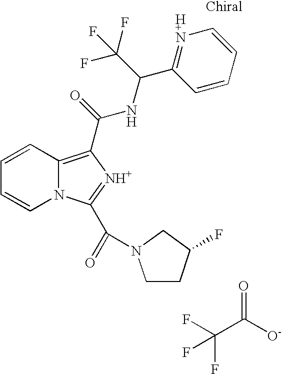 | 3-{[(3R)-3-fluoropyrrolidin-1- yl]carbonyl}-N-(2,2,2-trifluoro-1- pyridin-2-ylethyl)imidazo[1,5- a]pyridine-1-carboxamide | 436.1376 |
| 623 | 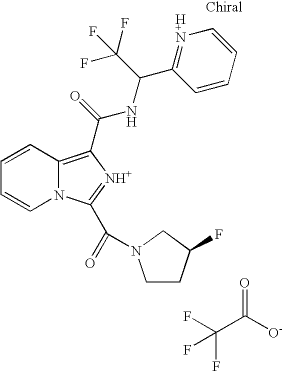 | 3-{[(3S)-3-fluoropyrrolidin-1- yl]carbonyl}-N-(2,2,2-trifluoro-1- pyridin-2-ylethyl)imidazo[1,5- a]pyridine-1-carboxamide | 436.1375 |
| 624 | 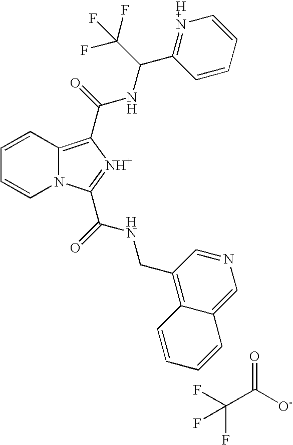 | N~3~-(isoquino1in-4-ylmethyl)- N~1~-(2,2,2-trifluoro-1-pyridin-2- ylethyl)imidazo[1,5-a]pyridine-1,3- dicarboxamide | 505.1574 |
| 625 | 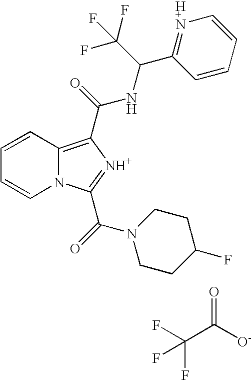 | 3-[(4-fluoropiperidin-1-yl)carbonyl]- N-(2,2,2-trifluoro-1-pyridin-2- ylethyl)imidazo[1,5-a]pyridine-1- carboxamide | 450.1532 |
| 626 | 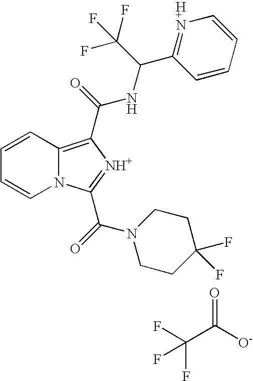 | 3-[(4,4-difluoropiperidin-1- yl)carbonyl]-N-(2,2,2-trifluoro-1- pyridin-2-ylethyl)imidazo[1,5- a]pyridine-1-carboxamide | 468.1435 |
| 627 | 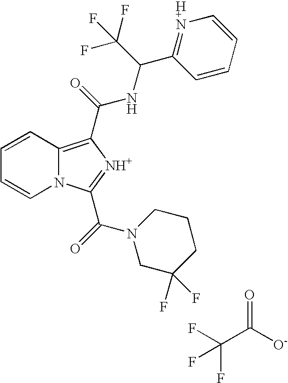 | 3-[(3,3-difluoropiperidin-1- yl)carbonyl]-N-(2,2,2-trifluoro-1- pyridin-2-ylethyl)imidazo[1,5- a]pyridine-1-carboxamide | 468.1438 |
| 628 | 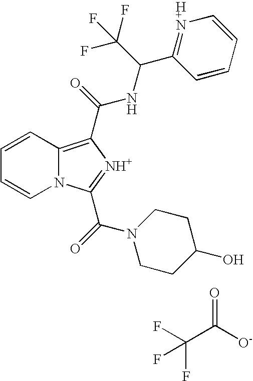 | 3-[(4-hydroxypiperidin-1- yl)carbonyl]-N-(2,2,2-trifluoro-1- pyridin-2-ylethyl)imidazo[1,5- a]pyridine-1-carboxamide | 448.1577 |
| 629 | 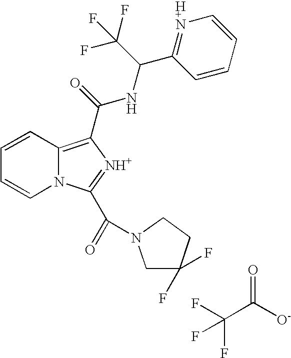 | 3-[(3,3-difluoropyrrolidin-1- yl)carbonyl]-N-(2,2,2-trifluoro-1- pyridin-2-ylethyl)imidazo[1,5- a]pyridine-1-carboxamide | 454.1283 |
| 630 | 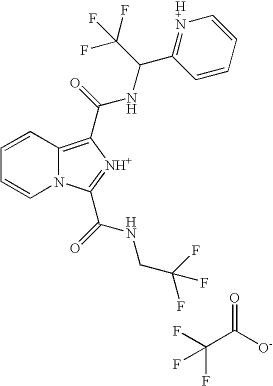 | N~3~-(2,2,2-trifluoroethyl)N~1~- (2,2,2-trifluoro-1-pyridin-2- ylethyl)imidazo[1,5-a]pyridine-1,3- dicarboxamide | 446.1032 |

What is claimed is:

1. A compound of Formula (I) or Formula (II): 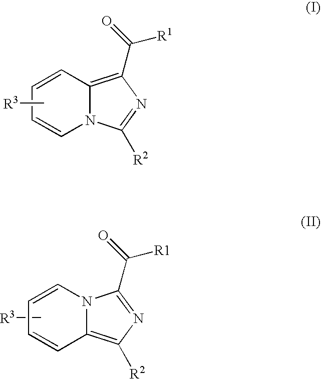
or a pharmaceutically acceptable salt thereof, wherein
R¹ is selected from the group consisting of:
(1) —NH—R⁴,
(2) —O—R⁴,
(3) —CH2-C(O)—R⁴, and
(4) —R⁴;
R² is selected from the group consisting of:
(1) H,
(2) halo,
(3) —CN,
(4) —CF₃,
(5) —C1-6alkyl,
(6) —C(O)—NH—C₁₋₃alkyl-CF₃,
(7) —C(O)—NH—C₁₋₃alkyl-heteroaryl, wherein the heteroaryl is optionally mono, di or tri-substituted with substituents independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl, is optionally mono or di-substituted with substituents independently selected from halo, —CH₃, —CF₃, —CN and —OC₁₋₆alkyl,
(8) —C(O)-heteroaryl, wherein the heteroaryl is optionally mono, di or tri-substituted with substituents independently selected from —CN, —CH₃, —CF₃, —CHF₂, —O₁₋₆alkyl, —O—CF₃, hydroxy, —CH₂—OH, halo, —S(O)₂—CH₃, —C(O)—O—C₁₋₆alkyl-C(O)—NHC₁₋₆alkyl, —C(O)—N(C₁₋₆alkyl)₂, —C(O)—O—C(CH₃)₃, —C₃₋₆cycloalkyl, —NH₂, —NH₂—C(O)—CF₃, —NH₂—C(O)—N(CH₃)₂, —NC(O)—NH₂, —NH—S(O)₂—CH₃ and —C(O)-heteroaryl optionally mono or di-substituted with substituents independently selected from halo, —CH₃, —CF₃, —CN and —OC₁₋₆alkyl,
(9) —C(O)-heterocycle, wherein the heterocycle is optionally mono, di- or tri-substituted with substituents selected from —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and oxo, wherein the heteroaryl portion of —C(O)-heteroaryl, is optionally mono or di-substituted with substituents independently selected from halo, —CH₃, —CF₃, —CN and —OC₁₋₆alkyl,
(10) —NR⁵R⁶,
(11) —C₁₋₄alkyl-NR⁵R⁶,
(12) —C₁₋₄alkyl-heterocycle, wherein the alkyl is optionally substituted with hydroxyl and wherein the heterocycle is optionally mono, di- or tri-substituted with substituents independently selected from —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, oxo and —C(O)-heteroaryl, wherein the heteroaryl portion of —C(O)-heteroaryl, is optionally mono or di-substituted with substituents independently selected from halo, —CH₃, —CF₃, —CN and —OC₁₋₆alkyl,
(13) —C₁₋₄alkyl-heteroaryl, optionally mono, di- or tri-substituted with substituents independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl, is optionally mono or di-substituted with substituents independently selected from halo, —CH₃, —CF₃, —CN and —OC₁₋₆alkyl,
(14) heterocycle, optionally mono, di- or tri-substituted with substituents independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, and oxo, wherein the heteroaryl portion of —C(O)-heteroaryl, is optionally mono or di-substituted with substituents independently selected from halo, —CH₃, —CF₃, —CN and —OC₁₋₆alkyl,
(15) heteroaryl, optionally mono, di- or tri-substituted with substituents independently selected from —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl, is optionally mono or di-substituted with substituents independently selected from halo, —CH₃, —CF₃, —CN and —OC₁₋₆alkyl,
(16) aryl, optionally mono, di- or tri-substituted with substituents independently selected from the group consisting of wherein the aryl is optionally mono or di-substituted with substituents selected from —CH₃, —O—C₁₋₆alkyl, hydroxy, —CH₂—OH, halo, —S(O)₂—CH₃, C(O)—O—C₁₋₆alkyl, —C(O)—NHC₁₋₆alkyl, —C(O)—N(C₁₋₆alkyl)₂, —(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, wherein the heteroaryl portion of —C(O)-heteroaryl, is optionally mono or di-substituted with substituents independently selected from halo, —CH₃, —CF₃, —CN and —OC₁₋₆alkyl,
(17) O-aryl, optionally mono, di- or tri-substituted with substituents independently selected from —CH₃, —OC₁₋₆alkyl, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, wherein the heteroaryl portion of —C(O)-heteroaryl, is optionally mono or di-substituted with substituents independently selected from halo, —CH₃, —CF₃, —CN and —OC₁₋₆alkyl,
(18) —O-heteroaryl, optionally mono, di- or tri-substituted with substituents independently selected from —CN, —CH₃, —CF₃, —CHF₂, —O—C₁₋₆alkyl, —O—CF₃, hydroxy, —CH₂—OH, halo, —S(O)₂—CH₃, —C(O)—O—C₁₋₆alkyl, —C(O)—N(CH₃)2, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl, is optionally mono or di-substituted with substituents independently selected from halo, —CH₃, —CF₃, —CN and —OC₁₋₆alkyl,
(19) —NH-heteroaryl, wherein the heteroaryl is optionally mono, di- or tri-substituted with substituents independently selected from —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl, is optionally mono or di-substituted with substituents independently selected from halo, —CH₃, —CF₃, —CN and —OC₁₋₆alkyl, and
(20) —C₃₋₆cycloalkyl, optionally mono, di- or tri-substituted with substituents selected —CH₃, —O—C₁₋₆alkyl, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, oxo, C(O)—O—C(CH₃)₃, —C₃₋₆cycloalkyl, —NH₂, —NH₂—C(O)—CF₃, —NH₂—C(O)—N(CH₃)₂—NC(O)—NH₂, —NH—S(O)₂—CH₃, —O—CF₃, —CF₃ and —CN;
R³ is selected from the group consisting of
(1) H,
(2) halo,
(3) —C₁₋₄alkyl, optionally substituted with hydroxyl,
(4) —CF₃, and
(5) —OC₁₋₆alkyl;
(6) —CN,
(7) —CHF₂,
(8) —O—CF₃,
(9) hydroxy,
(10) —S(O)₂—CH₃,
(11) —C(O)—O—C₁₋₆alkyl,
(12) —C(O)—NHC₁₋₆alkyl,
(13) —C(O)—N(C₁₋₆alkyl)₂,
(14) —C(O)—O—C(CH₃)₃,
(15) —C(O)-heteroaryl,
(16) —C₃₋₆cycloalkyl,
(17) —NH₂,
(18) —NH₂—C(O)—CF₃,
(19) —NH₂—C(O)—N(CH₃)₂,
(20) —NC(O)—NH₂,
(21) —NH—S(O)₂—CH₃,
(22) heteroaryl, optionally mono, di- or tri-substituted with substituents independently selected from —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl, is optionally mono or di-substituted with substituents independently selected from halo, —CH₃, —CF₃, —CN and —OC₁₋₆alkyl, and
(23) aryl, optionally mono, di- or tri-substituted with substituents selected from the group consisting of wherein the aryl is optionally mono or di-substituted with substituents selected from —CH₃, —O—C₁₋₆alkyl, hydroxy, —CH₂—OH, halo, —S(O)₂—CH₃, C(O)—O—C₁₋₆alkyl, —C(O)—NHC₁₋₆alkyl, —C(O)—N(C₁₋₆alkyl)₂, —(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, wherein the heteroaryl portion of —C(O)-heteroaryl, is optionally mono or di-substituted with substituents independently selected from halo, —CH₃, —CF₃, —CN and —OC₁₋₆alkyl;
R⁴ is selected from the group consisting of:
(1) adamantane, optionally mono and di-substituted with substituents independently selected from —CH₃ and hydroxyl,
(2) aryl, optionally mono, di- or tri-substituted with substituents independently selected from —CH₃, —OC₁₋₆alkyl, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, wherein the heteroaryl portion of —C(O)-heteroaryl is optionally mono, di- or tri-substituted with substituents selected from the group consisting of halo, —CH₃, —CF₃, —CN, —OC₁₋₆alkyl,
(3) heteroaryl, optionally mono, di- or tri-substituted with a substituents independently selected from —CN, —CH₃, —CF₃, —CHF₂, —OC₁₋₆alkyl, —O—CF3, hydroxy, —CH₂—OH, halo, —S(O)₂—CH₃, —C(O)—O—CH₃, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl is optionally mono, di- or tri-substituted with substituents selected from the group consisting of halo, —CH₃, —CF₃, —CN, —OC₁₋₆alkyl,
(4) heterocycle, optionally mono, di-, tri- or tetra substituted with a substituent selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, oxo and aryl, and wherein aryl and the heteroaryl portion of —C(O)-heteroaryl is optionally mono, di- or tri-substituted with substituents selected from the group consisting of halo, —CH₃, —CF₃, —CN, —OC₁₋₆alkyl,
(5) C₃₋₆cycloalkyl, optionally mono, di-, tri- or tetra substituted with substituents independently selected from the group consisting of hydroxyl, halo, phenyl,
(6) C₅₋₁₀carbocycle, wherein the carbocycle is optionally mono, di- or tri-substituted with substituents independently selected from halo, hydroxyl, —OC₁₋₆alkyl, CF₃,
(7) C₁₋₆alkyl, optionally mono or di-substituted with substituents independently selected from the group consisting of CF₃, hydroxyl, —CN, —CHF2,
(8) C₁₋₆alkyl-C₃₋₆cycloalkyl, wherein the cycloalkyl is optionally mono or di-substituted with substituents independently selected from hydroxyl, phenyl, —CH₂OH, and —C₃₋₆cycloalkyl,
(9) —C₁₋₆alkyl-heteroaryl, wherein the heteroaryl is optionally mono, di- or tri-substituted with substitutents independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and the C₁₋₆alkyl is optionally mono or di-substituted with a substitutent selected from the group consisting of —CF₃, —CHF₂, hydroxyl and —OC₁₋₆alkyl, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(10) —C₁₋₆alkyl-aryl, wherein the aryl is optionally mono, di- or tri-substituted with substitutents independently selected from —CH₃, —OC₁₋₆alkyl, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and the C₁₋₆alkyl is optionally mono or di-substituted with a substitutent selected from the group consisting of —CF₃, —CHF₂, hydroxyl and —OC₁₋₆alkyl, and wherein heteroaryl portion of —C(O)-heteroaryl is optionally mono, di- or tri-substituted with substituents selected from the group consisting of halo, —CH₃, —CF₃, —CN, —OC₁₋₆alkyl, and
(11) —C₁₋₆alkyl-heterocycle, wherein the heterocycle is optionally mono, di- or tri-substituted with substitutents independently selected from the —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, oxo and aryl, and the C₁₋₆alkyl is optionally mono or di-substituted with a substitutent selected from the group consisting of —CF₃, —CHF₂, hydroxyl and —OC₁₋₆alkyl and wherein aryl and the heteroaryl portion of —C(O)-heteroaryl is optionally mono, di- or tri-substituted with substituents independently selected from the group consisting of halo, —CH₃, —CF₃, —CN, —OC₁₋₆alkyl;
R⁵ is selected from the group consisting of hydrogen and methyl;
R⁶ is selected from the group consisting of:
(1) C₁₋₄alkyl, optionally mono or di-substituted, with substituents independently selected from the group consisting of C₃₋₆cycloalkyl, —CF₃, heteroaryl, —C₁₋₃alkyl-CF₃, CH₃, tetrahydrofuran, and
(2) —C₁₋₃alkyl-C₃₋₆cycloalkyl, wherein the cycloalkyl is optionally mono or di-substituted with substituents independently selected from the group consisting of halo, CF₃, CH₃, C₁₋₃alkyl, —OC₁₋₆alkyl, or
R5 and R6 are joined together so that along with the nitrogen to which they are attached, there is formed a heterocycle, wherein said heterocycle is optionally mono, di or tri-substituted with substituents independently selected from the group consisting of —OC₁₋₆alkyl, —NH—C(O)—O—C(CH₃)₃, hydroxy, —CH₃, —CF₃, —CH₂—OH, halo, —S(O)₂—CH₃, C(O)—O—C₁₋₆alkyl, —C(O)—N(CH₃)2, oxo, —C(O)—O—C(CH₃)₃, —C(O)-heteroaryl, —C₃₋₆cycloalkyl, —NH₂, —NH—C(O)—CF₃, —C(O)—NHC₁₋₆alkyl, —C(O)—N(C₁₋₆alkyl)₂, —NC(O)—NH₂, —NH—S(O)₂—CH₃, —O—CF₃, —S—CH₃, and wherein the heteroaryl portion of —C(O)-heteroaryl is optionally mono- di- or tri-substituted with substituents selected from the group consisting of halo, —CH₃, —CF₃, —CN and —O—C₁₋₆alkyl;
provided that when the compound is of Formula (II), and R¹ is optionally substituted aryl, then R² is other than hydrogen, halo, cyano, optionally substituted aryl, optionally substituted heteroaryl or NR⁵R⁶ wherein both of R⁵ and R⁶ are hydrogen, or unsubstituted alkyl.

2. A compound according to claim 1 wherein
R¹ is selected from the group consisting of:
(1) —NH—R⁴,
(2) —O—R⁴, and
(3) —R⁴.

3. A compound according to claim 2 wherein
R¹ is selected from the group consisting of:
(1) —NH—R⁴, and
(2) —R⁴.

4. A compound according to claim 1 wherein
R² is selected from the group consisting of:
(1) H,
(2) halo,
(3) —CN,
(4) —CF3,
(5) —C1-6alkyl,
(6) —C(O)-heterocycle, wherein the heterocycle is optionally mono, di- or tri-substituted with substituents independently selected from —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and oxo, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(7) —NR⁵R⁶,
(8) —C₁₋₄alkyl-NR⁵R⁶,
(9) —C₁₋₄alkyl-heterocycle, wherein the alkyl is optionally substituted with hydroxyl and wherein the heterocycle is optionally mono, di- or tri-substituted with substituents independently selected from —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, and oxo, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(10) —C₁₋₄alkyl-heteroaryl, optionally mono, di- or tri-substituted with substituents independently selected —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(11) heterocycle, optionally mono, di- or tri-substituted with substituents independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, and oxo, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(12) heteroaryl, optionally mono, di- or tri-substituted with substituents independently selected from —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and wherein the heteroaryl portion of —C(O)-heteroaryl is optionally mono, di- or tri-substituted with substituents selected from the group consisting of halo, —CH₃, —CF₃, —CN, —OC₁₋₆alkyl
(13) aryl, optionally mono, di- or tri-substituted with substituents independently selected from the group consisting of wherein the aryl is optionally mono or di-substituted with substituents selected from —CH₃, —O—C₁₋₆alkyl, hydroxy, —CH₂—OH, halo, —S(O)₂—CH₃, C(O)—O—C₁₋₆alkyl, —C(O)—NHC₁₋₆alkyl, —C(O)—N(C₁₋₆alkyl)₂, —(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and wherein the heteroaryl portion of —C(O)-heteroaryl is optionally mono, di- or tri-substituted with substituents selected from the group consisting of halo, —CH₃, —CF₃, —CN, —OC₁₋₆alkyl, and
(14) —C₃₋₆cycloalkyl, optionally mono or di-substituted with substituents independently selected from —CH₃, —O—C₁₋₆alkyl, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, oxo, C(O)—O—C(CH₃)₃, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN.

5. A compound according to claim 4 wherein
R² is selected from the group consisting of:
(1) H,
(2) —CF3,
(3) —C(O)-heterocycle, wherein the heterocycle is optionally mono, di- or tri-substituted with substituents independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and oxo, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(4) —NR⁵R⁶,
(5) —C₁₋₄alkyl-NR⁵R⁶,
(6) —C₁₋₄alkyl-heterocycle, wherein the alkyl is optionally substituted with hydroxyl and wherein the heterocycle is optionally mono, di- or tri-substituted with substituents independently selected from —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, and oxo, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(7) heterocycle, optionally mono, di- or tri-substituted with substituents independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, and oxo, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(8) heteroaryl, optionally mono, di- or tri-substituted with substituents independently selected from —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and wherein the heteroaryl portion of —C(O)-heteroaryl is optionally mono, di- or tri-substituted with substituents selected from the group consisting of halo, —CH₃, —CF₃, —CN, —OC₁₋₆alkyl, and
(9) aryl, optionally mono, di- or tri-substituted with substituents independently selected from the group consisting of —CH₃, —O—C₁₋₆alkyl, hydroxy, —CH₂—OH, halo, —S(O)₂—CH₃, C(O)—O—C₁₋₆alkyl, —C(O)—NHC₁₋₆alkyl, —C(O)—N(C₁₋₆alkyl)₂, —(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and wherein the heteroaryl portion of —C(O)-heteroaryl is optionally mono, di- or tri-substituted with substituents selected from the group consisting of halo, —CH₃, —CF₃, —CN, —OC₁₋₆alkyl.

6. A compound according to claim 5 wherein
R² is selected from the group consisting of:
(1) —CF3,
(2) —C₁₋₄alkyl-heterocycle, wherein the alkyl is optionally substituted with hydroxyl and wherein the heterocycle is optionally mono, di- or tri-substituted with substituents independently selected from —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, and oxo, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(3) heterocycle, optionally mono, di- or tri-substituted with substituents independently selected from —CN, —CH₃, —CF₃, —CHF₂—OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, and oxo, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(4) heteroaryl, optionally mono, di- or tri-substituted with substituents independently selected from —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and wherein the heteroaryl portion of —C(O)-heteroaryl is optionally mono, di- or tri-substituted with substituents selected from the group consisting of halo, —CH₃, —CF₃, —CN, —OC₁₋₆alkyl, and
(5) aryl, optionally mono, di- or tri-substituted with substituents independently selected from the group consisting of wherein the aryl is optionally mono or di-substituted with substituents selected from —CH₃, —O—C₁₋₆alkyl, hydroxy, —CH₂—OH, halo, —S(O)₂—CH₃, C(O)—O—C₁₋₆alkyl, —C(O)—NHC₁₋₆alkyl, —C(O)—N(C₁₋₆alkyl)₂, —(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and wherein the heteroaryl portion of —C(O)-heteroaryl is optionally mono, di- or tri-substituted with substituents selected from the group consisting of halo, —CH₃, —CF₃, —CN, —OC₁₋₆alkyl.

7. A compound according to claim 1 wherein
R³ is selected from the group consisting of
(1) H,
(2) halo,
(3) —C₁₋₄alkyl, optionally substituted with hydroxyl,
(4) —CF₃,
(5) —OC₁₋₆alkyl, and
(6) —CN.

8. A compound according to claim 7 wherein
R³ is selected from the group consisting of
(1) H, and
(2) halo.

9. A compound according to claim 1 wherein
R⁴ is selected from the group consisting of:
(1) adamantane, optionally mono and di-substituted with substituents independently selected from —CH₃ and hydroxyl,
(2) aryl, optionally mono, di- or tri-substituted with substituents independently selected from —CH₃, —OC₁₋₆alkyl, hydroxy, —CH₃—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and wherein the heteroaryl portion of —C(O)-heteroaryl is optionally mono, di- or tri-substituted with substituents independently selected from the group consisting of halo, —CH₃, —CF₃, —CN, —OC₁₋₆alkyl,
(3) heteroaryl, optionally mono or di-substituted with a substituents independently selected from —CN, —CH₃, —CF₃, —CHF₂, —OC₁₋₆alkyl, —O—CF3, hydroxy, —CH₃—OH, halo, —S(O)₂—CH₃, —C(O)—O—CH₃, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(4) heterocycle, optionally mono, di-, tri- or tetra substituted with substituents independently selected from —CN, —CH₃, —CF₃, —CHF₂, —OC₁₋₆alkyl, —O—CF3, hydroxy, —CH₃—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, oxo and aryl, and wherein aryl and the heteroaryl portion of —C(O)-heteroaryl is optionally mono, di- or tri-substituted with substituents selected from the group consisting of halo, —CH₃, —CF₃, —CN, —OC₁₋₆alkyl,
(5) C₁₋₆alkyl-C₃₋₆cycloalkyl, wherein the cycloalkyl is optionally mono or di-substituted with substituents selected from hydroxyl, phenyl, —CH₂OH, —C₃₋₆cycloalkyl,
(6) —C₁₋₆alkyl-heteroaryl, wherein the heteroaryl is optionally mono, di- or tri-substituted with substitutents independently selected from —CN, —CH₃, —CF₃, —CHF₂, —OC₁₋₆alkyl, —O—CF3, hydroxy, —CH₃—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and the C₁₋₆alkyl is optionally mono or di-substituted with substitutents independently selected from the group consisting of —CF₃, —CHF₂, hydroxyl and —OC₁₋₆alkyl, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(7) —C₁₋₆alkyl-aryl, wherein the aryl is optionally mono, di- or tri-substituted with substitutents independently selected from —CH₃, —OC₁₋₆alkyl, hydroxy, —CH₃—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and the C₁₋₆alkyl is optionally mono or di-substituted with a substitutent selected from the group consisting of —CF₃, —CHF₂, hydroxyl and —OC₁₋₆alkyl, and wherein the heteroaryl portion of —C(O)-heteroaryl is optionally mono, di- or tri-substituted with substituents selected from the group consisting of halo, —CH₃, —CF₃, —CN, —OC₁₋₆alkyl, and
(8) —C₁₋₆alkyl-heterocycle, wherein the heterocycle is optionally mono, di- or tri-substituted with substitutents independently selected from the —CN, —CH₃, —CF₃, —CHF₂, —OC₁₋₆alkyl, —O—CF3, hydroxy, —CH₃—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, oxo and aryl, and the C₁₋₆alkyl is optionally mono or di-substituted with a substitutent selected from the group consisting of —CF₃, —CHF₂, hydroxyl and —OC₁₋₆alkyl, and wherein aryl and the heteroaryl portion of —C(O)-heteroaryl is optionally mono, di- or tri-substituted with substituents selected from the group consisting of halo, —CH₃, —CF₃, —CN, —OC₁₋₆alkyl.

10. A compound according to claim 9 wherein
R⁴ is selected from the group consisting of:
(1) aryl, optionally mono, di- or tri-substituted with substituents independently selected from —CH₃, —OC₁₋₆alkyl, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and wherein the heteroaryl portion of —C(O)heteroaryl is optionally mono or di-substituted with substituents independently selected from the group consisting of halo, —CH₃, —CF₃, —CN, —OC₁₋₆alkyl,
(2) heterocycle, optionally mono, di-, tri- or tetra substituted with substituents independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, oxo and aryl, and wherein aryl and the heteroaryl portion of —C(O)-heteroaryl is optionally mono, di- or tri-substituted with substituents selected from the group consisting of halo, —CH₃, —CF₃, —CN, —OC₁₋₆alkyl,
(3) —C₁₋₆alkyl-heteroaryl, wherein the heteroaryl is optionally mono, di- or tri-substituted with substitutents independently selected from —CN, —CH₃, —CF₃, —CHF₂, —OC₁₋₆alkyl, —O—CF3, hydroxy, —CH₃—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and the C₁₋₆alkyl is optionally mono or di-substituted with substitutents independently selected from the group consisting of —CF₃, —CHF₂, hydroxyl and —OC₁₋₆alkyl, and wherein heteroaryl portion of —C(O)-heteroaryl is optionally mono, di- or tri-substituted with substituents selected from the group consisting of halo, —CH₃, —CF₃, —CN, —OC₁₋₆alkyl,
(4) —C₁₋₆alkyl-aryl, wherein the aryl is optionally mono, di- or tri-substituted with substitutents independently selected from —CH₃, —OC₁₋₆alkyl, hydroxy, —CH₃—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and the C₁₋₆alkyl is optionally mono or di-substituted with substitutents independently selected from the group consisting of —CF₃, —CHF₂, hydroxyl and —OC₁₋₆alkyl and wherein the heteroaryl portion of —C(O)-heteroaryl is optionally mono, di- or tri-substituted with substituents selected from the group consisting of halo, —CH₃, —CF₃, —CN, —OC₁₋₆alkyl.

11. A compound according to claim 1 wherein
R5 and R6 are joined together so that along with the nitrogen to which they are attached, there is formed a heterocycle, wherein said heterocycle is optionally mono, di or tri-substituted with substituents independently selected from the group consisting of —OC₁₋₆alkyl, —NH—C(O)—O—C(CH₃)₃, hydroxy, —CH₃, —CF₃, —CH₂—OH, halo, —S(O)₂—CH₃, C(O)—O—C₁₋₆alkyl, —C(O)—N(CH₃)₂, oxo, —C(O)—O—C(CH₃)₃, —C(O)-heteroaryl, —C₃₋₆cycloalkyl, —NH₂, —NH—C(O)—CF₃, —C(O)—NHC₁₋₆alkyl, —C(O)—N(C₁₋₆alkyl)₂, —NC(O)—NH₂, —NH—S(O)₂—CH₃, —O—CF₃, —S—CH₃, and wherein the heteroaryl portion of —C(O)-heteroaryl is optionally mono- or di-substituted with substituents independently selected from the group consisting of halo, —CH₃, —CF₃, —CN and —O—C₁₋₆alkyl.

12. A compound according to claim 1 wherein
R¹ is selected from the group consisting of:
(1) —NH—R⁴, and
(2) —R⁴;
R² is selected from the group consisting of:
(1) —CF3,
(2) —C₁₋₄alkyl-heterocycle, wherein the alkyl is optionally substituted with hydroxyl and wherein the heterocycle is optionally mono, di- or tri-substituted with substituents independently selected from —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, and oxo, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(3) heterocycle, optionally mono, di- or tri-substituted with substituents independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, and oxo, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(4) heteroaryl, optionally mono, di- or tri-substituted with substituents independently selected from —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and wherein the heteroaryl portion of —C(O)-heteroaryl is optionally mono, di- or tri-substituted with substituents selected from the group consisting of halo, —CH₃, —CF₃, —CN, —OC₁₋₆alkyl, and
(5) aryl, optionally mono, di- or tri-substituted with substituents independently selected from the group consisting of —CH₃, —O—C₁₋₆alkyl, hydroxy, —CH₂—OH, halo, —S(O)₂—CH₃, C(O)—O—C₁₋₆alkyl, —C(O)—NHC₁₋₆alkyl, —C(O)—N(C₁₋₆alkyl)₂, —(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and wherein the heteroaryl portion of —C(O)-heteroaryl is optionally mono, di- or tri-substituted with substituents selected from the group consisting of halo, —CH₃, —CF₃, —CN, —OC₁₋₆alkyl;
R³ is selected from the group consisting of
(1) H, and
(2) halo; and
R⁴ is selected from the group consisting of:
(1) aryl, optionally mono, di- or tri-substituted with substituents independently selected from —CH₃, —OC₁₋₆alkyl, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and wherein the heteroaryl portion of —C(O)-heteroaryl is optionally mono, di- or tri-substituted with substituents independently selected from the group consisting of halo, —CH₃, —CF₃, —CN, —OC₁₋₆alkyl,
(2) heterocycle, optionally mono, di-, tri- or tetra substituted with substituents independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, oxo and aryl, and wherein aryl and the heteroaryl portion of —C(O)-heteroaryl is optionally mono, di- or tri-substituted with substituents selected from the group consisting of halo, —CH₃, —CF₃, —CN, —OC₁₋₆alkyl,
(3) —C₁₋₆alkyl-heteroaryl, wherein the heteroaryl is optionally mono, di- or tri-substituted with substitutents independently selected from —CN, —CH₃, —CF₃, —CHF₂, —OC₁₋₆alkyl, —O—CF₃, hydroxy, —CH₃—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and the C₁₋₆alkyl is optionally mono or di-substituted with substitutents independently selected from the group consisting of —CF₃, —CHF₂, hydroxyl and —OC₁₋₆alkyl, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(4) —C₁₋₆alkyl-aryl, wherein the aryl is optionally mono, di- or tri-substituted with substitutents independently selected from —CH₃, —OC₁₋₆alkyl, hydroxy, —CH₃—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and the C₁₋₆alkyl is optionally mono or di-substituted with substitutents independently selected from the group consisting of —CF₃, —CHF₂, hydroxyl and —OC₁₋₆alkyl, and wherein the heteroaryl portion of —C(O)-heteroaryl is optionally mono, di- or tri-substituted with substituents selected from the group consisting of halo, —CH₃, —CF₃, —CN, —OC₁₋₆alkyl.

13. A compound according to claim 1 wherein
R¹ is selected from the group consisting of:
(1) —NH—R⁴, and
(2) —R⁴;
R² is selected from the group consisting of:
(1) —NR⁵R⁶,
(2) —C₁₋₄alkyl-NR⁵R⁶,
(3) heteroaryl, optionally mono, di- or tri-substituted with substituents independently selected from —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and wherein the heteroaryl portion of —C(O)-heteroaryl is optionally mono, di- or tri-substituted with substituents selected from the group consisting of halo, —CH₃, —CF₃, —CN, —OC₁₋₆alkyl and
(4) aryl, optionally mono, di- or tri-substituted with substituents independently selected from the group consisting of wherein the aryl is optionally mono or di-substituted with substituents selected from —CH₃, —O—C₁₋₆alkyl, hydroxy, —CH₂—OH, halo, —S(O)₂—CH₃, C(O)—O—C₁₋₆alkyl, —C(O)—NHC₁₋₆alkyl, —C(O)—N(C₁₋₆alkyl)₂, —(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and wherein the heteroaryl portion of —C(O)-heteroaryl is optionally mono, di- or tri-substituted with substituents selected from the group consisting of halo, —CH₃, —CF₃, —CN, —OC₁₋₆alkyl;
R³ is selected from the group consisting of
(1) H, and
(2) halo; and
R⁴ is selected from the group consisting of:
(1) aryl, optionally mono, di- or tri-substituted with substituents independently selected from —CH₃, —OC₁₋₆alkyl, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, C(O)-heteroaryl, —C3-6cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and wherein the heteroaryl portion of —C(o)-heteroaryl is optionally mono, di- or tri-substituted with substituents independently selected from the group consisting of halo, —CH₃, —CF₃, —CN, —OC₁₋₆alkyl,
(2) heterocycle, optionally mono, di-, tri- or tetra substituted with a substituent independently selected from —CN, —CH₃, —CF₃, —CHF₂, —OC₁₋₆alkyl, —O—CF3, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, oxo and aryl, and wherein aryl and the heteroaryl portion of —C(O)-heteroaryl is optionally mono, di- or tri-substituted with substituents selected from the group consisting of halo, —CH₃, —CF₃, —CN, —OC₁₋₆alkyl,
(3) —C₁₋₆alkyl-heteroaryl, wherein the heteroaryl is optionally mono, di- or tri-substituted with substitutents independently selected from —CN, —CH₃, —CF₃, —CHF₂, —OC₁₋₆alkyl, —O—CF3, hydroxy, —CH₃—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, and the C₁₋₆alkyl is optionally mono or di-substituted with substitutents independently selected from the group consisting of —CF₃, —CHF₂, hydroxyl and —OC₁₋₆alkyl, and wherein the heteroaryl portion of —C(O)-heteroaryl is optionally mono, di- or tri-substituted with substituents selected from the group consisting of halo, —CH₃, —CF₃, —CN, —OC₁₋₆alkyl,
(4) —C₁₋₆alkyl-aryl, wherein the aryl is optionally mono, di- or tri-substituted with substitutents independently selected from —CH₃, —OC₁₋₆alkyl, hydroxy, —CH₃—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, and the C₁₋₆alkyl is optionally mono or di-substituted with substitutents independently selected from the group consisting of —CF₃, —CHF₂, hydroxyl and —OC₁₋₆alkyl, and wherein the heteroaryl portion of —C(O)-heteroaryl is optionally mono, di- or tri-substituted with substituents selected from the group consisting of halo, —CH₃, —CF₃, —CN, —OC₁₋₆alkyl; and
R5 and R6 are joined together so that along with the nitrogen to which they are attached, there is formed a heterocycle, wherein said heterocycle is optionally mono, di or tri-substituted with substituents independently selected from the group consisting of —OC₁₋₆alkyl, —NH—C(O)—O—C(CH₃)₃, hydroxy, —CH₃, —CF₃, —CH₂—OH, halo, —S(O)₂—CH₃, C(O)—O—C₁₋₆alkyl, —C(O)—N(CH₃)2, oxo, —C(O)—O—C(CH₃)₃, —C(O)-heteroaryl, —C₃₋₆cycloalkyl, —NH₂, —NH—C(O)—CF₃, —C(O)—NHC₁₋₆alkyl, —C(O)—N(C₁₋₆alkyl)₂, —NC(O)—NH₂, —NH—S(O)₂—CH₃, —O—CF₃, —S—CH₃, and wherein the heteroaryl portion of —C(O)-heteroaryl is optionally mono- di- or tri-substituted with substituents independently selected from the group consisting of halo, —CH₃, —CF₃, —CN and —O—C₁₋₆alkyl.

14. A compound according to claim 1 selected from the group consisting of

15. A pharmaceutical composition comprising a compound of claim 1 and a pharmaceutically acceptable carrier.

16. A method of modulating the CB2 receptor in a patient in need of such modulation, comprising administering an effective amount of a compound according to claim 1.

17. A method of agonizing the CB2 receptor in a patient in need of such agonizing, comprising administering an effective amount of a compound according to claim 1.

18. A method of treating a disease mediated by agonizing the CB2 receptor in a patient in need of such treatment, comprising administering an effective amount of a compound according to claim 1.

19. A method of treating a disease selected from the group consisting inflammatory pain, osteoporosis, atheroschlerosis, immune disorders and arthritis comprising administering an effective amount of a compound according to claim 1.

20. A method according to claim 19, for the treatment of the acute and chronic pain.

21. A method according to claim 21, for the treatment of the pain of rheumatoid arthritis or osteoarthritis.
